(19) United States
(12) Patent Application Publication
(10) Pub. No.: US 2025/0179000 A1
(43) Pub. Date: Jun. 5, 2025

(54) METABOTROPIC GLUTAMATE RECEPTOR POSITIVE ALLOSTERIC MODULATORS (PAMS) AND USES THEREOF

(71) Applicant: Sanford Burnham Prebys Medical Discovery Institute, La Jolla, CA (US)

(72) Inventors: Nicholas David Peter COSFORD, San Diego, CA (US); Douglas J. SHEFFLER, San Diego, CA (US); Dhanya RAVEENDRA-PANICKAR, San Diego, CA (US)

(21) Appl. No.: 18/723,093

(22) PCT Filed: Dec. 22, 2022
(86) PCT No.: PCT/US2022/053814

§ 371 Date: Jun. 21, 2024

Related U.S. Application Data
(60) Provisional application No. 63/293,515, filed on Dec. 23, 2021.

(51) Int. Cl. C07C 65/24; C07C 65/28; C07C 229/66; C07C 255/29; C07C 271/16; C07C 317/18; C07C 317/32; C07C 323/16; C07C 323/18; C07C 323/20; C07D 213/80; C07D 237/08; C07D 239/26; C07D 241/12; C07D 271/06; C07D 271/107; C07D 311/58; C07D 401/04; C07D 403/10; C07D 405/12; C07D 409/04; C07D 413/10; C07D 417/10

(52) U.S. Cl. CPC C07C 65/24; C07C 65/28; C07C 229/66; C07C 255/29; C07C 271/16; C07C 317/18; C07C 317/32; C07C 323/16; C07C 323/18; C07C 323/20; C07D 213/80; C07D 237/08; C07D 239/26; C07D 241/12; C07D 271/06; C07D 271/107; C07D 311/58; C07D 401/04; C07D 403/10; C07D 405/12; C07D 409/04; C07D 413/10; C07D 417/10; C07C 2601/02; C07C 2601/16

(57) ABSTRACT

Provided herein are small molecule active metabotropic glutamate subtype-2 and -3 receptor positive allosteric modulators (PAMS), compositions comprising the compounds, and methods of using the compounds and compositions comprising the compounds.

### CROSS REFERENCE

This application claims the benefit of U.S. Provisional Patent Application Ser. No. 63/293,515, filed Dec. 23, 2021; which is incorporated herein by reference in its entirety.

### STATEMENT OF GOVERNMENT SUPPORT

This invention was made with government support under R01 DA023926, U01 DA041731, and U01 DA051077 awarded by the National Institutes of Health. The government has certain rights in the invention.

### TECHNICAL FIELD

Described herein are metabotropic glutamate subtype-2 and -3 (mGlu2/3) (collectively Group II mGlus) receptor positive allosteric modulators, methods of making such compounds, pharmaceutical compositions and medicaments comprising such compounds, and methods of using such compounds in the treatment of conditions, diseases, or disorders in which metabotropic glutamate receptors are involved.

### SUMMARY

Described herein are compounds and compositions, and methods of using these compounds and compositions, as positive allosteric modulators of the metabotropic glutamate receptor subtype 2 receptor (mGlu2), and of the metabotropic glutamate receptor subtype 3 receptor (mGlu3) (collectively Group II mGlus), and for treating CNS disorders associated with Group II mGlus.

In one aspect, presented herein is a compound, or a pharmaceutically acceptable salt or solvate thereof, having the structure of Formula (I):

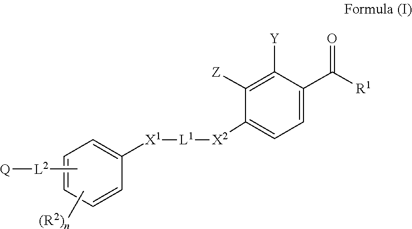

wherein:
R¹ is substituted or unsubstituted C₂-C₆ alkyl, substituted or unsubstituted C₁-C₆ fluoroalkyl, substituted or unsubstituted aryl, substituted or unsubstituted heteroaryl, substituted or unsubstituted cycloalkyl, or substituted or unsubstituted heterocycloalkyl;
X¹ is absent, —O—, —NR⁵—, —S—, —S(═O)—, or —S(═O)₂—;
X² is —O—, —NR⁶—, —S—, —S(═O)—, or —S(═O)₂—;
R⁵ and R⁶ are each independently hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
L¹ is substituted C₄ alkylene, substituted or unsubstituted C₅-C₁₀ alkylene, substituted or unsubstituted C₂-C₁₀ alkenylene, substituted or unsubstituted C₂-C₁₀ alkynylene, -L³-X³-L⁴-, or -L⁵-X⁴-L⁶-;
L³ and L⁴ are each independently substituted or unsubstituted C₁-C₆ alkylene, substituted or unsubstituted C₂-C₆ alkenylene, or optionally substituted or unsubstituted C₂-C₆ alkynylene;
X³ is —C(R⁸)₂—, —C(R⁹)═C(R⁹)—, —C≡C—, —O—, —NR⁷—, —S—, —S(═O)—, or —S(═O)₂—;
L⁵ and L⁶ are each —C(D)₂- and X⁴ is substituted or unsubstituted C₁-C₄ alkylene;
R⁷ is hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
each R⁸ is independently D, halogen, —OH, substituted or unsubstituted C₁-C₆alkyl, or substituted or unsubstituted C₁-C₆fluoroalkyl;
or two R⁸ are taken together with the carbon atom to which they are attached to form a substituted or unsubstituted cycloalkyl or a substituted or unsubstituted heterocycloalkyl;
each R⁹ is independently hydrogen, D, halogen, —OH, substituted or unsubstituted C₁-C₆alkyl, or substituted or unsubstituted C₁-C₆fluoroalkyl;
L² is absent or C₁-C₆ alkylene;
Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

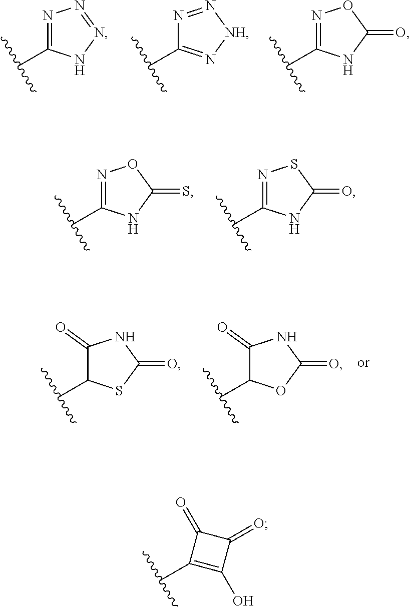

each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl;
n is 0, 1, 2, 3, or 4;
Y is —OH, —OR⁴, halogen, substituted or unsubstituted C₁-C₆alkyl, substituted or unsubstituted C₁-C₆fluoroalkyl, or substituted or unsubstituted cycloalkyl;
Z is —OH, —OR⁴, halogen, substituted or unsubstituted C₁-C₆alkyl, substituted or unsubstituted C₁-C₆fluoroalkyl, or substituted or unsubstituted cycloalkyl;
each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl;
or two R³ taken together with the nitrogen to which they are attached to form a substituted or unsubstituted C₂-C₈heterocycloalkyl;
each R⁴ is independently substituted or unsubstituted C₁-C₆alkyl, substituted or unsubstituted cycloalkyl substituted or unsubstituted aryl or substituted or unsubstituted heteroaryl.

In one embodiment, presented herein is a compound, or a pharmaceutically acceptable salt or solvate thereof, of Formula (I) wherein the compound of Formula (I) has the structure of Formula (Ia), or a pharmaceutically acceptable salt, or solvate thereof:

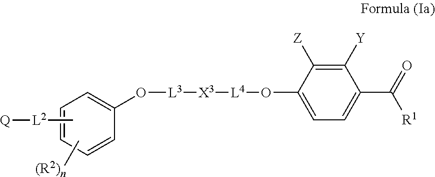

wherein:
L³ and L⁴ are each independently C₁-C₄alkylene;
X³ is —C(R⁸)₂, —C(R⁹)═C(R⁹)—, —C≡C—, —O—, —NR⁷—, —S—, —S(═O)—, or —S(═O)₂—;
each R⁸ is independently D, halogen, —OH, substituted or unsubstituted C₁-C₆alkyl, or substituted or unsubstituted C₁-C₆fluoroalkyl;
or two R⁸ are taken together with the carbon atom to which they are attached to form a substituted or unsubstituted cycloalkyl or a substituted or unsubstituted heterocycloalkyl;
each R⁹ is independently hydrogen, D, halogen, —OH, substituted or unsubstituted C₁-C₆alkyl, or substituted or unsubstituted C₁-C₆fluoroalkyl.

In one aspect, presented herein is a compound, or a pharmaceutically acceptable salt or solvate thereof, having the structure of Formula (II):

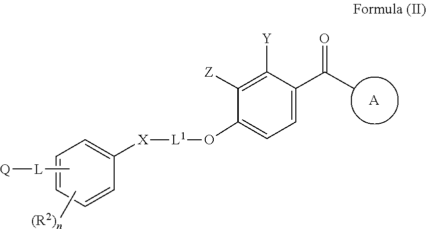

wherein:
ring A is a substituted or unsubstituted aryl, substituted or unsubstituted heteroaryl, substituted or unsubstituted cycloalkyl, or substituted or unsubstituted heterocycloalkyl;
X is —O— and L¹ is —(CH₂)₄—;
or X is substituted or unsubstituted arylene, substituted or unsubstituted heteroarylene, substituted or unsubstituted cycloalkylene, or substituted or unsubstituted heterocycloalkylene; and L¹ is —CH₂—;
L is absent or C₁-C₆ alkylene;
Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

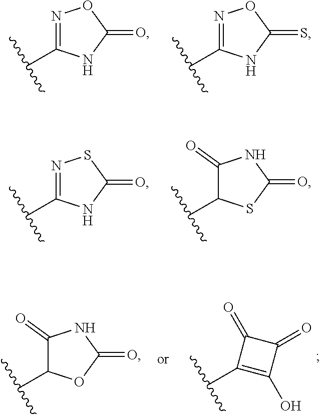

each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl;
n is 0, 1, 2, 3, or 4;
Y is —OH, —OR⁴, halogen, substituted or unsubstituted C₁-C₆alkyl, substituted or unsubstituted C₁-C₆fluoroalkyl, or substituted or unsubstituted cycloalkyl;
Z is —OH, —OR⁴, halogen, substituted or unsubstituted C₁-C₆alkyl, substituted or unsubstituted C₁-C₆fluoroalkyl, or substituted or unsubstituted cycloalkyl;
each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl;
or two R³ taken together with the nitrogen to which they are attached to form a substituted or unsubstituted C₂-C₈heterocycloalkyl; and
each R⁴ is independently substituted or unsubstituted C₁-C₆alkyl, substituted or unsubstituted cycloalkyl substituted or unsubstituted aryl or substituted or unsubstituted heteroaryl.

In one aspect, presented herein is a compound, or a pharmaceutically acceptable salt or solvate thereof, having the structure of Formula (III):

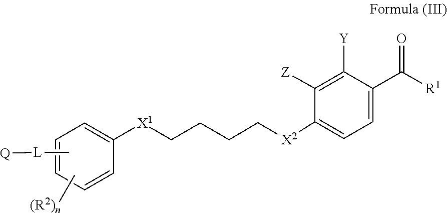

wherein;
R¹ is substituted or unsubstituted C₄-C₆ alkyl, substituted or unsubstituted C₁-C₆ fluoroalkyl, substituted or unsubstituted aryl, substituted or unsubstituted heteroaryl unsubstituted cycloalkyl, or substituted or unsubstituted heterocycloalkyl;
X¹ and X² are each independently —O—, —NR—, —S—, —S(═O)—, or —S(═O)₂—, wherein at least one of X¹ and X² is —NR⁵—, —S—, —S(═O)—, or —S(═O)₂—;
each R⁵ is independently hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
L is absent or C₁-C₆ alkylene;
Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

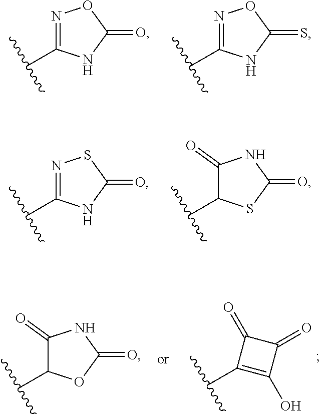

each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl;
n is 0, 1, 2, 3, or 4;
Y is —OH, —OR⁴, halogen, substituted or unsubstituted C₁-C₆alkyl, substituted or unsubstituted C₁-C₆fluoroalkyl, or substituted or unsubstituted cycloalkyl;
Z is —OH, —OR⁴, halogen, substituted or unsubstituted C₁-C₆alkyl, substituted or unsubstituted C₁-C₆fluoroalkyl, or substituted or unsubstituted cycloalkyl;
each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl;
or two R³ taken together with the nitrogen to which they are attached to form a substituted or unsubstituted C₂-C₈heterocycloalkyl; and
each R⁴ is independently substituted or unsubstituted C₁-C₆alkyl, substituted or unsubstituted cycloalkyl, substituted or unsubstituted aryl, or substituted or unsubstituted heteroaryl.

In one aspect, presented herein is a compound selected from the group consisting of:

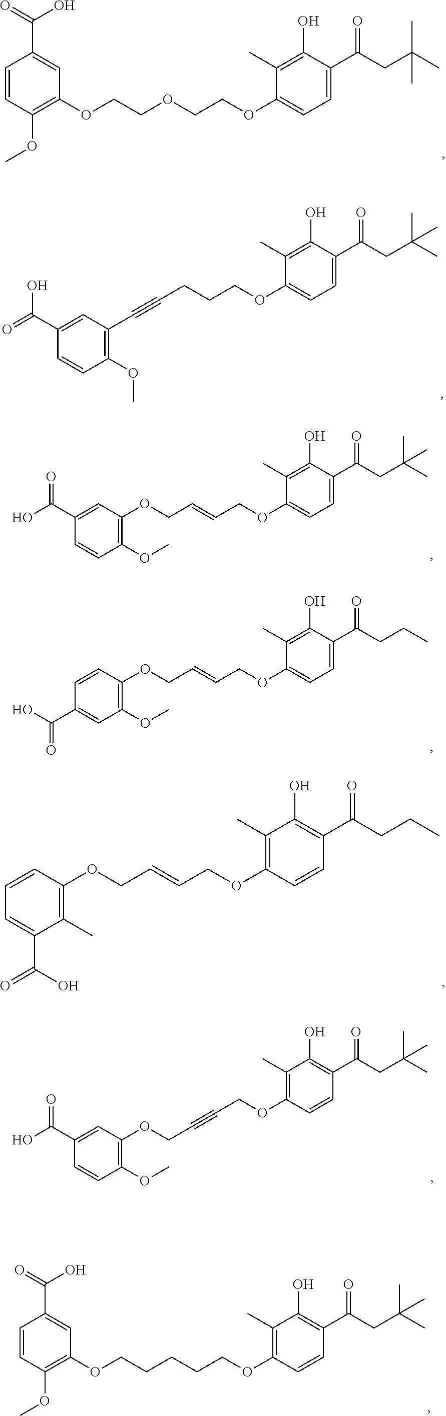

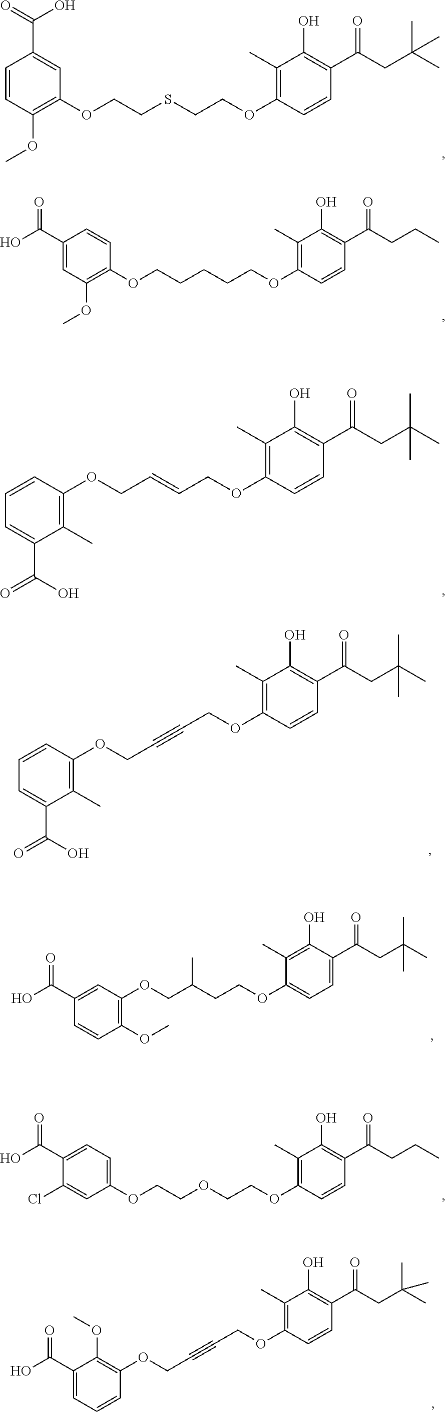

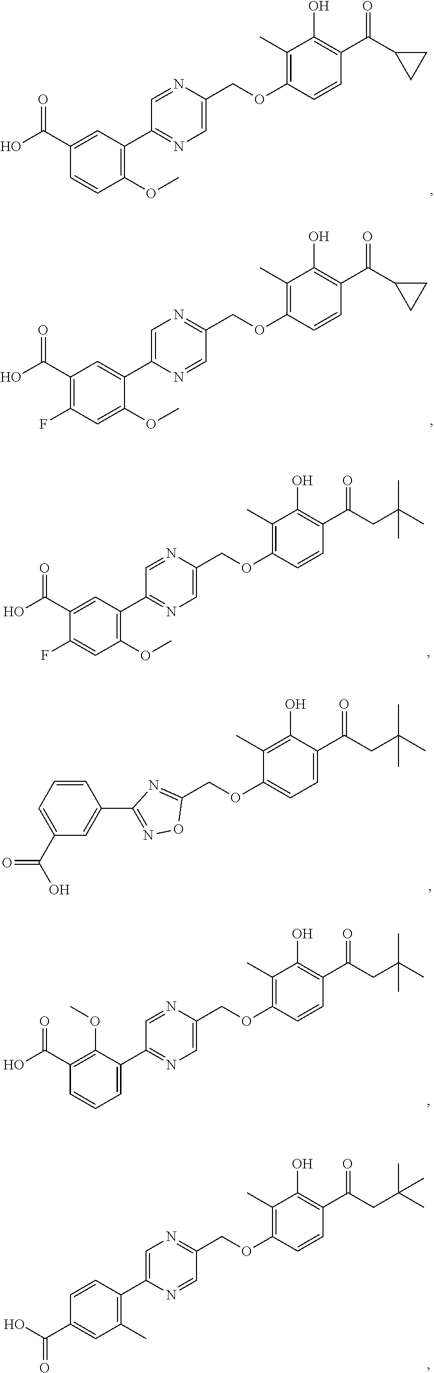 [SMILES: COc1ccc(C(=O)O)cc1-c1cnc(COc2ccc(C(=O)C3CC3)c(O)c2C)cn1 ; COc1cc(F)c(C(=O)O)cc1-c1cnc(COc2ccc(C(=O)C3CC3)c(O)c2C)cn1 ; COc1cc(F)c(C(=O)O)cc1-c1cnc(COc2ccc(C(=O)CC(C)(C)C)c(O)c2C)cn1 ; Cc1c(OCc2nc(-c3cccc(C(=O)O)c3)no2)ccc(C(=O)CC(C)(C)C)c1O ; COc1c(C(=O)O)cccc1-c1cnc(COc2ccc(C(=O)CC(C)(C)C)c(O)c2C)cn1 ; Cc1cc(C(=O)O)ccc1-c1cnc(COc2ccc(C(=O)CC(C)(C)C)c(O)c2C)cn1]

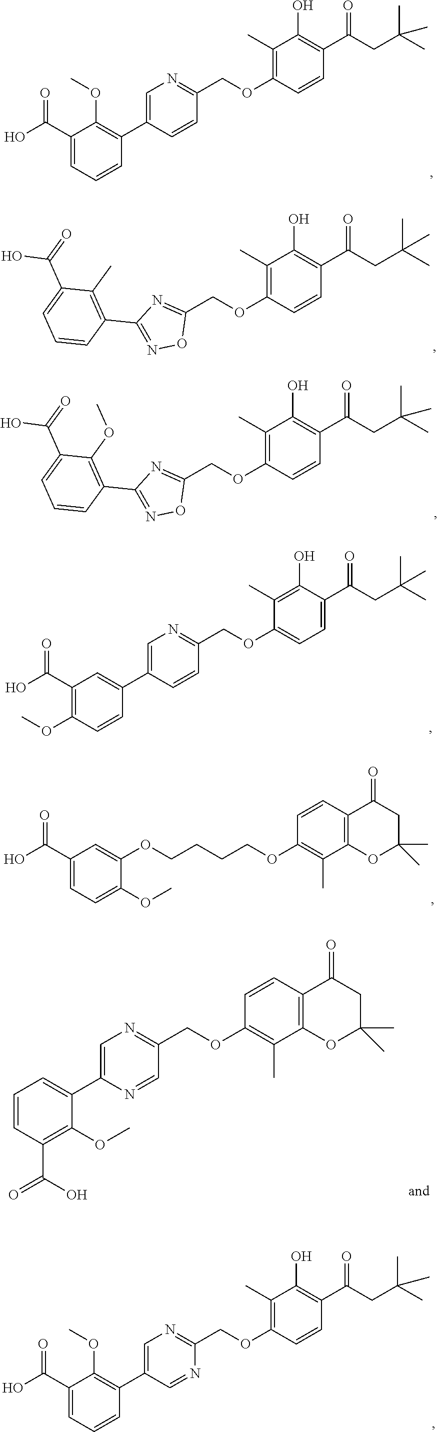

or a pharmaceutically acceptable salt or solvate thereof.

In one aspect, provided herein is a pharmaceutical composition comprising a compound of Formula (I), (Ia), (II), or (III), or a pharmaceutically acceptable salt thereof, and at least one pharmaceutically acceptable excipient.

In some embodiments, the pharmaceutical composition is formulated for administration to a mammal by intravenous administration, subcutaneous administration, oral administration, inhalation, nasal administration, dermal administration, or ophthalmic administration.

In some embodiments, the pharmaceutical composition is in the form of a tablet, a pill, a capsule, a liquid, a suspension, a gel, a dispersion, a solution, an emulsion, an ointment, or a lotion.

In one aspect, described herein is a method of treating a central nervous disorder (CNS) disorder, the method comprising the step of administering to a subject in need thereof, an effective amount of the compound of Formula (I), (Ia), (II), or (III), or a pharmaceutically acceptable salt thereof. In some embodiments, the CNS disorder is an addictive disorder. In some embodiments, the addictive disorder is nicotine addiction, alcohol addiction, opiate addiction, amphetamine addiction, methamphetamine addiction, or cocaine addiction. In some embodiments, the addictive disorder is nicotine addiction. In some embodiments, the addictive disorder is cocaine addiction. In some embodiments, the CNS disorder is schizophrenia. In some embodiments, the CNS disorder is a neurodegenerative disease. In some embodiments, the neurodegenerative disease is Alzheimer's disease, Parkinson's disease, Huntington's disease, or Lou Gehrig's disease (Amyotrophic Lateral Sclerosis or ALS).

In one aspect, described herein is a method of treating substance abuse, the method comprising the step of administering to a subject in need thereof, an effective amount of the compound of Formula (I), (Ia), (II), or (III), wherein the effective amount is sufficient to diminish, inhibit or eliminate desire for and/or consumption of the substance in the subject. In some embodiments, the substance is nicotine, alcohol, opiates, amphetamines, methamphetamines, or cocaine.

In one aspect, described herein is a method for treating an addictive disorder, the method comprising the steps of: a) administering to a subject in need thereof, an effective amount of the compound of Formula (I), (Ia), (II), or (III), during a first time period, wherein the first time period is a time period wherein the subject expects to be in an environment wherein, or exposed to stimuli in the presence of which, the subject habitually uses an addictive substance; and b) administering an effective amount of the compound of Formula (I), (Ia), (II), or (III), during a second time period, wherein the second time period is a time period wherein the subject is suffering from withdrawal.

In one aspect, described herein is a method of treating a disease or condition by modulation of the mGlu2 receptor in a subject in need thereof, which method comprises administering to the subject a therapeutically effective amount of a compound of Formula (I), (Ia), (II), or (III), or a pharmaceutically acceptable salt thereof. In some embodiments, the disease or condition is a CNS disorder.

In another aspect, described herein is a method of treating a disease or condition by modulation of the mGlu3 receptor in a subject in need thereof, which method comprises administering to the subject a therapeutically effective amount of a compound of Formula (I), (Ia), (II), or (III), or a pharmaceutically acceptable salt thereof. In some embodiments, the disease or condition is a CNS disorder.

In one aspect, described herein is a method of treating a disease or condition by dual modulation of the mGlu2/3 receptors in a subject in need thereof, which method comprises administering to the subject a therapeutically effective amount of a compound of Formula (I), (Ia), (II), or (III), or a pharmaceutically acceptable salt thereof. In some embodiments, the disease or condition is a CNS disorder.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION

Glutamate is the major excitatory neurotransmitter in the mammalian central nervous system (CNS), mediating fast synaptic transmission through ion channels, primarily the α-amino-3-hydroxy-5-methyl-4-isoxazolepropionic acid (AMPA) and kainate ionotropic glutamate receptor subtypes. The metabotropic glutamate (mGlu) receptors are a family of eight G protein-coupled receptors that are activated by glutamate and perform a modulatory function in the nervous system. The Group II mGlu receptors include the mGlu2 and mGlu3 receptor subtypes, which couple with G_{i/o} proteins to negatively regulate the activity of adenylyl cyclase. Localization studies suggest that mGlu2 receptors act predominantly as presynaptic autoreceptors to modulate the release of glutamate into the synaptic cleft (Cartmell, J. and Schoepp, D. D. J. Neurochem. 2000, 75, 889-907). On the other hand, mGlu3 receptors exhibit a broad distribution in the brain and have been shown to be present on astrocytes (Durand, D. et al. Neuropharmacology 2013, 66, 1-11). In addition, it has been shown that activation of mGlu3 receptors is required for the neuroprotective effects of mGlu2/3 agonists toward N-methyl-D-aspartate (NMDA) neurotoxicity in mixed cultures of astrocytes and neurons, whereas activation of mGlu2 receptors may be harmful (Corti, C. et al. J. Neurosci. 2007, 27, 8297-8308).

Various brain regions, including the cerebral cortex, hippocampus, striatum, amygdala, frontal cortex, and nucleus accumbens, display high levels of mGlu2 and mGlu3 receptor binding. This distribution pattern suggests a role for the mGlu2/3 receptor subtypes in the pathology of neuropsychiatric disorders such as anxiety, depression, schizophrenia, drug dependence, neuroprotection, Alzheimer's disease, and sleep/wake architecture. Thus, there is significant potential for the development of selective Group II mGlu receptor activators, including agonists and positive allosteric modulators (PAMs), for the treatment of CNS diseases caused by aberrant glutamatergic signaling.

### Compounds

In one aspect, provided herein is a compound having the structure of Formula (I), or a pharmaceutically acceptable salt or solvate, thereof:

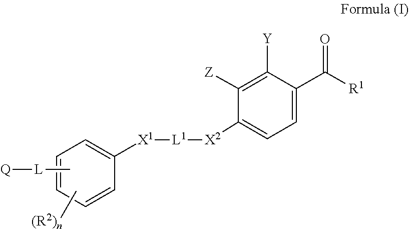

wherein:
R¹ is substituted or unsubstituted C₂-C₆ alkyl, substituted or unsubstituted C₁-C₆ fluoroalkyl, substituted or unsubstituted aryl, substituted or unsubstituted heteroaryl, substituted or unsubstituted cycloalkyl, or substituted or unsubstituted heterocycloalkyl;
X¹ is absent, —O—, —NR⁵—, —S—, —S(═O)—, or —S(═O)₂—;
X² is —O—, —NR⁶—, —S—, —S(═O)—, or —S(═O)₂—;
R⁵ and R⁶ are each independently hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
L¹ is substituted C₄ alkylene, substituted or unsubstituted C₅-C₁₀ alkylene, substituted or unsubstituted C₂-C₁₀ alkenylene, substituted or unsubstituted C₂-C₁₀ alkynylene, -L³-X³-L⁴-, or -L⁵-X⁴-L⁶-;
L³ and L⁴ are each independently substituted or unsubstituted C₁-C₆ alkylene, substituted or unsubstituted C₂-C₆ alkenylene, or optionally substituted or unsubstituted C₂-C₆ alkynylene;
L⁵ and L⁶ are each —C(D)₂- and X⁴ is substituted or unsubstituted C₁-C₄ alkylene; X³ is —C(R⁸)₂, —C(R⁹)═C(R⁹)—, —C≡C—, —O—, —NR⁷—, —S—, —S(═O)—, or —S(═O)₂—;
R⁷ is hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
each R⁸ is independently deuterium, halogen, —OH, substituted or unsubstituted C₁-C₆alkyl, or substituted or unsubstituted C₁-C₆fluoroalkyl;
or two R⁸ are taken together with the carbon atom to which they are attached to form a substituted or unsubstituted cycloalkyl or a substituted or unsubstituted heterocycloalkyl;
each R⁹ is independently hydrogen, deuterium, halogen, —OH, substituted or unsubstituted C₁-C₆alkyl, or substituted or unsubstituted C₁-C₆fluoroalkyl;
L² is absent or C₁-C₆ alkylene;
Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

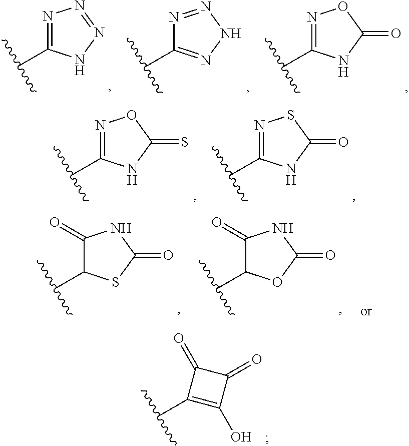

each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl;
n is 0, 1, 2, 3, or 4;
Y is —OH, —OR⁴, halogen, substituted or unsubstituted C₁-C₆alkyl, substituted or unsubstituted C₁-C₆fluoroalkyl, or substituted or unsubstituted cycloalkyl;
Z is —OH, —OR⁴, halogen, substituted or unsubstituted C₁-C₆alkyl, substituted or unsubstituted C₁-C₆fluoroalkyl, or substituted or unsubstituted cycloalkyl;
each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl; or two R³ taken together with the nitrogen to which they are attached to form a substituted or unsubstituted C₂-C₈heterocycloalkyl;
each R⁴ is independently substituted or unsubstituted C₁-C₆alkyl, substituted or unsubstituted cycloalkyl, substituted or unsubstituted aryl, or substituted or unsubstituted heteroaryl.

In some embodiments of a compound of Formula (I), L² is absent or C₁-C₆ alkylene. In some embodiments of a compound of Formula (I), L² is absent. In some embodiments of a compound of Formula (I), L² is C₁-C₆ alkylene.

In some embodiments, the compound of Formula (I), or a pharmaceutically acceptable salt or solvate thereof, Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

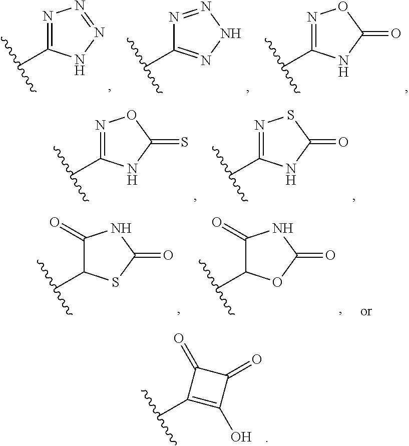

In some embodiments of a compound of Formula (I), Q is

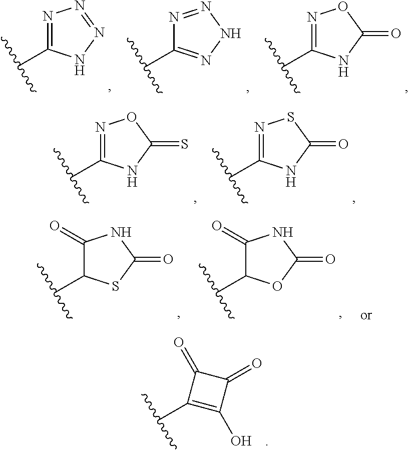

In some embodiments, the compound of Formula (I), or a pharmaceutically acceptable salt or solvate thereof, Q is —C(═O)OH,

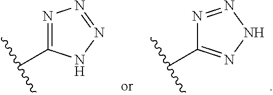 [SMILES: *c1nnn[nH]1 ; *c1nn[nH]n1]

In some embodiments of a compound of Formula (I), Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴. In some embodiments, Q is —C(═O)OH or —C(═O)OR⁴. In some embodiments of a compound of Formula (I), Q is C(═O)OH. In some embodiments of a compound of Formula (I), Q is —C(═O)OR⁴; wherein R⁴ is substituted or unsubstituted C₁-C₆alkyl. In some embodiments of a compound of Formula (I), Q is —C(═O)OR⁴; wherein R⁴ is substituted C₁-C₆alkyl. In some embodiments of a compound of Formula (I), Q is —C(═O)OR⁴; wherein R⁴ is methyl or ethyl. In some embodiments of a compound of Formula (I), Q is —C(═O)OR⁴; wherein R⁴ is methyl.

In some embodiments of the compound of Formula (I), R¹ is substituted or unsubstituted C₂-C₆ alkyl, substituted or unsubstituted C₁-C₆ fluoroalkyl, substituted or unsubstituted aryl, substituted or unsubstituted heteroaryl, substituted or unsubstituted cycloalkyl, or substituted or unsubstituted heterocycloalkyl. In some embodiments of a compound of Formula (I), R¹ is substituted or unsubstituted C₂-C₆ alkyl. In some embodiments of a compound of Formula (I), R¹ is unsubstituted C₂-C₆ alkyl. In some embodiments of a compound of Formula (I), R¹ is n-propyl, n-butyl, n-pentyl, tert-butyl, sec-butyl, iso-butyl, tert-pentyl, neopentyl, isopentyl, or sec-pentyl. In some embodiments of a compound of Formula (I), R¹ is n-propyl, n-butyl, or n-pentyl. In some embodiments of a compound of Formula (I), R¹ is n-propyl. In some embodiments of a compound of Formula (I), R¹ is tert-butyl, sec-butyl, iso-butyl, tert-pentyl, neopentyl, isopentyl, or sec-pentyl. In some embodiments of a compound of Formula (I), R¹ is neopentyl or isopentyl. In some embodiments of a compound of Formula (I), R¹ is neopentyl. In some embodiments of a compound of Formula (I), R¹ is isopentyl.

In some embodiments of the compound of Formula (I), each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl. In some embodiments of a compound of Formula (I), each R² is independently halogen, —OH, or unsubstituted C₁-C₆alkyl. In some embodiments of a compound of Formula (I), each R² is independently unsubstituted C₁-C₆alkyl. In some embodiments of a compound of Formula (I), each R² is independently methyl. In some embodiments of a compound of Formula (I), each R² is independently chloro or fluoro. In some embodiments of a compound of Formula (I), each R² is independently chloro. In some embodiments of a compound of Formula (I), each R² is independently fluoro. In some embodiments of a compound of Formula (I), each R² is independently —OR⁴; wherein R⁴ is methyl. In some embodiments of a compound of Formula (I), each R² is —OMe.

In some embodiments of the compound of Formula (I), X¹ is absent, —O—, —NR—, —S—, —S(═O)—, or —S(═O)₂—. In some embodiments of the compound of Formula (I), X¹ is —S—, —S(═O)—, or —S(═O)₂—. In some embodiments of the compound of Formula (I), X¹ is —S—. In some embodiments of the compound of Formula (I), X¹ is —O—. In some embodiments of the compound of Formula (I), X¹ is absent. In some embodiments of the compound of Formula (I), X¹ is —NR⁵—; wherein R⁵ is hydrogen or substituted or unsubstituted C₁-C₆alkyl. In some embodiments of the compound of Formula (I), X¹ is —NH, or —NMe. In some embodiments of the compound of Formula (I), X¹ is —NH. In some embodiments of the compound of Formula (I), X¹ is —NMe.

In some embodiments of the compound of Formula (I), X² is —O—, —NR⁶—, —S—, —S(═O)—, or —S(═O)₂—. In some embodiments, X² is —S—, —S(═O)—, or —S(═O)₂—. In some embodiments of the compound of Formula (I), X² is —S—. In some embodiments of the compound of Formula (I), X² is —O—. In some embodiments of the compound of Formula (I), X² is —NR⁶—; wherein R⁶ is hydrogen or substituted or unsubstituted C₁-C₆alkyl. In some embodiments of the compound of Formula (I), X² is —NH or —NMe.

In some embodiments of the compound of Formula (I), L¹ is substituted C₄ alkylene, substituted or unsubstituted C₅-C₁₀ alkylene, substituted or unsubstituted C₂-C₁₀ alkenylene, substituted or unsubstituted C₂-C₁₀ alkynylene, -L³-X³-L⁴-, or -L⁵-X⁴-L⁶-; wherein when substituted the substituent is independently deuterium, halogen, C₁-C₄ alkyl; or two substituents on the same carbon atom form a substituted or unsubstituted cycloalkyl or unsubstituted or substituted heterocycloalkyl. In some embodiments of the compound of Formula (I), L¹ is substituted C₄ alkylene, substituted or unsubstituted C₅-C₁₀ alkylene, substituted or unsubstituted C₂-C₁₀ alkenylene, or substituted or unsubstituted C₂-C₁₀ alkynylene. In some embodiments of the compound of Formula (I), L¹ is substituted C₄ alkylene or substituted or unsubstituted C₅-C₁₀ alkylene. In some embodiments of the compound of Formula (I), L¹ is substituted C₄ alkylene. In some embodiments of the compound of Formula (I), L¹ is unsubstituted C₅-C₁₀ alkylene. In some embodiments of the compound of Formula (I), L¹ is unsubstituted C₅ alkylene. In some embodiments of the compound of Formula (I), L¹ is unsubstituted C₆ alkylene. In some embodiments of the compound of Formula (I), L¹ is unsubstituted C₇ alkylene. In some embodiments of the compound of Formula (I), L¹ is unsubstituted C₈ alkylene. In some embodiments of the compound of Formula (I), L¹ is unsubstituted C₉ alkylene. In some embodiments of the compound of Formula (I), L¹ is unsubstituted C₁₀ alkylene. In some embodiments of the compound of Formula (I), L¹ is substituted or unsubstituted C₄ alkenylene. In some embodiments of the compound of Formula (I), L¹ is unsubstituted C₄ alkenylene. In some embodiments of the compound of Formula (I), L¹ is

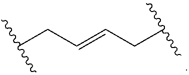 [SMILES: *C/C=C/C*]

In some embodiments of the compound of Formula (I), X¹ is —O—; X² is —O—; and L¹ is substituted or unsubstituted C₄ alkenylene. In some embodiments of the compound of Formula (I), X¹ is —O—; X² is —O—; and L¹ is unsubstituted C₄ alkenylene. In some embodiments of the compound of Formula (I), X¹ is —O—; X² is —O—; and L¹ is

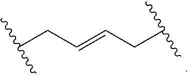 [SMILES: *C/C=C/C*]

In some embodiments, each R⁸ is independently D, halogen, —OH, substituted or unsubstituted C₂-C₆ alkyl, or substituted or unsubstituted C₁-C₆fluoroalkyl. In some embodiments of the compound of Formula (I), L¹ is —(CR^{8a}R^{8b})ₚ—; wherein each R^{8a} and R^{8b} is independently hydrogen, deuterium, halogen, C₁-C₄ alkyl; or R^{8a} and R^{8b} on the same carbon atom form a substituted or unsubstituted cycloalkyl or unsubstituted or substituted heterocycloalkyl; and p is 5-10. In some embodiments of the compound of Formula (I), p is 5. In some embodiments of the compound of Formula (I), p is 5 or 6. In some embodiments of the compound of Formula (I), p is 5-7. In some embodiments of the compound of Formula (I), p is 5-8.

In some embodiments of the compound of Formula (I), L¹ is

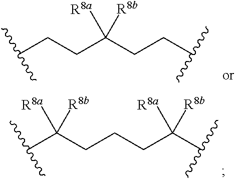

and each R^{8a} and R^{8b} is independently hydrogen, deuterium, halogen, C₁-C₄ alkyl; or R^{8a} and R^{8b} on the same carbon atom form a substituted or unsubstituted cycloalkyl or unsubstituted or substituted heterocycloalkyl. In some embodiments of the compound of Formula (I), each R^{8a} or R^{8b} is hydrogen, deuterium, or C₁-C₄ alkyl. In some embodiments of the compound of Formula (I), each R^{8a} or R^{8b} is hydrogen. In some embodiments of the compound of Formula (I), each R^{8a} or R^{8b} is deuterium. In some embodiments of the compound of Formula (I), each R^{8a} or R^{8b} is methyl. In some embodiments of the compound of Formula (I), R^{8a} and R^{8b} on the same carbon atom form a substituted or unsubstituted cycloalkyl or unsubstituted or substituted heterocycloalkyl. In some embodiments of the compound of Formula (I), R^{8a} and R^{8b} on the same carbon atom form an unsubstituted cycloalkyl.

In some embodiments of the compound of Formula (I), L¹ is —(CR^{8c}R^{8d})ₘ—; wherein each R^{8c} and R^{8d} is independently hydrogen, deuterium, halogen, C₁-C₄ alkyl; and m is 2-4; provided that one of R^{8c} and R^{8d} is not hydrogen or provided that R^{8c} and R^{8d} on the same carbon atom form a substituted or unsubstituted cycloalkyl or unsubstituted or substituted heterocycloalkyl. In some embodiments of the compound of Formula (I), m is 3 or 4. In some embodiments of the compound of Formula (I), m is 4.

In some embodiments of the compound of Formula (I), L¹ is

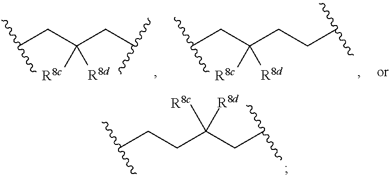

and each R^{8c} and R^{8d} is independently hydrogen, deuterium, halogen, C₁-C₄ alkyl; and m is 2-4; provided that one of R^{8c} and R^{8d} is not hydrogen or provided that R^{8c} and R^{8d} on the same carbon atom form a substituted or unsubstituted cycloalkyl or unsubstituted or substituted heterocycloalkyl.

In some embodiments of the compound of Formula (I), each R^{8c} or R^{8d} is deuterium. In some embodiments of the compound of Formula (I), each R^{8c} or R^{8d} is methyl. In some embodiments of the compound of Formula (I), R^{8c} and R^{8d} on the same carbon atom form a substituted or unsubstituted cycloalkyl or unsubstituted or substituted heterocycloalkyl. In some embodiments of the compound of Formula (I), R^{8c} and R^{8d} on the same carbon atom form an unsubstituted cycloalkyl.

In some embodiments of the compound of Formula (I), L¹ is -L³-X³-L⁴- or -L⁵-X⁴-L⁶-. In some embodiments, L⁵ and L⁶ are each —C(D)₂- and X⁴ is substituted or unsubstituted C₁-C₄ alkylene.

In another aspect, provided herein the compound of Formula (I), has the structure of Formula (Ia), or a pharmaceutically acceptable salt or solvate thereof:

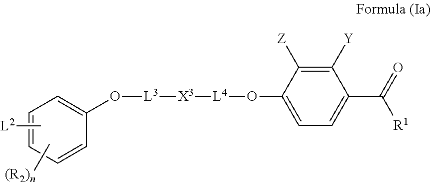

wherein:
L³ and L⁴ are each independently C₁-C₄alkylene;
X³ is —C(R⁸)₂, —C(R⁹)═C(R⁹)—, —C≡C—, —O—, —NR⁷—, —S—, —S(═O)—, or —S(═O)₂—;
each R⁸ is independently D, halogen, —OH, substituted or unsubstituted C₁-C₆alkyl, or substituted or unsubstituted C₁-C₆fluoroalkyl;
or two R⁸ are taken together with the carbon atom to which they are attached to form a substituted or unsubstituted cycloalkyl or a substituted or unsubstituted heterocycloalkyl;
each R⁹ is independently hydrogen, D, halogen, —OH, substituted or unsubstituted C₁-C₆alkyl, or substituted or unsubstituted C₁-C₆fluoroalkyl.

In some embodiments of the compound of Formula (I) or (Ia), X³ is —C(R⁸)₂, —C(R⁹)═C(R⁹)—, —C≡C—, —O—, —NR⁷—, —S—, —S(═O)—, or —S(═O)₂—. In some embodiments of the compound of Formula (I) or (Ia), X³ is —O—, —S—, —S(═O)—, or —S(═O)₂—. In some embodiments of the compound of Formula (I) or (Ia), X³ is —S—, —S(═O)—, or —S(═O)₂—. In some embodiments of the compound of Formula (I) or (Ia), X³ is —O—. In some embodiments of the compound of Formula (I) or (Ia), X³ is —S—. In some embodiments of the compound of Formula (I) or (Ia), X³ is —S(═O)—. In some embodiments of the compound of Formula (I) or (Ia), X³ is —S—. In some embodiments of the compound of Formula (I) or (Ia), X³ is —S(═O)₂—. In some embodiments of the compound of Formula (I) or (Ia), X³ is —C(R⁸)₂, —C(R⁹)═C(R⁹)—, or —C≡C—. In some embodiments of the compound of Formula (I) or (Ia), X³ is —C≡C—. In some embodiments of the compound of Formula (I) or (Ia), X³ is —C(R⁹)═C(R⁹)—; wherein each R⁹ is independently hydrogen or deuterium. In some embodiments of the compound of Formula (I) or (Ia), X³ is —C(R⁹)═C(R⁹)—; wherein each R⁹ is independently deuterium. In some embodiments of the compound of Formula (I) or (Ia), X³ is —C(R⁹)═C(R⁹)—; wherein each R⁹ is independently hydrogen. In some embodiments of the compound of Formula (I) or (Ia), X³ is —C(R⁸)₂; wherein each R⁸ is —OH or substituted or unsubstituted C₁-C₆alkyl. In some embodiments of the compound of Formula (I) or (Ia), X³ is —C(R⁸)₂; wherein each R⁸ is unsubstituted C₁-C₆alkyl.

In some embodiments of the compound of Formula (I) or (Ia), X³ is —NR⁷—; wherein R⁷ is hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³. In some embodiments of the compound of Formula (I) or (Ia), R⁷ is hydrogen, substituted or unsubstituted C₁-C₆alkyl, —S(═O)₂R⁴, or —C(═O)OR³. In some embodiments of the compound of Formula (I) or (Ia), R⁷ is hydrogen or substituted or unsubstituted C₁-C₆alkyl. In some embodiments of the compound of Formula (I) or (Ia), R⁷ is hydrogen or methyl. In some embodiments of the compound of Formula (I) or (Ia), R⁷ is —S(═O)₂R⁴; wherein R⁴ is substituted or unsubstituted C₁-C₆alkyl or substituted or unsubstituted aryl. In some embodiments of the compound of Formula (I) or (Ia), R⁷ is C(═O)OR³; wherein R³ is substituted or unsubstituted C₁-C₆alkyl.

In some embodiments of the compound of Formula (I) or (Ia), L³ and L⁴ are each independently C₁-C₄alkylene, substituted or unsubstituted C₁-C₆ alkylene, substituted or unsubstituted C₂-C₆ alkenylene, or optionally substituted or unsubstituted C₂-C₆ alkynylene; wherein when substituted is independently substituted with deuterium, halogen, C₁-C₄ alkyl; or two substituents on the same carbon atom form a substituted or unsubstituted cycloalkyl or substituted or unsubstituted heterocycloalkyl. In some embodiments of the compound of Formula (I) or (Ia), L³ and L⁴ are each independently substituted or unsubstituted C₂-C₆alkylene. In some embodiments of the compound of Formula (I) or (Ia), L³ and L⁴ are each independently unsubstituted C₂ alkylene. In some embodiments of the compound of Formula (I) or (Ia), L³ and L⁴ are each independently unsubstituted C₃ alkylene. In some embodiments of the compound of Formula (I) or (Ia), L³ and L⁴ are each independently unsubstituted C₄ alkylene. In some embodiments of the compound of Formula (I) or (Ia), L³ and L⁴ are each independently unsubstituted C₅ alkylene.

In another aspect, provided herein is a compound having the structure of Formula (II), or a pharmaceutically acceptable salt or solvate, thereof:

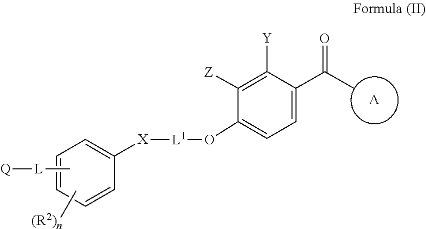

wherein:
ring A is a substituted or unsubstituted aryl, substituted or unsubstituted heteroaryl, substituted or unsubstituted cycloalkyl, or substituted or unsubstituted heterocycloalkyl;
X is —O— and L¹ is —(CH₂)₄—;
or X is substituted or unsubstituted arylene, substituted or unsubstituted heteroarylene, substituted or unsubstituted cycloalkylene, or substituted or unsubstituted heterocycloalkylene; and L¹ is —CH₂—;
L is absent or C₁-C₆ alkylene;
Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

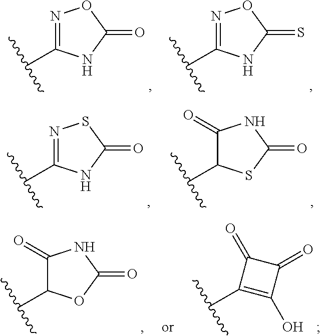 [SMILES: *c1noc(=O)[nH]1 ; *c1noc(=S)[nH]1 ; *c1nsc(=O)[nH]1 ; *C1SC(=O)NC1=O ; *C1OC(=O)NC1=O ; *c1c(O)c(=O)c1=O]

each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl;
n is 0, 1, 2, 3, or 4;
Y is —OH, —OR⁴, halogen, substituted or unsubstituted C₁-C₆alkyl, substituted or unsubstituted C₁-C₆fluoroalkyl, or substituted or unsubstituted cycloalkyl;
Z is —OH, —OR⁴, halogen, substituted or unsubstituted C₁-C₆alkyl, substituted or unsubstituted C₁-C₆fluoroalkyl, or substituted or unsubstituted cycloalkyl;
each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl; or two R³ taken together with the nitrogen to which they are attached to form a substituted or unsubstituted C₂-C₈heterocycloalkyl; and
each R⁴ is independently substituted or unsubstituted C₁-C₆alkyl, substituted or unsubstituted cycloalkyl, substituted or unsubstituted aryl, or substituted or unsubstituted heteroaryl.

In some embodiments of the compound of Formula (II), ring A is substituted or unsubstituted aryl, substituted or unsubstituted heteroaryl, substituted or unsubstituted cycloalkyl, or substituted or unsubstituted heterocycloalkyl. In some embodiments of the compound of Formula (II), ring A is substituted or unsubstituted aryl or substituted or unsubstituted cycloalkyl. In some embodiments of the compound of Formula (II), ring A is substituted or unsubstituted aryl. In some embodiments of the compound of Formula (II), ring A is unsubstituted aryl. In some embodiments of the compound of Formula (II), ring A is aryl substituted with one, two, or three halogen, C₁-C₆alkyl, C₁-C₆ alkoxy, or C₁-C₆fluoroalkyl. In some embodiments of the compound of Formula (II), ring A is aryl substituted independently with one, two, or three substituents from bromo, chloro, or fluoro. In some embodiments of the compound of Formula (II), ring A is aryl substituted with one fluoro. In some embodiments of the compound of Formula (II), ring A is aryl independently substituted with one, two, or three substituents from C₁-C₆alkyl or C₁-C₆ alkoxy. In some embodiments of the compound of Formula (II), ring A is aryl independently substituted with one, two, or three C₁-C₆ alkoxy. In some embodiments of the compound of Formula (II), ring A is aryl substituted with one, two, or three methoxy. In some embodiments of the compound of Formula (II), ring A is aryl substituted with one methoxy. In some embodiments of the compound of Formula (II), ring A is substituted or unsubstituted cycloalkyl. In some embodiments of the compound of Formula (II), ring A is unsubstituted cyclopropyl, cyclobutyl, cyclopentyl, or cyclohexyl. In some embodiments of the compound of Formula (II), ring A is unsubstituted cyclopropyl.

In some embodiments of the compound of Formula (II), X is —O— and L¹ is —(CH₂)₄—. In some embodiments of the compound of Formula (II), X is substituted or unsubstituted arylene, substituted or unsubstituted heteroarylene, substituted or unsubstituted cycloalkylene, or substituted or unsubstituted heterocycloalkylene; and L¹ is —CH₂—. In some embodiments of the compound of Formula (II), X is unsubstituted arylene or unsubstituted heteroarylene; and L¹ is —CH₂—. In some embodiments of the compound of Formula (II), X is unsubstituted arylene; and L¹ is —CH₂—. In some embodiments of the compound of Formula (II), X is unsubstituted heteroarylene; and L¹ is —CH₂—.

In some embodiments of the compound of Formula (II), X is

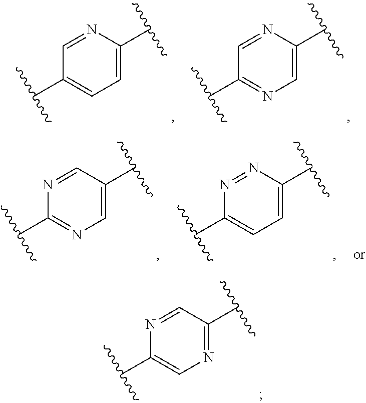 [SMILES: *c1ccc(*)nc1 ; *c1cnc(*)cn1 ; *c1cnc(*)nc1 ; *c1ccc(*)nn1 ; *c1cnc(*)cn1]

and L¹ is —CH₂—. In some embodiments of the compound of Formula (II), X is

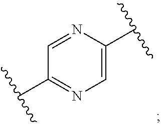 [SMILES: *c1cnc(*)cn1]

### and L is —CH₂—.

In some embodiments of the compound of Formula (II), Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

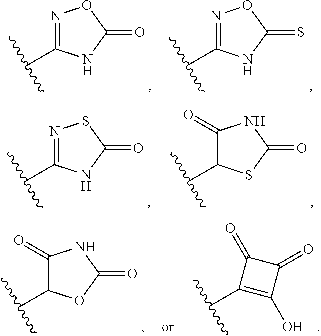 [SMILES: *c1noc(=O)[nH]1 ; *c1noc(=S)[nH]1 ; *c1nsc(=O)[nH]1 ; *C1SC(=O)NC1=O ; *C1OC(=O)NC1=O ; *c1c(O)c(=O)c1=O]

In some embodiments of the compound of Formula (II), Q is a carboxylic acid bioisostere having the structure

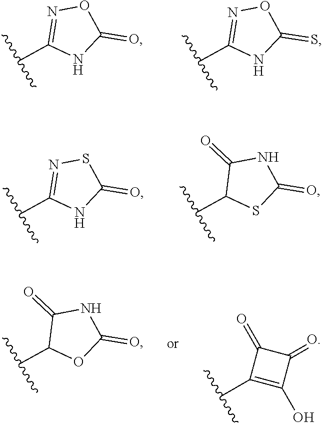 [SMILES: *c1noc(=O)[nH]1 ; *c1noc(=S)[nH]1 ; *c1nsc(=O)[nH]1 ; *C1SC(=O)NC1=O ; *C1OC(=O)NC1=O ; *c1c(O)c(=O)c1=O]

In some embodiments of the compound of Formula (II), Q is —C(═O)OH, —C(═O)OR⁴, —C(═O)NHOR³, —C(═O)NHCN, —C(═O)NR³R³, —C(═O)NHS(═O)₂R⁴, —C(═O)R⁴,
—S(═O)₂NR³R³, —S(═O)₂NHC(═O)R⁴, —NHC(═O)NHS(═O)₂R⁴; wherein each R and R⁴ is independently hydrogen or substituted or unsubstituted C₁-C₆alkyl. In some embodiments of the compound of Formula (II), Q is —C(═O)OH or —C(═O)OR⁴; wherein R⁴ is unsubstituted C₁-C₆alkyl. In some embodiments of the compound of Formula (II), Q is —C(═O)OH. In some embodiments of the compound of Formula (II), Q is —C(═O)OR⁴; wherein R⁴ is methyl or ethyl. In some embodiments of the compound of Formula (II), Q is —C(═O)OR⁴; wherein R⁴ is methyl.

In some embodiments of the compound of Formula (II), each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl. In some embodiments of the compound of Formula (II), each R² is substituted or unsubstituted C₁-C₆alkyl. In some embodiments of the compound of Formula (II), each R² is methyl. In some embodiments of the compound of Formula (II), each R² is independently —OH or —OR⁴. In some embodiments of the compound of Formula (II), each R² is —OH. In some embodiments of the compound of Formula (II), R² is —OR⁴; wherein R⁴ is substituted or unsubstituted C₁-C₆alkyl. In some embodiments of the compound of Formula (II), each R² is —OMe.

In some embodiments of the compound of Formula (II), L is absent or C₁-C₆ alkylene. In some embodiments of the compound of Formula (II), L is absent. In some embodiments of the compound of Formula (II), L is C₁-C₆ alkylene.

In another aspect of provided herein is a compound having the structure of Formula (III), or a pharmaceutically acceptable salt or solvate, thereof:

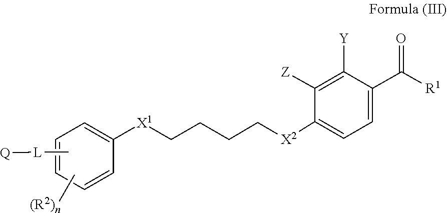

wherein:
R¹ is substituted or unsubstituted C₄-C₆ alkyl, substituted or unsubstituted C₁-C₆ fluoroalkyl, substituted or unsubstituted aryl, substituted or unsubstituted heteroaryl, substituted or unsubstituted cycloalkyl, or substituted or unsubstituted heterocycloalkyl;
X¹ and X² are each independently —O—, —NR—, —S—, —S(═O)—, or —S(═O)₂—, wherein at least one of X¹ and X² is —NR⁵—, —S—, —S(═O)—, or —S(═O)₂—;
each R⁵ is independently hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
L is absent or C₁-C₆ alkylene;
Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

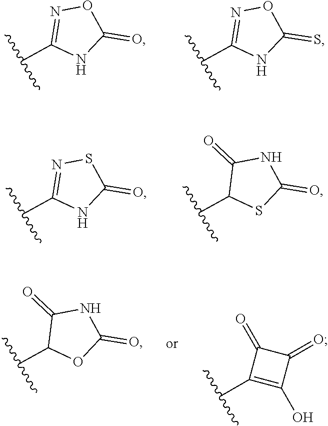 [SMILES: *c1noc(=O)[nH]1 ; *c1noc(=S)[nH]1 ; *c1nsc(=O)[nH]1 ; *C1SC(=O)NC1=O ; *C1OC(=O)NC1=O ; *c1c(O)c(=O)c1=O]

each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl;
n is 0, 1, 2, 3, or 4;
Y is —OH, —OR⁴, halogen, substituted or unsubstituted C₁-C₆alkyl, substituted or unsubstituted C₁-C₆fluoroalkyl, or substituted or unsubstituted cycloalkyl;
Z is —OH, —OR⁴, halogen, substituted or unsubstituted C₁-C₆alkyl, substituted or unsubstituted C₁-C₆fluoroalkyl, or substituted or unsubstituted cycloalkyl;
each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl; or two R³ taken together with the nitrogen to which they are attached to form a substituted or unsubstituted C₂-C₈heterocycloalkyl; and
each R⁴ is independently substituted or unsubstituted C₁-C₆alkyl, substituted or unsubstituted cycloalkyl, substituted or unsubstituted aryl, or substituted or unsubstituted heteroaryl.

In some embodiments of the compound of Formula (III), X¹ is —O—, —NR⁵—, —S—, —S(═O)—, or —S(═O)₂—; and X² is —NR⁵—, —S—, —S(═O)—, or —S(═O)₂—. In some embodiments of the compound of Formula (III), X¹ is —O—, —NR⁵—, —S—, —S(═O)—, or —S(═O)₂—; and X² is —S—, —S(═O)—, or —S(═O)₂—. In some embodiments of the compound of Formula (III), X¹ is —O—, —NR⁵—, —S—, —S(═O)—, or —S(═O)₂—; and X² is —S—. In some embodiments of the compound of Formula (III), X¹ is —S(═O)— or —S(═O)₂—; and X² is —S—. In some embodiments of the compound of Formula (III), X¹ is —O—; and X² is —S—. In some embodiments of the compound of Formula (III), X¹ is —S—; and X² is —S—. In some embodiments of the compound of Formula (III), X¹ is —NR⁵—; and X² is —S—; wherein R⁵ is hydrogen or substituted or unsubstituted C₁-C₆alkyl. In some embodiments of the compound of Formula (III), X¹ is —NR⁵—; and X² is —S—; wherein R⁵ is hydrogen. In some embodiments of the compound of Formula (III), X¹ is —NR⁵—; and X² is —NR⁵—; wherein each R⁵ is hydrogen or substituted or unsubstituted C₁-C₆alkyl. In some embodiments of the compound of Formula (III), X¹ is —NR—; and X² is —NR—; wherein R⁵ is hydrogen. In some embodiments of the compound of Formula (III), X¹ is —NR⁵—; and X² is —S—; wherein R⁵ is hydrogen. In some embodiments of the compound of Formula (III), X¹ is —O—; and X² is —NR⁵—; wherein each R is hydrogen or substituted or unsubstituted C₁-C₆alkyl. In some embodiments of the compound of Formula (III), X¹ is —O—; and X² is —NR⁵—; wherein R⁵ is hydrogen.

In some embodiments of the compound of Formula (III), X¹ is —NR⁵—, —S—, —S(═O)—, or —S(═O)₂—; and X² is —O—, —NR⁵—, —S—, —S(═O)—, or —S(═O)₂—. In some embodiments of the compound of Formula (III), X¹ is —S—, —S(═O)—, or —S(═O)₂—; and X² is —O—, —NR⁵—, —S—, —S(═O)—, or —S(═O)₂—. In some embodiments of the compound of Formula (III), X¹ is —S—; and X² is —O—. In some embodiments of the compound of Formula (III), X¹ is —S—, —S(═O)—, or —S(═O)₂—; and X² is —O—, —NR⁵—, —S—, —S(═O)—, or —S(═O)₂—. In some embodiments of the compound of Formula (III), X¹ is —S—; and X² is —NR⁵—; wherein R⁵ is hydrogen or substituted or unsubstituted C₁-C₆alkyl. In some embodiments of the compound of Formula (III), X¹ is —S—; and X² is —NR⁵—; wherein R is hydrogen. In some embodiments of the compound of Formula (III), X¹ is —NR⁵—; and X² is —O—; wherein R⁵ is hydrogen or substituted or unsubstituted C₁-C₆alkyl. In some embodiments of the compound of Formula (III), X¹ is —NR⁵—; and X² is —O—; wherein R⁵ is hydrogen.

In some embodiments of the compound of Formula (III), Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴,

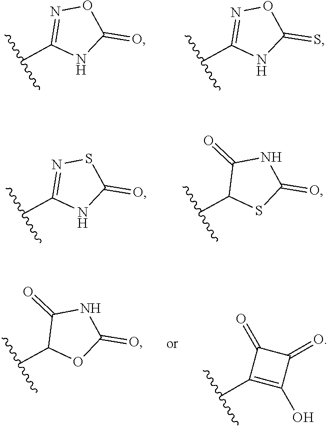 [SMILES: *c1noc(=O)[nH]1 ; *c1noc(=S)[nH]1 ; *c1nsc(=O)[nH]1 ; *C1SC(=O)NC1=O ; *C1OC(=O)NC1=O ; *c1c(O)c(=O)c1=O]

In some embodiments of the compound of Formula (III), Q is

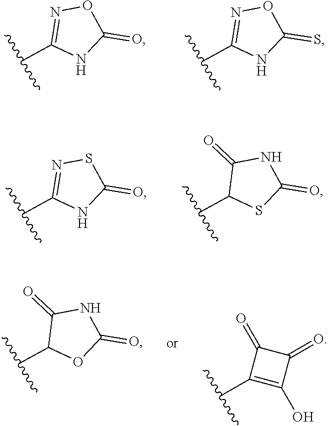 [SMILES: *c1noc(=O)[nH]1 ; *c1noc(=S)[nH]1 ; *c1nsc(=O)[nH]1 ; *C1SC(=O)NC1=O ; *C1OC(=O)NC1=O ; *c1c(O)c(=O)c1=O]

In some embodiments of the compound of Formula (III), Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, or —NHC(═O)NHS(═O)₂R⁴; wherein each or R and R⁴ are independently hydrogen, substituted or unsubstituted C₁-C₆alkyl, substituted or unsubstituted C₁-C₆fluoroalkyl, substituted or unsubstituted cycloalkyl, or substituted or unsubstituted aryl. In some embodiments of the compound of Formula (III), Q is —C(═O)OH or —C(═O)OR⁴. In some embodiments of the compound of Formula (III), Q is C(═O)OH. In some embodiments of the compound of Formula (III), Q is —C(═O)OR⁴; wherein R⁴ is substituted or unsubstituted C₁-C₆alkyl. In some embodiments of the compound of Formula (III), Q is —C(═O)OR⁴; wherein R⁴ is methyl.

In some embodiments of the compound of Formula (III), R¹ is substituted or unsubstituted C₄-C₆ alkyl, substituted or unsubstituted C₁-C₆ fluoroalkyl, substituted or unsubstituted aryl, substituted or unsubstituted heteroaryl, substituted or unsubstituted cycloalkyl, or substituted or unsubstituted heterocycloalkyl. In some embodiments of the compound of Formula (III), R¹ is substituted or unsubstituted C₄-C₆ alkyl. In some embodiments of the compound of Formula (III), R¹ is unsubstituted C₄-C₆ alkyl. In some embodiments of the compound of Formula (III), R¹ is n-butyl, n-pentyl, or n-hexyl. In some embodiments of the compound of Formula (III), R¹ is sec-butyl, iso-butyl, tert-pentyl, neopentyl, isopentyl, or sec-pentyl. In some embodiments of the compound of Formula (III), R¹ is neopentyl or isopentyl. In some embodiments of the compound of Formula (III), R¹ is neopentyl. In some embodiments of the compound of Formula (III), R¹ is isopentyl.

In some embodiments of the compound of Formula (III), each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl. In some embodiments of the compound of Formula (III), each R² is substituted or unsubstituted C₁-C₆alkyl. In some embodiments of the compound of Formula (III), each R² is methyl. In some embodiments of the compound of Formula (III), each R² is independently —OH or —OR⁴. In some embodiments of the compound of Formula (III), each R² is —OH. In some embodiments of the compound of Formula (III), each R² is —OR⁴; wherein R⁴ is substituted or unsubstituted C₁-C₆alkyl. In some embodiments of the compound of Formula (III), each R² is —OMe.

In some embodiments of the compound of Formula (III), L is absent or C₁-C₆ alkylene. In some embodiments of the compound of Formula (III), L is absent. In some embodiments of the compound of Formula (III), L is C₁-C₆ alkylene.

In some embodiments of the compound of Formula (I), (Ia), (II), or (III), Y is —OH, —OR⁴, halogen, substituted or unsubstituted C₁-C₆alkyl, substituted or unsubstituted C₁-C₆fluoroalkyl, or substituted or unsubstituted cycloalkyl. In some embodiments of the compound of Formula (I), (Ia), (II), or (III), Y is —OH or —OR⁴; wherein R⁴ is substituted or unsubstituted C₁-C₆alkyl, substituted or unsubstituted cycloalkyl, substituted or unsubstituted aryl, or substituted or unsubstituted heteroaryl. In some embodiments of the compound of Formula (I), (Ia), (II), or (III), Y is —OR⁴; wherein, R⁴ is substituted or unsubstituted C₁-C₆alkyl. In some embodiments of the compound of Formula (I), (Ia), (II), or (III), Y is —OR⁴; wherein, R⁴ is methyl. In some embodiments of the compound of Formula (I), (Ia), (II), or (III), Y is —OH.

In some embodiments of the compound of Formula (I), (Ia), (II), or (III), Z is —OH, —OR⁴, halogen, substituted or unsubstituted C₁-C₆alkyl, substituted or unsubstituted C₁-C₆fluoroalkyl, or substituted or unsubstituted cycloalkyl. In some embodiments of the compound of Formula (I), (Ia), (II), or (III), Z is substituted or unsubstituted C₁-C₆alkyl. In some embodiments of the compound of Formula (I), (Ia), (II), or (III), Z is methyl, ethyl, butyl, or pentyl. In some embodiments of the compound of Formula (I), (Ia), (II), or (III), Z is methyl.

In some embodiments of the compound of Formula (I), (Ia), (II), or (III), n is an integer 0, 1, 2, 3, or 4. In some embodiments of the compound of Formula (I), (Ia), (II), or (III), n is 0, 1, or 2. In some embodiments of the compound of Formula (I), (Ia), (II), or (III), n is 0 or 1. In some embodiments of the compound of Formula (I), (Ia), (II), or (III), or a pharmaceutically acceptable salt or solvate thereof, n is 1. In some embodiments of the compound of Formula (I), (Ia), (II), or (III), n is 0.

In some embodiments of the compound of Formula (I), (Ia), (II), or (III), each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl; or two R³ taken together with the nitrogen to which they are attached to form a substituted or unsubstituted C₂-C₆heterocycloalkyl. In some embodiments of the compound of Formula (I), (Ia), (II), or (III), each R³ is independently hydrogen, substituted or unsubstituted C₁-C₆alkyl, or substituted or unsubstituted aryl. In some embodiments of the compound of Formula (I), (Ia), (II), or (III), each R³ is independently substituted or unsubstituted aryl. In some embodiments of the compound of Formula (I), (Ia), (II), or (III), each R³ is independently hydrogen or substituted or unsubstituted C₁-C₆alkyl. In some embodiments of the compound of Formula (I), (Ia), (II), or (III), each R³ is independently substituted or unsubstituted C₁-C₆alkyl. In some embodiments of the compound of Formula (I), (Ia), (II), or (III), each R³ is independently hydrogen or methyl.

In some embodiments of the compound of Formula (I), (Ia), (II), or (III), each R⁴ is independently substituted or unsubstituted C₁-C₆alkyl, substituted or unsubstituted cycloalkyl, substituted or unsubstituted aryl, or substituted or unsubstituted heteroaryl. In some embodiments of the compound of Formula (I), (Ia), (II), or (III), each R⁴ is independently substituted or unsubstituted aryl or substituted or unsubstituted heteroaryl. In some embodiments of the compound of Formula (I), (Ia), (II), or (III), each R⁴ is independently substituted or unsubstituted C₁-C₆alkyl. In some embodiments of the compound of Formula (I), (Ia), (II), or (III), each R⁴ is independently hydrogen or methyl.

In some embodiments, the compound is any of the compounds represented in Table 1, or a pharmaceutically acceptable salt or solvate thereof.

TABLE 1

| Example | Structure |
|---|---|
| 1 | 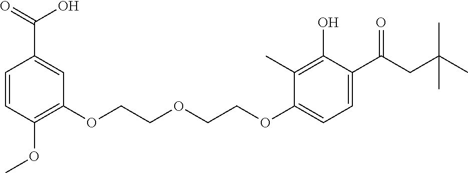 [SMILES: COc1ccc(C(=O)O)cc1OCCOCCOc1ccc(C(=O)CC(C)(C)C)c(O)c1C] |
| 2 | 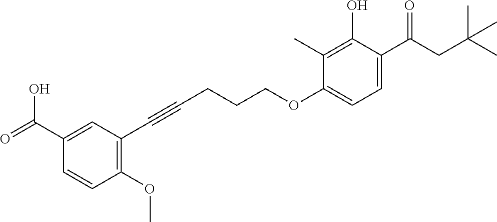 [SMILES: COc1ccc(C(=O)O)cc1C#CCCCOc1ccc(C(=O)CC(C)(C)C)c(O)c1C] |
| 3 | 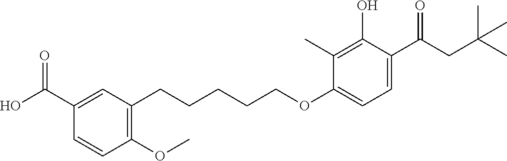 [SMILES: COc1ccc(C(=O)O)cc1CCCCCOc1ccc(C(=O)CC(C)(C)C)c(O)c1C] |
| 4 | 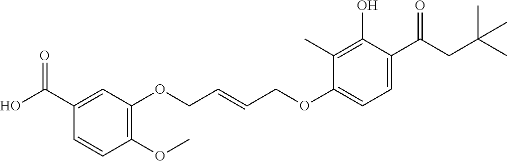 [SMILES: COc1ccc(C(=O)O)cc1OC/C=C/COc1ccc(C(=O)CC(C)(C)C)c(O)c1C] |
| 5 | 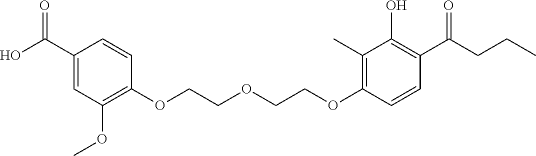 [SMILES: CCCC(=O)c1ccc(OCCOCCOc2ccc(C(=O)O)cc2OC)c(C)c1O] |
| 6 | 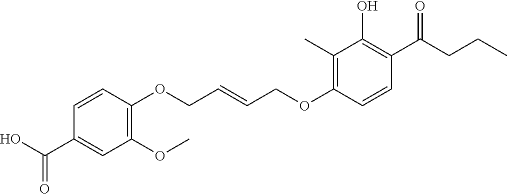 [SMILES: CCCC(=O)c1ccc(OC/C=C/COc2ccc(C(=O)O)cc2OC)c(C)c1O] |
| 7 | 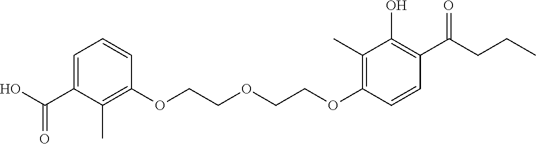 [SMILES: CCCC(=O)c1ccc(OCCOCCOc2cccc(C(=O)O)c2C)c(C)c1O] |
| 8 | 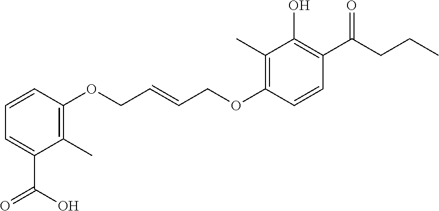 [SMILES: CCCC(=O)c1ccc(OC/C=C/COc2cccc(C(=O)O)c2C)c(C)c1O] |
| 9 | 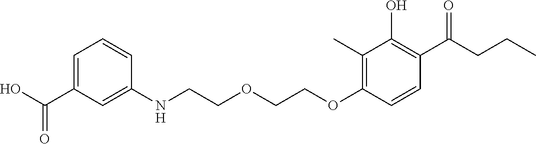 [SMILES: CCCC(=O)c1ccc(OCCOCCNc2cccc(C(=O)O)c2)c(C)c1O] |
| 10 | 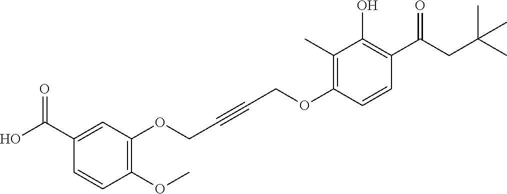 [SMILES: COc1ccc(C(=O)O)cc1OCC#CCOc1ccc(C(=O)CC(C)(C)C)c(O)c1C] |
| 11 | 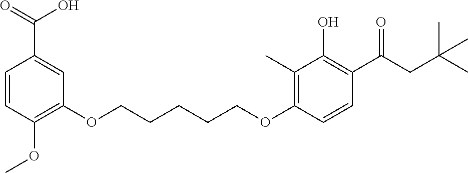 [SMILES: COc1ccc(C(=O)O)cc1OCCCCCOc1ccc(C(=O)CC(C)(C)C)c(O)c1C] |
| 12 | 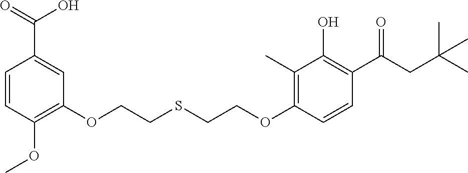 [SMILES: COc1ccc(C(=O)O)cc1OCCSCCOc1ccc(C(=O)CC(C)(C)C)c(O)c1C] |
| 13 | 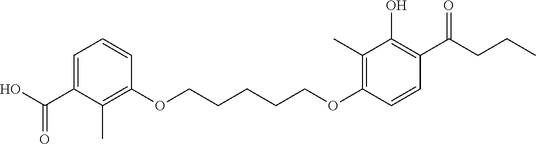 [SMILES: CCCC(=O)c1ccc(OCCCCCOc2cccc(C(=O)O)c2C)c(C)c1O] |
| 14 | 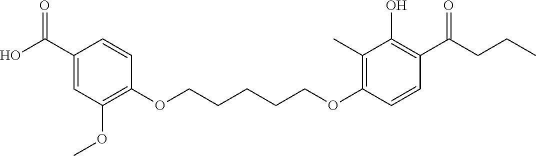 [SMILES: CCCC(=O)c1ccc(OCCCCCOc2ccc(C(=O)O)cc2OC)c(C)c1O] |
| 15 | 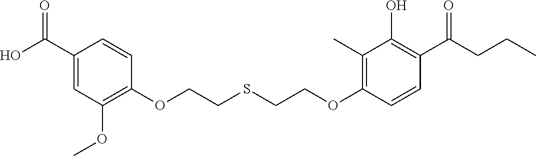 [SMILES: CCCC(=O)c1ccc(OCCSCCOc2ccc(C(=O)O)cc2OC)c(C)c1O] |
| 16 | 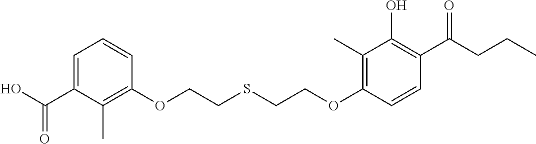 [SMILES: CCCC(=O)c1ccc(OCCSCCOc2cccc(C(=O)O)c2C)c(C)c1O] |
| 17 | 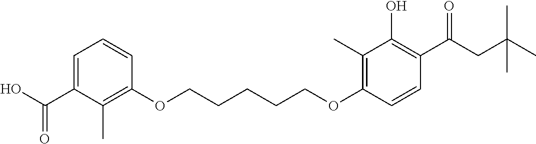 [SMILES: Cc1c(OCCCCCOc2ccc(C(=O)CC(C)(C)C)c(O)c2C)cccc1C(=O)O] |
| 18 | 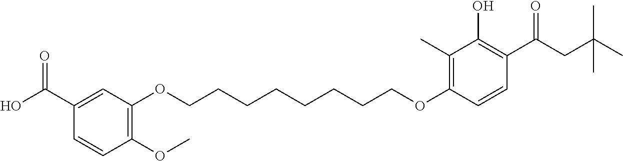 [SMILES: COc1ccc(C(=O)O)cc1OCCCCCCCCOc1ccc(C(=O)CC(C)(C)C)c(O)c1C] |
| 19 | 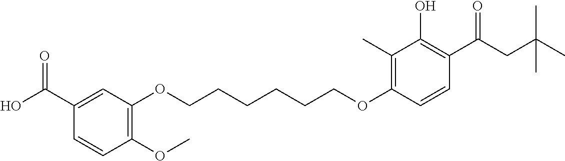 [SMILES: COc1ccc(C(=O)O)cc1OCCCCCCOc1ccc(C(=O)CC(C)(C)C)c(O)c1C] |
| 20 | 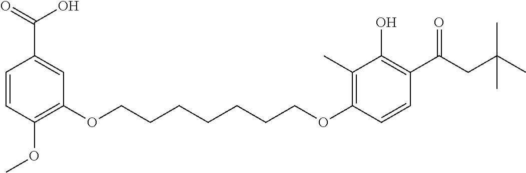 [SMILES: COc1ccc(C(=O)O)cc1OCCCCCCCOc1ccc(C(=O)CC(C)(C)C)c(O)c1C] |
| 21 | 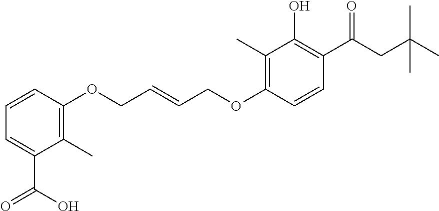 [SMILES: Cc1c(OC/C=C/COc2ccc(C(=O)CC(C)(C)C)c(O)c2C)cccc1C(=O)O] |
| 22 | 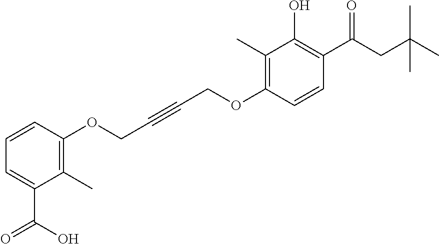 [SMILES: Cc1c(OCC#CCOc2ccc(C(=O)CC(C)(C)C)c(O)c2C)cccc1C(=O)O] |
| 23 | 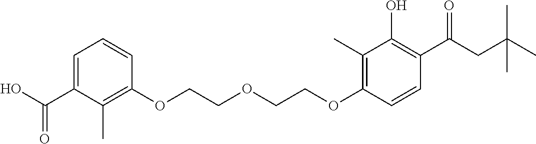 [SMILES: Cc1c(OCCOCCOc2ccc(C(=O)CC(C)(C)C)c(O)c2C)cccc1C(=O)O] |
| 24 | 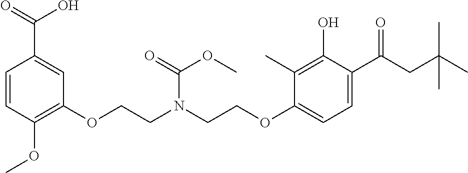 [SMILES: COC(=O)N(CCOc1cc(C(=O)O)ccc1OC)CCOc1ccc(C(=O)CC(C)(C)C)c(O)c1C] |
| 25 | 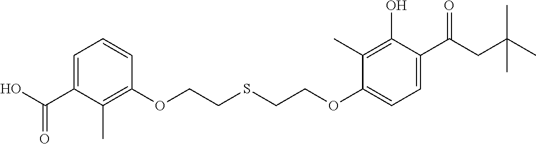 [SMILES: Cc1c(OCCSCCOc2ccc(C(=O)CC(C)(C)C)c(O)c2C)cccc1C(=O)O] |
| 26 | 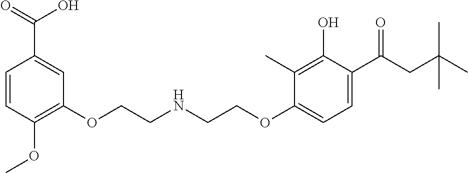 [SMILES: COc1ccc(C(=O)O)cc1OCCNCCOc1ccc(C(=O)CC(C)(C)C)c(O)c1C] |
| 27 | 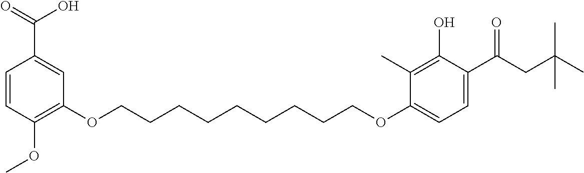 [SMILES: COc1ccc(C(=O)O)cc1OCCCCCCCCCOc1ccc(C(=O)CC(C)(C)C)c(O)c1C] |
| 28 | 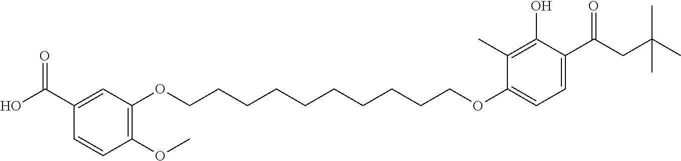 [SMILES: COc1ccc(C(=O)O)cc1OCCCCCCCCCCOc1ccc(C(=O)CC(C)(C)C)c(O)c1C] |
| 29 | 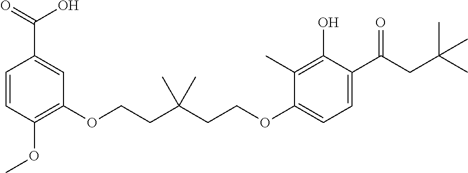 [SMILES: COc1ccc(C(=O)O)cc1OCCC(C)(C)CCOc1ccc(C(=O)CC(C)(C)C)c(O)c1C] |
| 30 | 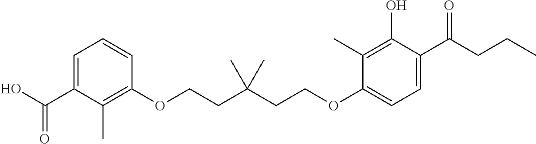 [SMILES: CCCC(=O)c1ccc(OCCC(C)(C)CCOc2cccc(C(=O)O)c2C)c(C)c1O] |
| 31 | 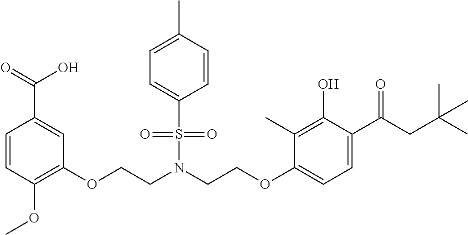 [SMILES: COc1ccc(C(=O)O)cc1OCCN(CCOc1ccc(C(=O)CC(C)(C)C)c(O)c1C)S(=O)(=O)c1ccc(C)cc1] |
| 32 | 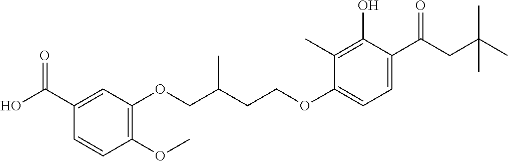 [SMILES: COc1ccc(C(=O)O)cc1OCC(C)CCOc1ccc(C(=O)CC(C)(C)C)c(O)c1C] |
| 33 | 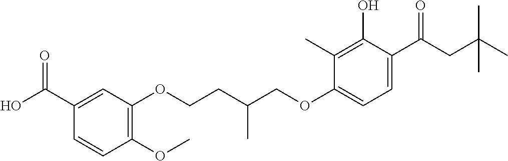 [SMILES: COc1ccc(C(=O)O)cc1OCCC(C)COc1ccc(C(=O)CC(C)(C)C)c(O)c1C] |
| 34 | 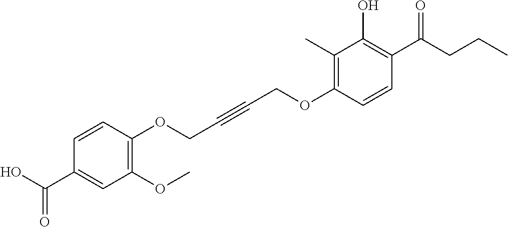 [SMILES: CCCC(=O)c1ccc(OCC#CCOc2ccc(C(=O)O)cc2OC)c(C)c1O] |
| 35 | 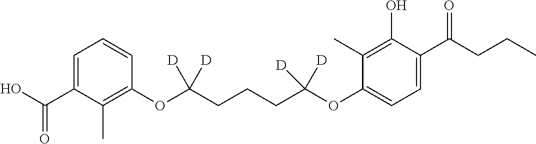 [SMILES: [2H]C([2H])(CCCC([2H])([2H])Oc1ccc(C(=O)CCC)c(O)c1C)Oc1cccc(C(=O)O)c1C] |
| 36 | 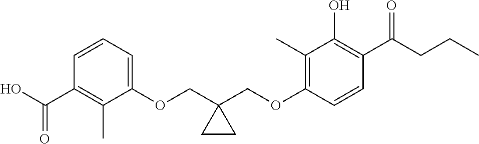 [SMILES: CCCC(=O)c1ccc(OCC2(COc3cccc(C(=O)O)c3C)CC2)c(C)c1O] |
| 37 | 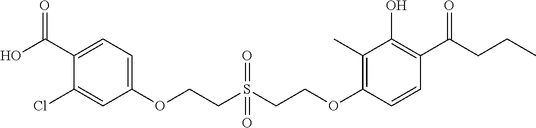 [SMILES: CCCC(=O)c1ccc(OCCS(=O)(=O)CCOc2ccc(C(=O)O)c(Cl)c2)c(C)c1O] |
| 38 | 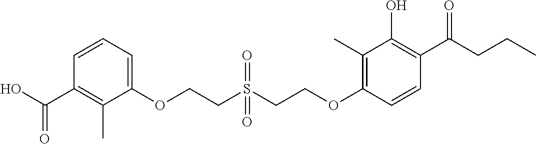 [SMILES: CCCC(=O)c1ccc(OCCS(=O)(=O)CCOc2cccc(C(=O)O)c2C)c(C)c1O] |
| 39 | 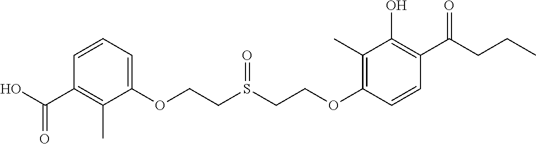 [SMILES: CCCC(=O)c1ccc(OCCS(=O)CCOc2cccc(C(=O)O)c2C)c(C)c1O] |
| 40 | 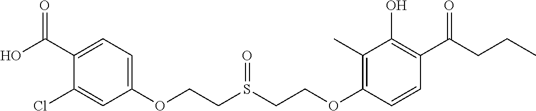 [SMILES: CCCC(=O)c1ccc(OCCS(=O)CCOc2ccc(C(=O)O)c(Cl)c2)c(C)c1O] |
| 41 | 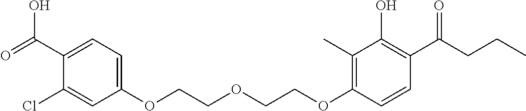 [SMILES: CCCC(=O)c1ccc(OCCOCCOc2ccc(C(=O)O)c(Cl)c2)c(C)c1O] |
| 42 | 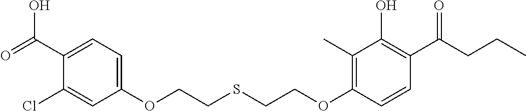 [SMILES: CCCC(=O)c1ccc(OCCSCCOc2ccc(C(=O)O)c(Cl)c2)c(C)c1O] |
| 43 | 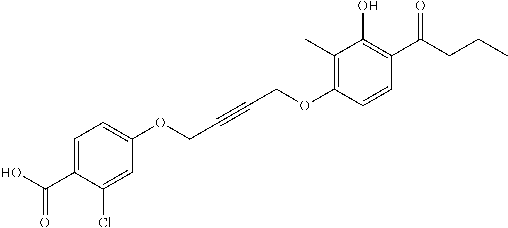 [SMILES: CCCC(=O)c1ccc(OCC#CCOc2ccc(C(=O)O)c(Cl)c2)c(C)c1O] |
| 44 | 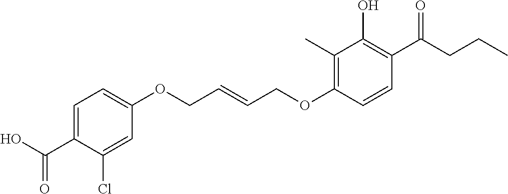 [SMILES: CCCC(=O)c1ccc(OC/C=C/COc2ccc(C(=O)O)c(Cl)c2)c(C)c1O] |
| 45 | 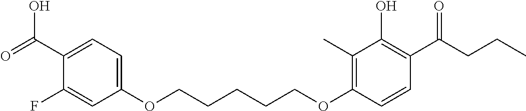 [SMILES: CCCC(=O)c1ccc(OCCCCCOc2ccc(C(=O)O)c(F)c2)c(C)c1O] |
| 46 | 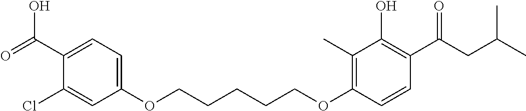 [SMILES: Cc1c(OCCCCCOc2ccc(C(=O)O)c(Cl)c2)ccc(C(=O)CC(C)C)c1O] |
| 47 | 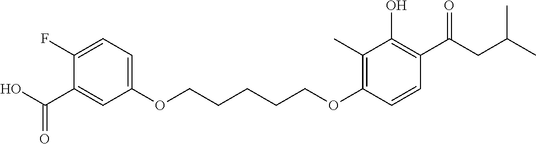 [SMILES: Cc1c(OCCCCCOc2ccc(F)c(C(=O)O)c2)ccc(C(=O)CC(C)C)c1O] |
| 48 | 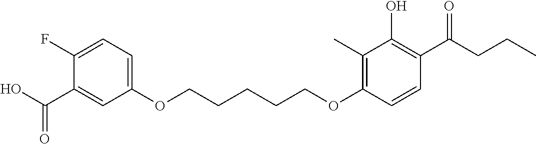 [SMILES: CCCC(=O)c1ccc(OCCCCCOc2ccc(F)c(C(=O)O)c2)c(C)c1O] |
| 49 | 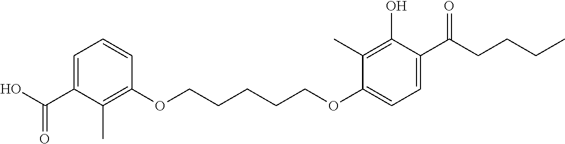 [SMILES: CCCCC(=O)c1ccc(OCCCCCOc2cccc(C(=O)O)c2C)c(C)c1O] |
| 50 | 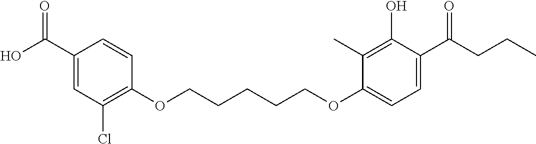 [SMILES: CCCC(=O)c1ccc(OCCCCCOc2ccc(C(=O)O)cc2Cl)c(C)c1O] |
| 51 | 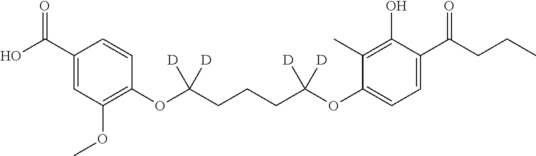 [SMILES: [2H]C([2H])(CCCC([2H])([2H])Oc1ccc(C(=O)CCC)c(O)c1C)Oc1ccc(C(=O)O)cc1OC] |
| 52 | 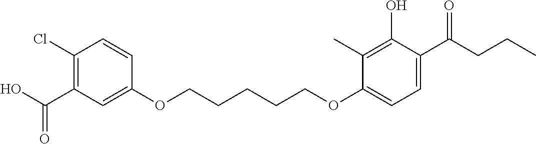 [SMILES: CCCC(=O)c1ccc(OCCCCCOc2ccc(Cl)c(C(=O)O)c2)c(C)c1O] |
| 53 | 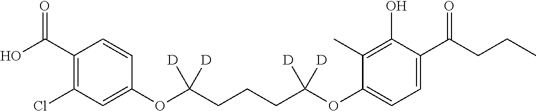 [SMILES: [2H]C([2H])(CCCC([2H])([2H])Oc1ccc(C(=O)CCC)c(O)c1C)Oc1ccc(C(=O)O)c(Cl)c1] |
| 54 | 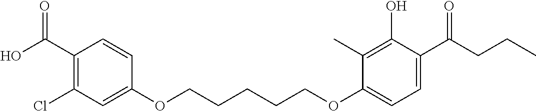 [SMILES: CCCC(=O)c1ccc(OCCCCCOc2ccc(C(=O)O)c(Cl)c2)c(C)c1O] |
| 55 | 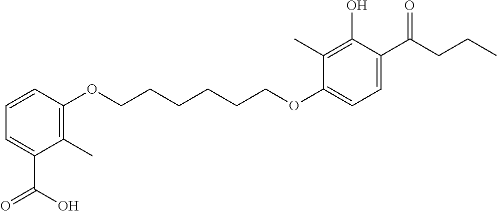 [SMILES: CCCC(=O)c1ccc(OCCCCCCOc2cccc(C(=O)O)c2C)c(C)c1O] |
| 56 | 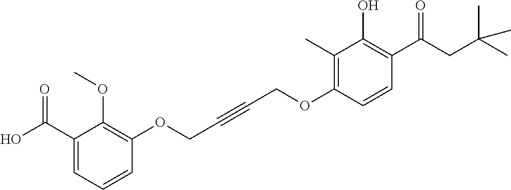 [SMILES: COc1c(OCC#CCOc2ccc(C(=O)CC(C)(C)C)c(O)c2C)cccc1C(=O)O] |
| 57 | 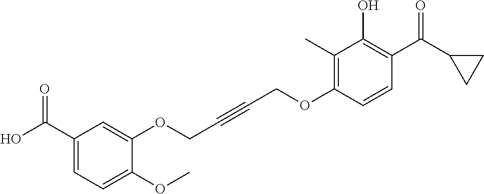 [SMILES: COc1ccc(C(=O)O)cc1OCC#CCOc1ccc(C(=O)C2CC2)c(O)c1C] |
| 58 | 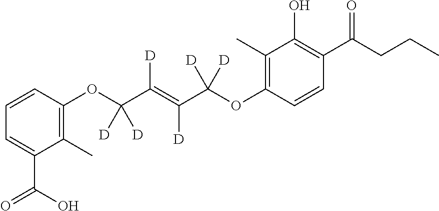 [SMILES: [2H]/C(=C(/[2H])C([2H])([2H])Oc1ccc(C(=O)CCC)c(O)c1C)C([2H])([2H])Oc1cccc(C(=O)O)c1C] |

In some embodiments, the compound is any of the compounds represented in Table 2, or a pharmaceutically acceptable salt or solvate thereof.

TABLE 2

| Cmpd. | Structure |
|---|---|
| 59 | 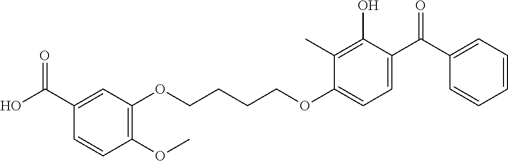 [SMILES: COc1ccc(C(=O)O)cc1OCCCCOc1ccc(C(=O)c2ccccc2)c(O)c1C] |
| 60 | 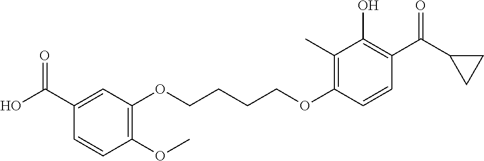 [SMILES: COc1ccc(C(=O)O)cc1OCCCCOc1ccc(C(=O)C2CC2)c(O)c1C] |
| 61 | 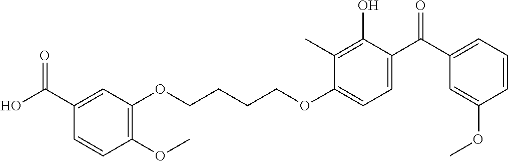 [SMILES: COc1cccc(C(=O)c2ccc(OCCCCOc3cc(C(=O)O)ccc3OC)c(C)c2O)c1] |
| 62 | 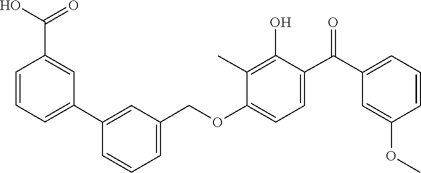 [SMILES: COc1cccc(C(=O)c2ccc(OCc3cccc(-c4cccc(C(=O)O)c4)c3)c(C)c2O)c1] |
| 63 | 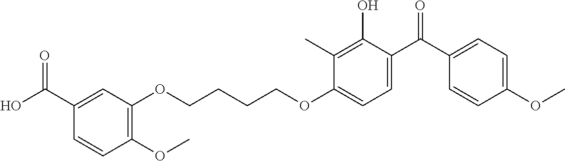 [SMILES: COc1ccc(C(=O)c2ccc(OCCCCOc3cc(C(=O)O)ccc3OC)c(C)c2O)cc1] |
| 64 | 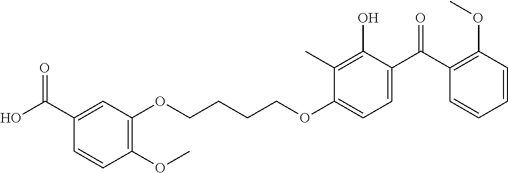 [SMILES: COc1ccc(C(=O)O)cc1OCCCCOc1ccc(C(=O)c2ccccc2OC)c(O)c1C] |
| 65 | 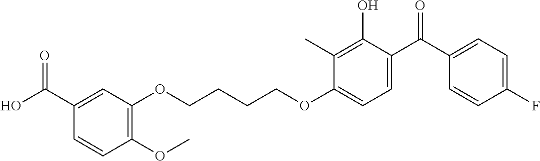 [SMILES: COc1ccc(C(=O)O)cc1OCCCCOc1ccc(C(=O)c2ccc(F)cc2)c(O)c1C] |
| 66 | 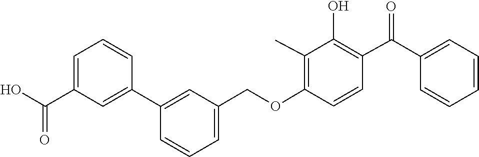 [SMILES: Cc1c(OCc2cccc(-c3cccc(C(=O)O)c3)c2)ccc(C(=O)c2ccccc2)c1O] |
| 67 | 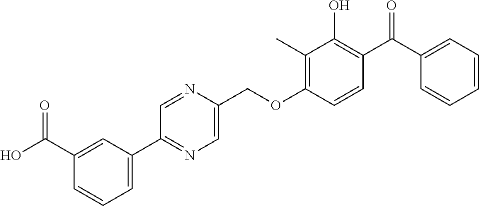 [SMILES: Cc1c(OCc2cnc(-c3cccc(C(=O)O)c3)cn2)ccc(C(=O)c2ccccc2)c1O] |
| 68 | 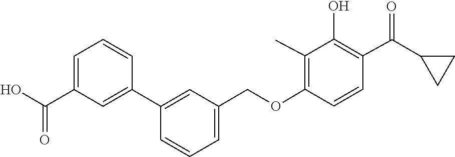 [SMILES: Cc1c(OCc2cccc(-c3cccc(C(=O)O)c3)c2)ccc(C(=O)C2CC2)c1O] |
| 69 | 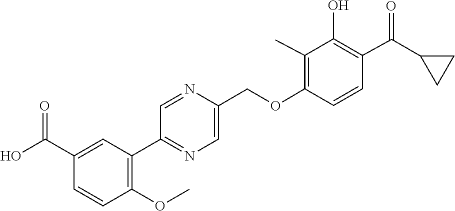 [SMILES: COc1ccc(C(=O)O)cc1-c1cnc(COc2ccc(C(=O)C3CC3)c(O)c2C)cn1] |
| 70 | 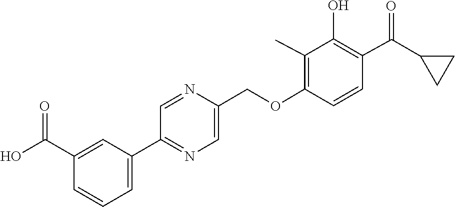 [SMILES: Cc1c(OCc2cnc(-c3cccc(C(=O)O)c3)cn2)ccc(C(=O)C2CC2)c1O] |
| 71 | 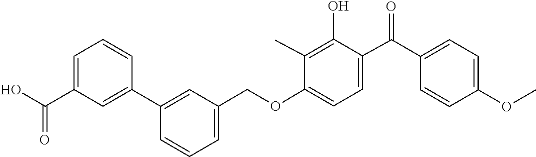 [SMILES: COc1ccc(C(=O)c2ccc(OCc3cccc(-c4cccc(C(=O)O)c4)c3)c(C)c2O)cc1] |
| 72 | 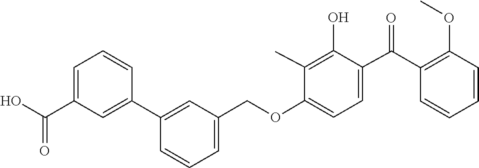 [SMILES: COc1ccccc1C(=O)c1ccc(OCc2cccc(-c3cccc(C(=O)O)c3)c2)c(C)c1O] |
| 73 | 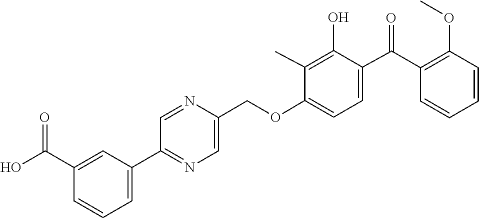 [SMILES: COc1ccccc1C(=O)c1ccc(OCc2cnc(-c3cccc(C(=O)O)c3)cn2)c(C)c1O] |
| 74 | 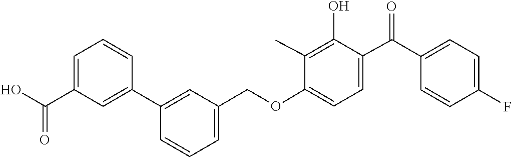 [SMILES: Cc1c(OCc2cccc(-c3cccc(C(=O)O)c3)c2)ccc(C(=O)c2ccc(F)cc2)c1O] |
| 75 | 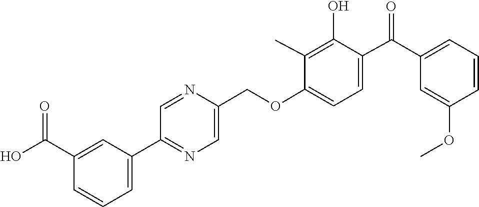 [SMILES: COc1cccc(C(=O)c2ccc(OCc3cnc(-c4cccc(C(=O)O)c4)cn3)c(C)c2O)c1] |
| 76 | 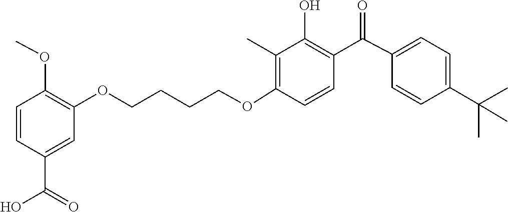 [SMILES: COc1ccc(C(=O)O)cc1OCCCCOc1ccc(C(=O)c2ccc(C(C)(C)C)cc2)c(O)c1C] |
| 77 | 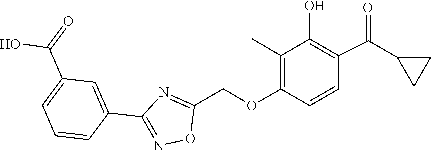 [SMILES: Cc1c(OCc2nc(-c3cccc(C(=O)O)c3)no2)ccc(C(=O)C2CC2)c1O] |
| 78 | 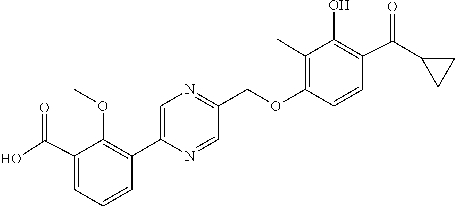 [SMILES: COc1c(C(=O)O)cccc1-c1cnc(COc2ccc(C(=O)C3CC3)c(O)c2C)cn1] |
| 79 | 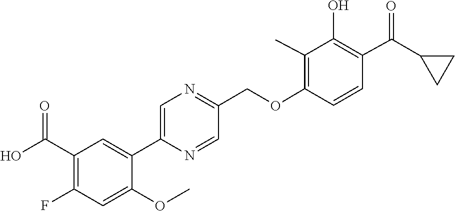 [SMILES: COc1cc(F)c(C(=O)O)cc1-c1cnc(COc2ccc(C(=O)C3CC3)c(O)c2C)cn1] |

In some embodiments, the compound is any of the compounds represented in Table 3, or a pharmaceutically acceptable salt or solvate thereof.

TABLE 3

| Cmpd. | Structure |
|---|---|
| 80 | 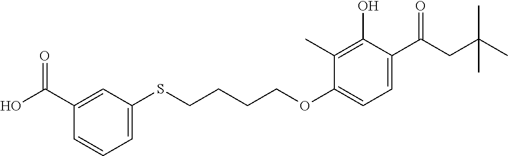 [SMILES: Cc1c(OCCCCSc2cccc(C(=O)O)c2)ccc(C(=O)CC(C)(C)C)c1O] |
| 81 | 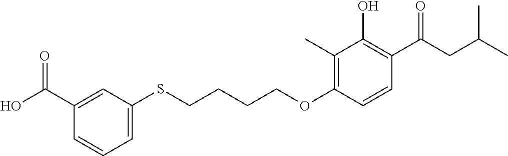 [SMILES: Cc1c(OCCCCSc2cccc(C(=O)O)c2)ccc(C(=O)CC(C)C)c1O] |
| 82 | 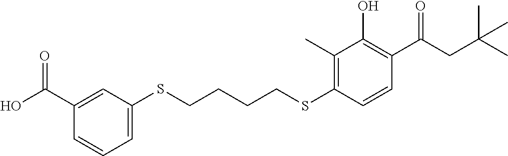 [SMILES: Cc1c(SCCCCSc2cccc(C(=O)O)c2)ccc(C(=O)CC(C)(C)C)c1O] |
| 83 | 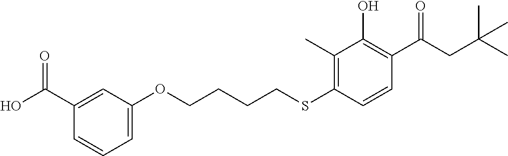 [SMILES: Cc1c(SCCCCOc2cccc(C(=O)O)c2)ccc(C(=O)CC(C)(C)C)c1O] |
| 84 | 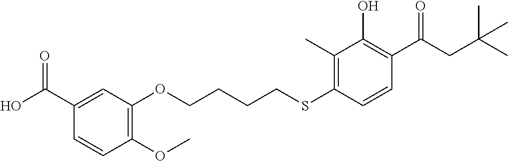 [SMILES: COc1ccc(C(=O)O)cc1OCCCCSc1ccc(C(=O)CC(C)(C)C)c(O)c1C] |
| 85 | 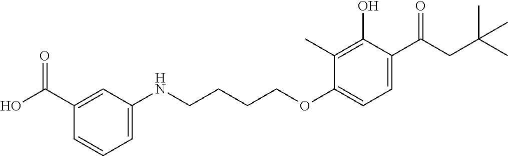 [SMILES: Cc1c(OCCCCNc2cccc(C(=O)O)c2)ccc(C(=O)CC(C)(C)C)c1O] |

In some embodiments, the compound is any of the compounds represented in Table 4, or a pharmaceutically acceptable salt or solvate thereof.

TABLE 4

| Cmpd. | Structure |
|---|---|
| 86 | 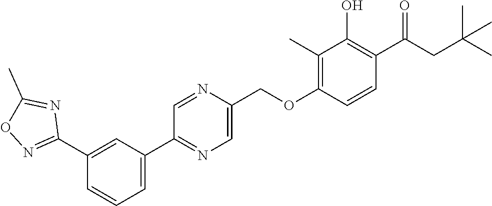 [SMILES: Cc1nc(-c2cccc(-c3cnc(COc4ccc(C(=O)CC(C)(C)C)c(O)c4C)cn3)c2)no1] |
| 87 | 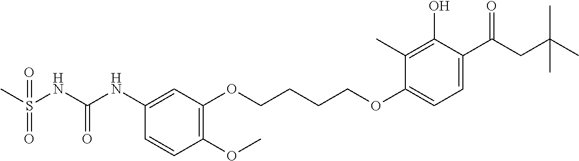 [SMILES: COc1ccc(NC(=O)NS(C)(=O)=O)cc1OCCCCOc1ccc(C(=O)CC(C)(C)C)c(O)c1C] |
| 88 | 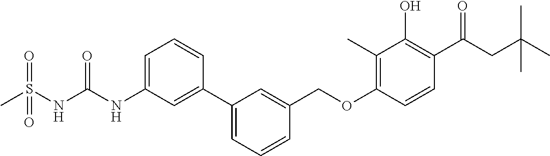 [SMILES: Cc1c(OCc2cccc(-c3cccc(NC(=O)NS(C)(=O)=O)c3)c2)ccc(C(=O)CC(C)(C)C)c1O] |
| 89 | 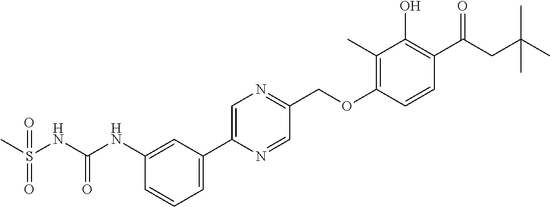 [SMILES: Cc1c(OCc2cnc(-c3cccc(NC(=O)NS(C)(=O)=O)c3)cn2)ccc(C(=O)CC(C)(C)C)c1O] |
| 90 | 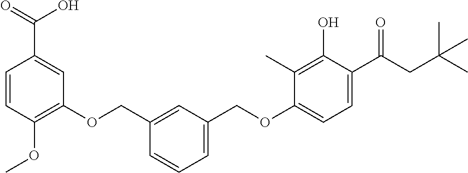 [SMILES: COc1ccc(C(=O)O)cc1OCc1cccc(COc2ccc(C(=O)CC(C)(C)C)c(O)c2C)c1] |
| 91 | 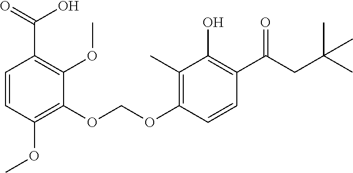 [SMILES: COc1ccc(C(=O)O)c(OC)c1OCOc1ccc(C(=O)CC(C)(C)C)c(O)c1C] |
| 92 | 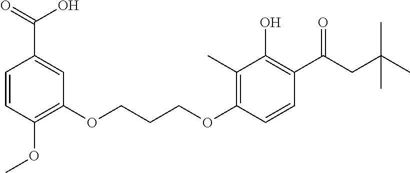 [SMILES: COc1ccc(C(=O)O)cc1OCCCOc1ccc(C(=O)CC(C)(C)C)c(O)c1C] |
| 93 | 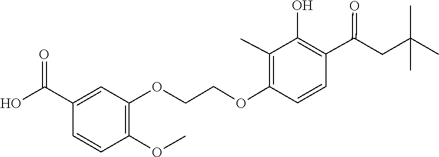 [SMILES: COc1ccc(C(=O)O)cc1OCCOc1ccc(C(=O)CC(C)(C)C)c(O)c1C] |
| 94 | 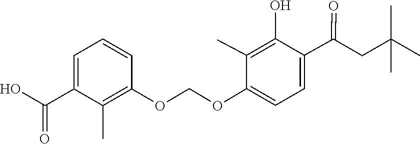 [SMILES: Cc1c(OCOc2ccc(C(=O)CC(C)(C)C)c(O)c2C)cccc1C(=O)O] |
| 95 | 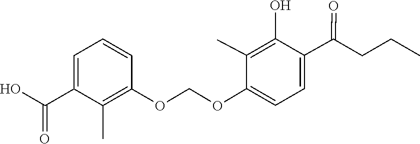 [SMILES: CCCC(=O)c1ccc(OCOc2cccc(C(=O)O)c2C)c(C)c1O] |
| 96 | 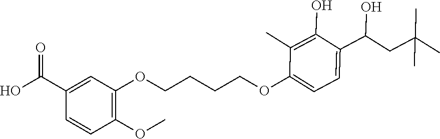 [SMILES: COc1ccc(C(=O)O)cc1OCCCCOc1ccc(C(O)CC(C)(C)C)c(O)c1C] |
| 97 | 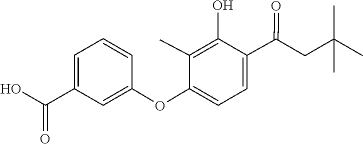 [SMILES: Cc1c(Oc2cccc(C(=O)O)c2)ccc(C(=O)CC(C)(C)C)c1O] |
| 98 | 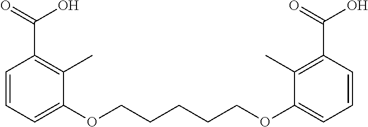 [SMILES: Cc1c(OCCCCCOc2cccc(C(=O)O)c2C)cccc1C(=O)O] |
| 99 | 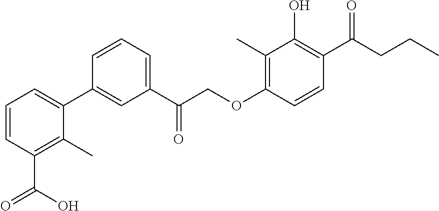 [SMILES: CCCC(=O)c1ccc(OCC(=O)c2cccc(-c3cccc(C(=O)O)c3C)c2)c(C)c1O] |
| 100 | 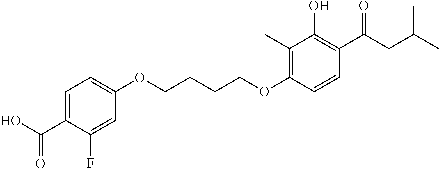 [SMILES: Cc1c(OCCCCOc2ccc(C(=O)O)c(F)c2)ccc(C(=O)CC(C)C)c1O] |
| 101 | 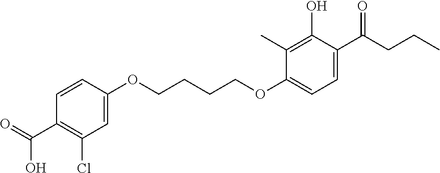 [SMILES: CCCC(=O)c1ccc(OCCCCOc2ccc(C(=O)O)c(Cl)c2)c(C)c1O] |
| 102 | 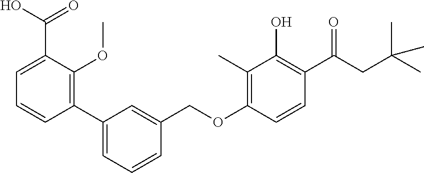 [SMILES: COc1c(C(=O)O)cccc1-c1cccc(COc2ccc(C(=O)CC(C)(C)C)c(O)c2C)c1] |
| 103 | 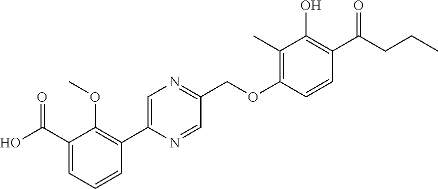 [SMILES: CCCC(=O)c1ccc(OCc2cnc(-c3cccc(C(=O)O)c3OC)cn2)c(C)c1O] |
| 104 | 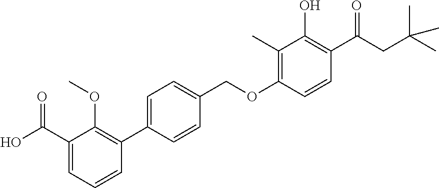 [SMILES: COc1c(C(=O)O)cccc1-c1ccc(COc2ccc(C(=O)CC(C)(C)C)c(O)c2C)cc1] |
| 105 | 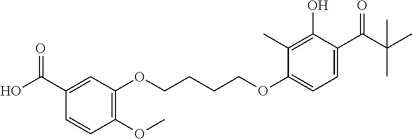 [SMILES: COc1ccc(C(=O)O)cc1OCCCCOc1ccc(C(=O)C(C)(C)C)c(O)c1C] |
| 106 | 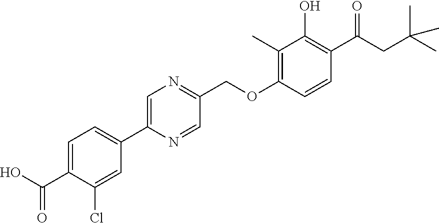 [SMILES: Cc1c(OCc2cnc(-c3ccc(C(=O)O)c(Cl)c3)cn2)ccc(C(=O)CC(C)(C)C)c1O] |
| 107 | 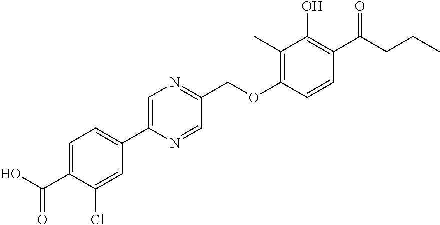 [SMILES: CCCC(=O)c1ccc(OCc2cnc(-c3ccc(C(=O)O)c(Cl)c3)cn2)c(C)c1O] |
| 108 | 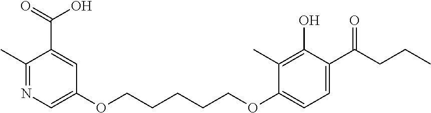 [SMILES: CCCC(=O)c1ccc(OCCCCCOc2cnc(C)c(C(=O)O)c2)c(C)c1O] |
| 109 | 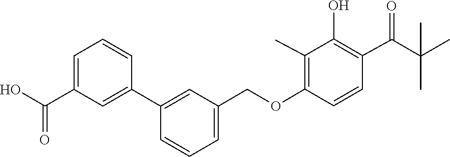 [SMILES: Cc1c(OCc2cccc(-c3cccc(C(=O)O)c3)c2)ccc(C(=O)C(C)(C)C)c1O] |
| 110 | 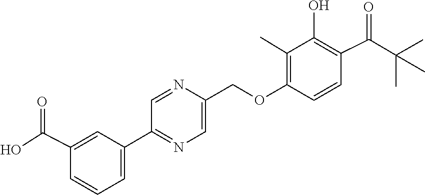 [SMILES: Cc1c(OCc2cnc(-c3cccc(C(=O)O)c3)cn2)ccc(C(=O)C(C)(C)C)c1O] |
| 111 | 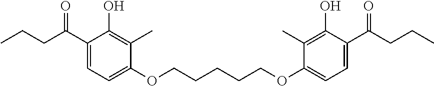 [SMILES: CCCC(=O)c1ccc(OCCCCCOc2ccc(C(=O)CCC)c(O)c2C)c(C)c1O] |
| 112 | 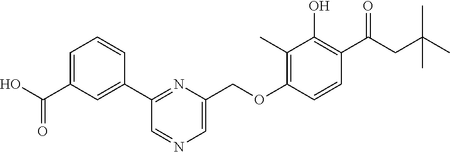 [SMILES: Cc1c(OCc2cncc(-c3cccc(C(=O)O)c3)n2)ccc(C(=O)CC(C)(C)C)c1O] |
| 113 | 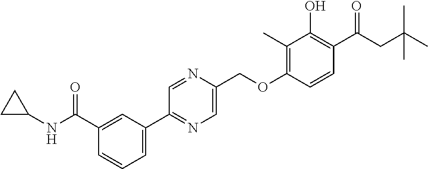 [SMILES: Cc1c(OCc2cnc(-c3cccc(C(=O)NC4CC4)c3)cn2)ccc(C(=O)CC(C)(C)C)c1O] |
| 114 | 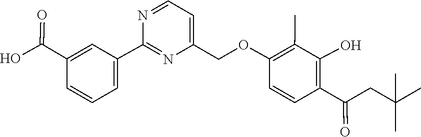 [SMILES: Cc1c(OCc2ccnc(-c3cccc(C(=O)O)c3)n2)ccc(C(=O)CC(C)(C)C)c1O] |
| 115 | 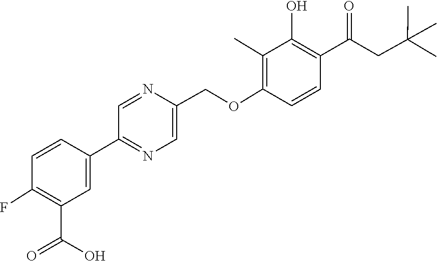 [SMILES: Cc1c(OCc2cnc(-c3ccc(F)c(C(=O)O)c3)cn2)ccc(C(=O)CC(C)(C)C)c1O] |
| 116 | 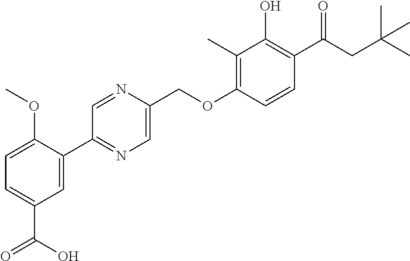 [SMILES: COc1ccc(C(=O)O)cc1-c1cnc(COc2ccc(C(=O)CC(C)(C)C)c(O)c2C)cn1] |
| 117 | 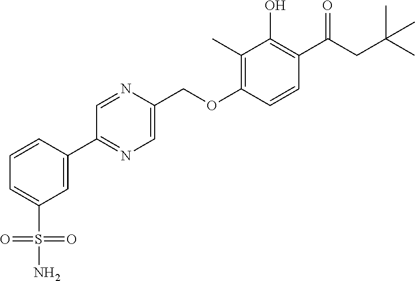 [SMILES: Cc1c(OCc2cnc(-c3cccc(S(N)(=O)=O)c3)cn2)ccc(C(=O)CC(C)(C)C)c1O] |
| 118 | 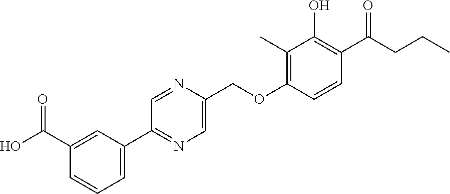 [SMILES: CCCC(=O)c1ccc(OCc2cnc(-c3cccc(C(=O)O)c3)cn2)c(C)c1O] |
| 119 | 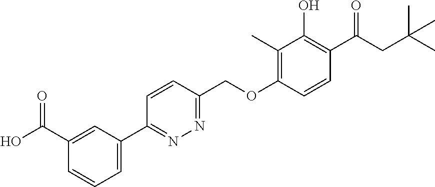 [SMILES: Cc1c(OCc2ccc(-c3cccc(C(=O)O)c3)nn2)ccc(C(=O)CC(C)(C)C)c1O] |
| 120 | 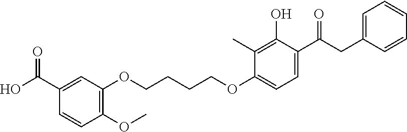 [SMILES: COc1ccc(C(=O)O)cc1OCCCCOc1ccc(C(=O)Cc2ccccc2)c(O)c1C] |
| 121 | 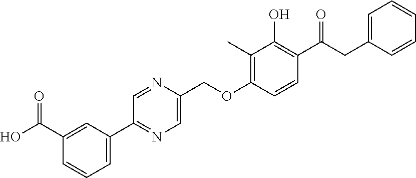 [SMILES: Cc1c(OCc2cnc(-c3cccc(C(=O)O)c3)cn2)ccc(C(=O)Cc2ccccc2)c1O] |
| 122 | 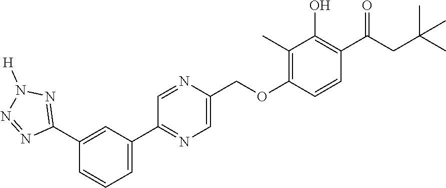 [SMILES: Cc1c(OCc2cnc(-c3cccc(-c4nn[nH]n4)c3)cn2)ccc(C(=O)CC(C)(C)C)c1O] |
| 123 | 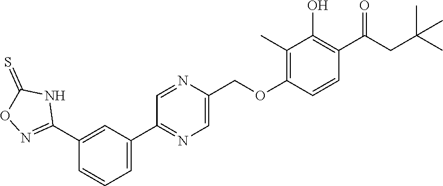 [SMILES: Cc1c(OCc2cnc(-c3cccc(-c4noc(=S)[nH]4)c3)cn2)ccc(C(=O)CC(C)(C)C)c1O] |
| 124 | 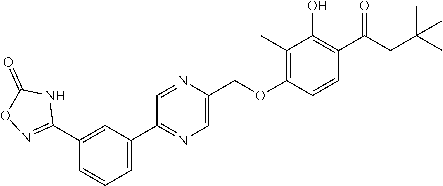 [SMILES: Cc1c(OCc2cnc(-c3cccc(-c4noc(=O)[nH]4)c3)cn2)ccc(C(=O)CC(C)(C)C)c1O] |
| 125 | 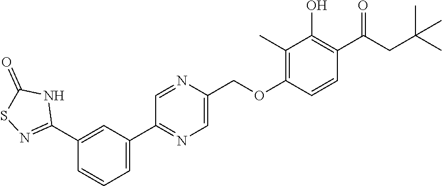 [SMILES: Cc1c(OCc2cnc(-c3cccc(-c4nsc(=O)[nH]4)c3)cn2)ccc(C(=O)CC(C)(C)C)c1O] |
| 126 | 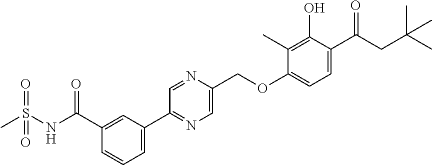 [SMILES: Cc1c(OCc2cnc(-c3cccc(C(=O)NS(C)(=O)=O)c3)cn2)ccc(C(=O)CC(C)(C)C)c1O] |
| 127 | 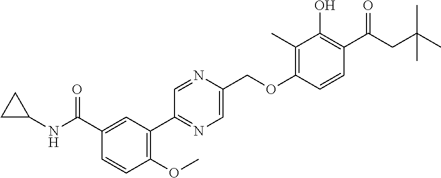 [SMILES: COc1ccc(C(=O)NC2CC2)cc1-c1cnc(COc2ccc(C(=O)CC(C)(C)C)c(O)c2C)cn1] |
| 128 | 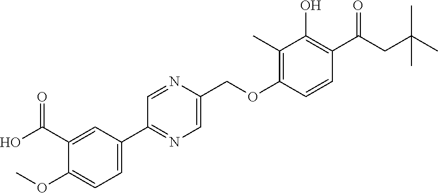 [SMILES: COc1ccc(-c2cnc(COc3ccc(C(=O)CC(C)(C)C)c(O)c3C)cn2)cc1C(=O)O] |
| 129 | 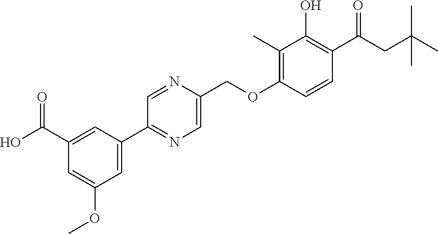 [SMILES: COc1cc(C(=O)O)cc(-c2cnc(COc3ccc(C(=O)CC(C)(C)C)c(O)c3C)cn2)c1] |
| 130 | 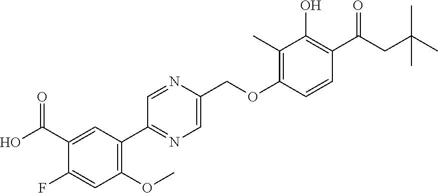 [SMILES: COc1cc(F)c(C(=O)O)cc1-c1cnc(COc2ccc(C(=O)CC(C)(C)C)c(O)c2C)cn1] |
| 131 | 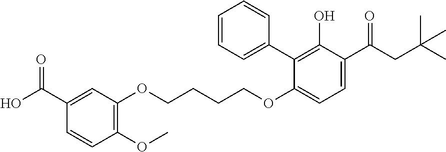 [SMILES: COc1ccc(C(=O)O)cc1OCCCCOc1ccc(C(=O)CC(C)(C)C)c(O)c1-c1ccccc1] |
| 132 | 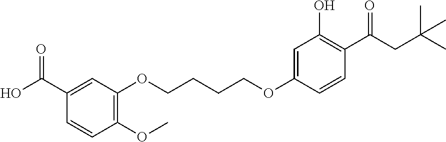 [SMILES: COc1ccc(C(=O)O)cc1OCCCCOc1ccc(C(=O)CC(C)(C)C)c(O)c1] |
| 133 | 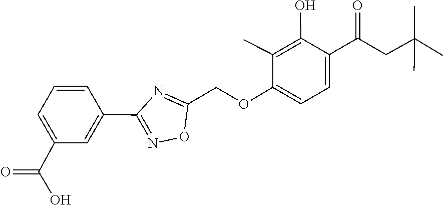 [SMILES: Cc1c(OCc2nc(-c3cccc(C(=O)O)c3)no2)ccc(C(=O)CC(C)(C)C)c1O] |
| 134 | 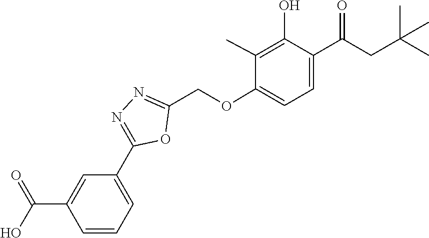 [SMILES: Cc1c(OCc2nnc(-c3cccc(C(=O)O)c3)o2)ccc(C(=O)CC(C)(C)C)c1O] |
| 135 | 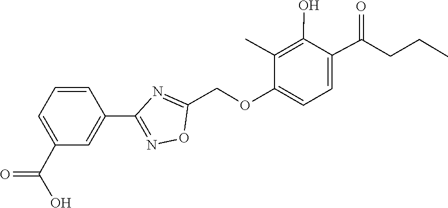 [SMILES: CCCC(=O)c1ccc(OCc2nc(-c3cccc(C(=O)O)c3)no2)c(C)c1O] |
| 136 | 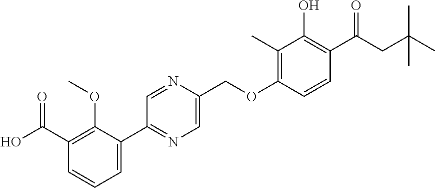 [SMILES: COc1c(C(=O)O)cccc1-c1cnc(COc2ccc(C(=O)CC(C)(C)C)c(O)c2C)cn1] |
| 137 | 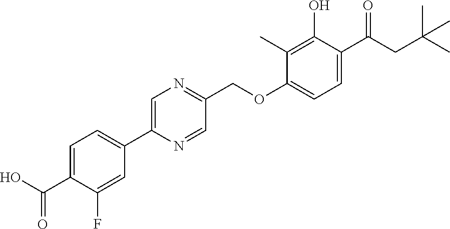 [SMILES: Cc1c(OCc2cnc(-c3ccc(C(=O)O)c(F)c3)cn2)ccc(C(=O)CC(C)(C)C)c1O] |
| 138 | 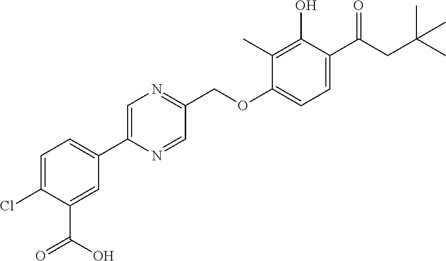 [SMILES: Cc1c(OCc2cnc(-c3ccc(Cl)c(C(=O)O)c3)cn2)ccc(C(=O)CC(C)(C)C)c1O] |
| 139 | 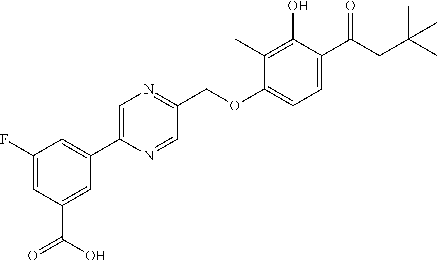 [SMILES: Cc1c(OCc2cnc(-c3cc(F)cc(C(=O)O)c3)cn2)ccc(C(=O)CC(C)(C)C)c1O] |
| 140 | 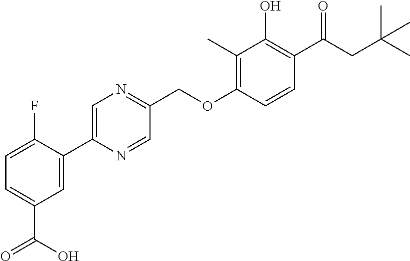 [SMILES: Cc1c(OCc2cnc(-c3cc(C(=O)O)ccc3F)cn2)ccc(C(=O)CC(C)(C)C)c1O] |
| 141 | 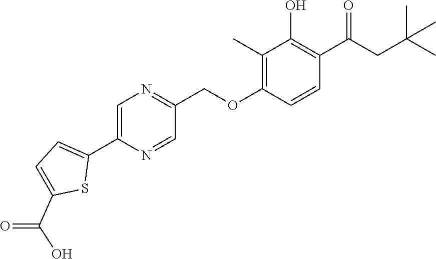 [SMILES: Cc1c(OCc2cnc(-c3ccc(C(=O)O)s3)cn2)ccc(C(=O)CC(C)(C)C)c1O] |
| 142 | 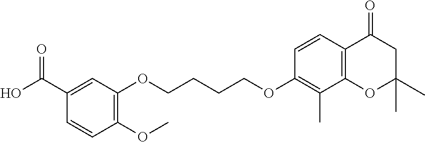 [SMILES: COc1ccc(C(=O)O)cc1OCCCCOc1ccc2c(c1C)OC(C)(C)CC2=O] |
| 143 | 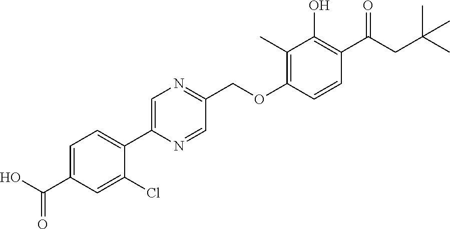 [SMILES: Cc1c(OCc2cnc(-c3ccc(C(=O)O)cc3Cl)cn2)ccc(C(=O)CC(C)(C)C)c1O] |
| 144 | 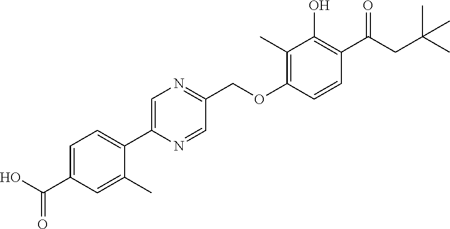 [SMILES: Cc1cc(C(=O)O)ccc1-c1cnc(COc2ccc(C(=O)CC(C)(C)C)c(O)c2C)cn1] |
| 145 | 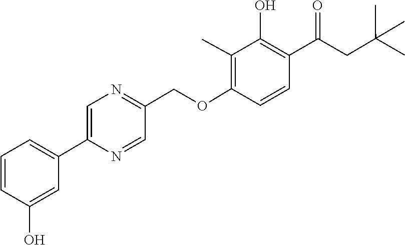 [SMILES: Cc1c(OCc2cnc(-c3cccc(O)c3)cn2)ccc(C(=O)CC(C)(C)C)c1O] |
| 146 | 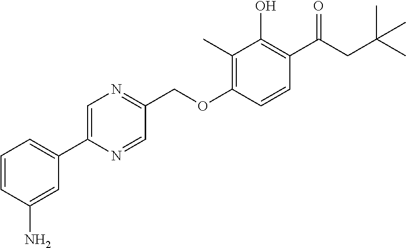 [SMILES: Cc1c(OCc2cnc(-c3cccc(N)c3)cn2)ccc(C(=O)CC(C)(C)C)c1O] |
| 147 | 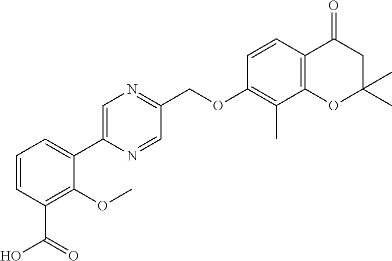 [SMILES: COc1c(C(=O)O)cccc1-c1cnc(COc2ccc3c(c2C)OC(C)(C)CC3=O)cn1] |
| 148 | 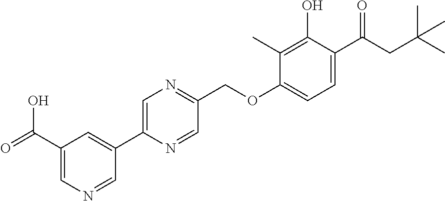 [SMILES: Cc1c(OCc2cnc(-c3cncc(C(=O)O)c3)cn2)ccc(C(=O)CC(C)(C)C)c1O] |
| 149 | 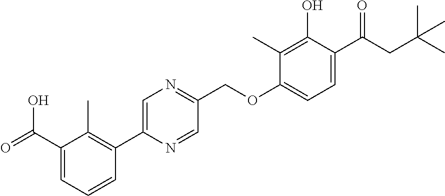 [SMILES: Cc1c(C(=O)O)cccc1-c1cnc(COc2ccc(C(=O)CC(C)(C)C)c(O)c2C)cn1] |
| 150 | 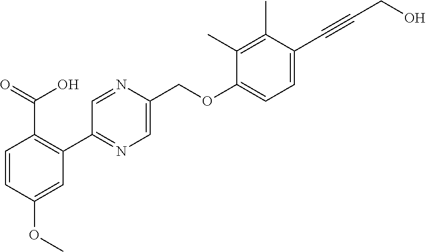 [SMILES: COc1ccc(C(=O)O)c(-c2cnc(COc3ccc(C#CCO)c(C)c3C)cn2)c1] |
| 151 | 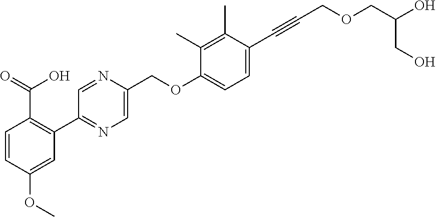 [SMILES: COc1ccc(C(=O)O)c(-c2cnc(COc3ccc(C#CCOCC(O)CO)c(C)c3C)cn2)c1] |
| 152 | 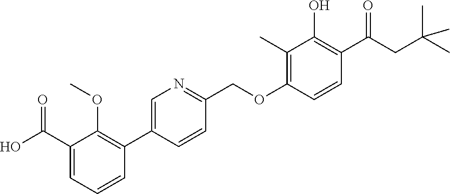 [SMILES: COc1c(C(=O)O)cccc1-c1ccc(COc2ccc(C(=O)CC(C)(C)C)c(O)c2C)nc1] |
| 153 | 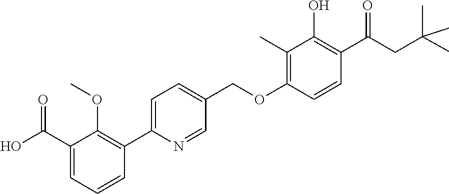 [SMILES: COc1c(C(=O)O)cccc1-c1ccc(COc2ccc(C(=O)CC(C)(C)C)c(O)c2C)cn1] |
| 154 | 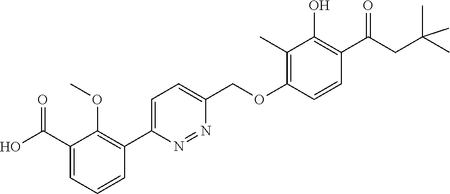 [SMILES: COc1c(C(=O)O)cccc1-c1ccc(COc2ccc(C(=O)CC(C)(C)C)c(O)c2C)nn1] |
| 155 | 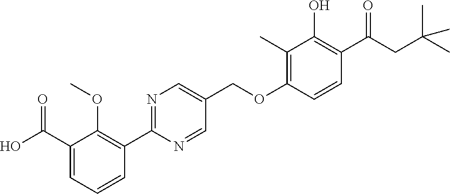 [SMILES: COc1c(C(=O)O)cccc1-c1ncc(COc2ccc(C(=O)CC(C)(C)C)c(O)c2C)cn1] |
| 156 | 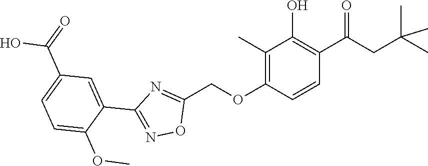 [SMILES: COc1ccc(C(=O)O)cc1-c1noc(COc2ccc(C(=O)CC(C)(C)C)c(O)c2C)n1] |
| 157 | 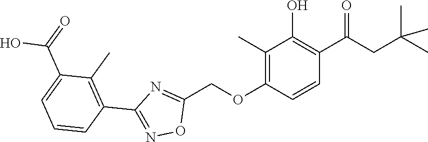 [SMILES: Cc1c(C(=O)O)cccc1-c1noc(COc2ccc(C(=O)CC(C)(C)C)c(O)c2C)n1] |
| 158 | 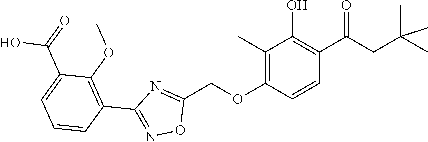 [SMILES: COc1c(C(=O)O)cccc1-c1noc(COc2ccc(C(=O)CC(C)(C)C)c(O)c2C)n1] |
| 159 | 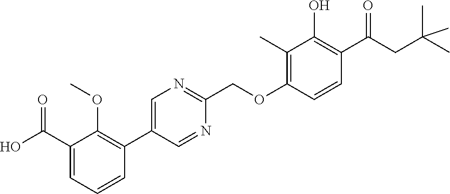 [SMILES: COc1c(C(=O)O)cccc1-c1cnc(COc2ccc(C(=O)CC(C)(C)C)c(O)c2C)nc1] |
| 160 | 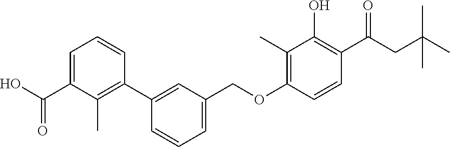 [SMILES: Cc1c(C(=O)O)cccc1-c1cccc(COc2ccc(C(=O)CC(C)(C)C)c(O)c2C)c1] |
| 161 | 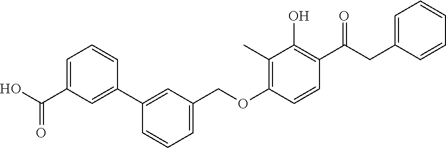 [SMILES: Cc1c(OCc2cccc(-c3cccc(C(=O)O)c3)c2)ccc(C(=O)Cc2ccccc2)c1O] |
| 162 | 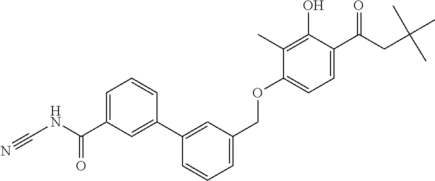 [SMILES: Cc1c(OCc2cccc(-c3cccc(C(=O)NC#N)c3)c2)ccc(C(=O)CC(C)(C)C)c1O] |
| 163 | 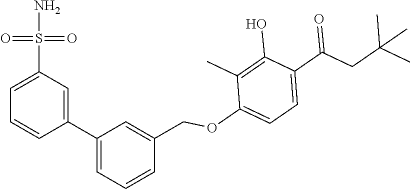 [SMILES: Cc1c(OCc2cccc(-c3cccc(S(N)(=O)=O)c3)c2)ccc(C(=O)CC(C)(C)C)c1O] |
| 164 | 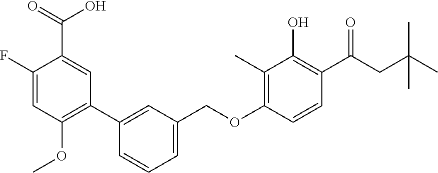 [SMILES: COc1cc(F)c(C(=O)O)cc1-c1cccc(COc2ccc(C(=O)CC(C)(C)C)c(O)c2C)c1] |
| 165 | 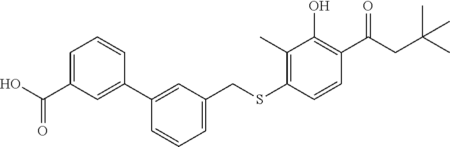 [SMILES: Cc1c(SCc2cccc(-c3cccc(C(=O)O)c3)c2)ccc(C(=O)CC(C)(C)C)c1O] |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine, and chloride, such as ²H (D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts or solvates thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Certain isotopically-labeled compounds, for example those into which radioactive isotopes such as ¹⁸F and ¹⁴C are incorporated, are useful in Positron Emission Tomography (PET), Positron Emission Tomography-Computed Tomography (PET/CT) scanning. The use of radiotracers allows for evaluation of drug distribution, receptor occupancy, diagnosis, treatment, and evaluation of various diseases

In some embodiments, the abundance of deuterium in each of the substituents disclosed herein is independently at least 1%, at least 10%, at least 20%, at least 30%, at least 40%, at least 50%, at least 60%, at least 70%, at least 80%, at least 90%, or 100% of a total number of hydrogen and deuterium. In some embodiments, one or more of the substituents disclosed herein comprise deuterium at a percentage higher than the natural abundance of deuterium. In some embodiments, one or more hydrogens are replaced with one or more deuteriums in one or more of the substituents disclosed herein.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, or solvate thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Synthesis of Compounds

In some embodiments, the synthesis of compounds described herein are accomplished using means described in the chemical literature, using the methods described herein, or by a combination thereof. In addition, solvents, temperatures, and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio, or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

In some embodiments, compounds described herein are prepared as shown in Scheme A and B.

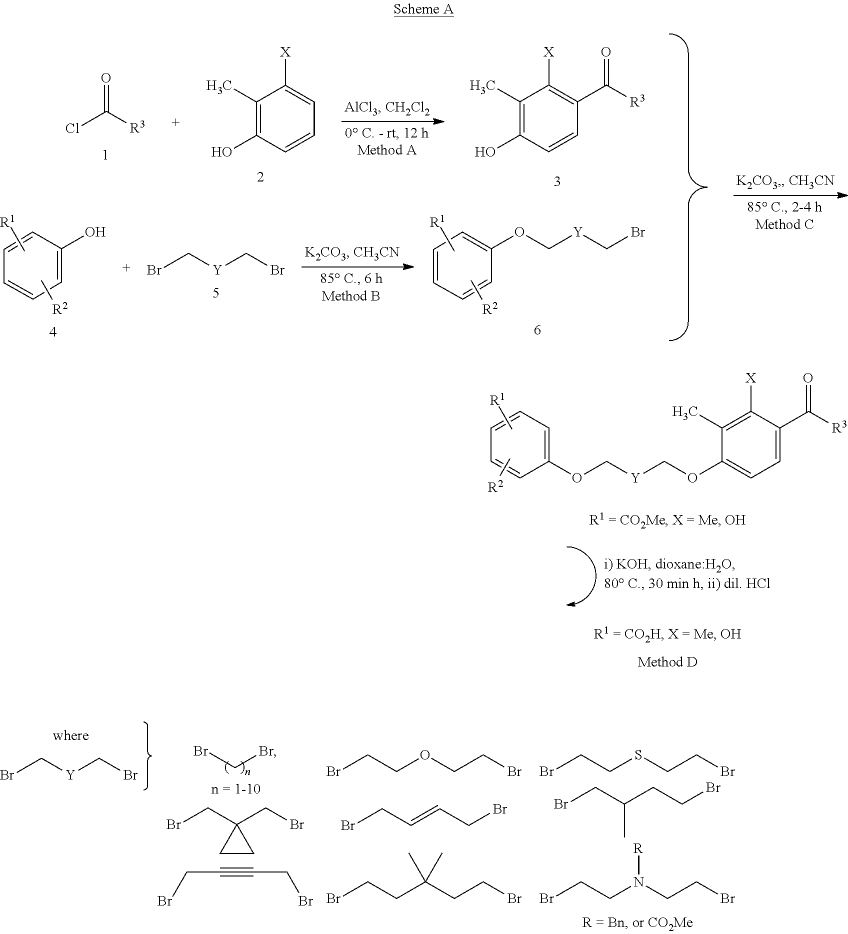

Commercially available acyl chloride derivatives (1) were employed in a Friedel-Crafts acylation of substituted phenols (2) using aluminum chloride to provide the key acetophenone derivatives 3. The hydroxy benzoate derivatives (4) were coupled with appropriate dibromo compounds 5 by heating with potassium carbonate in acetonitrile to provide the corresponding bromo intermediate derivative (6). Finally, Finkelstein alkylation of intermediate 3 with 6 under reflux conditions delivered the ester derivatives which were saponified with lithium hydroxide to provide the target carboxylic acid derivatives.

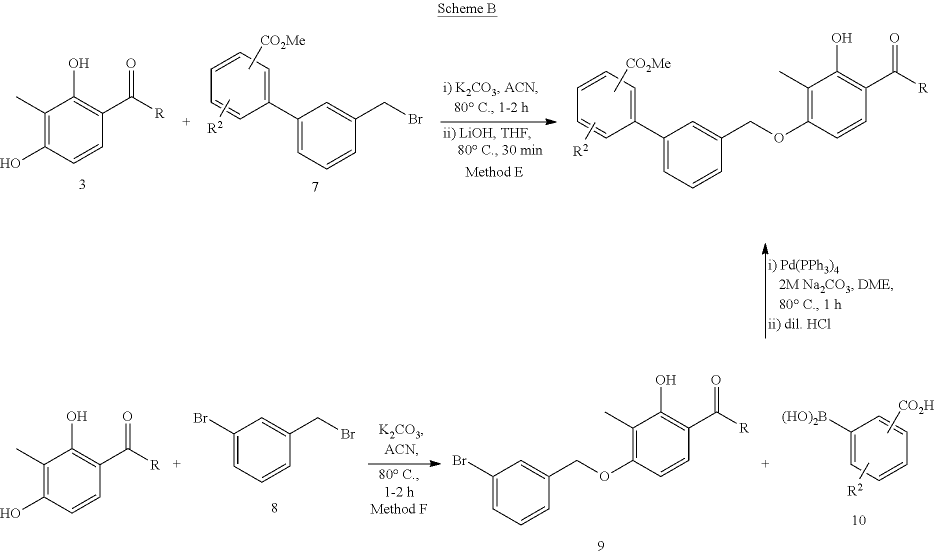

Resorcinol derivatives (3) and biphenyl derivatives (7) were reacted under basic conditions to afford the ester derivatives of which were saponified to afford the acids. Alternatively, the resorcinol derivatives (3) were reacted with bromo-benzyl bromide derivatives (8) followed by a Suzuki cross coupling reaction of the intermediate (9) with borono benzoic acid derivatives (10) to afford final targeted compounds.

It will be understood that the reactions shown above are illustrative.

In one aspect, compounds are synthesized as described in the Examples section.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

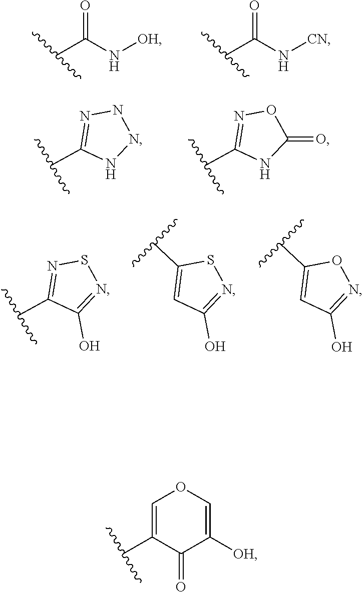

and the like.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound of Formula (I), (Ia), (II), or (III) and a co-agent, are both administered to a patient simultaneously in the form of a single entity or dosage. The term “non-fixed combination” means that the active ingredients, e.g. a compound of Formula (I), (Ia), (II), or (III), and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “subject” or “patient” encompasses mammals. Examples of mammals include, but are not limited to, humans. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

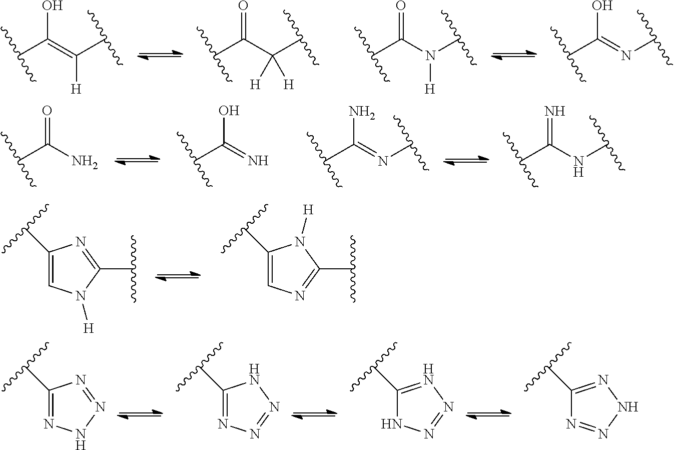

### Dosing

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of or risk factor for the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the subject every 12 hours; (v) the compound is administered to the subject every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically. In yet other embodiments, the compound described herein is administered by inhalation. In some embodiments, the compounds disclosed herein are formulated for intranasal administration. Such formulations include nasal sprays, nasal mists, and the like.

### Pharmaceutical Compositions/Formulations

The compounds described herein are administered to a subject in need thereof, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

In another aspect, provided herein are pharmaceutical compositions comprising a compound described herein, or a pharmaceutically acceptable salt or solvate thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt or solvate thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt or solvate thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Method of Treatment

Disclosed herein is a method of treating a central nervous disorder (CNS) disorder, the method comprising the step of administering to a subject in need thereof, an effective amount of the compound disclosed herein, thereby treating the disorder. In some embodiments, the disorder is an addictive disorder. In some embodiments, the addictive disorder is nicotine addiction, alcohol addiction, opiate addiction, amphetamine addiction, methamphetamine addiction, or cocaine addiction. In some embodiments, the addictive disorder is nicotine addiction. In some embodiments, the addictive disorder is cocaine addiction. In some embodiments, the CNS disorder is schizophrenia. In some embodiments, the CNS disorder is a neurodegenerative disease. In some embodiments, the neurodegenerative disease is Alzheimer's disease, Parkinson's disease, Huntington's disease, or Lou Gehrig's disease (Amyotrophic Lateral Sclerosis or ALS).

Also disclosed herein is a method of treating substance abuse, the method comprising the step of administering to a subject in need thereof, an effective amount of the compound of any one of claims 1-127, wherein the effective amount is sufficient to diminish, inhibit or eliminate desire for and/or consumption of the substance in the subject. In some embodiments, the substance is nicotine, alcohol, opiates, amphetamines, methamphetamines, or cocaine. In some embodiments, the substance is nicotine. In some embodiments, the substance is alcohol. In some embodiments, the substance is opiates. In some embodiments, the substance is amphetamines. In some embodiments, the substance is methamphetamines. In some embodiments, the substance is cocaine.

Also disclosed herein is a method for treating an addictive disorder, the method comprising the steps of: a) administering to a subject in need thereof, an effective amount of a compound disclosed herein, during a first time period, wherein the first time period is a time period wherein the subject expects to be in an environment wherein, or exposed to stimuli in the presence of which, the subject habitually uses an addictive substance; and b) administering an effective amount of the compound during a second time period, wherein the second time period is a time period wherein the subject is suffering from withdrawal.

### Positive Allosteric Modulator (PAM)

Allosteric modulators are substances which indirectly influence (modulates) the effects of an agonist or inverse agonist at a receptor. Allosteric modulators bind to a site distinct from that of the orthosteric agonist binding site. Usually they induce a conformational change within the protein structure. A positive allosteric modulator (PAM), which is also called an allosteric enhancer, induces an amplification of the agonist's effect. PAMs, through their interaction at allosteric sites on the mGlu receptor, positively modulate (i.e., potentiate) the effects of the endogenous orthosteric mGlu agonist glutamate. The advantages of PAMs compared with orthosteric agonists includes enhanced subtype-selectivity, the potential for spatial and temporal modulation of receptor activation, and ease of optimization and fine-tuning of drug-like properties. Studies showed that selectively activating mGlu2 receptors on cocaine or nicotine dependence, unlike mGlu2/3 orthosteric agonists, decreased cocaine self-administration in rats at doses that did not affect responding for food. Data suggests that mGlu2 receptor PAMs have the potential for therapeutic utility in the treatment of drug dependence.

There have been many accounts in the literature describing selective mGlu2 receptor PAMs, whereas very little has been reported on compounds which potentiate the effects of glutamate at mGlu3 receptors. This is somewhat surprising given the significant sequence homology (approximately 75%) within the transmembrane regions of mGlu2 and mGlu3 receptors. Considering the dearth of information on mixed mGlu2/3 receptor PAMs, the development of such compounds would provide valuable pharmacological tools. For example, a CNS penetrant mGlu2/3 receptor PAM could facilitate investigations into whether effects on food responding in rats are due to general activation of mGlu3 receptors or an effect specific to direct activation of the mGlu receptor by agonists that act at the mGlu orthosteric binding site.

In some embodiments, the compounds described herein are mGlu2/3 receptor PAMs. In some embodiments, the compounds described herein are used to treat a CNS disorder. In some embodiments, the CNS disorder is anxiety. In some embodiments, the CNS disorder is depression. In some embodiments, the CNS disorder is schizophrenia. In another some embodiments, the CNS disorder is an addictive disorder. In some embodiments, the addictive disorder is nicotine addiction, alcohol addiction, opiate addiction, amphetamine addiction, methamphetamine addiction, or cocaine addiction. In some embodiments, the addictive disorder is nicotine addiction. In some embodiments, the addictive disorder is cocaine addiction.

In another aspect the disclosure provides methods for treating substance abuse, by administering to a subject in need thereof, an effective amount of a compound having Formula (I), (Ia), (II), or (III), wherein the effective amount is sufficient to diminish, inhibit or eliminate desire for and/or consumption of the substance in the subject.

In another aspect the disclosure provides methods for treating substance abuse, wherein the substance is nicotine, alcohol, opiates, amphetamines, methamphetamines, or cocaine.

In another aspect the disclosure provides a method for treating an addictive disorder, by a) administering to a subject in need thereof, an effective amount of a compound having Formula (I), (Ia), (II), or (III), during a first time period, wherein the first time period is a time period wherein the subject expects to be in an environment wherein, or exposed to stimuli in the presence of which, the subject habitually uses an addictive substance; and b) administering an effective amount of a compound having Formula I during a second time period, wherein the second time period is a time period wherein the subject is suffering from withdrawal.

In some embodiments, the CNS disorder is a neurodegenerative disease. In some embodiments, the neurodegenerative disease is Alzheimer's disease. In some embodiments, the neurodegenerative disease is Parkinson's disease. In some embodiments, the neurodegenerative disease is Huntington's disease. In some embodiments, the neurodegenerative disease is Lou Gehrig's disease (Amyotrophic Lateral Sclerosis or ALS). In some embodiments, the compounds described herein provide neuroprotection.

### Anxiety

Anxiety is an unpleasant state of inner turmoil, often accompanied by nervous behavior, such as pacing back and forth, somatic complaints and rumination. It is the subjectively unpleasant feelings of dread over anticipated events, such as the feeling of imminent death. Anxiety is a feeling of fear, worry, and uneasiness, usually generalized and unfocused as an overreaction to a situation that is only subjectively seen as menacing. It is often accompanied by muscular tension, restlessness, fatigue and problems in concentration. Anxiety can be appropriate, but when experienced regularly the individual may suffer from an anxiety disorder.

In some embodiments, the compounds described herein are mGlu2/3 receptor PAM used for treating anxiety symptoms. The method includes administering to a subject in need thereof, an effective amount of at least one mGlu2/3 receptor PAM, thereby treating the anxiety symptoms.

### Depression

Depression is a state of low mood and aversion to activity that can affect a person's thoughts, behavior, feelings and sense of well-being. People with depressed mood can feel sad, anxious, empty, hopeless, helpless, worthless, guilty, irritable or restless. They may lose interest in activities that were once pleasurable, experience loss of appetite or overeating, have problems concentrating, remembering details or making decisions, and may contemplate, attempt or commit suicide. Insomnia, excessive sleeping, fatigue, aches, pains, digestive problems or reduced energy may also be present. Depressed mood is a feature of some psychiatric syndromes such as major depressive disorder, but it may also be a normal reaction to life events such as bereavement, a symptom of some bodily ailments or a side effect of some drugs and medical treatments.

Blockade of mGlu2/3 receptors has antidepressant properties as reflected in reversal of the negative affective (depression-like) aspects of nicotine withdrawal. Thus, blockade of mGlu2 and mGlu3 reverses depression-like symptoms observed during drug withdrawal, and possibly depression observed during drug dependence (Ahmed, S. H., et al. Nature Neuroscience, 5: 625-626 (2002)). Therefore, administration of an effective amount of an antagonist of mGlu2 and mGlu3 is likely to be efficacious for treating non-drug-induced depressions, based on the known neurobiological similarities mediating drug- and non-drug-induced depressions (Markou et al. 1998; Barr et al., 2002; Cryan et al., 2002; Harrison et al., Neuropsychopharmacology, 25:55-71 (2001); Markou A and Kenny P J 2002, Neurotoxicity Research, 4(4), 297-313).

In some embodiments, the compounds described herein are mGlu2/3 receptor PAM used for treating depressive symptoms and anxiety symptoms of depression. The method includes administering to a subject in need thereof, an effective amount of at least one antagonist which modulates the mGlu2 and/or mGlu3 receptor, thereby treating the depressive symptoms and anxiety symptoms. In another aspect, the disclosure provides at least one mGlu2/3 receptor PAM that can be administered during a depressed time period, wherein the subject experiences symptoms of depression.

### Nicotine Addiction

Nicotine dependence is an addiction to tobacco products caused by the drug nicotine. Nicotine dependence means a person can't stop using the substance, even though it's causing harm. Nicotine produces physical and mood-altering effects in your brain that are temporarily pleasing. These effects make you want to use tobacco and lead to dependence. At the same time, stopping tobacco use causes withdrawal symptoms, including irritability and anxiety.

In certain aspects, the effective amount of at least one mGlu2/3 receptor PAM is administered to decrease nicotine consumption. For example, in one aspect an effective amount of a PAM of mGlu2 and/or mGlu3, can be administered to decrease nicotine consumption. In certain aspects of the disclosure, a PAM of mGlu2 and/or mGlu3 is administered while a subject is experiencing withdrawal. In another aspect of the disclosure, a PAM of mGlu2 and/or mGlu3 is administered during a time period when a subject is actively using an addictive substance. In another aspect of the disclosure, a PAM of mGlu2 and/or mGlu3 is administered during a time period when a subject is actively experiencing depression associated with drug use or not associated with drug use.

### Cocaine Addiction

Cocaine addiction remains a major public health problem in the United States. There are several sources of motivation that contribute to the continuance of cocaine abuse, including: the positive reinforcing effects of cocaine; and the alleviation of the negative affective aspects of cocaine withdrawal. Conditioned stimuli previously associated with cocaine administration may also elicit conditioned “cravings” leading to the reinstatement of cocaine-seeking behavior even after a prolonged period of abstinence. Recent studies indicate that the neuronal mechanisms underlying various aspects of drug abuse may differ necessitating the use of different treatments for specific aspects of drug dependence. To date, a safe and effective pharmacological treatment for cocaine dependence has yet to be identified. Thus, there remains a need for the design of new chemical entities that can be used as novel medications for cocaine addiction.

It has been found that repeated cocaine exposure may alter the function of Group II metabotropic glutamate receptors (mGluII receptors), pointing to a possible role of these mGlu subtypes in the development of cocaine dependence. The mGluII receptor positive modulators may decrease the reinforcing effects of self-administered cocaine in rats that had extended access to cocaine, a putative model of cocaine dependence while having no effect in rats with limited access to cocaine. Positive mGluII receptor modulators may attenuate discriminatory cue-induced reinstatement of cocaine self-administration. In contrast, mGluII receptor negative modulators may reverse the reward deficits associated with early cocaine abstinence.

Cocaine addiction is a chronic relapsing disorder and remains a major public health problem in the United States. The number of cases of cocaine abuse has steadily risen in the past decade. To date, a safe and effective pharmacological treatment for cocaine dependence has yet to be identified, which highlights the need to design new chemical entities that may become future novel medications for cocaine addiction. Recent evidence suggests that mGlus play a significant role in the abuse-related effects of cocaine. For example, repeated administration of cocaine has been shown to alter the function of mGlus, as well as their regulation by cysteine/glutamate exchange in the nucleus accumbens. These findings suggest that mGlu2 may be involved in the development of cocaine dependence and may represent a possible target for drug discovery against different aspects of cocaine abuse and dependence. There are several sources of motivation that contribute to the maintenance of cocaine abuse. These include the positive reinforcing effects of cocaine and alleviation of the negative affective aspects of cocaine withdrawal. Further, conditioned stimuli previously associated with cocaine administration may elicit conditioned “cravings” leading to the reinstatement of cocaine-seeking behavior even after a prolonged period of abstinence. Recent studies suggest that the neuronal mechanisms underlying drug self-administration are different from those mediating relapse vulnerability during abstinence, and different from those mediating the negative effects of early drug withdrawal. Therefore, it is important to explore concurrently the neurochemical mechanisms that contribute to the different aspects of cocaine dependence using animal models assessing the positive reinforcing effects of cocaine, the negative affective symptoms of early withdrawal, and cue-induced reinstatement of cocaine-seeking behavior after prolonged abstinence from drug intake. The discovery and preclinical testing of highly selective mGluII receptor modulators with good brain penetration may significantly contribute to the discovery of novel therapeutic treatments for different aspects cocaine dependence.

The intravenous drug self-administration procedure provides a reliable and robust model of human drug consumption. This procedure in animals provides a valid model of human drug abuse as studied in a controlled laboratory situation. Self-administration of drugs of abuse is thought to provide an operational measure of the rewarding effects of the drug. Increases in excitatory glutamatergic transmission are likely to contribute to the positive reinforcing properties of addictive drugs. Neurochemical studies indicate that systemic cocaine administration increase glutamate levels in the ventral tegmental area (VTA) and the nucleus accumbens, brain structures that are integral components of the extended amygdala, a brain circuit mediating the reward effects of all major drugs of abuse. The administration of a positive modulator of mGluII receptors may decrease cocaine self-administration in rats with extended access to cocaine by decreasing glutamate neurotransmission in limbic structures similar to the effects of mGlu2/3 agonists. In contrast, a negative modulator of mGluII receptors will most likely have no effect on cocaine self-administration, or possibly will shift the dose-response curve to the left, potentiating the reinforcing effects of cocaine.

Another challenge for the treatment of drug addiction is chronic vulnerability to relapse. One of the factors that precipitates drug craving and relapse to drug taking behavior in humans is environmental stimuli previously associated with drug-taking. These drug-associated stimuli can be divided into two categories: discrete drug cues (e.g., drug paraphernalia) that are associated with the rewarding effects of the drug, and discriminatory and contextual drug cues (e.g., specific environmental stimuli or specific environments) that predicts drug availability. In animals, discrete, discriminative and contextual conditioned cues can reinstate drug-seeking behavior, measured by variables derived from the reinstatement procedure. Recent data showed that reinstatement of cocaine-seeking was attenuated by systemic injections of N-acetylcysteine that leads to a tonic increase in nucleus accumbens glutamate levels in rats. Preliminary results in humans suggest that N-acetylcysteine attenuated cocaine craving in addicted humans. Further, exposure to environmental cues previously paired with cocaine injections increased glutamate in the nucleus accumbens. A potential use for mGlu2/3 agonists as pharmacotherapeutic agents to inhibit relapse was recently shown using different rodent models of reinstatement. In some embodiments, mGlu2/3 agonists attenuate cocaine-seeking behavior induced by discriminative cocaine-associated cues or by cocaine priming. In addition, mGlu2/3 agonists have been shown to inhibit cue-induced reinstatement of heroin-seeking, alcohol-seeking, nicotine-seeking, and also inhibited food-seeking behavior. The decreases in cue-induced food responding suggest that the administration of mGlu2/3 agonist decreased motivation for a natural reinforcer also. Further, it has been hypothesized that susceptibility to relapse due to cue reactivity increases gradually over periods of weeks or months. Thus, the administration of a positive modulator of mGluII receptors during prolonged abstinence from cocaine self-administration will decrease, while a negative modulator of mGluII receptors will have no effect on cocaine-seeking behavior induced by discriminative stimuli associated with cocaine availability.

Avoidance and alleviation of the negative affective state of early drug withdrawal with further drug abuse is hypothesized to be an important source of motivation that contributes significantly to the development of compulsive drug use and relapse during early abstinence. It has been hypothesized that susceptibility to relapse due to affective withdrawal symptoms peaks within days of cessation reflecting early rise in withdrawal symptoms. Thus, pharmacological treatments that reverse the depression-like aspects of early cocaine withdrawal would remove an important source of motivation that contributes to relapse to drug abuse shortly after the initial cessation of drug administration. Abrupt abstinence following chronic exposure to drugs of abuse, including cocaine results in a negative affective state reflected in significant elevations in intracranial self-stimulation (ICSS) thresholds. ICSS thresholds are thought to provide an operational measure of brain reward function; thus elevations in ICSS thresholds reflect deficits in brain reward function. This threshold elevation is opposite to the lowering of ICSS thresholds observed after cocaine administration that reflects an increase in brain reward function that most likely underlies, or at least relates to, cocaine's euphorigenic effects. This increase in brain reward function associated with cocaine consumption is considered essential for the establishment and maintenance of cocaine self-administration behavior. The mechanisms that contribute to withdrawal-induced reward deficits or reward facilitation remain unclear. Group II mGlus have been implicated in the synaptic adaptations that occur in response to chronic drug exposure and contribute to the aversive behavioral withdrawal syndrome. The role of glutamate transmission in the early phase of cocaine withdrawal has not been studied extensively. However, based on the nicotine dependence findings and the hypothesis of overlapping mechanisms of withdrawal from different drugs of abuse, one may hypothesize that decreased glutamatergic neurotransmission will also partly mediate cocaine withdrawal in cocaine-dependent subjects.

In some embodiments, the compounds described herein are mGlu2/3 receptor PAM used for treating cocaine addiction.

### Schizophrenia

Schizophrenia is a devastating psychiatric illness that afflicts approximately 1% of the worldwide population. The core symptoms observed in schizophrenic patients include positive symptoms (thought disorder, delusions, hallucinations, paranoia), negative symptoms (social withdrawal, anhedonia, apathy, paucity of speech) and cognitive impairments such as deficits in perception, attention, learning, short- and long-term memory and executive function. The cognitive deficits in schizophrenia are one of the major disabilities associated with the illness and are considered a reliable predictor of long-term disability and treatment outcome. Currently available antipsychotics effectively treat the positive symptoms, but provide modest effects on the negative symptoms and cognitive impairments. Furthermore, some patients are unresponsive to current antipsychotic treatments and several of these agents are associated with adverse side effects, including disturbances in motor function, weight gain, and sexual dysfunction. Thus, there is a need for new treatment strategies for schizophrenia that provide major improvements in efficacy across multiple symptom clusters and have fewer adverse effects.

Although the underlying pathophysiology of schizophrenia remains unknown, accumulating evidence points to disruptions in multiple neurotransmitter systems that modulate neural circuits important for normal affect, sensory processing, and cognition. In particular, early clinical findings demonstrated that changes in glutamatergic transmission produced by antagonists of the N-methyl-D-aspartate (NMDA) subtype of ionotropic glutamate receptors, including phencyclidine (PCP), result in a state of psychosis in humans that is similar to that observed in schizophrenic patients. These studies suggest that agents that increase NMDA receptor function have potential as therapeutics for the treatment of all major symptom clusters (positive, negative, cognitive) of the disease. More recently, studies indicate that reduced NMDA receptor function induces complex changes in transmission through cortical and subcortical circuits that involve both glutamatergic and GABAergic synapses and include downstream increases in transmission at glutamatergic synapses in the prefrontal cortex. Importantly, these circuit changes might share common features with changes in brain circuit activities that occur in schizophrenia patients. One hypothesis is that NMDA receptors involved in these symptoms might reside at glutamatergic synapses on GABAergic projection neurons in midbrain regions as well as GABAergic interneurons and glutamatergic projection neurons in key cortical and limbic regions For example, under normal conditions the activation of NMDA receptors localized on GABAergic projection neurons in subcortical regions, such as the nucleus accumbens, provides inhibitory control on excitatory glutamatergic thalamocortical neurons that project to pyramidal neurons in the prefrontal cortex (PFC). Hypofunction or blockade of these NMDA receptors on midbrain inhibitory GABAergic neurons could result in a disinhibition of glutamatergic thalamocortical inputs to pyramidal neurons in the PFC. This disinhibition would lead to a subsequent increased activity of glutamatergic thalamic neurons and increased activity mediated by the DL-a-amino-3-hydroxy-5-methylisoxasole-4-propionate (AMPA) subtype of glutamate receptors at thalamocortical synapses in the PFC. Based on this model, manipulations that enhance NMDA receptor function, such as activation of metabotropic glutamate receptor subtype 5 (mGlu5) located on GABAergic neurons, have potential as a novel approach to the treatment of schizophrenia. An alternative approach might be to reduce excitatory glutamatergic transmission at key synapses, such as thalamocortical synapses in the PFC, by activation of metabotropic glutamate receptor subtypes 2 and 3 (mGlu2 and mGlu3) presynaptically located in these synapses. Although other viable models of circuit changes associated with schizophrenia exist, this hypothesis provides one possible framework within which to consider effects of ligands at mGlu receptors that might be relevant to schizophrenia.

A large number of preclinical and clinical studies provide strong evidence that agonists of mGlu2 and mGlu3 (group II mGlu receptors) also have potential as a novel approach to the treatment of schizophrenia. Consistent with the animal studies, clinical studies reveal that a highly selective agonist of group II mGlu receptors has robust efficacy in improving ratings for positive and negative symptoms in patients with schizophrenia. Unlike currently marketed antipsychotic agents, there were no major adverse events reported for the mGlu2/3 agonist in the clinical studies to date. However, further clinical studies will be required to fully establish safety of these compounds after long-term dosing in schizophrenic patients, as well as assess possible efficacy on the cognitive impairments in these patients. Taken together, these findings represent an important breakthrough and could ultimately lead to introduction of group II mGlu receptor activators as a fundamentally novel approach to the treatment of schizophrenia. As mentioned above, animal studies reveal that the psychotomimetic agents increase activity of glutamatergic synapses in the PFC, and hyperactivity of glutamate neurotransmission in the PFC and limbic structures has been postulated to play a critical role in the pathophysiology of schizophrenia. Interestingly, effects of psychotomimetic agents on glutamatergic transmission in the PFC are blocked by group II mGlu receptor agonists. Although it is not yet clear whether this action of group II mGlu receptor agonists is mechanistically related to the antipsychotic actions of these compounds, these actions fit well with current models of disruptions in subcortical and cortical circuits that might be involved in the psychotomimetic effects of NMDA receptor antagonists and the range of symptoms observed in schizophrenia patients. Despite advances in development of group II mGlu receptor agonists, it is not yet clear whether orthosteric agonists of these receptors will reach the market for broad clinical use. Long-term administration of group II mGlu receptor agonists induces robust tolerance in at least one rodent model that has been used to predict antipsychotic efficacy. These orthosteric agonists also activate both mGlu2 and mGlu3 and do not provide insights into which of these group II mGlu receptor subtypes is most important for clinical efficacy. Although, recent findings demonstrate that the antipsychotic-like effects of mGlu2/3 receptor agonists are absent in mGlu2-knockout, but not mGlu3-knockout, mice. Thus, it is possible that positive allosteric modulators of mGlu2 might be an alternative approach that could provide greater selectivity and other potential advantages to orthosteric agonists.

In some embodiments, group II mGlu receptor agonists are useful in the treatment of schizophrenia. In some embodiments, selective mGlu2 PAMs represent a novel approach to the treatment of these disorders that is devoid of the adverse effects associated with currently available drugs.

In some embodiments, the compounds described herein are mGlu2/3 receptor PAM used for treating schizophrenia. The method includes administering to a subject in need thereof, an effective amount of at least one mGlu2/3 receptor PAM, thereby treating schizophrenia.

### Alzheimer's Disease

Alzheimer's disease (AD), also known as Alzheimer disease, or just Alzheimer's, accounts for 60% to 70% of cases of dementia. It is a chronic neurodegenerative disease that usually starts slowly and gets worse over time. The most common early symptom is difficulty in remembering recent events (short term memory loss). As the disease advances, symptoms can include: problems with language, disorientation (including easily getting lost), mood swings, loss of motivation, not managing self-care, and behavioral issues. As a person's condition declines, she or he often withdraws from family and society. Gradually, bodily functions are lost, ultimately leading to death. Although the speed of progression can vary, the average life expectancy following diagnosis is three to nine years.

Various brain regions, including the cerebral cortex, hippocampus, striatum, amygdala, frontal cortex and nucleus accumbens, display high levels of mGlu2 and mGlu3 receptor binding. This distribution pattern suggests a role for the mGlu2/3 receptor subtypes in the pathology of neuropsychiatric disorders such as Alzheimer's disease.

In some embodiments, the compounds described herein are mGlu2/3 receptor PAM used for treating Alzheimer's disease. The method includes administering to a subject in need thereof, an effective amount of at least one mGlu2/3 receptor PAM, thereby treating Alzheimer's disease.

### Huntington's Disease

Huntington's disease (HD) is a neurodegenerative genetic disorder that affects muscle coordination and leads to mental decline and behavioral symptoms. Symptoms of the disease can vary between individuals and affected members of the same family, but usually progress predictably. The earliest symptoms are often subtle problems with mood or cognition. A general lack of coordination and an unsteady gait often follows. As the disease advances, uncoordinated, jerky body movements become more apparent, along with a decline in mental abilities and behavioral symptoms. Physical abilities gradually worsen until coordinated movement becomes difficult. Mental abilities generally decline into dementia. Complications such as pneumonia, heart disease, and physical injury from falls reduce life expectancy to around twenty years from the point at which symptoms begin. Physical symptoms can begin at any age from infancy to old age, but usually begin between 35 and 44 years of age.

Excitotoxic injury to striatum by dysfunctional cortical input or aberrant glutamate uptake may contribute to Huntington's disease (HD) pathogenesis. Daily subcutaneous injection with a maximum tolerated dose (MTD) of the mGlu2/3 agonist LY379268 (20 mg/kg) beginning at 4 weeks has been found to dramatically improves the phenotype in R6/2 mice (the most commonly used animal model of Huntington's disease) (Reiner et al. Brain Research 1473 (2012) 161-172). For example, normalization of motor function in distance traveled, speed, the infrequency of pauses, and the ability to locomote in a straight line, and a rescue of a 15-20% striatal neuron loss at 10 weeks were observed.

In some embodiments, the compounds described herein are mGlu2/3 receptor PAM used for treating Huntington's disease. The method includes administering to a subject in need thereof, an effective amount of at least one mGlu2/3 receptor PAM, thereby treating Huntington's disease.

### Lou Gehrig's Disease (ALS)

Amyotrophic lateral sclerosis (ALS) is a debilitating disorder characterized by rapidly progressive motor neuron degeneration, which results into weakness, muscle atrophy and spasticity. Riluzole is the only drug that improves survival of ALS patients, only to a modest extent. Thus, there is an urgent need for treatments that slow the progression of ALS. Familial ALS (FALS) is caused by mutations of several genes including SOD1 (type-1 superoxide dismutase). Although SOD1 mutations account for only 20% of FALS and about 2% of sporadic ALS, SOD1 mutant mice recapitulate several features of human ALS, and are widely employed as model for ALS. The validity of this model is strengthened by the evidence that SOD1 aggregates are detected in the spinal cord of people with sporadic ALS or with ALS caused by mutations of genes other than SOD1. The mechanisms by which SOD1 misfolding damages motor neurons are only partially elucidated and involve glutamate excitotoxicity, mitochondrial dysfunction, disruption of axonal transport, and abnormalities in astrocytes and microglia. One of the potential mechanisms of excitotoxicity in ALS is a reduced expression of the glutamate transporter, GLT-1, which clears glutamate from the synapses.

Enhancement of glial-derived neurotrophic factor (GDNF) is an established therapeutic target for amyotrophic lateral sclerosis (ALS). Activation of group II metabotropic glutamate (mGlu) receptors with the orthosteric agonist, LY379268, enhanced GDNF levels in cultured spinal cord astrocytes from wild-type mice and mGlu2 knockout mice, but not in astrocytes from mGlu3 knockout mice. LY379268 protected Sternberger monoclonal incorporated antibody-32 (SMI-32)⁺ motor neurons against excitotoxic death in mixed cultures of spinal cord cells, and its action was abrogated by anti-GDNF antibodies. Acute systemic injection of LY379268 (0.5, 1 or 5 mg/kg, i.p.) enhanced spinal cord GDNF levels in wild-type and mGlu2 knockout mice, but not in mGlu3 knockout mice. No tolerance developed to the GDNF-enhancing effect of LY379268 when the drug was continuously delivered for 28 days by means of s.c. osmotic minipumps (0.5-5 mg/day). Continuous infusion of LY379268 also enhanced the expression of the glutamate transporter GLT-1, in the spinal cord. Continuous treatment with 1 or 5 mg/kg/day with LY379268 had a beneficial effect on neurological disability in SOD1G93A mice. At day 40 of treatment, LY379268 enhanced spinal cord levels of GDNF and GLT-1, and rescued spinal cordmotor neurons, as assessed by stereologic counting of SMI-32⁺ cells.

In some embodiments, the compounds described herein are mGlu2/3 receptor PAM used for treating ALS. The method includes administering to a subject in need thereof, an effective amount of at least one mGlu2/3 receptor PAM, thereby treating ALS.

### Parkinson's Disease

Parkinson's disease (PD) is a chronic movement disorder resulting from a disturbance in the normal functioning of the basal ganglia, a collection of subcortical nuclei that are essential for the initiation and control of motor activity. The underlying pathology of the disease is a progressive degeneration of the dopaminergic nigrostriatal tract that manifests as a range of motor deficits including akinesia or bradykinesia, tremor, rigidity and postural instability. Current therapies for PD are essentially based on dopamine replacement and include levodapa (L-DOPA), a precursor of dopamine, and dopamine receptor agonists. These agents are effective in treating the symptoms of the disease in the early stages, but are less effective as the disease progresses when debilitating side-effects such as “on-off” fluctuations in efficacy and uncontrollable dyskinesias (involuntary muscle movements) ensue. More importantly, dopaminergic treatments do not halt the disease progression. For these reasons, several investigators have started to focus on nondopaminergic interventions as symptomatic and neuroprotecive strategies in PD.

Studies have shown that Group II mGlu receptors play some role in alleviating akinesia in the rat. In functional studies (Murray et al. Pharmacology, Biochemistry and Behavior 73 (2002) 455-466), intracerebroventricular administration of LY379268 (1, 5, 10, 20 nmol/2 l) produced a dose-dependent increase in locomotor activity in the reserpine (5 mg/kg ip)-treated rat. In contrast, systemic administration of LY379268 (0.1, 1, 10 mg/kg ip) did not reverse reserpine-induced akinesia and failed to effect rotational behaviour 1 month after unilateral lesioning of the nigrostriatal tract by 6-hydroxydopamine (6-OHDA; 4 mg infused into the substantia nigra (SN)). These results suggest that mGlus may offer a nondopaminergic approach to the treatment of PD.

In some embodiments, the compounds described herein are mGlu2/3 receptor PAM used for treating Parkinson's disease. The method includes administering to a subject in need thereof, an effective amount of at least one mGlu2/3 receptor PAM, thereby treating Parkinson's disease.

### Neuroprotection

In neuroprotective studies (Murray et al. Pharmacology, Biochemistry and Behavior 73 (2002) 455-466), animals were treated with LY379268 (10 mg/kg/day ip) either for 7 days following 6-OHDA injection into the SN (4 mg) or for 21 days following 6-OHDA injection into the striatum (10 mg) before measurement of tyrosine hydroxylase immunoreactivity in the striatum and/or SN as an index of neuroprotection. LY379268 provided some protection against nigral infusion of 6-OHDA and also some functional improvement and correction of dopamine turnover was observed. The compound also provided significant protection in the striatum and some protection in the SN against striatal infusion of 6-OHDA.

Low doses of the mGlu2/3 metabotropic glutamate receptor agonist, LY379268 (0.25-3 mg/kg, i.p.) increased glial cell line-derived neurotrophic factor (GDNF) mRNA and protein levels in the mouse brain, as assessed by in situ hybridization, real-time PCR, immunoblotting, and immunohistochemistry. This increase was prominent in the striatum, but was also observed in the cerebral cortex. GDNF mRNA levels peaked at 3 h and declined afterwards, whereas GDNF protein levels progressively increased from 24 to 72 h following LY379268 injection. The action of LY379268 was lost in mGlu3 receptor knockout mice, but not in mGlu2 receptor knockout mice. In pure cultures of striatal neurons, the increase in GDNF induced by LY379268 required the activation of the mitogen-activated protein kinase and phosphatidylinositol-3-kinase pathways, as shown by the use of specific inhibitors of the two pathways. Both in vivo and in vitro studies led to the conclusion that neurons were the only source of GDNF in response to mGlu3 receptor activation. Remarkably, acute or repeated injections of LY379268 at doses that enhanced striatal GDNF levels (0.25 or 3 mg/kg, i.p.) were highly protective against nigro-striatal damage induced by 1-methyl-4-phenyl-1,2,3,6-tetrahydropyridine in mice, as assessed by stereological counting of tyrosine hydroxylase-positive neurons in the pars compacta of the substantia nigra. It is speculated that selective mGlu3 receptor agonists or enhancers are potential candidates as neuroprotective agents in Parkinson's disease, and their use might circumvent the limitations associated with the administration of exogenous GDNF. Hence, selective mGlu3 receptor agonists or positive allosteric modulators (PAMs) would potentially be helpful in the treatment of chronic neurodegenerative disorder by providing neuroprotection.

### Combination Treatments

In certain instances, it is appropriate to administer at least one compound of Formula (I), (Ia), (II), or (III) in combination with another therapeutic agent.

In one specific embodiment, a compound of Formula (I), (Ia), (II), or (III) is co-administered with a second therapeutic agent, wherein the compound of Formula (I), (Ia), (II), or (III) and the second therapeutic agent modulate different aspects of the disease, disorder or condition being treated, thereby providing a greater overall benefit than administration of either therapeutic agent alone.

For combination therapies described herein, dosages of the co-administered compounds vary depending on the type of co-drug(s) employed, on the specific drug(s) employed, on the disease or condition being treated and so forth. In additional embodiments, when co-administered with one or more other therapeutic agents, the compound provided herein is administered either simultaneously with the one or more other therapeutic agents, or sequentially.

If administration is simultaneous, the multiple therapeutic agents are, by way of example only, provided in a single, unified form, or in multiple forms.

In some embodiments, compounds of Formula (I), (Ia), (II), or (III) are administered to a mammal in combination with one or more additional neurodegenerative disease or disorder therapeutic agent. In some embodiments, the neurodegenerative disease or disorder is Alzheimer's disease, Parkinson's disease, Huntington's disease, or Lou Gehrig's Disease (Amyotrophic Lateral Sclerosis or ALS). In some embodiments, compounds of Formula (I), (Ia), (II), or (III) are administered to a mammal in combination with one or more additional therapeutic agent that alleviate the symptoms or side effects of a neurodegenerative disease or disorder. In some embodiments the symptoms or side effects a neurodegenerative disease or disorder are dementia, memory loss, dyskinesias, cognitive impairment, tremors, rigidity, slowness of movement, postural instability, involuntary jerking or writhing movements (chorea), slow or abnormal eye movements, difficulty with the physical production of speech or swallowing, psychiatric disorders, muscle cramps and spasms, spasticity, constipation, fatigue, excessive salivation, excessive phlegm, pain, depression, sleep problems, uncontrolled outbursts of laughing or crying.

In some embodiments, the additional therapeutic agent is an Alzheimer's disease therapeutic agent. In some embodiments, the additional therapeutic agent is a cholinesterase inhibitor. In some embodiments, the cholinesterase inhibitor is donepezil, galantamine, or rivastigmine. In some embodiments, the additional therapeutic agent is memantine. In some embodiments, the additional therapeutic agent is latrepirdine, idalopridine, or cerlapirdine.

In some embodiments, the additional therapeutic agent is a Parkinson's disease therapeutic agent. In some embodiments, the additional therapeutic agent is levodopa. In some embodiments, the additional therapeutic agent is carbidopa-levodopa. In some embodiments, the additional therapeutic agent is a Dopamine agonist. In some embodiments, the dopamine agonist is ropinirole, pramipexole, or rotigotine. In some embodiments, the additional therapeutic agent is a MAO-B inhibitor. In some embodiments, the MAO-B inhibitor is selegiline or rasagiline. In some embodiments, the additional therapeutic agent is a catechol O-methyltransferase (COMT) inhibitor. In some embodiments, the COMT inhibitor is entacapone or tolcapone. In some embodiments, the additional therapeutic agent is an Anticholinergic. In some embodiments, the anticholinergic is benztropine or trihexyphenidyl. In some embodiments, the additional therapeutic agent is amantadine.

In some embodiments, compounds of Formula (I), (Ia), (II), or (III) are administered to a mammal in combination with deep brain stimulation.

In some embodiments, the additional therapeutic agent is a Huntington's disease therapeutic agent. In some embodiments, the additional therapeutic agent is tetrabenazine. In some embodiments, the additional therapeutic agent is an antipsychotic drug. In some embodiments, the antipsychotic drug is haloperidol, chlorpromazine, risperidone, olanzapine or quetiapine. In some embodiments, the additional therapeutic agent is amantadine, levetiracetam, or clonazepam. In some embodiments, the additional therapeutic agent is an antidepressant. In some embodiments, the antidepressant is citalopram, fluoxetine, or sertraline. In some embodiments, the additional therapeutic agent is a mood-stabilizing drug. In some embodiments, the mood-stabilizing drug is valproate, carbamazepine, or lamotrigine.

In some embodiments, compounds of Formula (I), (Ia), (II), or (III) are administered to a mammal in combination with psychotherapy, speech therapy, physical therapy or occupational therapy.

In some embodiments, the additional therapeutic agent is a Lou Gehrig's Disease (Amyotrophic Lateral Sclerosis or ALS) therapeutic agent. In some embodiments, the additional therapeutic agent is riluzole. In some embodiments, the additional therapeutic agent is baclofen, diazepam, trihexyphenidyl or amitriptyline.

In some embodiments, compounds of Formula (I), (Ia), (II), or (III) are administered to a mammal in combination with one or more additional neuropsychiatric disease or disorder therapeutic agent. In some embodiments, the neuropsychiatric disease or disorder is schizophrenia, anxiety, sleep disorder, eating disorder, psychosis, or addictions.

In some embodiments, compounds of Formula (I), (Ia), (II), or (III) are administered in combination with one or more additional anti-addiction therapeutic agent. In some embodiments, compounds of Formula (I), (Ia), (II), or (III) are administered in combination with one or more additional anti-addiction therapeutic agent for the treatment of a substance use and/or substance abuse disorder. In some embodiments, the substance use disorder is a nicotine use disorder, a stimulant use disorder, an alcohol use disorder, or an opioid use disorder. In some embodiments, the anti-addiction therapeutic agent is selected from the group consisting of buprenorphine, methadone, naltrexone, subozone, naloxone, acamprosate, disulfiram, bupropion, varenicline, and a nicotine replacement therapy (NRT).

In some embodiments, the additional therapeutic agent is an antipsychotic. In some embodiments, the antipsychotic is aripiprazole, asenapine, clozapine, iloperidone, lurasidone, olanzapine, paliperidone, quetiapine, risperidone, ziprasidone, chlorpromazine, fluphenazine, haloperidol, or perphenazine. In some embodiments, the additional therapeutic agent is an antidepressant. In some embodiments, the antidepressant is a selective serotonin reuptake inhibitor (SSRI) or a serotonin norepinephrine reuptake inhibitor (SNRI). In some embodiments, the antidepressant is escitalopram, duloxetine, venlafaxine, or paroxetine. In some embodiments, the additional therapeutic agent is an anti-anxiety medication. In some embodiments, the anti-anxiety medication is buspirone. In some embodiments, the additional therapeutic agent is a benzodiazepine. In some embodiments the benzodiazepine is alprazolam, chlordiazepoxide, diazepam, or lorazepam.

In some embodiments, the additional therapeutic agent is a medication used to treat dependence. In some embodiments, the medication used to treat dependence is buprenorphine, methadone, naltrexone, subozone, naloxone, acamprosate, disulfiram, bupropion, varenicline, or a nicotine replacement therapy (NRT).

### EXAMPLES

The following examples are intended to illustrate but not limit the disclosed embodiments.

All reactions were performed in oven-dried glassware under an atmosphere of argon with magnetic stirring. All solvents and chemicals used were purchased from Sigma-Aldrich or Acros, and were used as received without further purification. Purity of compounds was established by liquid chromatography-mass spectroscopy (HPLC-MS) and was >95% for all tested compounds. Silica gel column chromatography was carried out using prepacked silica cartridges from RediSep (ISCO Ltd.) and eluted using an Isco Companion system. ¹H- and ¹³C-NMR spectra were obtained on a Jeol 400 spectrometer at 400 MHz and 100 MHz, respectively. Chemical shifts are reported in δ (ppm) relative to residual solvent peaks or TMS as internal standards. Coupling constants are reported in Hz. High-resolution ESI-TOF mass spectra were acquired from the Mass Spectrometry Core at The Sanford-Burnham Medical Research Institute (Orlando, Florida). HPLC-MS analyses were performed on a Shimadzu 2010EV LCMS using the following conditions: Kromisil C18 column (reverse phase, 4.6 mm×50 mm); a linear gradient from 10% acetonitrile and 90% water to 95% acetonitrile and 5% water over 4.5 min; flow rate of 1 mL/min; UV photodiode array detection from 200 to 300 nm.

General Methods for the Synthesis of mGlu2/3 Receptor PAMs.

General method A: A solution of appropriate chloride 1 (80 mmol, 1 equiv) in CH₂Cl₂ (DCM; 20 mL) was added dropwise a stirred solution of AlCl₃ (10.64 g, 80 mmol, 1 equiv) in CH₂Cl₂ (500 mL) at 0° C. under nitrogen. Substituted resorcinol derivative 2 (80 mmol, 1 equiv) was added to the reaction mixture, and the reaction was gradually warmed to room temperature (rt) and stirred at room temperature for 12 h. The pre-cooled reaction mixture was quenched by the dropwise addition of HCl (5% aq.) and diluted with water. The organic layer was separated, and the aqueous layer was extracted with CH₂Cl₂ (3×100 mL) and the combined organic extracts were washed with water, brine and dried over anhyd. sodium sulfate, filtered and concentrated under reduced pressure to obtain the crude product, which was filtered with hexane and dried under vacuum to yield the substituted acylated derivatives 3 as a white/tan solid.

General method B: A mixture of appropriate methyl hydroxybenzoate derivative 4 (2 mmol), dibromo compound (6 mmol), and potassium carbonate (4 mmol) in acetonitrile (ACN; 15 mL) was refluxed for 6 h under an atmosphere of nitrogen. After cooled to room temperature, volatiles were removed under reduced pressure. The crude reaction mixture was diluted with water and DCM. The organic layer collected, and the aqueous layer extracted twice with DCM. Combined organic extracts were washed with water, brine, dried over anhyd. sodium sulfate, filtered and concentrated to give the crude product, which was purified by flash column chromatography (hexanes to 30% EtOAc in hexanes) to give intermediate derivatives 6.

General method C: To a stirred solution of the product 6 from general method B (1 mmol) and the substituted acylated derivative 3 from general method A (1 mmol) in acetonitrile was added potassium carbonate (2 mmol). The resulting mixture was heated at 80° C. for 2 h (reaction monitored by LC-MS) under inert atmosphere. The precipitated solids were filtered off and the solvent removed under reduced pressure. The crude product was partitioned between water and ethyl acetate. The organic layer was collected, and the aqueous layer extracted with ethyl acetate twice. Combined organic layers were washed with water, brine, and dried over anhyd. sodium sulfate, filtered and concentrated under reduced pressure to obtain the crude product which was used for next step without further purification.

General method D: To a solution of the above ester (1 mmol) in dioxane (10 mL) was added 2M LiOH aq. solution (2.5 mL, 5 mmol), and the resulting mixture was heated at 50° C. until the starting material was consumed as determined by LC-MS (˜1 h). After completion of the reaction, the reaction mixture was cooled to rt and diluted with water. The pH was adjusted to 1 by the addition of 1N HCl and ethyl acetate (50 mL) was added. The organic layer was separated and dried over Na₂SO₄. The solvents were removed by rotary evaporation and the products were isolated by reverse phase HPLC and lyophilized to provide the final compounds which were determined to be >95% pure by HPLC-UV, HPLC-MS, and ¹H NMR.

General method E: Potassium carbonate (1 mmol) was added to a solution of appropriate resorcinol derivative (0.5 mmol) and methyl 3′-(bromomethyl)-biphenyl-3-carboxylate (0.5 mmol in CH₃CN (5 mL). After stirring for 2 h at 80° C., the organic phase was evaporated under reduced pressure and the crude material was partitioned between water and CH₂Cl₂. The aqueous layer was extracted with CH₂Cl₂ (3×15 mL). The organic layer was dried using Na₂SO₄ and evaporated to give the ester derivatives in quantitative yield. The crude ester derivative was used in the next step without further purification. 2M LiOH (0.25 mL g, 0.5 mmol) was added to a solution of the crude product (0.5 mmol) in THF (5 mL). The reaction mixture was heated under reflux for 30 min. and then cooled to room temperature and acidified with dil. HCl. and ethyl acetate (50 mL) was added. The organic layer was separated and dried over Na₂SO₄. The solvents were removed by rotary evaporation and the products were isolated by reverse phase HPLC and lyophilized to provide the final compounds which were determined to be >95% pure by HPLC-UV, HPLC-MS, and ¹H NMR.

General method F: Potassium carbonate (2.76 g, 20 mmol) was added to a solution of 1-(2,4-dihydroxy-3-methylphenyl)-3,3-dimethylbutan-1-one (2.23 g, 10 mmol) and 1-bromo-3-(bromomethyl)benzene (2.5 g, 10 mmol) in CH₃CN (100 mL). After stirring for 2 h at 80° C., the organic phase was evaporated under reduced pressure and the crude material was partitioned between water and CH₂Cl₂. The aqueous layer was extracted with CH₂Cl₂ (3×50 mL). The organic phase was dried using Na₂SO₄ and evaporated to give 1-(4-(3-bromobenzyloxy)-2-hydroxy-3-methylphenyl)-3,3-dimethylbutan-1-one (3.87 g, 98%). The crude product was used for the next step without further purification. LC-MS m/z calcd for C₂₀H₂₃BrO₃ [M+H]⁺: 391.08. Found: 391.00. A mixture of 1-(4-(3-bromobenzyloxy)-2-hydroxy-3-methylphenyl)-3,3-dimethylbutan-1-one (0.100 g, 0.25 mmol), boronic acid (0.0.375 mmol) and Pd(PPh₃)₄ (0.028 mg, 0.025 mmol) were taken in DME (2 mL). To this solution was added 2M Na₂CO₃ (0.5 mL) and the resulting mixture was heated at reflux under an atmosphere of N₂ for 1 h. The reaction mixture was cooled to room temperature, diluted with water and neutralized using 1M HCl. A usual work up with EtOAc followed by preparative HPLC yielded the desired compounds.

### Example 1

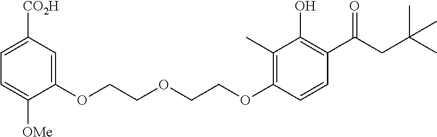 [SMILES: COc1ccc(C(=O)O)cc1OCCOCCOc1ccc(C(=O)CC(C)(C)C)c(O)c1C]

### 3-(2-(2-(4-(3,3-Dimethylbutanoyl)-3-hydroxy-2-methylphenoxy)ethoxy)ethoxy)-4-methoxybenzoic acid

Methyl 3-hydroxy-4-methoxybenzoate, bis(2-bromomethyl)ether, 1-(2,4-dihydroxy-3-methylphenyl)-3,3-dimethylbutan-1-one, K₂CO₃ and LiOH were processed according to general method B, C and D. White solid. ¹H NMR (DMSO-d₆): δ7.83 (d, J=9.2 Hz, 1H), 7.53 (dd, J=1.8 Hz, 8.2 Hz, 1H), 7.41 (d, J=1.8 Hz, 1H), 7.00 (d, J=9.2 Hz, 1H), 6.58 (d, J=9.2 Hz, 1H), 4.21-4.19 (m, 2H), 4.11-4.09 (m, 2H), 3.84-3.80 (m, 4H), 3.77 (s, 3H), 2.80 (s, 2H), 1.92 (s, 3H), 0.96 (s, 9H). ¹³C NMR (DMSO-d₆): δ 205.9, 167.0, 162.3, 161.1, 152.8, 147.4, 131.1, 123.4, 122.9, 114.5, 113.2, 112.1, 111.2, 103.2, 69.1, 68.9, 67.9, 55.6, 48.5, 31.6, 29.9, 7.6. LC-MS (ESI) Calcd for C₂₅H₃₂O₈ [M+H]⁺: 461.21. Found: 461.00. HRMS (ESI) Calcd for C₂₅₉H₃₂O₈ [M+H]⁺: 461.2112. Found: 461.2182.

### Example 2

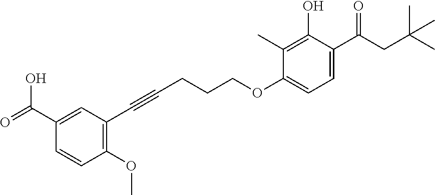 [SMILES: COc1ccc(C(=O)O)cc1C#CCCCOc1ccc(C(=O)CC(C)(C)C)c(O)c1C]

### 3-(5-(4-(3,3-Dimethylbutanoyl)-3-hydroxy-2-methylphenoxy)pent-1-yn-1-yl)-4-methoxybenzoic acid

A flask containing a mixture of methyl 3-iodo-4-methoxybenzoate (2.103 g, 7.2 mmol), Cu(I)I (0.137 g, 0.720 mmol) and Pd (PPh₃)₂Cl₂ (0.253 g, 0.360 mmol) in Et₃N (36 mL) was placed under nitrogen. The mixture was heated at 60° C. for 30 min and then pent-4-yn-1-ol (0.606 g, 7.2 mmol) was added in dropwise. After 2 h, the mixture was concentrated and the residue was purified by silica column chromatography (1:4 EtOAc:Hexanes) to yield methyl 3-(5-hydroxypent-1-yn-1-yl)-4-methoxybenzoate as a yellow viscous oil (1 g, 55.9%). To a solution of methyl 3-(5-hydroxypent-1-yn-1-yl)-4-methoxybenzoate (0.540 g, 2.175 mmol) and PPh₃ (0.628 g, 2.392 mmol) in DCM (50 mL) at 0° C., was added NBS (0.387 g, 2.175 mmol). The reaction mixture was gradually warmed to rt and stirred overnight. The solvent was removed under reduced pressure and the residue purified by column chromatography (SiO₂, 1:4 EtOAc:Hexanes) to yield methyl 3-(5-bromopent-1-yn-1-yl)-4-methoxybenzoate as a viscous oil (0.332 g, 49.1%). To a stirred solution of 1-(2,4-dihydroxy-3-methylphenyl)-3,3-dimethylbutan-1-one (0.11 g, 0.5 mmol) and methyl 3-(5-bromopent-1-yn-1-yl)-4-methoxybenzoate (0.156 g, 0.5 mmol) in ACN (5 mL) was added K₂CO₃ (0.138 g, 1 mmol) and heated at 80° C. for 2 h. After cooling to rt, volatiles were removed under reduced pressure. The crude reaction mixture was diluted with water and DCM. The organic layer collected, and the aqueous layer extracted twice with DCM. Combined organic extracts were washed with water, brine, dried over Na₂SO₄, filtered and concentrated to give the crude product, which was purified by flash column chromatography (hexanes to 30% EtOAc in hexanes) to give methyl 3-(5-(4-(3,3-dimethylbutanoyl)-3-hydroxy-2-methylphenoxy)pent-1-yn-1-yl)-4-methoxybenzoate. To a solution of the above ester (0.5 mmol) in dioxane (5 mL) was added 2M LiOH (1.25 mL, 2.5 mmol), and the resulting mixture was heated at 50° C. until the starting material was consumed as determined by LC-MS (A1 h). After completion of the reaction, the mixture was cooled to rt and diluted with water. The pH was adjusted to 1 by the addition of 1N HCl and EtOAc (50 mL) was added. The organic layer was separated and dried over Na₂SO₄. The solvents were removed by rotary evaporation and the products were isolated by reverse phase HPLC. White solid (0.149 g, 68%). ¹H NMR (DMSO-d₆): δ 7.88-7.79 (m, 3H), 7.07 (d, J=8.7 Hz, 1H), 6.62 (d, J=9.2 Hz, 1H), 4.20 (t, J=6.4 Hz, 2H), 3.80 (s, 3H), 2.80 (s, 2H), 2.59 (t, J=6.8 Hz, 2H), 2.01-1.97 (overlapping multiplet and singlet, 5H), 0.96 (s, 9H). ¹³C NMR (DMSO-d₆): δ 205.9, 166.4, 162.9, 162.4, 161.6, 134.0, 131.3, 131.0, 122.9, 114.5, 112.1, 112.0, 111.0, 103.0, 93.9, 76.6, 66.7, 55.9, 48.5, 31.6, 29.9, 27.8, 15.7, 7.5. LC-MS (ESI) Calcd for C₂₆H₃₀O₆ [M+H]⁺: 439.20. Found: 439.00. HRMS (ESI) Calcd for C₂₆H₃₀O₆ [M+H]⁺: 439.2068. Found: 449.2136.

### Example 3

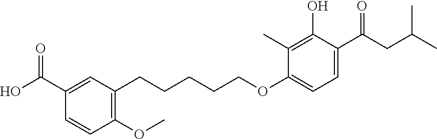 [SMILES: COc1ccc(C(=O)O)cc1CCCCCOc1ccc(C(=O)CC(C)C)c(O)c1C]

### 3-(5-(4-(3,3-Dimethylbutanoyl)-3-hydroxy-2-methylphenoxy)pentyl)-4-methoxybenzoic acid

3-(5-(4-(3,3-Dimethylbutanoyl)-3-hydroxy-2-methylphenoxy)pent-1-yn-1-yl)-4-methoxybenzoic acid (0.025 g, 0.057 mmol) was taken in methanol (MeOH, 5 mL) and treated with H₂ (1 atm) in presence of Pd/C (10 wt %) for 2 h. The reaction mixture was passed through a short pad of celite and washed with MeOH. Upon concentration of the filtrate under reduced pressure yield the crude product, which was further purified by reverse phase HPLC. White solid (0.019 g, 75%). ¹H NMR (DMSO-d₆): δ 7.83 (d, J=89.2 Hz, 1H), 7.76 (dd, J=2.2 Hz, 8.7 Hz, 1H), 7.68 (d, J=1.8 Hz, 1H), 6.98 (d, J=8.7 Hz, 1H), 6.56 (d, J=9.2 Hz, 1H), 4.04 (t, J=6.4 Hz, 2H), 3.79 (s, 3H), 2.80 (s, 2H), 2.56 (t, J=7.3 Hz, 2H), 1.91 (s, 3H), 1.76-1.69 (m, 2H), 1.59-1.52 (m, 2H), 1.45-1.39 (m, 2H), 0.96 (s, 9H). ¹³C NMR (DMSO-d₆): δ 205.8, 167.2, 162.5, 161.5, 160.6, 131.2, 130.8, 129.9, 129.2, 122.6, 114.3, 111.8, 110.3, 103.1, 67.9, 55.6, 48.5, 31.6, 29.3, 28.8, 28.2, 25.2, 7.7. LC-MS (ESI) Calcd for C₂₆H₃₄O₆ [M+H]⁺: 443.24. Found: 443.00. HRMS (ESI) Calcd for C₂₆H₃₄O₆ [M+H]⁺: 443.2377. Found: 443.2429.

### Example 4

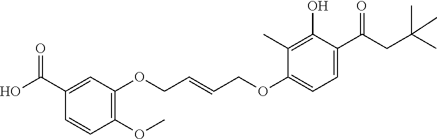 [SMILES: COc1ccc(C(=O)O)cc1OC/C=C/COc1ccc(C(=O)CC(C)(C)C)c(O)c1C]

### (E)-3-((4-(4-(3,3-Dimethylbutanoyl)-3-hydroxy-2-methylphenoxy)but-2-en-1-yl)oxy)-4-methoxybenzoic acid

Methyl 3-hydroxy-4-methoxybenzoate, trans-1,4-dibromo-but-2-ene, 1-(2,4-dihydroxy-3-methylphenyl)-3,3-dimethylbutan-1-one, K₂CO₃ and LiOH were processed according to general method B, C and D. White solid. ¹H NMR (DMSO-d₆): δ 7.85 (d, J=9.2 Hz, 1H), 7.54 (dd, J=1.8 Hz, 8.2 Hz, 1H), 7.43 (d, J=1.8 Hz, 1H), 7.02 (d, J=9.2 Hz, 1H), 6.59 (d, J=9.2 Hz, 1H), 6.06-6.03 (m, 2H), 4.70-4.61 (m, 4H), 3.75 (s, 3H), 2.81 (s, 2H), 1.97 (s, 3H), 0.97 (s, 9H). ¹³C NMR (DMSO-d₆): δ 205.9, 167.1, 162.0, 161.6, 152.8, 146.9, 131.1, 128.3, 128.1, 123.4, 114.5, 113.6, 112.0, 111.2, 110.4, 67.8, 67.6, 55.6, 48.5, 31.6, 29.8, 7.6. LC-MS (ESI) Calcd for C₂₅H₃₀O₇ [M+H]⁺: 443.20. Found: 443.00. HRMS (ESI) Calcd for C₂₅H₃₀O₇ [M+H]: 443.2035. Found: 443.2107.

### Example 5

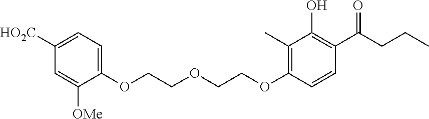 [SMILES: CCCC(=O)c1ccc(OCCOCCOc2ccc(C(=O)O)cc2OC)c(C)c1O]

### 4-(2-(2-(4-Butyryl-3-hydroxy-2-methylphenoxy)ethoxy)ethoxy)-3-methoxybenzoic acid

Methyl 4-hydroxy-3-methoxybenzoate, bis(2-bromomethyl)ether, 1-(2,4-dihydroxy-3-methylphenyl)butan-1-one, K₂CO₃ and LiOH were processed according to general method B, C and D. White solid. ¹H NMR (DMSO-d₆): δ 7.77 (d, J=9.2 Hz, 1H), 7.47 (d, J=8.2 Hz, 1H), 7.39 (s, 1H), 6.99 (d, J=8.2 Hz, 1H), 6.59 (d, J=8.7 Hz, 1H), 4.20-4.12 (m, 4H), 3.85-3.81 (m, 4H), 3.74 (s, 3H), 2.94 (t, J=7.3 Hz, 2H), 1.97 (s, 3H), 1.64-1.55 (m, 2H), 0.89 (t, J=8.2 Hz, 3H). LC-MS (ESI) Calcd for C₂₃H₂₈O₈ [M+H]⁺: 433.18. Found: 433.05. HRMS (ESI) Calcd for C₂₃H₂₈O₈ [M+H]⁺: 433.1784. Found: 433.1908.

### Example 6

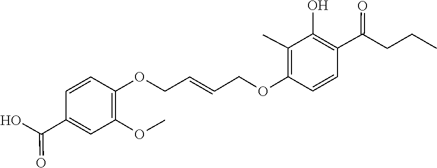 [SMILES: CCCC(=O)c1ccc(OC/C=C/COc2ccc(C(=O)O)cc2OC)c(C)c1O]

### (E)-4-((4-(4-Butyryl-3-hydroxy-2-methylphenoxy)but-2-en-1-yl)oxy)-3-methoxybenzoic acid

Methyl 4-hydroxy-3-methoxybenzoate, trans-1,4-dibromo-but-2-ene, 1-(2,4-dihydroxy-3-methylphenyl)butan-1-one, K₂CO₃ and LiOH were processed according to general method B, C and D. White solid. ¹H NMR (DMSO-d₆): δ 7.77 (d, J=9.2 Hz, 1H), 7.48 (dd, J=1.8 Hz, 8.7 Hz, 1H), 7.40 (d, J=2.2 Hz, 1H), 7.00 (d, J=8.7 Hz, 1H), 6.59 (d, J=8.7 Hz, 1H), 6.05-6.03 (m, 2H), 4.69-4.63 (m, 4H), 3.79 (s, 3H), 2.94 (t, J=7.1 Hz, 2H), 2.00 (s, 3H), 1.63-1.57 (m, 2H), 0.89 (t, J=7.8 Hz, 3H). LC-MS (ESI) Calcd for C₂₃H₂₆O₇ [M+H]⁺: 415.18. Found: 415.05. HRMS (ESI) Calcd for C₂₃H₂₆O₇ [M+H]⁺: 415.1709. Found: 415.1777.

### Example 7

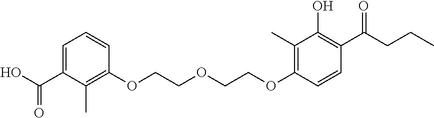 [SMILES: CCCC(=O)c1ccc(OCCOCCOc2cccc(C(=O)O)c2C)c(C)c1O]

### 3-(2-(2-(4-Butyryl-3-hydroxy-2-methylphenoxy)ethoxy)ethoxy)-2-methylbenzoic acid

Methyl 3-hydroxy-2-methylbenzoate, bis(2-bromomethyl)ether, 1-(2,4-dihydroxy-3-methylphenyl)butan-1-one, K₂CO₃ and LiOH were processed according to general method B, C and D. White solid. ¹H NMR (DMSO-d₆): δ 7.79 (d, J=9.2 Hz, 1H), 7.29-7.27 (m, 1H), 7.18 (t, J=8.2 Hz, 1H), 7.11 (d, J=8.2 Hz, 1H), 6.62 (d, J=9.2 Hz, 1H), 4.23-4.10 (m, 4H), 3.86-3.83 (m, 4H), 2.96 (t, J=7.3 Hz, 2H), 2.29 (s, 3H), 1.97 (s, 3H), 1.65-1.59 (m, 2H), 0.91 (t, J=7.3 Hz, 3H). LC-MS (ESI) Calcd for C₂₃H₂₈O₇ [M+H]⁺: 417.18. Found: 417.05. HRMS (ESI) Calcd for C₂₃H₂₈O₇[M−H]⁺: 415.1777. Found: 415.1703.

### Example 8

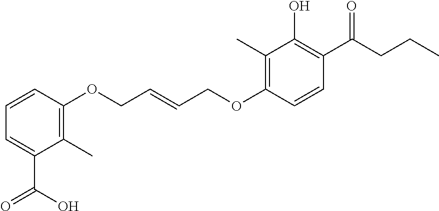 [SMILES: CCCC(=O)c1ccc(OC/C=C/COc2cccc(C(=O)O)c2C)c(C)c1O]

### (E)-3-((4-(4-Butyryl-3-hydroxy-2-methylphenoxy)but-2-en-1-yl)oxy)-2-methylbenzoic acid

Methyl 3-hydroxy-2-methylbenzoate, trans-1,4-dibromo-but-2-ene, 1-(2,4-dihydroxy-3-methylphenyl)butan-1-one, K₂CO₃ and LiOH were processed according to general method B, C and D. White solid. ¹H NMR (DMSO-d₆): δ 7.82 (d, J=8.7 Hz, 1H), 7.30 (d, J=7.3 Hz, 1H), 7.21 (t, J=8.2 Hz, 1H), 7.12 (d, J=7.8 Hz, 1H), 6.64 (d, J=9.2 Hz, 1H), 6.10-6.09 (m, 2H), 4.73-4.64 (m, 4H), 2.97 (t, J=7.3 Hz, 2H), 2.34 (s, 3H), 2.00 (s, 3H), 1.66-1.60 (m, 2H), 0.94 (t, J=7.8 Hz, 3H). LC-MS (ESI) Calcd for C₂₃H₂₆O₆ [M+H]⁺: 399.17. Found: 399.05. HRMS (ESI) Calcd for C₂₃H₂₆O₆ [M+H]⁺: 399.1752. Found: 399.1830.

### Example 9

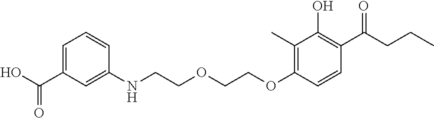 [SMILES: CCCC(=O)c1ccc(OCCOCCNc2cccc(C(=O)O)c2)c(C)c1O]

### 3-({2-[2-(4-Butanoyl-3-hydroxy-2-methylphenoxy)ethoxy]ethyl}amino)benzoic acid

3-Aminobenzoate, bis(2-bromomethyl)ether, 1-(2,4-dihydroxy-3-methylphenyl)butan-1-one, K₂CO₃ and LiOH were processed according to general method B, C and D. White solid (0.03 g, 14.9%). ¹H NMR (400 MHz, DMSO-d₆) δ 7.82 (d, J=9.0 Hz, 1H), 7.18-7.11 (m, 2H), 6.82-6.78 (m, 1H), 6.64 (d, J=9.0 Hz, 1H), 5.85 (t, J=5.8 Hz, 1H), 4.25-4.19 (m, 2H), 3.83-3.76 (m, 2H), 3.66 (t, J=5.7 Hz, 1H), 3.28-3.18 (m, 4H), 2.98 (d, J=7.2 Hz, 2H), 1.99 (s, 3H), 1.64-1.55 (m, 2H), 0.94 (t, J=7.4 Hz, 3H). LC-MS (ESI) Calcd for C₂₂H₂₇NO₆ [M+H]⁺: 402.18. Found: 402.00. HRMS (ESI) Calcd for C₂₂H₂₇NO₆ [M+H]⁺: 402.1814. Found: 404.1930.

### Example 10

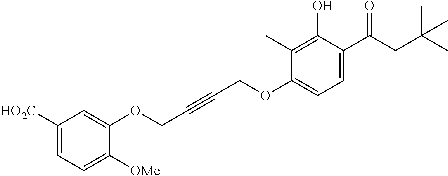 [SMILES: COc1ccc(C(=O)O)cc1OCC#CCOc1ccc(C(=O)CC(C)(C)C)c(O)c1C]

### 3-{4-[4-(3,3-Dimethylbutanoyl)-3-hydroxy-2-methylphenoxy]but-2-ynyloxy}-4-methoxybenzoic acid

Methyl 3-hydroxy-4-methoxybenzoate, 1,4-dibromobut-2-yne, dihydroxy-3-methylphenyl)-3,3-dimethylbutan-1-one, K₂CO₃ and LiOH were processed according to general method B, C and D. White solid. ¹H NMR (400 MHz, DMSO-d₆) δ 7.82 (d, J=9.1 Hz, 1H), 7.59-7.49 (m, 2H), 7.03 (d, J=8.2 Hz, 1H), 6.58 (d, J=9.2 Hz, 1H), 4.96 (s, 2H), 4.87 (s, 2H), 3.78 (s, 3H), 2.84 (s, 2H), 1.93 (s, 3H), 0.97 (s, 9H). LC-MS (ESI) Calcd for C₂₅H₂₈O₇ [M+H]⁺: 441.18. Found: 441.00. HRMS (ESI) Calcd for C₂₂H₂₇NO₆ [M+H]⁺: 441.1816. Found: 441.1920.

### Example 11

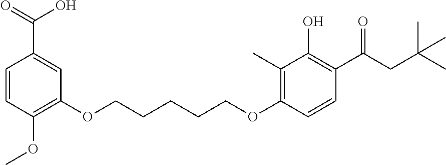 [SMILES: COc1ccc(C(=O)O)cc1OCCCCCOc1ccc(C(=O)CC(C)(C)C)c(O)c1C]

### 3-{5-[4-(3,3-Dimethylbutanoyl)-3-hydroxy-2-methylphenoxy]pentyloxy}-4-methoxybenzoic acid

Methyl 3-hydroxy-4-methoxybenzoate, 1,5-dibropentane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆) δ 7.88 (d, J=9.2 Hz, 1H), 7.55 (dd, J=1.8 Hz, 8.4 Hz, 1H), 7.39 (d, J=1.8 Hz, 1H), 6.99 (d, J=8.7 Hz, 1H), 6.57 (d, J=9.2 Hz, 1H), 4.07 (t, J=6.4 Hz, 2H), 3.97 (t, J=6.4 Hz, 2H), 3.77 (s, 3H), 2.79 (s, 2H), 1.95 (s, 3H), 1.79-1.72 (m, 4H), 1.57-1.54 (m, 2H), 0.96 (s, 9H). LC-MS (ESI) Calcd for C₂₆H₃₄O₇ [M+H]⁺: 459.23. Found: 459.10. HRMS (ESI) Calcd for C₂₆H₃₄O₇ [M+H]⁺: 459.2348. Found: 459.2476.

### Example 12

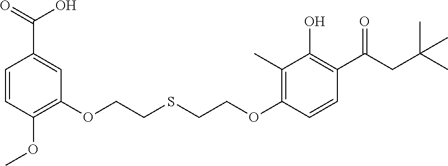 [SMILES: COc1ccc(C(=O)O)cc1OCCSCCOc1ccc(C(=O)CC(C)(C)C)c(O)c1C]

### 3-(2-{2-[4-(3,3-Dimethylbutanoyl)-3-hydroxy-2-methylphenoxy]ethylthio}ethoxy)-4-methoxybenzoic acid

Methyl 3-hydroxy-4-methoxybenzoate, bis(2-bromoethyl)sulfane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.84 (d, J=9.2 Hz, 1H), 7.57 (dd, J=1.9 Hz, 8.4 Hz, 1H), 7.45 (d, J=1.8 Hz, 1H), 7.00 (d, J=8.2 Hz, 1H), 6.58 (d, J=9.2 Hz, 1H), 4.30 (t, J=6.4 Hz, 2H), 4.19 (t, J=6.4 Hz, 2H), 3.75 (s, 3H), 3.07 (t, J=6.4 Hz, 2H), 3.01 (t, J=6.3 Hz, 2H), 2.85 (s, 2H), 1.99 (s, 3H), 0.96 (s, 9H). LC-MS (ESI) Calcd for C₂₅H₃₂O₇S [M+H]⁺: 477.19. Found: 477.05. HRMS (ESI) Calcd for C₂₅H₃₂O₇S [M+H]⁺: 477.1883. Found: 477.1951.

### Example 13

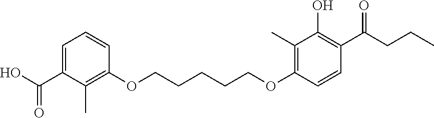 [SMILES: CCCC(=O)c1ccc(OCCCCCOc2cccc(C(=O)O)c2C)c(C)c1O]

### 3-{5-[4-(3,3-Dimethylbutanoyl)-3-hydroxy-2-methylphenoxy]pentyloxy}-2-methylbenzoic acid

Methyl 3-hydroxy-2-methybenzoate, 1,5-dibromopentane, 1-(2,4-dihydroxy-3-methylphenyl)butan-1-one, K₂CO₃ and LiOH were processed according to general method B, C and D. White solid. ¹H NMR (400 MHz, DMSO-d₆) δ 7.80 (d, J=9.2 Hz, 1H), 7.29 (d, J=7.8 Hz, 1H), 7.20 (t, J=7.9 Hz, 1H), 7.13-7.09 (m, 1H), 6.63 (d, J=9.2 Hz, 1H), 4.11 (t, J=6.0 Hz, 2H), 4.00 (t, J=6.0 Hz, 2H), 2.96 (t, J=7.3 Hz, 2H), 2.33 (s, 3H), 1.99 (s, 3H), 1.82-1.79 (m, 4H), 1.65-1.58 (m, 4H), 0.93 (t, J=7.4 Hz, 3H). LC-MS (ESI) Calcd for C₂₄H₃₀O₆ [M+H]⁺: 415.20. Found: 415.05. HRMS (ESI) Calcd for C₂₄H₃₀O₆ [M+H]⁺: 415.2058. Found: 415.2116.

### Example 14

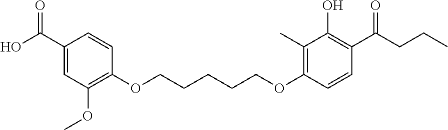 [SMILES: CCCC(=O)c1ccc(OCCCCCOc2ccc(C(=O)O)cc2OC)c(C)c1O]

### 4-[5-(4-Butanoyl-3-hydroxy-2-methylphenoxy)pentyloxy]-3-methoxybenzoic acid

Methyl 4-hydroxy-3-methoxybenzoate, 1,5-dibromopentane, 1-(2,4-dihydroxy-3-methylphenyl)butan-1-one, K₂CO₃ and LiOH were processed according to general method B, C and D. White solid. ¹H NMR (400 MHz, DMSO-d₆) δ 7.79 (d, J=9.0 Hz, 1H), 7.49-7.47 (m, 1H), 7.38-7.37 (m, 1H), 7.04 (d, J=8.2 Hz, 1H), 6.63 (d, J=9.2 Hz, 1H), 4.08 (t, J=6.3 Hz, 2H), 3.99 (t, J=6.4 Hz, 2H), 3.78 (s, 3H), 2.97 (t, J=7.4 Hz, 2H), 1.99 (s, 3H), 1.81-1.77 (m, 4H), 1.62-1.53 (m, 4H), 0.93 (t, J=7.4, 0.9 Hz, 3H). LC-MS (ESI) Calcd for C₂₄H₃₀O₇ [M+H]⁺: 431.20. Found: 431.10. HRMS (ESI) Calcd for C₂₄H₃₀O₇ [M−H]⁺: 429.1965. Found: 429.1862.

### Example 15

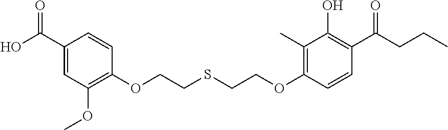 [SMILES: CCCC(=O)c1ccc(OCCSCCOc2ccc(C(=O)O)cc2OC)c(C)c1O]

### 4-{2-[2-(4-Butanoyl-3-hydroxy-2-methylphenoxy)ethylthio]ethoxy}-3-methoxybenzoic acid

Methyl 4-hydroxy-3-methoxybenzoate, bis(2-bromoethyl)sulfane, 1-(2,4-dihydroxy-3-methylphenyl)butan-1-one, K₂CO₃ and LiOH were processed according to general method B, C and D. White solid. ¹H NMR (400 MHz, DMSO-d₆): δ 7.78 (dd, J=2.0 Hz, 9.2 Hz, 1H), 7.53 (dt, J=2.2 Hz, 8.4 Hz, 1H), 7.39 (t, J=2.2 Hz, 1H), 7.05 (dd, J=2.1 Hz, 8.2 Hz, 1H), 6.65 (d, J=9.2 Hz, 1H), 4.26 (t, J=6.4 Hz, 2H), 4.17 (t, J=6.4 Hz, 2H), 3.78 (s, 3H), 3.13-2.93 (m, 6H), 1.99 (s, 3H), 1.62-1.57 (m, 2H), 0.94 (t, J=7.4, Hz, 3H). LC-MS (ESI) Calcd for C₂₃H₂₈O₇S [M+H]⁺: 449.16. Found: 449.00.

### Example 16

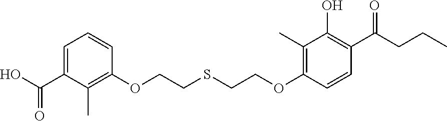 [SMILES: CCCC(=O)c1ccc(OCCSCCOc2cccc(C(=O)O)c2C)c(C)c1O]

### 3-{2-[2-(4-Butanoyl-3-hydroxy-2-methylphenoxy)ethylthio]ethoxy}-2-methylbenzoic acid

Methyl 4-hydroxy-2-methylbenzoate, bis(2-bromoethyl)sulfane, 1-(2,4-dihydroxy-3-methylphenyl)butan-1-one, K₂CO₃ and LiOH were processed according to general method B, C and D. White solid. ¹H NMR (400 MHz, DMSO-d₆): δ 7.82 (d, J=9.2 Hz, 1H), 7.31 (d, J=7.7, 1.0 Hz, 1H), 7.21 (t, J=7.9 Hz, 1H), 7.15-7.12 (m, 1H), 6.64 (d, J=9.2 Hz, 1H), 4.29 (t, J=6.3 Hz, 2H), 4.19 (t, J=6.3 Hz, 2H), 3.04 (t, J=6.3 Hz, 4H), 3.00-2.95 (m, 2H), 2.33 (s, 3H), 1.99 (s, 3H), 1.64-1.58 (m, 2H), 0.93 (t, J=7.4 Hz, 3H). LC-MS (ESI) Calcd for C₂₃H₂₈O₆S [M+H]⁺: 433.16. Found: 433.05. HRMS (ESI) Calcd for C₂₃H₂₈O₆S [M+H]⁺: 433.1627. Found: 433.1695.

### Example 17

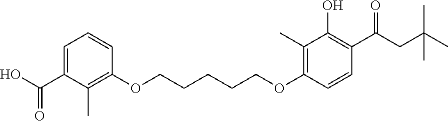 [SMILES: Cc1c(OCCCCCOc2ccc(C(=O)CC(C)(C)C)c(O)c2C)cccc1C(=O)O]

### 3-{5-[4-(3,3-Dimethylbutanoyl)-3-hydroxy-2-methylphenoxy]pentyloxy}-2-methylbenzoic acid

Methyl 3-hydroxy-2-methylbenzoate, 1,5-dibromopentane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.87 (d, J=9.2 Hz, 1H), 7.26-7.05 (m, 3H), 6.58 (d, J=9.2 Hz, 1H), 4.11 (t, J=6.4 Hz, 2H), 3.94 (t, J=6.4 Hz, 2H 2.79 (s, 2H), 2.26 (s, 3H), 1.90 (s, 3H), 1.81-1.78 (m, 4H), 1.60-1.57 (m, 2H), 0.96 (s, 9H). LC-MS (ESI) Calcd for C₂₆H₃₄O₆ [M+H]⁺: 443.24. Found: 443.05. HRMS (ESI) Calcd for C₂₆H₃₄O₆ [M+H]⁺:443.2353. Found: 443.2430.

### Example 18

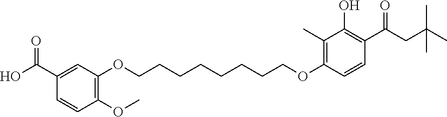 [SMILES: COc1ccc(C(=O)O)cc1OCCCCCCCCOc1ccc(C(=O)CC(C)(C)C)c(O)c1C]

### 3-{8-[4-(3,3-Dimethylbutanoyl)-3-hydroxy-2-methylphenoxy]octyloxy}-4-methoxybenzoic acid

Methyl 3-hydroxy-4-methoxybenzoate, 1,8-dibromooctane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.84 (d, J=9.2 Hz, 1H), 7.51 (dd, J=1.8 Hz, 8.2 Hz, 1H), 7.38 (s, 1H), 6.98 (d, J=8.2 Hz, 1H), 6.57 (d, J=9.2 Hz, 1H), 4.11 (t, J=6.4 Hz, 2H), 3.94 (t, J=6.4 Hz, 2H), 3.76 (s, 3H), 2.79 (s, 2H), 1.97 (s, 3H), 1.71-1.65 (m, 4H), 1.39-1.31 (m, 8H), 0.96 (s, 9H). LC-MS (ESI) Calcd for C₂₉H₄₀O₇ [M+H]⁺: 501.28. Found: 501.15. HRMS (ESI) Calcd C₂₉H₄₀O₇ [M+H]⁺: 501.2774. Found: 501.2846.

### Example 19

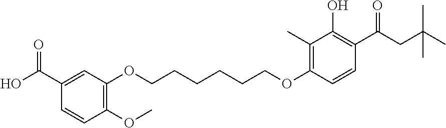 [SMILES: COc1ccc(C(=O)O)cc1OCCCCCCOc1ccc(C(=O)CC(C)(C)C)c(O)c1C]

### 3-{6-[4-(3,3-Dimethylbutanoyl)-3-hydroxy-2-methylphenoxy]hexyloxy}-4-methoxybenzoic acid

Methyl 3-hydroxy-4-methoxybenzoate, 1,6-dibromohexane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.85 (d, J=9.2 Hz, 1H), 7.51 (dd, J=1.8 Hz, 8.2 Hz, 1H), 7.38 (s, 1H), 6.99 (d, J=8.7 Hz, 1H), 6.56 (d, J=9.2 Hz, 1H), 4.06 (t, J=6.4 Hz, 2H), 3.94 (t, J=6.4 Hz, 2H), 3.76 (s, 3H), 2.83 (s, 2H), 1.97 (s, 3H), 1.72-1.69 (m, 4H), 1.46-1.45 (m, 4H), 0.96 (s, 9H). LC-MS (ESI) Calcd for C₂₇H₃₆O₇ [M+H]⁺: 473.25. Found: 473.15. HRMS (ESI) Calcd C₂₇H₃₆O₇ [M+H]⁺: 473.2454. Found: 473.2531.

### Example 20

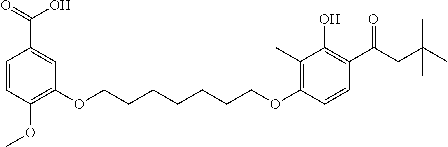 [SMILES: COc1ccc(C(=O)O)cc1OCCCCCCCOc1ccc(C(=O)CC(C)(C)C)c(O)c1C]

### 3-{7-[4-(3,3-Dimethylbutanoyl)-3-hydroxy-2-methylphenoxy]heptyloxy}-4-methoxybenzoic acid

Methyl 3-hydroxy-4-methoxybenzoate, 1,7-dibromoheptane, 1-(2,4-dihydroxy-3-methyl phenyl)-3,3-dimethylbutan-1-one, K₂CO₃ and LiOH were processed according to general method B, C and D. White solid. ¹H NMR (400 MHz, DMSO-d₆): δ 7.84 (d, J=9.2 Hz, 1H), 7.51 (dd, J=1.8 Hz, 8.2 Hz, 1H), 7.38 (s, 1H), 6.99 (d, J=8.7 Hz, 1H), 6.56 (d, J=9.2 Hz, 1H), 4.06 (t, J=6.4 Hz, 2H), 3.94 (t, J=6.4 Hz, 2H), 3.76 (s, 3H), 2.83 (s, 2H), 1.97 (s, 3H), 1.72-1.69 (m, 4H), 1.56-1.37 (m, 6H), 0.96 (s, 9H). LC-MS (ESI) Calcd for C₂₈H₃₈O₇ [M+H]⁺: 487.26. Found: 487.15. HRMS (ESI) Calcd C₂₈H₃₈O₇ [M+H]⁺: 487.2623. Found: 487.2681.

### Example 21

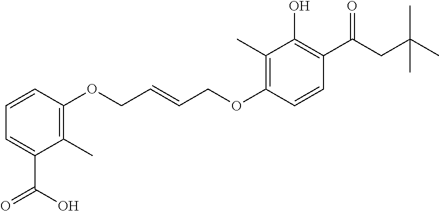 [SMILES: Cc1c(OC/C=C/COc2ccc(C(=O)CC(C)(C)C)c(O)c2C)cccc1C(=O)O]

### 3-{(2E)-4-[4-(3,3-Dimethylbutanoyl)-3-hydroxy-2-methylphenoxy]but-2-enyloxy}-2-methylbenzoic acid

Methyl 3-hydroxy-2-methylbenzoate, trans-1,4-dibromo-but-2-ene, 1-(2,4-dihydroxy-3-methyl phenyl)-3,3-dimethylbutan-1-one, K₂CO₃ and LiOH were processed according to general method B, C and D. White solid. ¹H NMR (400 MHz, DMSO-d₆): δ 7.85 (d, J=9.2 Hz, 1H), 7.27-7.08 (m, 3H), 6.60 (d, J=9.2 Hz, 1H), 6.06 (s, 2H), 4.70 (s, 2H), 34.60 (s, 2H), 2.85 (s, 2H), 2.30 (s, 3H), 1.97 (s, 3H), 0.96 (s, 9H). LC-MS (ESI) Calcd for C₂₅H₃₀O₆ [M+H]⁺: 427.20. Found: 427.05. HRMS (ESI) Calcd C₂₅H₃₀O₆ [M+H]⁺: 427.2058. Found: 427.2118.

### Example 22

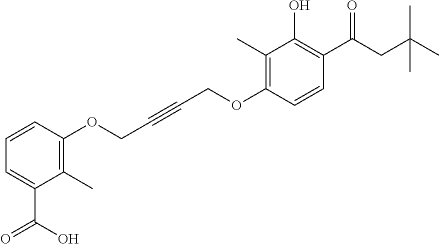 [SMILES: Cc1c(OCC#CCOc2ccc(C(=O)CC(C)(C)C)c(O)c2C)cccc1C(=O)O]

### 3-{4-[4-(3,3-Dimethylbutanoyl)-3-hydroxy-2-methylphenoxy]but-2-ynyloxy}-2-methylbenzoic acid

Methyl 3-hydroxy-2-methylbenzoate, 1,4-dibromobut-2-yne, 1-(2,4-dihydroxy-3-methyl phenyl)-3,3-dimethylbutan-1-one, K₂CO₃ and LiOH were processed according to general method B, C and D. White solid. ¹H NMR (400 MHz, DMSO-d₆) δ 7.85 (d, J=9.1 Hz, 1H), 7.36 (d, J=1.6 Hz, 1H), 7.23-7.13 (m, 2H), 6.63 (d, J=9.1 Hz, 1H), 5.02 (s, 2H), 4.93 (s, 2H), 2.86 (s, 2H), 2.33 (s, 3H), 1.99 (s, 3H), 1.01 (s, 9H). LC-MS (ESI) Calcd for C₂₅H₂₈O₆ [M+H]⁺: 425.19. Found: 425.05. HRMS (ESI) Calcd for C₂₅H₂₈O₆ [M+H]⁺: 425.1912. Found: 425.1972.

### Example 23

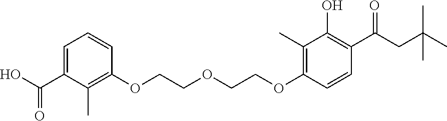 [SMILES: Cc1c(OCCOCCOc2ccc(C(=O)CC(C)(C)C)c(O)c2C)cccc1C(=O)O]

### 3-(2-{2-[4-(3,3-Dimethylbutanoyl)-3-hydroxy-2-methylphenoxy]ethoxy}ethoxy)-2-methylbenzoic acid

Methyl 3-hydroxy-2-methylbenzoate, bis(2-bromomethyl)ether, 1-(2,4-dihydroxy-3-methyl phenyl)-3,3-dimethylbutan-1-one, K₂CO₃ and LiOH were processed according to general method B, C and D. White solid. ¹H NMR (400 MHz, DMSO-d₆): δ 7.87 (d, J=9.1 Hz, 1H), 7.31 (dd, J=1.2 Hz, 7.8 Hz, 1H), 7.20 (t, J=7.9 Hz, 1H), 7.12 (dd, J=1.3 Hz, 8.2 Hz, 1H), 6.63 (d, J=9.1 Hz, 1H), 4.26-4.22 (m, 2H), 4.16-4.11 (m, 2H), 3.90-3.83 (m, 4H), 2.84 (s, 2H), 2.32 (s, 3H), 1.98 (s, 3H), 1.00 (s, 9H). LC-MS (ESI) Calcd for C₂₅H₃₂O₇ [M+H]⁺: 445.21. Found: 445.10. HRMS (ESI) Calcd for C₂₅H₃₂O₇ [M+H]⁺: 445.2181. Found: 445.2238.

### Example 24

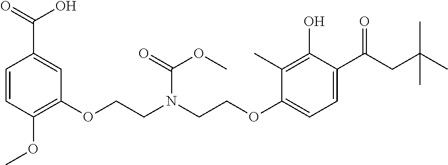 [SMILES: COC(=O)N(CCOc1cc(C(=O)O)ccc1OC)CCOc1ccc(C(=O)CC(C)(C)C)c(O)c1C]

### 3-[2-(N-{2-[4-(3,3-Dimethylbutanoyl)-3-hydroxy-2-methylphenoxy]ethyl}methoxycarbonylamino)ethoxy]-4-methoxybenzoic acid

Methyl 3-hydroxy-4-methoxybenzoate, methyl bis(2-bromoethyl)carbamate, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.88 (d, J=9.1 Hz, 1H), 7.56 (dd, J=1.9 Hz, 8.4 Hz, 1H), 7.44 (d, J=5.6 Hz, 1H), 7.03 (d, J=8.5 Hz, 1H), 6.61 (d, J=9.2 Hz, 1H), 4.25 (t, J=5.8 Hz, 2H), 4.17-4.11 (m, 2H), 3.79 (s, 3H), 3.71-3.67 (m, 4H), 3.61 (s, 3H), 2.84 (s, 2H), 1.96 (s, 3H), 1.00 (s, 9H). LC-MS (ESI) Calcd for C₂₇H₃₅O₉ [M+H]⁺: 518.23. Found: 518.05. HRMS (ESI) Calcd for C₂₇H₃₅O₉ [M+H]⁺: 518.2197. Found: 518.2210.

### Example 25

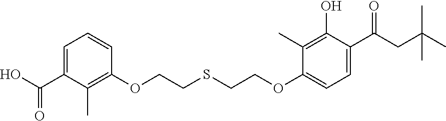 [SMILES: Cc1c(OCCSCCOc2ccc(C(=O)CC(C)(C)C)c(O)c2C)cccc1C(=O)O]

### 2-(2-{2-[4-(3,3-Dimethylbutanoyl)-3-hydroxy-2-methylphenoxy]ethylthio}ethoxy)benzoic acid

Methyl 4-hydroxy-2-methylbenzoate, bis(2-bromoethyl)sulfane, 1-(2,4-dihydroxy-3-methyl phenyl)-3,3-dimethylbutan-1-one, K₂CO₃ and LiOH were processed according to general method B, C and D. White solid. ¹H NMR (400 MHz, DMSO-d₆): δ 7.87 (d, J=9.1 Hz, 1H), 7.32-7.29 (m, 1H), 7.20 (t, J=7.9 Hz, 1H), 7.14-7.11 (m, 1H), 6.62 (d, J=9.1 Hz, 1H), 4.29 (t, J=6.3 Hz, 2H), 4.18 (t, J=6.3 Hz, 2H), 3.04 (t, J=6.3 Hz, 4H), 2.84 (s, 2H), 2.33 (s, 3H), 1.99 (s, 3H), 1.00 (s, 9H). LC-MS (ESI) Calcd for C₂₅H₃₂O₆S [M+H]⁺: 461.19. Found: 461.05. HRMS (ESI) Calcd for C₂₅H₃₂O₆S [M+H]⁺: 461.1946. Found: 461.2017.

### Example 26

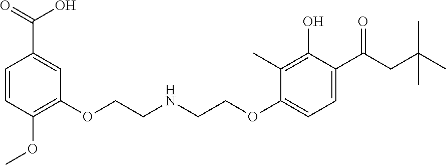 [SMILES: COc1ccc(C(=O)O)cc1OCCNCCOc1ccc(C(=O)CC(C)(C)C)c(O)c1C]

### 3-[2-({2-[4-(3,3-Dimethylbutanoyl)-3-hydroxy-2-methylphenoxy]ethyl}amino)ethoxy]-4-methoxybenzoic acid

A solution of 3-(2-((2-(4-(3,3-dimethylbutanoyl)-3-hydroxy-2-methylphenoxy)ethyl) (methoxycarbonyl)amino)ethoxy)-4-methoxybenzoic acid (40 mg, 0.077 mmol) and TBAF (0.38 mL, 1 M in THF, 0.38 mmol) in dry THF (2.5 mL) was stirred under argon at reflux. After 40 min water (5 mL) was added and the aqueous phase was extracted with EtOAc (3×5 mL). The combined organic layers were dried over Na₂SO₄, filtered and the solvent was removed under reduced pressure. The crude product was purified by reverse phase HPLC to afford the title compound as colorless solid (0.005 g, 14.1%). LC-MS (ESI) Calcd for C₂₅H₃₃NO₇ [M+H]⁺: 460.23. Found: 460.10. HRMS (ESI) Calcd for C₂₅H₃₃NO₇ [M+H]⁺: 460.2226. Found: 460.2333.

### Example 27

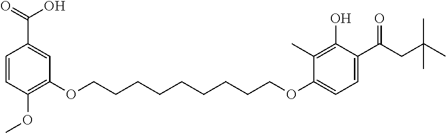 [SMILES: COc1ccc(C(=O)O)cc1OCCCCCCCCCOc1ccc(C(=O)CC(C)(C)C)c(O)c1C]

### 3-{9-[4-(3,3-Dimethylbutanoyl)-3-hydroxy-2-methylphenoxy]nonyloxy}-4-methoxybenzoic acid

Methyl 3-hydroxy-4-methoxybenzoate, 1,9-dibromononane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.87 (d, J=9.1 Hz, 1H), 7.55 (dd, J=1.9 Hz, 8.4 Hz, 1H), 7.42 (d, J=2.0 Hz, 1H), 7.02 (d, J=8.5 Hz, 1H), 6.59 (d, J=9.1 Hz, 1H), 4.07 (t, J=6.4 Hz, 2H), 3.96 (t, J=6.6 Hz, 2H), 3.81 (s, 3H), 2.83 (s, 2H), 1.98 (s, 3H), 1.75-1.70 (m, 4H), 1.41-1.36 (m, 10H), 1.00 (s, 9H). LC-MS (ESI) Calcd for C₃₀H₄₂O₇ [M+H]⁺: 515.29. Found: 515.15. HRMS (ESI) Calcd for C₃₀₁H₄₂O₇ [M+H]⁺: 515.2914. Found: 515.2991.

### Example 28

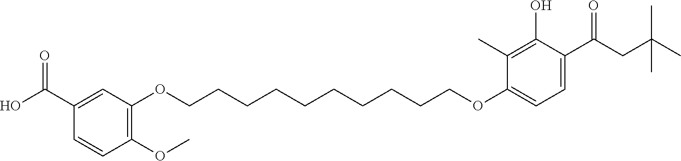 [SMILES: COc1ccc(C(=O)O)cc1OCCCCCCCCCCOc1ccc(C(=O)CC(C)(C)C)c(O)c1C]

### 3-{10-[4-(3,3-Dimethylbutanoyl)-3-hydroxy-2-methylphenoxy]decyloxy}-4-methoxybenzoic acid

Methyl 3-hydroxy-4-methoxybenzoate, 1,10-dibromodecane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ ¹H NMR (400 MHz, DMSO-d₆) δ 7.87 (d, J=9.1 Hz, 1H), 7.55 (dd, J=1.9 Hz, 8.4 Hz, 1H), 7.42 (d, J=2.0 Hz, 1H), 7.03 (d, J=8.5 Hz, 1H), 6.59 (d, J=9.1 Hz, 1H), 4.07 (t, J=6.4 Hz, 2H), 3.96 (t, J=6.6 Hz, 2H), 3.82 (s, 3H), 2.83 (s, 2H), 1.98 (s, 3H), 1.75-1.70 (m, 4H), 1.43-1.26 (m, 12H), 1.00 (s, 9H). LC-MS (ESI) Calcd for C₃₁H₄₄O₇ [M+H]⁺: 529.31. Found: 529.00. HRMS (ESI) Calcd for C₃₁H₄₄O₇ [M+H]⁺: 529.3091. Found: 529.3157.

### Example 29

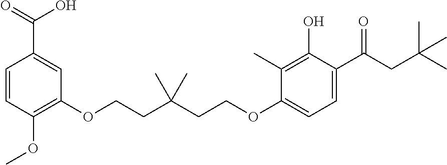 [SMILES: COc1ccc(C(=O)O)cc1OCCC(C)(C)CCOc1ccc(C(=O)CC(C)(C)C)c(O)c1C]

### 3-{5-[4-(3,3-Dimethylbutanoyl)-3-hydroxy-2-methylphenoxy]-3,3-dimethylpentyloxy}-4-methoxybenzoic acid

3,3-Dimethylpentane-1,5-diol (2.0 g, 15.1 mmol) was dissolved in dry CH₂Cl₂ (75 mL) under argon atmosphere and the solution was cooled to −20° C. Et₃N (8.4 mL, 60 mmol) was added and then MsCl (3.5 mL, 45 mmol) was added slowly over 5 minutes. The mixture was stirred for 1 h, then quenched with H₂O and the mixture was extracted with CH₂Cl₂ (3×20 mL). The combined organic layers were washed with brine, dried over Na₂SO₄, filtered, and concentrated under reduced pressure. To a solution of the crude mixture in acetone (150 mL) was added LiBr (3.1 g, 151 mmol). The resulting suspension was stirred for 6 h at reflux. After cooling to rt, Et₂O was added and the organic layer was washed with H₂O and brine, dried over Na₂SO₄, filtered, and concentrated under reduced pressure, to give 1,5-dibromo-3,3-dimethylpentane. The crude mixture was used for the next step without purification. Pale yellow oil (3 g, 77%), ¹H NMR (400 MHz, CDCl₃) δ: 3.32-3.36 (m, 41H), 1.82-1.85 (m, 41), 0.92 (s, 6H).

Methyl 3-hydroxy-4-methoxybenzoate, 1,5-dibromo-3,3-dimethylpentane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.88 (d, J=9.1 Hz, 1H), 7.56 (dd, J=1.9 Hz, 8.2 Hz, 1H), 7.47 (d, J=2.0 Hz, 1H), 7.02 (d, J=8.5 Hz, 1H), 6.67 (d, J=9.1 Hz, 1H), 4.20 (t, J=6.8 Hz, 2H), 4.08 (t, J=7.0 Hz, 2H), 3.77 (s, 3H), 1.96 (s, 3H), 2.84 (s, 2H), 1.80 (q, J=6.8 Hz, 4H), 1.05 (s, 6H), 1.00 (s, 9H). LC-MS (ESI) Calcd for C₂₈H₃₈O₇ [M+H]⁺: 487.26. Found: 487.15.

### Example 30

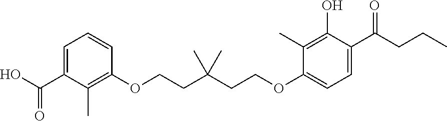 [SMILES: CCCC(=O)c1ccc(OCCC(C)(C)CCOc2cccc(C(=O)O)c2C)c(C)c1O]

### 3-[5-(4-Butanoyl-3-hydroxy-2-methylphenoxy)-3,3-dimethylpentyloxy]-2-methylbenzoic acid

Methyl 3-hydroxy-2-methylbenzoate, 1,5-dibromo-3,3-dimethylpentane, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.82 (dd, J=1.8 Hz, 9.1 Hz, 1H), 7.28 (dd, J=1.4 Hz, 7.6 Hz, 1H), 7.20 (t, J=7.8 Hz, 1H), 7.15 (dd, J=1.5 Hz, 8.2 Hz, 1H), 6.68 (d, J=9.1 Hz, 1H), 4.19 (t, J=6.8 Hz, 2H), 4.07 (t, J=6.8 Hz, 2H), 2.97 (t, J=7.2 Hz, 2H), 2.30 (s, 3H), 1.96 (s, 3H), 1.81 (t, J=6.9 Hz, 4H), 1.69-1.59 (m, 2H), 1.05 (s, 6H), 0.93 (t, J=7.4 Hz, 3H). LC-MS (ESI) Calcd for C₂₆H₃₄O₆ [M+H]⁺: 443.24. Found: 443.00.

### Example 31

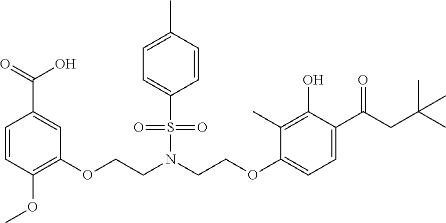 [SMILES: COc1ccc(C(=O)O)cc1OCCN(CCOc1ccc(C(=O)CC(C)(C)C)c(O)c1C)S(=O)(=O)c1ccc(C)cc1]

### 3-[2-({2-[4-(3,3-Dimethylbutanoyl)-3-hydroxy-2-methylphenoxy]ethyl}[(4-methylphenyl)sulfonyl]amino)ethoxy]-4-methoxybenzoic acid

To a solution of (tosylazanediyl)bis(ethane-2,1-diyl) bis(4-methylbenzenesulfonate) (2.84 g, 5 mmol) in 15 mL of DMF was added NaBr (2.57 g, 25 mmol). The resulting suspension was stirred at 120° C. for 4 h. After cooling to rt, the reaction mixture was concentrated to about 2 mL. The viscous milky product was poured into rapidly stirred mixture of ice-water (100 mL) and extracted with EtOAc. The organic phase was dried (Na₂SO₄) and concentrated under reduced pressure. The crude product was purified by column chromatography (10% EtOAc in hexane) to yield N,N-bis(2-bromoethyl)-4-methylbenzenesulfonamide as a light yellow solid (0.082 g, 42.6%).

To a solution of 1-(2,4-dihydroxy-3-methylphenyl)-3,3-dimethylbutan-1-one (0.176 g, 0.794 mmol) and methyl 3-(2-((N-(2-bromoethyl)-4-methylphenyl)sulfonamido)ethoxy)-4-methoxybenzoate (0.386 g, 0.794 mmol) in ACN (5 mL) was added K₂CO₃ (0.219 g, 1.588 mmol) and heated at 80° C. for 2 h. After cooling to rt, the solvent was removed under reduced pressure, and partitioned between DCM and water, the organic phase collected, aqueous phase extracted three times with DCM (3×10 mL). The combined organic phases were washed with water, brine and dried over Na₂SO₄, upon removal of the solvent yielded methyl 3-(2-((N-(2-(4-(3,3-dimethylbutanoyl)-3-hydroxy-2-methylphenoxy)ethyl)-4-methylphenyl)sulfonamido)ethoxy)-4-methoxybenzoate. White solid (0.330 g, 66.2%). LCMS (ESI) Calcd for C₃₃H₄₁NO₉S [M+H]⁺: 628.25. Found: 628.00. To a stirred solution of methyl 3-(2-((N-(2-(4-(3,3-dimethylbutanoyl)-3-hydroxy-2-methylphenoxy)ethyl)-4-methylphenyl)sulfonamido)ethoxy)-4-methoxybenzoate (0.063 g, 0.1 mmol) in dioxane (5 mL) was added 2M LiOH (0.5 mL), the resulting mixture was heated at reflux for 30 min. Upon completion of the reaction, the crude mixture was diluted with water, acidified using dil. HCl and extracted with EtOAc (3×10 mL). Removal of the solvent followed by reverse phase HPLC yielded 3-(2-((N-(2-(4-(3,3-dimethylbutanoyl)-3-hydroxy-2-methylphenoxy)ethyl)-4-methylphenyl)sulfonamido)ethoxy)-4-methoxybenzoic acid (0.013 g, 21.2%). White solid. LC-MS (ESI) Calcd for C₃₂H₃₉NO₉S [M+H]⁺: 614.23. Found: 614.15.

### Example 32

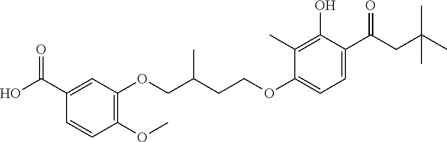 [SMILES: COc1ccc(C(=O)O)cc1OCC(C)CCOc1ccc(C(=O)CC(C)(C)C)c(O)c1C]

### 3-{4-[4-(3,3-Dimethylbutanoyl)-3-hydroxy-2-methylphenoxy]-2-methylbutoxy}-4-methoxybenzoic acid

To α-methyl-γ-butyrolactone (1.6 g, 26 mmol) was added PBr₃ (1.7 mL, 29.3 mol) and this was then heated at reflux for 8 hours. The reaction was slowly poured onto iced water (500 mL). DCM (500 mL) was added, and this was stirred overnight at rt. The layers were separated, dried over Na₂SO₄, filtered, and evaporated to dryness. The resultant yellow oil was passed through a pad of silica (3:1 hexane: EtOAc). ¹H (CDCl₃): 3.51 (t, J=12.0 Hz, 2H), 2.76 (m, 1H), 2.36-1.95 (m, 2H), 1.26 (d, J=15.0 Hz, 3H).

A solution of the above acid (1.55 g, 8.67 mmol) in dry ether (50 mL) was added dropwise over 30 min to a suspension of LAH (0.329 g, 8.67 mmol) in dry ether (100 mL) at 0° C. under N₂. The reaction mixture was stirred for 2 h at 0° C. MeOH (1 mL) was added dropwise to the suspension, and the mixture was poured into ice-cold 2 N HCl (200 mL). The mixture was extracted with ether, dried over Na₂SO₄ and concentrated to give 4-bromo-2-methylbutan-1-ol as a viscous liquid (2.2 g, 82%). ¹H NMR: 4.57 (br s, I H, OH), 3.62-3.41 (m, 4H), 2.13-1.69 (m, 3H), 0.96 (d, J=14.0 Hz, 3H).

To a mixture of 4-bromo-2-methylbutan-1-ol (1 g, 5.99 mmol) and Et₃N (0.909 g, 8.98 mmol) in DCM (25 mL) at 0° C. was added MsCl (1.029 g, 8.98 mmol). The resulting mixture was gradually warmed to rt and stirred for another 2 h. The mixture was washed with water, brine and dried over Na₂SO₄. Removal of the solvent yielded 4-bromo-2-methylbutyl methanesulfonate, which was used for next steps without further purification. Pale yellow viscous liquid (0.980 g, 66.8%).

4-Bromo-2-methylbutyl methanesulfonate (0.368 g, 1.5 mmol) and 1-(2,4-dihydroxy-3-methylphenyl)-3,3-dimethylbutan-1-one (0.222 g, 1 mmol) were taken in ACN (10 mL). and heated at reflux in presence of potassium (0.276 g, 2 mmol). After the consumption of the starting material, the mixture cooled to rt, the solvent removed and extracted using DCM and water. The combined organic layer washed with water brine and dried over Na₂SO₄. Removal of the solvent followed by silica column yielded 4-(4-(3,3-dimethylbutanoyl)-3-hydroxy-2-methylphenoxy)-2-methylbutyl methanesulfonate as a yellow viscous liquid (0.320 g, 82.8%). LC-MS (ESI) Calcd for C₁₉H₃₀O₆S [M+H]⁺: 387.17. Found: 387.00.

4-(4-(3,3-Dimethylbutanoyl)-3-hydroxy-2-methylphenoxy)-2-methylbutyl methanesulfonate (0.32 g, 0.83 mmol) was taken in acetone (25 mL) and LiBr (0.434 g, 5 mmol) added to it. The resulting mixture was heated at reflux for 3 h, cooled and removed the solvent and extracted with DCM (3×20 mL). The combined organic layers washed with water brine and dried over Na₂SO₄. Removal of the solvent followed by silica column yielded 1-(4-(4-bromo-3-methylbutoxy)-2-hydroxy-3-methylphenyl)-3,3-dimethylbutan-1-one as a viscous liquid (0.248 g, 66.8%). LC-MS (ESI) Calcd for C₁₈H₂₇BrO₃ [M+H]⁺: 371.11 Found: 371.00.

Methyl 3-hydroxy-4-methoxybenzoate (0.091 g, 0.5 mmol), 1-(4-(4-bromo-3-methylbutoxy)-2-hydroxy-3-methylphenyl)-3,3-dimethylbutan-1-one (0.186 g, 0.5 mmol), K₂CO₃ (0.138 g, 1 mmol) and 2M LiOH (2.5 mL) were processed according to general method C and D. White solid (0.121 g, 52.8%). ¹H NMR (400 MHz, DMSO-d₆); δ 7.92-7.87 (m, 1H), 7.56 (dd, J=8.4, 2.0 Hz, 1H), 7.45 (d, J=2.0 Hz, 1H), 7.04 (d, J=8.5 Hz, 1H), 6.64 (d, J=9.1 Hz, 1H), 4.23 (t, J=6.5, 1.9 Hz, 2H), 3.97-3.87 (m, 2H), 3.81 (s, 3H), 2.85 (s, 2H), 2.20 (q, J=6.5 Hz, 1H), 2.00 (d, J=7.3 Hz, 1H), 1.97 (s, 3H), 1.79-1.72 (m, 1H), 1.07 (d, J=6.8 Hz, 3H), 1.01 (s, 9H). LC-MS (ESI) Calcd for C₂₆H₃₄O₇ [M+H]⁺: 459.23. Found: 459.10.

### Example 33

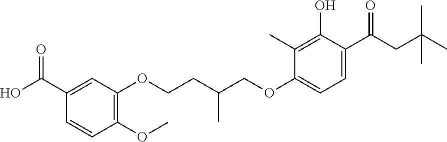 [SMILES: COc1ccc(C(=O)O)cc1OCCC(C)COc1ccc(C(=O)CC(C)(C)C)c(O)c1C]

### 3-{4-[4-(3,3-Dimethylbutanoyl)-3-hydroxy-2-methylphenoxy]-3-methylbutoxy}-4-methoxybenzoic acid

Methyl 3-hydroxy-4-methoxybenzoate (0.182 g, 1 mmol) and 4-bromo-2-methylbutyl methanesulfonate (0.245 g, 1 mmol) were taken in ACN (10 mL) and heated at reflux in presence of K₂CO₃ (0.276 g, 2 mmol). After the consumption of the starting material, the mixture was cooled to rt, the solvent removed and extracted using DCM (3×25 mL). The combined organic layers washed with water brine and dried over Na₂SO₄. Removal of the solvent followed by silica column yielded methyl 4-methoxy-3-(3-methyl-4-((methylsulfonyl)oxy)butoxy)benzoate as a yellow viscous liquid (0.250 g, 72%).

Methyl 4-methoxy-3-(3-methyl-4-((methylsulfonyl)oxy)butoxy)benzoate as a yellow viscous liquid (0.250 g, 0.72 mmol) taken in acetone (25 mL) and LiBr (0.434 g, 5 mmol) was added. Refluxed for 3 h, cooled, the solvent removed and extracted with DCM. The combined organic layer washed with water brine and dried over Na₂SO₄. Removal of the solvent followed by silica column yielded methyl 3-(4-bromo-3-methylbutoxy)-4-methoxybenzoate. Yellow viscous liquid (0.210 g, 88%).

Methyl 3-(4-bromo-3-methylbutoxy)-4-methoxybenzoate (0.166 g, 1 mmol), 1-(2,4-dihydroxy-3-methylphenyl)-3,3-dimethylbutan-1-one (0.222 g, 1 mmol), K₂CO₃ (0.276 g, 2 mmol) and 2M LiOH (2.5 mL) were processed according to general method C and D. White solid (0.132 g, 57.6%). ¹H NMR (400 MHz, DMSO-d₆): δ 7.91-7.87 (m, 1H), 7.55 (dd, J=2.0 Hz, 8.4 Hz, 1H), 7.46 (d, J=2.0 Hz, 1H), 7.03 (d, J=8.6 Hz, 1H), 6.62 (d, J=9.1 Hz, 1H), 4.16-4.05 (m, 4H), 3.98-3.96 (m, 1H), 3.80 (s, 3H), 2.85 (s, 2H), 2.19 (dq, J=12.9, 6.5 Hz, 1H), 1.98 (s, 3H), 1.74 (dd, J=13.7, 6.9 Hz, 1H), 1.08 (d, J=6.8 Hz, 3H), 1.00 (s, 9H). LC-MS (ESI) Calcd for C₂₆H₃₄O₇ [M+H]⁺: 459.23. Found: 459.10.

### Example 34

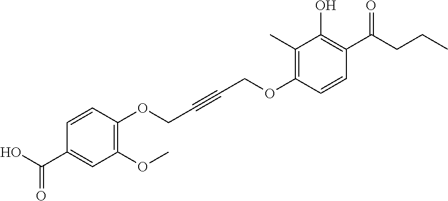 [SMILES: CCCC(=O)c1ccc(OCC#CCOc2ccc(C(=O)O)cc2OC)c(C)c1O]

### 4-[4-(4-Butanoyl-3-hydroxy-2-methylphenoxy)but-2-ynyloxy]-3-methoxybenzoic acid

Methyl 4-hydroxy-3-methoxybenzoate, 1,4-dibromobut-2-yne, 1-(2,4-dihydroxy-3-methyl phenyl)butan-1-one, K₂CO₃ and LiOH were processed according to general method B, C and D. White solid. ¹H NMR (400 MHz, DMSO-d₆): δ 7.77 (d, J=9.0 Hz, 1H), 7.49 (dd, J=2.0 Hz, 8.4 Hz, 1H), 7.45 (d, J=2.0 Hz, 1H), 7.04 (d, J=8.4 Hz, 1H), 6.63 (d, J=9.1 Hz, 1H), 5.02 (s, 2H), 4.96 (s, 2H), 3.79 (s, 3H), 2.98 (t, J=7.2 Hz, 2H), 1.98 (s, 3H), 1.65-1.60 (m, 2H), 0.94 (t, J=7.4 Hz, 3H). LC (ESI) Calcd for C₂₄H₂₄O₇ [M+H]⁺: 413.15. Found: 413.10.

### Example 35

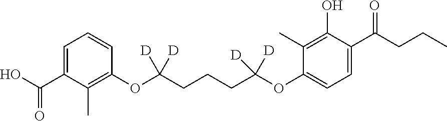 [SMILES: [2H]C([2H])(CCCC([2H])([2H])Oc1ccc(C(=O)CCC)c(O)c1C)Oc1cccc(C(=O)O)c1C]

### 3-((5-(4-Butyryl-3-hydroxy-2-methylphenoxy)pentyl-1,1,5,5-d4)oxy)-2-methylbenzoic acid

LiAlD₄ (2.35 g, 56.19) was suspended in THF (112 mL) and cooled to 0° C. in an ice bath. A solution of dimethyl glutarate (6 g, 37.46 mmol) in ether (5.7 mL) was added dropwise via syringe. The reaction slurry was allowed to warm to rt overnight and then quenched by the subsequent addition of 50 mL EtOAc and 3×5 mL of Rochelle salt solution (RM kept in an ice bath) and the clear solution was filtered, and the residue washed 6× with 50 mL of EtOAc. The solution was dried over Na₂SO₄ and the solvent was removed in vacuo to yield pentane-1,1,5,5-d₄-1,5-diol which was used without further purification. Colorless viscous liquid (3.2 g, 79%). ¹H NMR (400 MHz, CDCl₃): δ 1.62-1.53 (m, 4H), 1.45-1.40 (m, 2H).

Pentane-1,1,5,5-d₄-1,5-diol (3.0 g, 27.37 mmol) was dissolved in dry CH₂Cl₂ (75 mL) under argon atmosphere and the solution was cooled to −20° C. Et₃N (5.6 g, 55.5 mmol) was added and then MsCl (6.35 g, 55.5 mmol) was added slowly over 5 minutes. The mixture was stirred for 1 h. The reaction was quenched with H₂O and the mixture was extracted with CH₂Cl₂ (3×20 mL). The combined organic layers were washed with brine, dried over Na₂SO₄, filtered, and concentrated under reduced pressure to yield pentane-1,5-diyl-1,1,5,5-d₄ dimethanesulfonate as a light-yellow viscous liquid (6.06 g, 82.66%), ¹HNMR (400 MHz, CDCl₃): δ 3.10 (s, 6H), 1.79-1.36 (m, 6H). To a solution of the crude mixture (5.2 g, 19.7 mmol) in acetone (150 mL) was added LiBr (17 g, 196 mmol). The resulting suspension was stirred for 6 h at reflux. After cooling to rt, Et₂O was added and the organic layer was washed with H₂O and brine, dried over Na₂SO₄, filtered, and concentrated in vacuo, to give 1,5-dibromopentane-1,1,5,5-d₄. The crude mixture was used without purification. Pale yellow oil (445 g, 96.7%).

Methyl 3-hydroxy-2-methybenzoate, 1,5-dibromopentane-1,1,5,5-d₄, 1-(2,4-dihydroxy-3-methylphenyl)butan-1-one, K₂CO₃ and LiOH were processed according to general method B, C and D. White solid. ¹H NMR (400 MHz, DMSO-d₆): δ 7.82 (d, J=9.1 Hz, 1H), 7.29 (dd, J=1.3 Hz, 7.8 Hz, 1H), 7.23-7.18 (m, 1H), 7.11 (dd, J=1.3 Hz, 8.2 Hz, 1H), 6.63 (d, J=9.0 Hz, 1H), 2.97 (t, J=7.2 Hz, 2H), 2.32 (s, 3H), 1.99 (s, 3H), 1.84-1.77 (m, 4H), 1.69-1.58 (m, 4H), 0.93 (t, J=7.4 Hz, 3H). LC-MS (ESI) Calcd for C₂₄H₂₆D₄O₆ [M+H]⁺: 419.23. Found: 419.20.

### Example 36

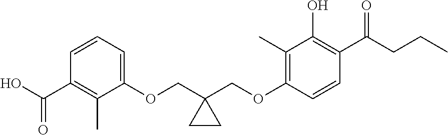 [SMILES: CCCC(=O)c1ccc(OCC2(COc3cccc(C(=O)O)c3C)CC2)c(C)c1O]

### 3-({[(4-Butanoyl-3-hydroxy-2-methylphenoxy)methyl]cyclopropyl}methoxy)-2-methylbenzoic acid

To a stirred mixture of methyl 3-((5-bromopentyl)oxy)-2-methylbenzoate (0.313 g, 1 mmol) and K₂CO₃ (0.276 g, 2 mmol) in ACN (10 mL) was added 1-(2,4-dihydroxy-3-methylphenyl)butan-1-one (0.194 g, 1 mmol). The mixture was heated at 80° C. for 4-6 h under N₂ (reaction monitored by LC-MS), then cooled to rt and the solids were filtered and washed with EtOAc. Upon concentration of the filtrate under reduced pressure, the ester was obtained and used for next step without further purification. Off-White solid (0.412 g, quantitative).

To stirred solution of above ester (0.412 g, 1 mmol) in dioxane (10 mL) was added 2M LiOH (2.5 mL, 5 mmol) and the mixture was heated at 80° C. for 30 min (reaction monitored by LC-MS). After completion of the reaction, the mixture was cooled to rt and diluted with water. The pH was adjusted to 1 by the addition of 1N HCl and the mixture was extracted with EtOAc (3×20 mL). The organic layer was washed successively with water and saturated brine and dried over anhydrous MgSO₄. The solvent was evaporated under reduced pressure to yield the crude product which was purified by reverse phase HPLC to yield the desired product as a white solid (0.34 g, 82%). ¹H NMR (400 MHz, DMSO-d₆): δ 7.81 (d, J=9.0 Hz, 1H), 7.29 (dd, J=1.2 Hz, 7.8 Hz, 1H), 7.21-7.16 (m, 1H), 7.10 (dd, J=1.3 Hz, 8.3 Hz, 1H), 6.63 (d, J=9.0 Hz, 1H), 4.14 (s, 2H), 4.01 (s, 2H), 2.96 (t, J=7.3 Hz, 2H), 2.32 (s, 3H), 1.98 (s, 3H), 1.64-1.58 (m, 2H), 0.92 (t, J=7.4 Hz, 3H), 0.75 (s, 4H). LC-MS (ESI) Calcd for C₂₄H₂₈O₆ [M+H]⁺: 413.19. Found: 413.20.

### Example 37

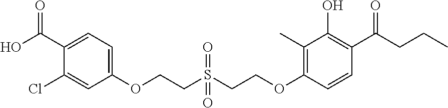 [SMILES: CCCC(=O)c1ccc(OCCS(=O)(=O)CCOc2ccc(C(=O)O)c(Cl)c2)c(C)c1O]

### 4-(2-{[2-(4-Butanoyl-3-hydroxy-2-methylphenoxy)ethyl]sulfonyl}ethoxy)-2-chlorobenzoic acid

4-(2-((2-(4-Butyryl-3-hydroxy-2-methylphenoxy)ethyl)thio)ethoxy)-2-chlorobenzoic acid (0.010 mg, 0.022 mmol) in DCM (5 mL) was added mCPBA (0.008 mg, 0.044 mmol) at 0° C., then warmed to rt and stirred for 1 h. The reaction was quenched with aq. NaHSO₃ and the mixture was stirred at rt for 30 min. Then the mixture was extracted with EtOAc (10 mL×2). The extracts were dried over NaSO₄ and concentrated to dryness in vacuum. The residue was purified by reverse phase HPLC to give the title compound. White solid (0.006 mg, 56%). ¹H NMR (400 MHz, DMSO-d₆): δ 7.88 (d, J=9.0 Hz, 1H), 7.79 (d, J=8.7 Hz, 1H), 7.14 (d, J=2.5 Hz, 1H), 7.02 (dd, J=2.5 Hz, 8.8 Hz, 1H), 6.75 (d, J=9.1 Hz, 1H), 4.62-4.40 (m, 4H), 3.50-3.18 (m, 4H), 3.00 (t, J=7.3 Hz, 2H), 2.01 (s, 3H), 1.69-1.60 (m, 2H), 0.95 (t, J=7.4 Hz, 3H). LC-MS (ESI) Calcd for C₂₂H₂₅ClO₈S [M+H]⁺: 485.10. Found: 485.15.

### Example 38

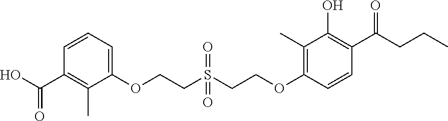 [SMILES: CCCC(=O)c1ccc(OCCS(=O)(=O)CCOc2cccc(C(=O)O)c2C)c(C)c1O]

### 3-(2-{[2-(4-Butanoyl-3-hydroxy-2-methylphenoxy)ethyl]sulfonyl}ethoxy)-2-methylbenzoic acid

3-(2-((2-(4-Butyryl-3-hydroxy-2-methylphenoxy)ethyl)thio)ethoxy)-2-methylbenzoic acid (0.012 mg, 0.028 mmol) in DCM (5 mL) was added mCPBA (0.010 mg, 0.055 mmol) at 0° C., then warmed to rt and stirred for 1 h. The reaction was quenched with aq. NaHSO₃ and the mixture was stirred at rt for 30 min. Then the mixture was extracted with EtOAc (10 mL×2). The extracts were dried over NaSO₄ and concentrated to dryness in vacuo. The residue was purified by reverse phase HPLC to give the title compound. White solid (0.008 mg, 62%). ¹H NMR (400 MHz, DMSO-d₆): δ 7.88-7.84 (m, 1H), 7.30 (dd, J=1.3 Hz, 7.6 Hz, 1H), 7.21 (t, J=8.0 Hz, 1H), 7.16 (dd, J=1.4 Hz, 8.3 Hz, 1H), 6.70 (d, J=9.1 Hz, 1H), 4.53 (t, J=5.5 Hz, 2H), 4.42 (t, J=5.5 Hz, 2H), 3.78 (q, J=6.0 Hz, 4H), 2.98 (t, J=7.3 Hz, 2H), 2.31 (s, 3H), 1.99 (s, 3H), 1.69-1.60 (m, 2H), 0.93 (t, J=7.4 Hz, 3H). LC-MS (ESI) Calcd for C₂₃H₂₈O₈S [M+H]: 465.15. Found: 465.15.

### Example 39

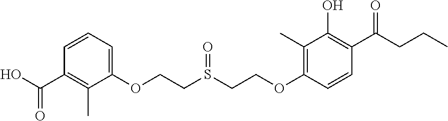 [SMILES: CCCC(=O)c1ccc(OCCS(=O)CCOc2cccc(C(=O)O)c2C)c(C)c1O]

### 3-(2-{[2-(4-Butanoyl-3-hydroxy-2-methylphenoxy)ethyl]sulfinyl}ethoxy)-2-methylbenzoic acid

3-(2-((2-(4-Butyryl-3-hydroxy-2-methylphenoxy)ethyl)thio)ethoxy)-2-methylbenzoic acid (0.010 g, 0.023 mmol) in DCM (5 mL) was added mCPBA (0.004 mg, 0.023 mmol) at 0° C., then warmed to rt and stirred for 1 h. The reaction was quenched with aq. NaHSO₃ and the mixture was stirred at rt for 30 min. Then the mixture was extracted with EtOAc (10 mL×2). The extracts were dried over Na₂SO₄ and concentrated to dryness in vacuo. The residue was purified by reverse phase HPLC to give the title compound. White solid (0.007 mg, 68%). ¹H NMR (400 MHz, DMSO-d₆): δ 7.88 (d, J=9.1 Hz, 1H), 7.33-7.31 (m, 3H), 6.76 (d, J=9.1 Hz, 1H), 4.61-4.40 (m, 2H), 4.35 (t, J=5.1 Hz, 2H), 3.78-3.73 (m, 4H), 3.01 (t, J=7.3 Hz, 2H), 2.34 (s, 3H), 2.02 (s, 3H), 1.69-1.60 (m, 2H), 0.95 (t, J=7.4 Hz, 3H). 3LC-MS (ESI) Calcd for C₂₃H₂₈O₇S [M+H]⁺: 449.16. Found: 449.15.

### Example 40

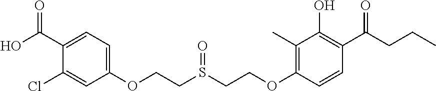 [SMILES: CCCC(=O)c1ccc(OCCS(=O)CCOc2ccc(C(=O)O)c(Cl)c2)c(C)c1O]

### 4-(2-{[2-(4-Butanoyl-3-hydroxy-2-methylphenoxy)ethyl]sulfinyl}ethoxy)-2-chlorobenzoic acid

4-(2-((2-(4-Butyryl-3-hydroxy-2-methylphenoxy)ethyl)thio)ethoxy)-2-chlorobenzoic acid (0.010 mg, 0.022 mmol) in DCM (5 mL) was added mCPBA (0.004 mg, 0.022 mmol) at 0° C., then warmed to rt and stirred for 1 h. The reaction was quenched with aq. NaHSO₃, the mixture was stirred at rt for 30 min., then extracted with EtOAc (10 mL×2). The extracts were dried over Na₂SO₄ and concentrated to dryness in vacuo. The residue was purified by reverse phase HPLC to give the title compound. White solid (0.007 mg, 70%). ¹H NMR (400 MHz, DMSO-d₆): δ 7.90-7.85 (m, 1H), 7.80 (dd, J=1.7 Hz, 8.7 Hz, 1H), 7.08-6.96 (m, 2H), 6.72 (dd, J=1.7 Hz, 9.1 Hz, 1H), 4.54 (t, J=5.1 Hz, 2H), 4.48 (t, J=5.3 Hz, 2H), 3.78-3.73 (m, 4H), 3.00 (t, J=7.3 Hz, 2H), 1.98 (s, 3H), 1.69-1.60 (m, 2H), 0.94 (t, J=7.5 Hz, 3H). LC-MS (ESI) Calcd for C₂₂H₂₅ClO₇S [M+H]⁺: 469.10. Found: 469.15.

### Example 41

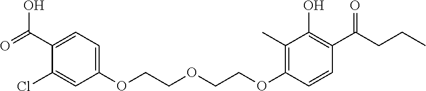 [SMILES: CCCC(=O)c1ccc(OCCOCCOc2ccc(C(=O)O)c(Cl)c2)c(C)c1O]

### 4-{2-[2-(4-Butanoyl-3-hydroxy-2-methylphenoxy)ethoxy]ethoxy}-2-chlorobenzoic acid

Methyl 2-chloro-4-hydroxybenzoate, bis(2-bromomethyl)ether, 1-(2,4-dihydroxy-3-methylphenyl)-3,3-dimethylbutan-1-one, K₂CO₃ and LiOH were processed according to general method B, C and D. White solid. ¹H NMR (400 MHz, DMSO-d₆): δ 7.81 (dd, J=3.4 Hz, 8.4 Hz, 2H), 7.07 (d, J=2.5 Hz, 1H), 6.97 (dd, J=2.5 Hz, 8.8 Hz, 1H), 6.64 (d, J=9.0 Hz, 1H), 4.25-4.19 (m, 4H), 3.87-3.81 (m, 4H), 2.97 (t, J=7.2 Hz, 2H), 1.97 (s, 3H), 1.69-1.59 (m, 2H), 0.93 (t, J=7.4 Hz, 3H). LC-MS (ESI) Calcd for C₂₂H₂₅ClO₇ [M+H]⁺: 437.13. Found: 437.10.

### Example 42

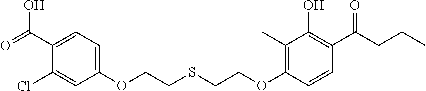 [SMILES: CCCC(=O)c1ccc(OCCSCCOc2ccc(C(=O)O)c(Cl)c2)c(C)c1O]

### 2-Chloro-4-{2-[2-(3-hydroxy-2-methyl-4-propanoylphenoxy)ethylthio]ethoxy}benzoic acid

Methyl 2-chloro-4-hydroxybenzoate, bis(2-bromoethyl)sulfane, 1-(2,4-dihydroxy-3-methylphenyl)butan-1-one, K₂CO₃ and LiOH were processed according to general method B, C and D. White solid. ¹H NMR (400 MHz, DMSO-d₆): δ 7.82 (dd, J=3.7 Hz, 8.9 Hz, 1H), 7.08 (d, J=2.5 Hz, 2H), 6.98 (dd, J=2.5 Hz, 8.8 Hz, 1H), 6.65 (d, J=9.0 Hz, 1H), 4.31-4.34 (m, 4H), 3.08-2.94 (m, 6H), 1.99 (s, 3H), 1.72-1.58 (m, 2H), 0.94 (t, J=7.4 Hz, 3H). LC-MS (ESI) Calcd for C₂₂H₂₅ClO₆S [M+H]⁺: 453.1946. Found: 453.10.

### Example 43

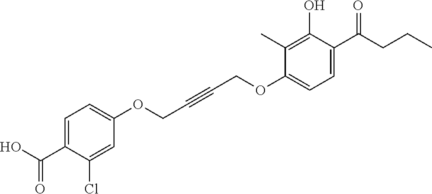 [SMILES: CCCC(=O)c1ccc(OCC#CCOc2ccc(C(=O)O)c(Cl)c2)c(C)c1O]

### 4-[4-(4-Butanoyl-3-hydroxy-2-methylphenoxy)but-2-ynyloxy]-2-chlorobenzoic acid

Methyl 2-chloro-4-hydroxybenzoate, 1,4-dibromobut-2-yne, 1-(2,4-dihydroxy-3-methylphenyl)butan-1-one, K₂CO₃ and LiOH were processed according to general method B, C and D. White solid. ¹H NMR (400 MHz, DMSO-d₆): δ 7.80 (d, J=8.8 Hz, 1H), 7.74 (dd, J=0.7 Hz, 9.1 Hz, 1H), 7.12 (d, J=2.5 Hz, 1H), 6.99 (dd, J=2.5 Hz, 8.8 Hz, 1H), 6.61 (d, J=9.1 Hz, 1H), 5.02 (s, 2H), 5.00 (s, 2H), 2.97 (t, J=7.2 Hz, 2H), 1.98 (s, 3H), 1.69-1.60 (m, 2H), 0.94 (t, J=7.4 Hz, 3H). LC-MS (ESI) Calcd for C₂₂H₂₁ClO₆ [M+H]⁺: 417.10. Found: 417.10.

### Example 44

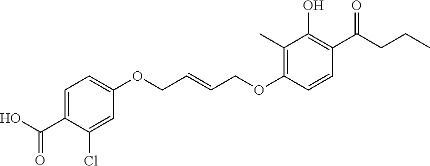 [SMILES: CCCC(=O)c1ccc(OC/C=C/COc2ccc(C(=O)O)c(Cl)c2)c(C)c1O]

### 4-[(2E)-4-(4-Butanoyl-3-hydroxy-2-methylphenoxy)but-2-enyloxy]-2-chlorobenzoic acid

Methyl 2-chloro-4-hydroxybenzoate, trans-1,4-dibromo-but-2-ene, 1-(2,4-dihydroxy-3-methylphenyl)butan-1-one, K₂CO₃ and LiOH were processed according to general method B, C and D. White solid. ¹H NMR (400 MHz, DMSO-d₆): δ 7.80 (d, J=8.8 Hz, 1H), 7.74 (dd, J=0.7 Hz, 9.1 Hz, 1H), 7.12 (d, J=2.5 Hz, 1H), 6.99 (dd, J=2.5 Hz, 8.8 Hz, 1H), 6.61 (d, J=9.1 Hz, 1H), 6.14-6.05 (m, 2H), 4.75-4.68 (m, 4H), 2.97 (t, J=7.2 Hz, 2H), 2.01 (s, 3H), 1.69-1.60 (m, 2H), 0.93 (t, J=7.4 Hz, 3H). LC-MS (ESI) Calcd for C₂₂H₂₃ClO₆ [M+H]⁺: 419.12. Found: 419.10.

### Example 45

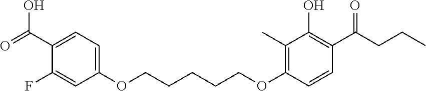 [SMILES: CCCC(=O)c1ccc(OCCCCCOc2ccc(C(=O)O)c(F)c2)c(C)c1O]

### 4-[5-(4-Butanoyl-3-hydroxy-2-methylphenoxy)pentyloxy]-2-fluorobenzoic acid

Methyl 2-fluoro-4-hydroxybenzoate, 1,5-dibromopentane, 1-(2,4-dihydroxy-3-methylphenyl)butan-1-one, K₂CO₃ and LiOH were processed according to general method B, C and D. White solid (0.336 g, 80.3%). ¹H NMR (400 MHz, DMSO-d₆): δ 7.86-7.78 (m, 2H), 6.91-6.82 (m, 2H), 6.64 (d, J=9.0 Hz, 1H), 4.13-4.07 (m, 4H), 2.97 (t, J=7.3 Hz, 2H), 1.99 (s, 3H), 1.90-1.76 (m, 4H), 1.70-1.54 (m, 4H), 0.94 (t, J=7.4 Hz, 3H). LC-MS (ESI) Calcd for C₂₃H₂₇FO₆ [M+H]⁺: 419.18. Found: 419.15.

### Example 46

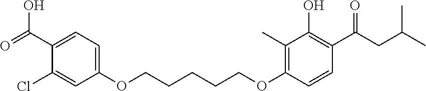 [SMILES: Cc1c(OCCCCCOc2ccc(C(=O)O)c(Cl)c2)ccc(C(=O)CC(C)C)c1O]

### 2-Chloro-4-{5-[3-hydroxy-2-methyl-4-(3-methylbutanoyl)phenoxy]pentyloxy}benzoic acid

Methyl 2-chloro-4-hydroxybenzoate, 1,5-dibromopentane, 1-(2,4-dihydroxy-3-methylphenyl)-3-methylbutan-1-one, K₂CO₃ and LiOH were processed according to general method B, C and D. White solid. ¹H NMR (400 MHz, DMSO-d₆) δ 7.84-7.80 (m, 2H), 7.07 (d, J=2.4 Hz, 1H), 6.96 (dd, J=2.7 Hz, 8.7 Hz, 1H), 6.63 (d, J=9.1 Hz, 1H), 4.13-4.10 (m, 4H), 2.85 (d, J=7.0 Hz, 2H), 2.19-2.07 (m, 1H), 1.99 (s, 3H), 1.85-1.76 (m, 4H), 1.62-1.54 (m, 2H), 0.93 (d, J=6.5 Hz, 6H). LC-MS (ESI) Calcd for C₂₄H₂₉ClO₆ [M+H]⁺: 449.17. Found: 449.15.

### Example 47

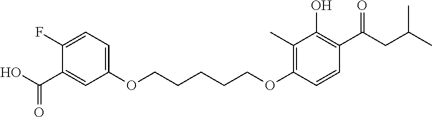 [SMILES: Cc1c(OCCCCCOc2ccc(F)c(C(=O)O)c2)ccc(C(=O)CC(C)C)c1O]

### 2-Fluoro-5-{5-[3-hydroxy-2-methyl-4-(3-methylbutanoyl)phenoxy]pentyloxy}benzoic acid

Methyl 2-fluoro-5-hydroxybenzoate, 1,5-dibromopentane, 1-(2,4-dihydroxy-3-methylphenyl)-3-methylbutan-1-one, K₂CO₃ and LiOH were processed according to general method B, C and D. White solid. ¹H NMR (400 MHz, DMSO-d₆) δ 7.83 (d, J=9.0 Hz, 1H), 7.30 (dd, J=3.1 Hz, 5.9 Hz, 1H), 7.25-7.14 (m, 2H), 6.63 (d, J=9.1 Hz, 1H), 4.14-3.97 (m, 4H), 2.84 (d, J=6.9 Hz, 2H), 2.19-2.07 (m, 1H), 1.99 (s, 3H), 1.85-1.76 (m, 4H), 1.62-1.54 (m, 2H), 0.93 (d, J=6.5 Hz, 6H). LC-MS (ESI) Calcd for C₂₄H₂₉FO₆ [M+H]⁺: 433.19. Found: 433.20.

### Example 48

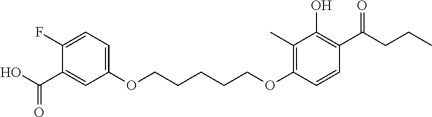 [SMILES: CCCC(=O)c1ccc(OCCCCCOc2ccc(F)c(C(=O)O)c2)c(C)c1O]

### 5-[5-(4-Butanoyl-3-hydroxy-2-methylphenoxy)pentyloxy]-2-fluorobenzoic acid

Methyl 2-fluoro-5-hydroxybenzoate, 1,5-dibromopentane, 1-(2,4-dihydroxy-3-methylphenyl)butan-1-one, K₂CO₃ and LiOH were processed according to general method B, C and D. ¹H NMR (400 MHz, DMSO-d₆) δ 7.83 (d, J=9.0 Hz, 1H), 7.30 (dd, J=3.1 Hz, 5.8 Hz, 1H), 7.25-7.13 (m, 2H), 6.63 (d, J=9.0 Hz, 1H), 4.11 (t, J=6.3 Hz, 2H), 4.00 (t, J=6.3 Hz, 2H), 3.00-2.94 (m, 2H), 1.99 (s, 3H), 1.85-1.74 (m, 4H), 1.69-1.56 (m, 4H), 0.93 (t, J=7.4 Hz, 3H). LC-MS (ESI) Calcd for C₂₃H₂₇FO₆ [M+H]⁺: 419.18. Found: 419.60.

### Example 49

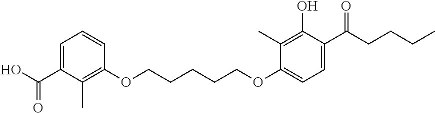 [SMILES: CCCCC(=O)c1ccc(OCCCCCOc2cccc(C(=O)O)c2C)c(C)c1O]

### 3-[5-(3Hydroxy-2-methyl-4-pentanoylphenoxy)pentyloxy]-2-methylbenzoic acid

Methyl 3-hydroxy-2-methyl-benzoate, 1,5-dibromopentane, 1-(2,4-dihydroxy-3-methyl phenyl)pentan-1-one, K₂CO₃ and LiOH were processed according to general method B, C and D. White solid. ¹H NMR (400 MHz, DMSO-d₆) δ 7.81 (d, J=9.1 Hz, 1H), 7.29 (dd, J=1.3 Hz, 7.7 Hz, 1H), 7.20 (t, J=7.9 Hz, 1H), 7.10 (dd, J=1.3 Hz, 8.2 Hz, 1H), 6.62 (d, J=9.1 Hz, 1H), 4.11 (t, J=6.2 Hz, 2H), 4.01 (t, J=6.2 Hz, 2H), 2.97 (t, J=7.4 Hz, 2H), 2.33 (s, 3H), 1.98 (s, 3H), 1.87-1.79 (m, 4H), 1.68-1.55 (m, 4H), 1.39-1.29 (m, 2H), 0.92-0.87 (t, J=7.3 Hz, 3H). LC-MS (ESI) Calcd for C₂₅H₃₂O₆ [M+H]⁺: 429.22. Found: 429.60.

### Example 50

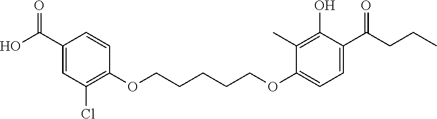 [SMILES: CCCC(=O)c1ccc(OCCCCCOc2ccc(C(=O)O)cc2Cl)c(C)c1O]

### 4-[5-(4-Butanoyl-3-hydroxy-2-methylphenoxy)pentyloxy]-3-chlorobenzoic acid

Methyl 3-chloro-4-hydroxybenzoate, 1,6-dibromohexane, 1-(2,4-dihydroxy-3-methylphenyl) butan-1-one, K₂CO₃ and LiOH were processed according to general method B, C and D. White solid. ¹H NMR (400 MHz, DMSO-d₆) δ 7.90-7.78 (m, 3H), 7.24 (d, J=8.5 Hz, 1H), 6.64 (d, J=9.1 Hz, 1H), 4.18 (t, J=6.3 Hz, 2H), 4.12 (t, J=6.2 Hz, 2H), 2.97 (t, J=7.3 Hz, 2H), 1.98 (s, 3H), 1.91-1.78 (m, 4H), 1.69-1.60 (m, 4H), 0.93 (t, J=7.4 Hz, 3H). LC-MS (ESI) Calcd for C₂₃H₂₇ClO₆ [M+H]⁺: 435.15. Found: 435.20.

### Example 51

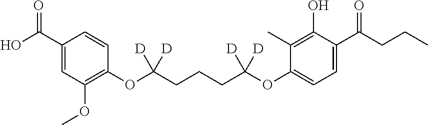 [SMILES: [2H]C([2H])(CCCC([2H])([2H])Oc1ccc(C(=O)CCC)c(O)c1C)Oc1ccc(C(=O)O)cc1OC]

### 4-((5-(4-Butyryl-3-hydroxy-2-methylphenoxy)pentyl-1,1,5,5-d4)oxy)-3-methoxybenzoic acid

Methyl 4-hydroxy-3-methoxybenzoate, 1,5-dibromopentane-1,1,5,5-d₄, 1-(2,4-dihydroxy-3-methylphenyl)butan-1-one, K₂CO₃ and LiOH were processed according to general method B, C and D. White solid. ¹H NMR (400 MHz, DMSO-d₆) δ 7.83 (d, J=9.1 Hz, 1H), 7.53 (dd, J=2.0 Hz, 8.4 Hz, 1H), 7.43 (d, J=1.9 Hz, 1H), 7.03 (d, J=8.4 Hz, 1H), 6.64 (d, J=9.1 Hz, 1H), 3.78 (s, 3H), 2.97 (t, J=7.2 Hz, 2H), 1.99 (s, 3H), 1.82-1.787 (m, 4H), 1.69-1.56 (m, 4H), 0.93 (t, J=7.4 Hz, 3H). LC-MS (ESI) Calcd for C₂₄H₂₆D₄O₇ [M+H]⁺: 435.23. Found: 435.25.

### Example 52

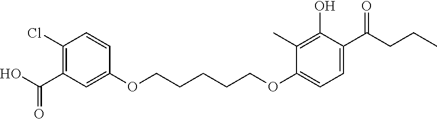 [SMILES: CCCC(=O)c1ccc(OCCCCCOc2ccc(Cl)c(C(=O)O)c2)c(C)c1O]

### 5-[5-(4-Butanoyl-3-hydroxy-2-methylphenoxy)pentyloxy]-2-chlorobenzoic acid

Methyl 2-chloro-5-hydroxybenzoate, 1,5-dibromopentane, 1-(2,4-dihydroxy-3-methylphenyl) butan-1-one, K₂CO₃ and LiOH were processed according to general method B, C and D. ¹H NMR (400 MHz, DMSO-d₆) δ 7.82 (d, J=9.1 Hz, 1H), 7.41 (d, J=8.7 Hz, 1H), 7.27 (d, J=3.2 Hz, 1H), 7.09 (dd, J=3.2 Hz, 9.1 Hz, 1H), 6.63 (d, J=9.1 Hz, 1H), 4.11 (t, J=6.3 Hz, 2H), 4.03 (t, J=6.3 Hz, 2H), 2.97 (t, J=7.2 Hz, 2H), 1.99 (s, 3H), 1.86-1.54 (m, 8H), 0.93 (t, J=7.5 Hz, 3H). LC-MS (ESI) Calcd for C₂₃H₂₇ClO₆ [M+H]⁺: 435.15. Found: 435.20.

### Example 53

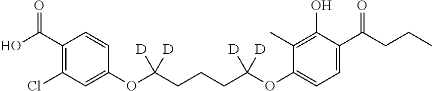 [SMILES: [2H]C([2H])(CCCC([2H])([2H])Oc1ccc(C(=O)CCC)c(O)c1C)Oc1ccc(C(=O)O)c(Cl)c1]

### 4-((5-(4-Butyryl-3-hydroxy-2-methylphenoxy)pentyl-1,1,5,5-d4)oxy)-2-chlorobenzoic acid

Methyl 2-chloro-4-hydroxybenzoate, 1,5-dibromopentane-1,1,5,5-d₄, 1-(2,4-dihydroxy-3-methylphenyl)butan-1-one, K₂CO₃ and LiOH were processed according to general method B, C and D. White solid. ¹H NMR (400 MHz, DMSO-d₆) δ 7.83 (dd, J=4.8 Hz, 8.9 Hz, 2H), 7.08 (d, J=2.7 Hz, 1H), 6.97 (dd, J=2.7 Hz, 8.9 Hz, 1H), 6.64 (d, J=9.1 Hz, 1H), 2.98 (t, J=7.2 Hz, 2H), 2.00 (s, 3H), 1.83-1.56 (m, 8H), 0.94 (t, J=7.3 Hz, 3H). LC-MS (ESI) Calcd for C₂₃H₂₃D₄ClO₆ [M+H]⁺: 439.17. Found: 439.15.

### Example 54

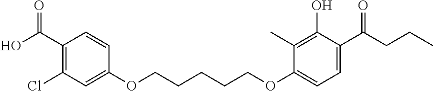 [SMILES: CCCC(=O)c1ccc(OCCCCCOc2ccc(C(=O)O)c(Cl)c2)c(C)c1O]

### 4-[5-(4-Butanoyl-3-hydroxy-2-methylphenoxy)pentyloxy]-2-chlorobenzoic acid

Methyl 2-chloro-4-hydroxy-benzoate, 1,5-dibromopentane, 1-(2,4-dihydroxy-3-methylphenyl) butan-1-one, K₂CO₃ and LiOH were processed according to general method B, C and D. White solid. ¹H NMR (400 MHz, DMSO-d₆) δ 7.84-7.81 (m, 2H), 7.07 (d, J=2.5 Hz, 1H), 6.97 (dd, J=2.5 Hz, 8.8 Hz, 1H), 6.64 (d, J=9.0 Hz, 1H), 4.13-4.07 (m, 4H), 2.97 (t, J=7.3 Hz, 2H), 1.99 (s, 3H), 1.85-1.76 (m, 4H), 1.69-1.54 (m, 4H), 0.93 (t, J=7.4 Hz, 3H). LC-MS (ESI) Calcd for C₂₃H₂₇ClO₆ [M+H]⁺: 435.15. Found: 435.20.

### Example 55

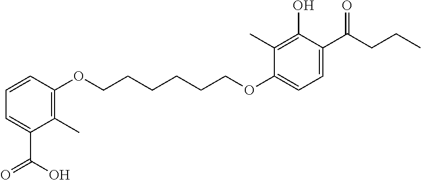 [SMILES: CCCC(=O)c1ccc(OCCCCCCOc2cccc(C(=O)O)c2C)c(C)c1O]

### 3-[6-(4-Butanoyl-3-hydroxy-2-methylphenoxy)hexyloxy]-2-methylbenzoic acid

Methyl 3-hydroxy-2-methylbenzoate, 1,6-dibromohexane, 1-(2,4-dihydroxy-3-methylphenyl) butan-1-one, K₂CO₃ and LiOH were processed according to general method B, C and D. White solid. ¹H NMR (400 MHz, DMSO-d₆) δ 7.82 (dd, J=0.7 Hz, 9.0 Hz, 1H), 7.29 (dd, J=1.2 Hz, 7.8 Hz, 1H), 7.20 (t, J=8.0 Hz, 1H), 7.10 (dd, J=1.3 Hz, 8.2 Hz, 1H), 6.62 (d, J=9.1 Hz, 1H), 4.09 (t, J=6.3 Hz, 2H), 3.99 (t, J=6.3 Hz, 2H), 2.97 (t, J=7.2 Hz, 2H), 2.31 (s, 3H), 1.97 (s, 3H), 1.83-1.48 (m, 10H), 0.93 (t, J=7.4 Hz, 3H). LC-MS (ESI) Calcd for C₂₅H₃₂O₆ [M+H]⁺: 429.22. Found: 429.20.

### Example 56

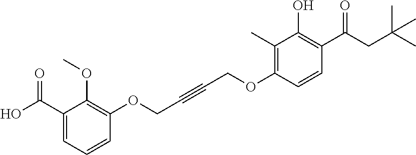 [SMILES: COc1c(OCC#CCOc2ccc(C(=O)CC(C)(C)C)c(O)c2C)cccc1C(=O)O]

### 3-((4-(4-(3,3-Dimethylbutanoyl)-3-hydroxy-2-methylphenoxy)but-2-yn-1-yl)oxy)-2-methoxybenzoic acid

A mixture of methyl 3-((4-(4-(3,3-dimethylbutanoyl)-3-hydroxy-2-methylphenoxy)but-2-yn-1-yl)oxy)-2-methoxybenzoate (201 mg, 0.44 mmol, 1.0 equiv) and lithium hydroxide (31.9 mg, 1.33 mmol, 3.0 equiv) in water (1 mL) was added to THF:MeOH (4:2 mL) and stirred at room temperature overnight. Then solvents were diluted with water (5 mL) and evaporated under reduced pressure. The mixture was adjusted to pH=3 with 1 N HCl aqueous solution. The precipitate was collected and purified with prep-HPLC to afford 3-((4-(4-(3,3-dimethylbutanoyl)-3-hydroxy-2-methylphenoxy)but-2-yn-1-yl)oxy)-2-methoxybenzoic acid (141.3 mg, 72.5%) as a white solid. ¹H NMR (400 MHz, DMSO-d₆) δ 13.22 (s, 1H), 12.86 (s, 1H), 7.82 (d, J=8.8 Hz, 1H), 7.22 (t, J=7.6 Hz, 2H), 7.07 (t, J=8.0 Hz, 1H), 6.61 (d, J=8.8 Hz, 1H), 5.02 (s, 2H), 4.96 (s, 2H), 3.73 (s, 3H), 2.85 (s, 2H), 1.98 (s, 3H), 1.01 (s, 9H). LC-MS: 440.2 [M+H]⁺.

### Example 57

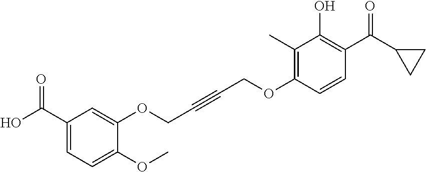 [SMILES: COc1ccc(C(=O)O)cc1OCC#CCOc1ccc(C(=O)C2CC2)c(O)c1C]

### 3-((4-(4-(Cyclopropanecarbonyl)-3-hydroxy-2-methylphenoxy)but-2-yn-1-yl)oxy)-4-methoxybenzoic acid

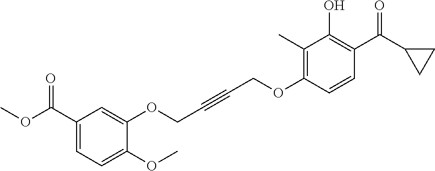 [SMILES: COC(=O)c1ccc(OC)c(OCC#CCOc2ccc(C(=O)C3CC3)c(O)c2C)c1]

### Step 1: Methyl 3-((4-(4-(cyclopropanecarbonyl)-3-hydroxy-2-methylphenoxy)but-2-yn-1-yl)oxy)-4-methoxybenzoate

To a solution of cyclopropyl(2,4-dihydroxy-3-methylphenyl)methanone (263.2 mg, 1.37 mmol, 1.1 equiv.) in DMF (6 mL) was added potassium carbonate (344 mg, 2.49 mmol, 2 equiv.) and methyl 3-((4-chlorobut-2-yn-1-yl)oxy)-4-methoxybenzoate (334 mg, 1.25 mmol, 1 equiv) successively. The resulting mixture was stirred at 60° C. under N₂ for 3 h. The reaction mixture was poured into water (30 mL) and extracted with ethyl acetate (15 mL×3). The combined organic layers were washed with water (30 ml×3) and brine (10 mL), dried over Na₂SO₄ and filtered. The filtrate was concentrated and purified by flash column chromatography (silica gel, eluting with 30% EA in PE) to afford methyl 3-((4-(4-(cyclopropanecarbonyl)-3-hydroxy-2-methylphenoxy)but-2-yn-1-yl)oxy)-4-methoxybenzoate (96 mg, 18.2%) as a white solid. LC-MS (ESI): 424.2 [M+H]⁺.

### Step 2: 3-((4-(4-(Cyclopropanecarbonyl)-3-hydroxy-2-methylphenoxy)but-2-yn-1-yl)oxy)-4-methoxybenzoic acid

A mixture of methyl 3-((4-(4-(cyclopropanecarbonyl)-3-hydroxy-2-methylphenoxy)but-2-yn-1-yl)oxy)-4-methoxybenzoate (3) (96 mg, 0.23 mmol, 1 equiv.) and lithium hydroxide (16.3 mg, 0.68 mmol, 3.0 equiv) in water (1 mL) was added to THF:MeOH (4:2 mL) and stirred at room temperature overnight. The solvents were evaporated under reduced pressure and diluted with water (5 mL). The mixture was adjusted to pH=3 with 1 N HCl aqueous solution. The precipitate was collected and purified by prep-HPLC to afford the title compound (16 mg, 14.9%) as a white solid. ¹H NMR (400 MHz, DMSO-d₆) δ 13.08 (s, 1H), 12.66 (s, 1H), 8.01 (d, J=9.2 Hz, 1H), 7.58 (dd, J=8.4, 8.4 Hz, 1H), 7.53 (s, 1H), 7.06 (d, J=8.4 Hz, 1H), 6.66 (d, J=8.8 Hz, 1H), 5.02 (s, 2H), 4.93 (s, 2H), 3.83 (s, 3H), 2.92 (dd, J=7.2, 7.2 Hz, 1H), 1.97 (s, 3H), 1.10 (t, J=3.6 Hz, 4H). LC-MS: 394.2 [M+H]⁺.

### Example 58

### (E)-3-((4-(4-Butyryl-3-hydroxy-2-methylphenoxy)but-2-en-1-yl-1,1,2,3,4,4-d6)oxy)-2-methylbenzoic acid

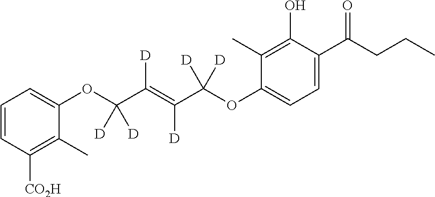 [SMILES: [2H]/C(=C(/[2H])C([2H])([2H])Oc1ccc(C(=O)CCC)c(O)c1C)C([2H])([2H])Oc1cccc(C(=O)O)c1C]

Methyl 3-hydroxy-2-methylbenzoate, (E)-1,4-dibromobut-2-ene-1,1,2,3,4,4-d₆, 1-(2,4-dihydroxy-3-methylphenyl)butan-1-one, potassium carbonate and lithium hydroxide were processed according to general method B, C and D. White solid (0.063 g, 65%) ¹H NMR (400 MHz, CDCl₃) δ 7.61 (d, J=8.6 Hz, 2H), 7.21 (t, J=8.0 Hz, 1H), 7.03 (d, J=8.2 Hz, 1H), 6.42 (d, J=8.9 Hz, 1H), 2.90 (t, J=7.4 Hz, 2H), 2.55 (s, 3H), 2.14 (s, 3H), 1.77 (m, 2H), 1.01 (t, J=7.4 Hz, 3H). LC-MS (ESI) Calcd for C₂₃H₂₀O₆D₆ [M+H]⁺: 405.49. Found: 405.20.

### Example 59

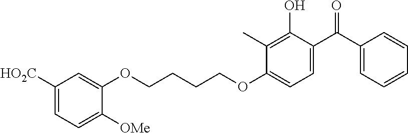 [SMILES: COc1ccc(C(=O)O)cc1OCCCCOc1ccc(C(=O)c2ccccc2)c(O)c1C]

### 3-(4-(4-Benzoyl-3-hydroxy-2-methylphenoxy)butoxy)-4-methoxybenzoic acid

Methyl 3-hydroxy-4-methoxybenzoate, 1,4-dibromobutane, 1-(2,4-dihydroxy-3-methylphenyl) (phenyl)methanone, K₂CO₃ and LiOH were processed according to general method B, C and D. White solid (0.288 g, 64%). ¹H NMR (DMSO-d₆): δ 7.62-7.56 (m, 6H), 7.44 (s, 1H), 7.36 (d, J=9.2 Hz, 1H), 6.98 (d, J=8.4 Hz, 1H), 6.62 (d, J=8.5 Hz, 1H), 4.13-4.03 (m, 4H), 3.76 (s, 3H), 2.01 (s, 3H), 1.88-1.87 (m, 4H). ¹³C NMR (DMSO-d₆): δ 200.5, 167.6, 163.3, 162.2, 153.3, 148.0, 138.4, 133.5, 132.2, 129.2, 128.9, 123.7, 123.6, 113.7, 113.3, 111.7, 104.0, 68.5, 56.2, 28.8, 8.2. LC-MS (ESI) Calcd for C₂₆H₂₆O₇ [M+H]⁺: 451.17. Found: 451.15. HRMS (ESI) Calcd for C₂₆H₂₆O₇ [M+H]⁺: 451.1647. Found: 451.170.

### Example 60

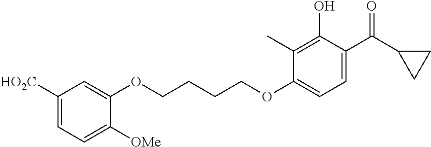 [SMILES: COc1ccc(C(=O)O)cc1OCCCCOc1ccc(C(=O)C2CC2)c(O)c1C]

### 3-(4-(4-(Cyclopropanecarbonyl)-3-hydroxy-2-methylphenoxy)butoxy)-4-methoxybenzoic acid

Methyl 3-hydroxy-4-methoxybenzoate, 1,4-dibromobutane and cyclopropyl(2,4-dihydroxy-3-methylphenyl)methanone, K₂CO₃ and LiOH were processed according to general method B, C and D. White solid (0.280 g, 67.7%). ¹H NMR (DMSO-d₆): δ 8.05 (d, J=9.2 Hz, 1H), 7.52 (dd, J=1.8 Hz, 8.2 Hz, 1H), 7.41 (d, J=1.8 Hz, 1H), 6.99 (d, J=8.2 Hz, 1H), 6.64 (d, J=9.2 Hz, 1H), 4.15-4.04 (m, 4H), 3.78 (s, 3H), 2.91-2.87 (m, 1H), 1.94 (s, 3H), 1.93-1.88 (m, 4H), 1.07-1.01 (m, 4H). ¹³C NMR (DMSO-d₆): δ 204.9, 167.6, 163.1, 161.5, 153.3, 148.1, 130.6, 123.7, 123.4, 114.2, 113.6, 112.4, 111.7, 103.9, 68.4, 56.2, 26.0, 25.9, 16.2, 8.1. LC-MS (ESI) Calcd for C₂₃H₂₆O₇ [M+H]⁺: 415.18. Found: 415.15. HRMS (ESI) Calcd for C₂₃H₂₆O₇ [M+H]⁺: 415.1664. Found: 415.1715.

### Example 61

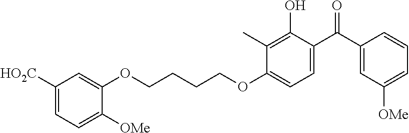 [SMILES: COc1cccc(C(=O)c2ccc(OCCCCOc3cc(C(=O)O)ccc3OC)c(C)c2O)c1]

### 3-(4-(3-Hydroxy-4-(3-methoxybenzoyl)-2-methylphenoxy)butoxy)-4-methoxybenzoic acid

Methyl 3-hydroxy-4-methoxybenzoate, 1,4-dibromobutane and 1-(2,4-dihydroxy-3-methylphenyl)(3-methoxyphenyl)methanone, K₂CO₃ and LiOH were processed according to general method B, C and D. White solid (0.280 g, 58%). ¹H NMR (DMSO-d₆): δ 7.52 (dd, J=1.8 Hz, 8.2 Hz, 1H), 7.44-7.41 (m, 2H), 7.37 (d, J=9.2 Hz, 1H), 7.17-7.10 (m, 3H), 6.99 (d, J=8.2 Hz, 1H), 6.62 (d, J=9.2 Hz, 1H), 4.14-4.04 (m, 4H), 3.77 (s, 3H), 3.76 (s, 3H), 2.01 (s, 3H), 1.91-1.82 (m, 4H). ¹³C NMR (DMSO-d₆): δ 199.7, 167.1, 162.8, 161.7, 159.0, 152.7, 147.5, 139.2, 132.9, 129.6, 123.2, 122.9, 120.9, 117.4, 113.6, 113.1, 112.8, 112.3, 111.1, 103.5, 55.6, 55.3, 25.4, 7.6. LC-MS (ESI) Calcd for C₂₇H₂₈O₈ [M+H]⁺: 481.18. Found: 481.00. HRMS (ESI) Calcd for C₂₇H₂₈O₈ [M+H]⁺: 481.1747. Found: 481.1794.

### Example 62

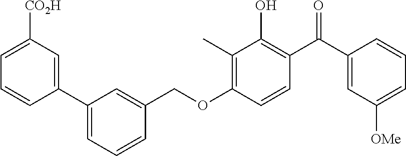 [SMILES: COc1cccc(C(=O)c2ccc(OCc3cccc(-c4cccc(C(=O)O)c4)c3)c(C)c2O)c1]

### 3′-((3-Hydroxy-4-(3-methoxybenzoyl)-2-methylphenoxy)methyl)-[1,1′-biphenyl]-3-carboxylic acid

1-(2,4-Dihydroxy-3-methylphenyl)(3-methoxyphenyl)methanone and methyl 3′-(bromomethyl)-[1,1′-biphenyl]-3-carboxylate, K₂CO₃ and LiOH were processed according to general method E. White solid (0.176 g, 75%). ¹H NMR (DMSO-d₆): δ 12.45 (s, 1H), 8.17 (s, 1H), 7.90 (t, J=7.3 Hz, 2H), 7.78 (s, 1H), 7.65 (d, J=7.3 Hz, 1H), 7.57 (t, J=7.3 Hz, 1H), 7.52-7.38 (m, 4H), 7.17-7.10 (m, 3H), 6.76 (d, J=9.2 Hz, 1H), 5.31 (s, 2H), 3.72 (s, 3H), 2.09 (s, 3H). ¹³C NMR (DMSO-d₆): δ 199.7, 167.2, 162.4, 161.6, 159.0, 140.1, 139.4, 139.1, 137.8, 132.9, 131.0, 129.8, 129.4, 128.4, 127.3, 126.8, 125.9, 120.9, 117.4, 113.7, 113.1, 112.7, 103.9, 69.6, 55.3, 7.9. LC-MS (ESI) m/z calcd for C₂₉H₂₄O₆ [M+H]⁺: 469.15. Found: 469.00. HRMS (ESI) m/z calcd for C₂₉H₂₄O₆ [M+H]⁺: 469.1535. Found: 469.1584.

### Example 63

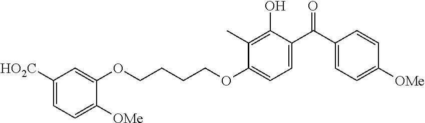 [SMILES: COc1ccc(C(=O)c2ccc(OCCCCOc3cc(C(=O)O)ccc3OC)c(C)c2O)cc1]

### 3-(4-(3-Hydroxy-4-(4-methoxybenzoyl)-2-methylphenoxy)butoxy)-4-methoxybenzoic acid

Methyl 3-hydroxy-4-methoxybenzoate, 1,4-dibromobutane, 1-(2,4-dihydroxy-3-methylphenyl) (4-methoxyphenyl)methanone, K₂CO₃ and LiOH were processed according to general method B, C and D. White solid (0.156 g, 63%). ¹H NMR (DMSO-d₆): δ 7.62 (d, J=6.9 Hz, 2H), 7.52 (dd, J=1.8 Hz, 8.2 Hz, 1H), 7.42-7.39 (m, 2H), 7.06 (d, J=6.9 Hz, 2H), 6.99 (d, J=8.2 Hz, 1H), 6.63 (d, J=9.2 Hz, 1H), 4.14-4.04 (m, 4H), 3.82 (s, 3H), 3.77 (s, 3H), 2.00 (s, 3H), 1.92-1.83 (m, 4H). ¹³C NMR (DMSO-d₆): δ 199.0, 167.6, 162.8, 161.9, 153.3, 148.0, 133.1, 131.9, 130.7, 123.7, 114.3, 113.6, 113.5, 112.8, 111.6, 103.8, 68.5, 56.2, 56.0, 25.9, 8.2. LC-MS (ESI) Calcd for C₂₇H₂₈O₈ [M+H]⁺: 481.18. Found: 481.00. HRMS (ESI) Calcd for C₂₇H₂₈O₈ [M+H]⁺: 481.1751. Found: 481.1799.

### Example 64

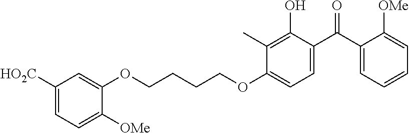 [SMILES: COc1ccc(C(=O)O)cc1OCCCCOc1ccc(C(=O)c2ccccc2OC)c(O)c1C]

### 3-(4-(3-Hydroxy-4-(2-methoxybenzoyl)-2-methylphenoxy)butoxy)-4-methoxybenzoic acid

Methyl 3-hydroxy-4-methoxybenzoate, 1,4-dibromobutane, 10(2,4-dihydroxy-3-methylphenyl) (2-methoxyphenyl)methanone, K₂CO₃ and LiOH were processed according to general method B, C and D. White solid (0.100 g, 40.4%). ¹H NMR (DMSO-d₆): δ 7.52-7.47 (m, 2H), 7.41 (d, J=1.8 Hz, 1H), 7.24 (dd, J=1.8 Hz, 7.3 Hz, 1H), 7.14 (d, J=8.2 Hz, 1H), 7.06-7.02 (m, 2H), 6.98 (d, J=8.2 Hz, 1H), 6.55 (d, J=9.2 Hz, 1H), 4.11-4.03 (m, 4H), 3.77 (s, 3H), 3.68 (s, 3H), 1.99 (s, 3H), 1.89-1.78 (m, 4H). ¹³C NMR (DMSO-d₆): δ 200.8, 167.6, 163.5, 161.9, 156.2, 153.3, 148.0, 133.5, 132.2, 128.6, 128.1, 123.7, 123.4, 120.9, 114.3, 113.6, 112.5, 112.4, 111.6, 104.2, 68.5, 68.4, 56.2, 56.1, 25.9, 25.8, 8.0. LC-MS (ESI) Calcd for C₂₇H₂₈O₈ [M+H]⁺: 481.18. Found: 481.00. HRMS (ESI) Calcd for C₂₇H₂₈O₈ [M+H]⁺: 481.1747. Found: 481.1797.

### Example 65

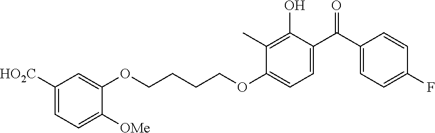 [SMILES: COc1ccc(C(=O)O)cc1OCCCCOc1ccc(C(=O)c2ccc(F)cc2)c(O)c1C]

### 3-(4-(4-(4-Fluorobenzoyl)-3-hydroxy-2-methylphenoxy)butoxy)-4-methoxybenzoic acid

Methyl 3-hydroxy-4-methoxybenzoate, 1,4-dibromobutane, 1-(2,4-dihydroxy-3-methylphenyl) (4-fluorophenyl)methanone, K₂CO₃ and LiOH were processed according to general method B, C and D. White solid (0.13 g, 54%). ¹H NMR (DMSO-d₆): δ 12.33 (s, 1H), 7.70-7.67 (m, 2H), 7.52 (dd, J=1.8 Hz, 8.2 Hz, 1H), 7.41-7.33 (m, 4H), 6.99 (d, J=8.2 Hz, 1H), 6.63 (d, J=9.2 Hz, 1H), 4.21-4.04 (m, 4H), 3.77 (s, 3H), 2.01 (s, 3H), 1.90-1.82 (m, 4H). ¹³C NMR (DMSO-d₆): δ 201.7, 170.3, 168.4, 165.9, 164.7, 155.9, 150.7, 136.0, 134.9, 134.8, 126.3, 118.8, 118.6, 116.3, 114.3, 106.7, 71.2, 58.8, 28.6, 28.5, 10.8. LC-MS (ESI) Calcd for C₂₆H₂₅FO₇ [M+H]V: 469.16. Found: 469.00. HRMS (ESI) Calcd for C₂₆H₂₅FO₇ [M+H]⁺: 469.1575. Found: 469.1628.

### Example 66

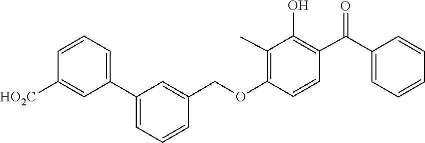 [SMILES: Cc1c(OCc2cccc(-c3cccc(C(=O)O)c3)c2)ccc(C(=O)c2ccccc2)c1O]

### 3′-((4-Benzoyl-3-hydroxy-2-methylphenoxy)methyl)-[1,1′-biphenyl]-3-carboxylic acid

Prepared according to general method E using 1-(2,4-dihydroxy-3-methylphenyl)(phenyl) methanone methyl 3′-(bromomethyl)-[1,1′-biphenyl]-3-carboxylate, K₂CO₃ and LiOH. White solid (0.165 g, 75%). ¹H NMR (DMSO-d₆): δ 8.18 (s, 1H), 7.90 (t, J=7.8 Hz, 2H), 7.78 (s, 1H), 7.64-7.50 (m, 9H), 7.36 (d, J=9.2 Hz, 1H), 6.77 (d, J=9.2 Hz, 1H), 5.31 (s, 2H), 2.09 (s, 3H). ¹³C NMR (DMSO-d₆): δ 200.6, 167.7, 162.9, 162.2, 140.7, 139.9, 138.3, 138.1, 133.5, 132.2, 132.1, 131.6, 129.9, 129.3, 128.9, 127.9, 127.4, 126.9, 126.4, 113.7, 113.3, 70.1, 8.4. LC-MS (ESI) m/z calcd for C₂₈H₂₂O₅ [M+H]⁺: 439.15. Found: 439.00. HRMS (ESI) m/z calcd for C₂₈H₂₂O₅ [M+H]⁺: 439.1462. Found: 439.1538.

### Example 67

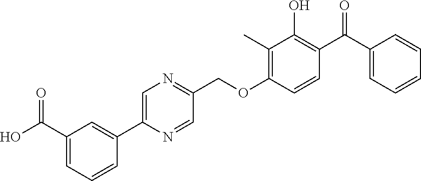 [SMILES: Cc1c(OCc2cnc(-c3cccc(C(=O)O)c3)cn2)ccc(C(=O)c2ccccc2)c1O]

### 3-(5-((4-Benzoyl-3-hydroxy-2-methylphenoxy)methyl)pyrazin-2-yl)benzoic acid

Prepared according to general method F using 1-(2,4-dihydroxy-3-methylphenyl)(phenyl) methanone (0.057 g, 0.25 mmol), 2-bromo-5-(bromomethyl)pyrazine (0.063 g, 0.25 mmol), K₂CO₃ (0.069 g, 0.5 mmol), 3-borono benzoic acid (0.062 g, 0.375 mmol), Pd(PPh₃)₄ (0.029 g, 0.025 mmol) and 2M Na₂CO₃. White solid (0.063 g, 57%). ¹H NMR (DMSO-d₆): δ 9.32 (s, 1H), 8.90 (s, 1H), 8.69 (s, 1H), 8.36 (d, J=7.3 Hz, 1H), 8.04 (d, J=7.3 Hz, 1H), 7.66-7.52 (m, 6H), 7.35 (d, J=8.7 Hz, 1H), 6.80 (d, J=8.7 Hz, 1H), 5.43 (s, 2H), 2.09 (s, 3H). ¹³C NMR (DMSO-d₆): δ 200.6, 162.4, 162.2, 150.9, 150.5, 143.3, 141.8, 138.2, 136.4, 133.4, 132.3, 131.4, 129.9, 129.3, 128.9, 128.1, 113.9, 113.5, 104.4, 69.3, 8.4. LC-MS (ESI) m/z calcd for C₂₆H₂₀N₂O₅ [M+H]⁺: 441.14. Found: 441.10. HRMS (ESI) m/z calcd for C₂₆H₂₀N₂O₅ [M+H]⁺: 441.1351. Found: 441.1406.

### Example 68

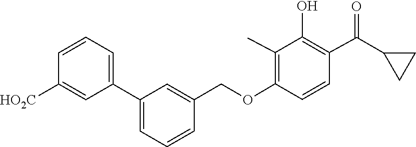 [SMILES: Cc1c(OCc2cccc(-c3cccc(C(=O)O)c3)c2)ccc(C(=O)C2CC2)c1O]

### 3′-((4-(Cyclopropanecarbonyl)-3-hydroxy-2-methylphenoxy)methyl)-[1,1′-biphenyl]-3-carboxylic acid

Prepared according to general method E using 1-(2,4-dihydroxy-3-methylphenyl)(cyclopropyl) methanone, methyl 3′-(bromomethyl)-[1,1′-biphenyl]-3-carboxylate, K₂CO₃ and LiOH. White solid (0.136 g, 67%). ¹H NMR (DMSO-d₆): δ 8.18 (t, J=1.8 Hz, 1H), 8.06 (d, J=9.2 Hz, 2H), 7.93-7.88 (m, 2H), 7.78 (s, 1H), 7.64-7.63 (m, 1H), 7.58 (t, J=7.8 Hz, 1H), 7.49-7.45 (m, 2H), 6.76 (d, J=9.2 Hz, 1H), 5.32 (s, 2H), 2.90-2.85 (m, 1H), 2.02 (s, 3H), 1.08-0.99 (m, 4H). ¹³C NMR (DMSO-d₆): δ 205.0, 167.7, 162.6, 161.5, 140.7, 139.9, 138.8, 132.1, 131.6, 130.6, 129.9, 128.9, 127.8, 127.4, 126.9, 126.4, 114.4, 112.9, 104.5, 70.0, 16.4, 12.3, 8.3. LC-MS (ESI) m/z calcd for C₂₅H₂₂O₅ [M+H]f: 403.15. Found: 403.00. HRMS (ESI) m/z calcd for C₂₅H₂₂O₅S [M+H]⁺: 403. 1453. Found: 403.1515.

### Example 69

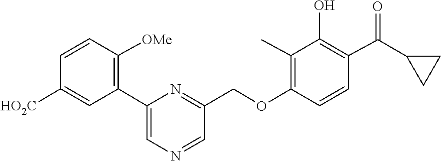 [SMILES: COc1ccc(C(=O)O)cc1-c1cncc(COc2ccc(C(=O)C3CC3)c(O)c2C)n1]

### 3-(6-((4-(Cyclopropanecarbonyl)-3-hydroxy-2-methylphenoxy)methyl)pyrazin-2-yl)-4-methoxybenzoic acid

Prepared according to general method F using 1-(2,4-dihydroxy-3-methylphenyl)(cyclopropyl) methanone 2-bromo-5-(bromomethyl)pyrazine, K₂CO₃, 3-borono-4-methoxybenzoic acid, Pd(PPh₃)₄) and 2M Na₂CO₃. White solid. ¹H NMR (DMSO-d₆): δ 9.12 (s, 1H), 8.89 (s, 1H), 8.37 (s, 1H), 8.09 (d, J=9.2 Hz, 1H), 8.06 (d, J=9.2 Hz, 1H), 7.48 (d, J=8.7 Hz, 1H), 6.80 (d, J=9.2 Hz, 1H), 5.44 (s, 2H), 3.98 (s, 3H), 2.90-2.89 (m, 1H), 2.05 (s, 3H), 1.10-0.99 (m, 4H). ¹³C NMR (DMSO-d₆): δ 205.2, 167.3, 162.3, 161.2, 160.8, 150.2, 149.8, 145.2, 143.4, 133.2, 132.8, 130.6, 125.2, 123.9, 114.7, 113.0, 112.6, 104.4, 69.2, 56.8, 16.5, 12.3, 8.3. LC-MS (ESI) m/z calcd for C₂₄H₂₂N₂O₆ [M+H]⁺: 435.15. Found: 435.00. HRMS (ESI) m/z calcd for C₂₄H₂₂N₂O₆ [M+H]⁺: 435. 1488. Found: 435.1559.

### Example 70

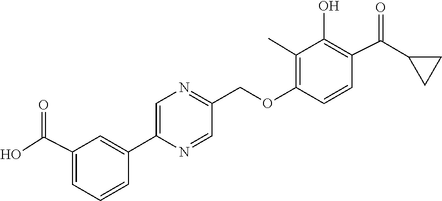 [SMILES: Cc1c(OCc2cnc(-c3cccc(C(=O)O)c3)cn2)ccc(C(=O)C2CC2)c1O]

### 3-(5-((4-(Cyclopropanecarbonyl)-3-hydroxy-2-methylphenoxy)methyl)pyrazin-2-yl)benzoic acid

Prepared according to general method F using 1-(2,4-dihydroxy-3-methylphenyl)(cyclopropyl) methanone 2-bromo-5-(bromomethyl)pyrazine, K₂CO₃, 3-borono benzoic acid, Pd(PPh₃)₄) and 2M Na₂CO₃. White solid. ¹H NMR (DMSO-d₆): δ 9.31 (s, 1H), 8.88 (s, 1H), 8.68 (s, 1H), 8.38 (d, J=7.8 Hz, 1H), 8.06 (d, J=9.2 Hz, 1H), 8.02 (d, J=7.8 Hz, 1H), 7.64 (t, J=7.8 Hz, 1H), 6.80 (d, J=9.2 Hz, 1H), 5.44 (s, 2H), 2.90-2.89 (m, 1H), 2.05 (s, 3H), 1.10-0.99 (m, 4H). ¹³C NMR (DMSO-d₆): δ 205.2, 167.5, 162.2, 161.5, 151.1, 150.3, 143.4, 141.8, 136.3, 132.3, 13.4, 131.2, 130.6, 129.9, 128.0, 114.2, 113.1, 104.5, 69.2, 16.5, 12.4, 8.3. LC-MS (ESI) m/z calcd for C₂₃H₂₀N₂O₅ [M+H]⁺: 405.15. Found: 405.00. HRMS (ESI) m/z calcd for C₂₃H₂₀N₂O₅ [M+H]⁺: 405.1388. Found: 405.1459.

### Example 71

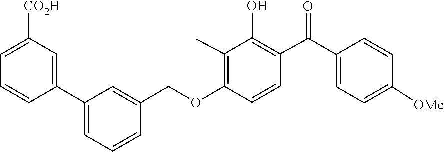 [SMILES: COc1ccc(C(=O)c2ccc(OCc3cccc(-c4cccc(C(=O)O)c4)c3)c(C)c2O)cc1]

### 3′-((3-Hydroxy-4-(4-methoxybenzoyl)-2-methylphenoxy)methyl)-[1,1′-biphenyl]-3-carboxylic acid

Prepared according to general method E using 1-(2,4-dihydroxy-3-methylphenyl)(4-methoxy phenyl)methanone, methyl 3′-(bromomethyl)-[1,1′-biphenyl]-3-carboxylate, K₂CO₃ and LiOH. White solid (0.169 g, 72%). ¹H NMR (DMSO-d₆): δ 12.47 (s, 1H), 8.16 (t, J=1.8 Hz, 1H), 7.97-7.88 (m, 2H), 7.77 (s, 1H), 7.65-7.41 (m, 7H), 7.06-7.03 (m, 2H), 6.74 (d, J=8.7 Hz, 1H), 5.29 (s, 2H), 3.81 (s, 3H), 2.07 (s, 3H). ¹³C NMR (DMSO-d₆): 198.5, 167.2, 162.3, 161.9, 161.3, 140.2, 139.4, 137.6, 132.5, 131.6, 131.4, 131.1, 129.4, 128.4, 127.3, 126.9, 126.4, 125.8, 113.8, 113.4, 112.7, 103.7, 69.5, 55.5, 7.9. LC-MS (ESI) m/z calcd for C₂₉H₂₄O₆ [M+H]⁺: 469.16. Found: 469.00. HRMS (ESI) m/z calcd for C₂₉H₂₄O₆ [M+H]⁺: 469.1554. Found: 469.1603.

### Example 72

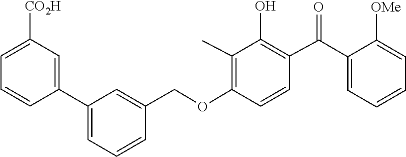 [SMILES: COc1ccccc1C(=O)c1ccc(OCc2cccc(-c3cccc(C(=O)O)c3)c2)c(C)c1O]

### 3′-((3-Hydroxy-4-(2-methoxybenzoyl)-2-methylphenoxy)methyl)-[1,1′-biphenyl]-3-carboxylic acid

Prepared according to general method E using 1-(2,4-dihydroxy-3-methylphenyl)(3-methoxy phenyl)methanone, methyl 3′-(bromomethyl)-[1,1′-biphenyl]-3-carboxylate, K₂CO₃ and LiOH. White solid (0.13 g, 55.5%). ¹H NMR (DMSO-d₆): δ 8.15 (t, J=1.8 Hz, 1H), 7.89 (d, J=9.2 Hz, 2H), 7.76 (s, 1H), 7.63 (t, J=7.8 Hz, 1H), 7.50-7.46 (m, 4H), 7.24 (dd, J=1.8 Hz, 9.2 Hz, 1H), 7.13 (d, J=8.2 Hz, 1H), 7.06-7.02 (m, 2H), 6.68 (d, J=9.2 Hz, 1H), 5.23 (s, 2H), 3.67 (s, 3H), 2.06 (s, 3H). ¹³C NMR (DMSO-d₆): δ 200.7, 162.6, 161.4, 157.7, 140.1, 139.4, 137.5, 132.9, 131.7, 131.6, 131.1, 129.4, 128.1, 127.5, 127.3, 126.9, 126.4, 125.9, 120.4, 114.1, 112.3, 111.8, 104.2, 66.3, 55.6, 7.7. LC-MS (ESI) m/z calcd for C₂₉H₂₄O₆ [M+H]⁺: 469.16. Found: 469.00. HRMS (ESI) m/z calcd for C₂₉H₂₄O₆ [M+H]⁺: 469.1554. Found: 469.1625.

### Example 73

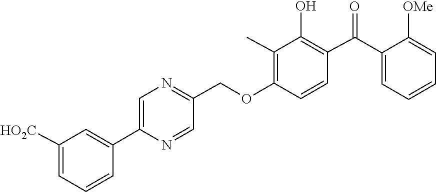 [SMILES: COc1ccccc1C(=O)c1ccc(OCc2cnc(-c3cccc(C(=O)O)c3)cn2)c(C)c1O]

### 3-(5-((3-Hydroxy-4-(2-methoxybenzoyl)-2-methylphenoxy)methyl)pyrazin-2-yl)benzoic acid

Prepared according to general method F using 1-(2,4-dihydroxy-3-methylphenyl)(2-methoxy phenyl)methanone, 2-bromo-5-(bromomethyl)pyrazine, K₂CO₃, 3-borono benzoic acid, Pd(PPh₃)₄) and 2M Na₂CO₃. White solid. ¹H NMR (DMSO-d₆): δ 9.28 (s, 1H), 8.86 (s, 1H), 8.66 (s, 1H), 8.34 (d, J=7.8 Hz, 1H), 8.01 (d, J=7.8 Hz, 1H), 7.62 (t, J=7.8 Hz, 1H), 7.46 (t, J=9.2 Hz, 1H), 7.24 (dd, J=1.8 Hz, 7.3 Hz, 1H), 7.11 (d, J=8.2 Hz, 1H), 7.05 (t, J=9.2 Hz, 2H), 6.71 (d, J=9.2 Hz, 1H), 5.34 (s, 2H), 3.67 (s, 3H), 2.09 (s, 3H). ¹³C NMR (DMSO-d₆): δ 200.5, 167.0, 162.1, 161.4, 155.7, 150.4, 149.8, 142.8, 141.2, 135.9, 132.9, 131.9, 131.7, 130.8, 130.6, 129.4, 128.1, 127.5, 127.4, 120.4, 114.3, 112.5, 11.9, 104.1, 68.8, 55.6, 7.7. LC-MS (ESI) m/z calcd for C₂₇H₂₂N₂O₆ [M+H]⁺: 471.15. Found: 471.00. HRMS (ESI) m/z calcd for C₂₇H₂₂N₂O₆ [M+H]⁺: 471.1496. Found: 471.1573.

### Example 74

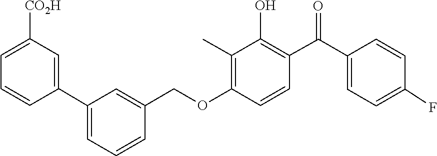 [SMILES: Cc1c(OCc2cccc(-c3cccc(C(=O)O)c3)c2)ccc(C(=O)c2ccc(F)cc2)c1O]

### 3′-((4-(4-Fluorobenzoyl)-3-hydroxy-2-methylphenoxy)methyl)-[1,1′-biphenyl]-3-carboxylic acid

Prepared according to general method E using 1-(2,4-dihydroxy-3-methylphenyl)(4-fluoro phenyl)methanone, methyl 3′-(bromomethyl)-[1,1′-biphenyl]-3-carboxylate, K₂CO₃ and LiOH. White solid (0.12 g, 52.6%). ¹H NMR (DMSO-d₆): δ 12.31 (s, 1H), 8.17 (t, J=1.8 Hz, 1H), 7.90-7.87 (m, 2H), 7.68-7.64 (m, 2H), 7.56 (t, J=7.8 Hz, 1H), 7.49-7.46 (m, 2H), 7.36-7.32 (m, 2H), 6.76 (d, J=9.2 Hz, 1H), 5.30 (s, 2H), 3.72 (s, 3H), 2.09 (s, 3H). ¹³C NMR (DMSO-d₆): δ 198.5, 167.2, 161.5, 140.1, 139.4, 137.6, 134.3, 132.7, 131.7, 131.6, 131.1, 129.4, 128.4, 127.3, 126.8, 126.4, 125.8, 115.6, 115.4, 113.2, 112.8, 104.0, 69.6, 7.9. LC-MS (ESI) m/z calcd for C₂₈H₂₁FO₅ [M+H]⁺: 457.14. Found: 457.00. HRMS (ESI) m/z calcd for C₂₈H₂₁FO₅ [M+H]⁺: 457.1371. Found: 457.1433.

### Example 75

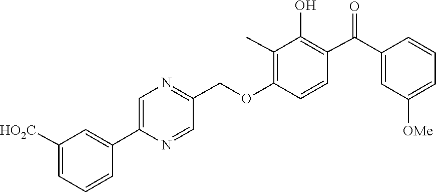 [SMILES: COc1cccc(C(=O)c2ccc(OCc3cnc(-c4cccc(C(=O)O)c4)cn3)c(C)c2O)c1]

### 3-(5-((3-Hydroxy-4-(3-methoxybenzoyl)-2-methylphenoxy)methyl)pyrazin-2-yl)benzoic acid

Prepared according to general method F using 1-(2,4-dihydroxy-3-methylphenyl)(3-methoxy phenyl)methanone, 2-bromo-5-(bromomethyl)pyrazine, K₂CO₃, 3-borono benzoic acid, Pd(PPh₃)₄) and 2M Na₂CO₃. White solid. ¹H NMR (DMSO-d₆): δ 12.44 (s, 1H), 9.29 (s, 1H), 8.89 (s, 1H), 8.67 (s, 1H), 8.32 (d, J=7.8 Hz, 1H), 8.03 (d, J=8.7 Hz, 1H), 7.63 (t, J=7.8 Hz, 1H), 7.43-7.39 (m, 2H), 7.15-7.13 (m, 3H), 6.81 (d, J=9.2 Hz, 1H), 5.42 (s, 2H), 3.77 (s, 3H), 2.12 (s, 3H). HRMS (ESI) m/z calcd for C₂₇H₂₂N₂O₆ [M+H]⁺: 471.1478. Found: 471.1571.

### Example 76

### 3-[4-(4-{[4-(tert-Butyl)phenyl]carbonyl}-3-hydroxy-2-methylphenoxy)butoxy]-4-methoxybenzoic acid

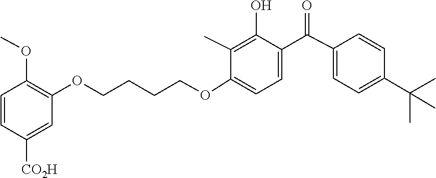 [SMILES: COc1ccc(C(=O)O)cc1OCCCCOc1ccc(C(=O)c2ccc(C(C)(C)C)cc2)c(O)c1C]

Methyl 3-hydroxy-4-methoxybenzoate, 1,4-dibromobutane and (4-(tert-butyl)phenyl)(2,4-dihydroxy-3-methylphenyl)methanone, potassium carbonate and lithium hydroxide were processed according to general method B, C and D. White solid (0.186 g, 73.4%). ¹H NMR (400 MHz, DMSO-d₆) δ 7.58 (s, 4H), 7.55 (dd, J=7.6 Hz, 2.0 Hz, 1H), 7.45 (d, J=2.0 Hz, 1H), 7.42 (d, J=9.0 Hz, 1H), 7.03 (d, J=8.5 Hz, 1H), 6.66 (d, J=9.1 Hz, 1H), 4.18 (m, 4H), 3.80 (s, 3H), 2.04 (s, 3H), 1.96-1.90 (m, 4H), 1.32 (s, 9H). LC-MS (ESI) Calcd for C₃₀H₃₄O₇ [M+H]⁺: 507.59. Found: 507.25.

### Example 77

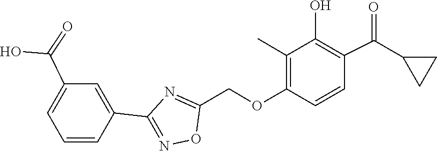 [SMILES: Cc1c(OCc2nc(-c3cccc(C(=O)O)c3)no2)ccc(C(=O)C2CC2)c1O]

### 3-(5-((4-(Cyclopropanecarbonyl)-3-hydroxy-2-methylphenoxy)methyl)-1,2,4-oxadiazol-3-yl)benzoic acid

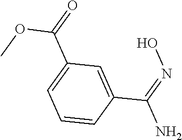

### Step 1: (E)-Methyl 3-(N-hydroxycarbamimidoyl)benzoate

To a solution of methyl 3-cyanobenzoate (1.0 g, 6.21 mmol, 1 equiv) in ethanol (10 mL) was added hydroxylamine (50% wt in water) (1025 mg, 31.1 mmol, 5 equiv.). The resulting mixture was stirred at 100° C. for 1 h. After cooling to room temperature, the mixture was concentrated in vacuo to afford (E)-methyl 3-(N′-hydroxycarbamimidoyl)benzoate (1.02 g, 83.3%) as a yellow solid. LC-MS (ESI): 194.2 [M+H]⁺.

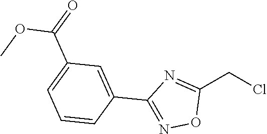 [SMILES: COC(=O)c1cccc(-c2noc(CCl)n2)c1]

### Step 2: Methyl 3-(5-(chloromethyl)-1,2,4-oxadiazol-3-yl)benzoate

To a mixture of (E)-methyl 3-(N-hydroxycarbamimidoyl)benzoate (1.02 g, 5.26 mmol, 1 equiv.) in toluene (10 mL) was added 2-chloroacetyl chloride (707 mg, 6.26 mmol, 1.2 equiv). The resulting mixture was stirred at 110° C. overnight. After cooling to room temperature, the mixture was concentrated under reduced pressure to obtain a residue which was purified by flash column chromatography (silica gel, eluting with 25% EA in PE) to afford methyl 3-(5-(chloromethyl)-1,2,4-oxadiazol-3-yl)benzoate (383.2 mg, 60.1%) as a white solid. LC-MS (ESI): 252.1 [M+H]⁺.

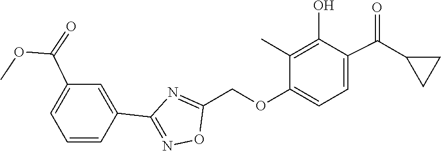 [SMILES: COC(=O)c1cccc(-c2noc(COc3ccc(C(=O)C4CC4)c(O)c3C)n2)c1]

### Step 3: Methyl 3-(5-((4-(cyclopropanecarbonyl)-3-hydroxy-2-methylphenoxy)methyl)-1,2,4-oxadiazol-3-yl)benzoate

To a solution of cyclopropyl(2,4-dihydroxy-3-methylphenyl)methanone (163 mg, 0.85 mmol, 1.2 equiv.) in DMF (6 mL) were added potassium carbonate (196 mg, 1.42 mmol, 2 equiv) and methyl 3-(5-(chloromethyl)-1,2,4-oxadiazol-3-yl)benzoate (200 mg, 0.71 mmol, 1 equiv) successively. The mixture was stirred at 60° C. under N₂ (g) for 3 h. The reaction mixture was poured into water (30 mL) and extracted with ethyl acetate (15 mL×3). The combined organic layers were washed by water (30 mL×3) and brine (10 mL), dried over Na₂SO₄ and filtered. The filtrate was concentrated and purified by flash column chromatography (silica gel, eluting with 30% EA in PE) to afford methyl 3-(5-((4-(cyclopropanecarbonyl)-3-hydroxy-2-methylphenoxy)methyl)-1,2,4-oxadiazol-3-yl)benzoate (112 mg, 34.6%) as a white solid. LC-MS (ESI): 408.1 [M+H]⁺.

### Step 4: 3-(5-((4-(Cyclopropanecarbonyl)-3-hydroxy-2-methylphenoxy)methyl)-1,2,4-oxadiazol-3-yl)benzoic acid

A mixture of methyl 3-(5-((4-(cyclopropanecarbonyl)-3-hydroxy-2-methylphenoxy)methyl)-1,2,4-oxadiazol-3-yl)benzoate (112 mg, 0.27 mmol, 1 equiv.) and lithium hydroxide (19.8 mg, 0.82 mmol, 3 equiv) in water (1 mL) was added to THF:MeOH (4:2 mL) and stirred at room temperature overnight. The solvents were evaporated under reduced pressure and diluted with water (5 mL). The mixture was adjusted to pH=3 with 1 N HCl aqueous solution. The precipitate was collected and purified with prep-HPLC to afford the title compound (16 mg, 14.9%) as a white solid. ¹H NMR (400 MHz, DMSO-d₆) δ 13.12 (s, 1H), 8.56 (d, J=1.2 Hz, 1H), 8.25 (dd, J=7.6, 7.6 Hz, 1H), 8.17-8.14 (m, 2H), 7.73 (t, J=7.8 Hz, 1H), 6.85 (d, J=8.8 Hz, 2H), 5.80 (s, 2H), 2.96 (dd, J=9.6, 8.4 Hz, 1H), 2.10 (s, 3H), 1.23-1.08 (m, 4H). LC-MS: 394.2 [M+H]⁺.

### Example 78

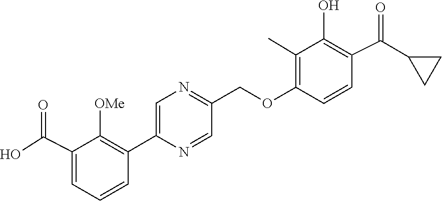 [SMILES: COc1c(C(=O)O)cccc1-c1cnc(COc2ccc(C(=O)C3CC3)c(O)c2C)cn1]

### 3-(5-((4-(Cyclopropanecarbonyl)-3-hydroxy-2-methylphenoxy)methyl)pyrazin-2-yl)-2-methoxybenzoic acid

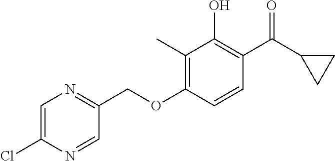 [SMILES: Cc1c(OCc2cnc(Cl)cn2)ccc(C(=O)C2CC2)c1O]

### Step 1: (4-((5-Chloropyrazin-2-yl)methoxy)-2-hydroxy-3-methylphenyl)(cyclopropyl)methanone

To a solution of (5-chloropyrazin-2-yl)methanol (250 mg, 1.74 mmol, 1 equiv.), triphenylphosphine (500 mg, 1.91 mmol, 1.1 equiv.) and cyclopropyl(2,4-dihydroxy-3-methylphenyl)methanone (330 mg, 1.74 mmol, 1 equiv) in THF (5 mL) was added diethyl azodicarboxylate (325 mg, 1.91 mmol, 1.1 equiv) dropwise at 0° C. under N₂ (g). The resulting mixture was stirred for 3 h at room temperature. The reaction mixture was then concentrated under reduced pressure and the residue purified with silica gel column chromatography using 0˜20% EtOAc/PE to afford (4-((5-chloropyrazin-2-yl)methoxy)-2-hydroxy-3-methylphenyl)(cyclopropyl)methanone (250 mg, 45.3%) as a white solid. LC-MS (ESI): 318.1 [M+H]⁺.

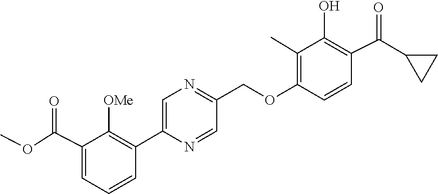 [SMILES: COC(=O)c1cccc(-c2cnc(COc3ccc(C(=O)C4CC4)c(O)c3C)cn2)c1OC]

### Step 2: Methyl 3-(5-((4-(cyclopropanecarbonyl)-3-hydroxy-2-methylphenoxy)methyl)pyrazin-2-yl)-2-methoxybenzoate

A mixture of (4-((5-chloropyrazin-2-yl)methoxy)-2-hydroxy-3-methylphenyl)(cyclopropyl)methanone (250 mg, 0.78 mmol, 1.0 equiv.), sodium carbonate (167 mg, 1.57 mmol, 2 equiv.), methyl 2-methoxy-3-(4,4,5,5-tetramethyl-1,3,2-dioxaborolan-2-yl)benzoate (276 mg, 0.94 mmol, 1.2 equiv.) and tetrakis(triphenylphosphine)palladium(0) (91 mg, 0.08 mmol, 0.1 equiv.) in dioxane/H₂O (6:1, 3:0.5 mL) was stirred at 100° C. overnight. After cooling to room temperature, the mixture was concentrated in vacuo to give a residue which was purified with silica gel column chromatography (silica gel, 0˜25% EtOAc/PE) to afford methyl 3-(5-((4-(cyclopropanecarbonyl)-3-hydroxy-2-methylphenoxy)methyl)pyrazin-2-yl)-2-methoxybenzoate (240 mg, 68.1%) as a white solid. LC-MS (ESI): 448.2 [M+H]⁺.

### Step 3: 3-(5-((4-(Cyclopropanecarbonyl)-3-hydroxy-2-methylphenoxy)methyl)pyrazin-2-yl)-2-methoxybenzoic acid

A mixture of methyl 3-(5-((4-(cyclopropanecarbonyl)-3-hydroxy-2-methylphenoxy)methyl)pyrazin-2-yl)-2-methoxybenzoate (100 mg, 0.22 mmol, 1 equiv) and lithium hydroxide (16 mg, 0.67 mmol, 3 equiv.) in water (1 mL) was added to THF:H₂O (4:2 mL) and stirred overnight at room temperature. The resulting mixture was concentrated in vacuo and then diluted with water (4 mL). The mixture was adjusted to pH=3 with 1 N HCl aqueous solution. The precipitate was collected and purified with prep-HPLC to afford the title compound (34.2 mg, 35.3%) as a white solid. ¹H NMR (400 MHz, DMSO-d₆) δ 13.13 (s, 1H), 9.07 (d, J=2.0 Hz, 1H), 8.95 (d, J=3.0 Hz, 1H), 8.14 (d, J=8.8 Hz, 1H), 7.90 (dd, J=7.6, 7.6 Hz, 1H), 7.82 (dd, J=7.6, 7.6 Hz, 1H), 7.37 (t, J=7.6 Hz, 1H), 6.87 (d, J=9.2 Hz, 1H), 5.49 (s, 2H), 3.63 (s, 3H), 2.96 (dd, J=9.6, 7.6 Hz, 1H), 2.01 (d, J=11.6 Hz, 3H), 1.13-1.07 (m, 4H). LC-MS: 434.2 [M+H]⁺.

### Example 79

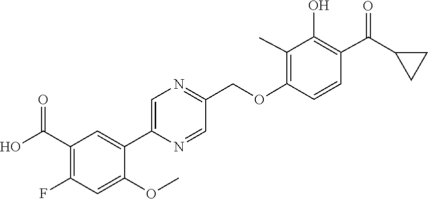 [SMILES: COc1cc(F)c(C(=O)O)cc1-c1cnc(COc2ccc(C(=O)C3CC3)c(O)c2C)cn1]

### 5-(5-((4-(Cyclopropanecarbonyl)-3-hydroxy-2-methylphenoxy)methyl)pyrazin-2-yl)-2-fluoro-4-methoxybenzoic acid

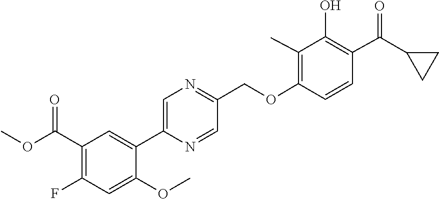 [SMILES: COC(=O)c1cc(-c2cnc(COc3ccc(C(=O)C4CC4)c(O)c3C)cn2)c(OC)cc1F]

### Step 1: Methyl 2-fluoro-4-methoxy-5-(4,4,5,5-tetramethyl-1,3,2-dioxaborolan-2-yl)benzoate

A mixture of methyl 2-fluoro-4-methoxy-5-(4,4,5,5-tetramethyl-1,3,2-dioxaborolan-2-yl)benzoate (250 mg, 0.81 mmol, 1 equiv.), (4-((5-chloropyrazin-2-yl)methoxy)-2-hydroxy-3-methylphenyl)(cyclopropyl)methanone (308 mg, 0.97 mmol, 1.2 equiv.), tetrakistriphenylphosphine palladium (93 mg, 0.08 mmol, 0.1 equiv.) and sodium carbonate (180 mg, 1.61 mmol, 2 equiv.) in dioxane:H₂O (6:1, 3:0.5 mL) was stirred at 100° C. for 16 h. After cooling to room temperature, the solvents were removed under reduced pressure. The residue was purified by flash column chromatography (silica gel, eluting with 25% EA in PE) to afford methyl 2-fluoro-4-methoxy-5-(4,4,5,5-tetramethyl-1,3,2-dioxaborolan-2-yl)benzoate (225 mg, 60.0%) as a white solid. LC-MS (ESI): 466.2 [M+H]⁺.

### Step 2: 5-(5-((4-(Cyclopropanecarbonyl)-3-hydroxy-2-methylphenoxy)methyl)pyrazin-2-yl)-2-fluoro-4-methoxybenzoic acid

A mixture of methyl 2-fluoro-4-methoxy-5-(4,4,5,5-tetramethyl-1,3,2-dioxaborolan-2-yl)benzoate (80 mg, 0.17 mmol, 1 equiv.) and sodium hydroxide (21 mg, 0.52 mmol, 3 equiv) in THF:MeOH:H₂O (4:2:0.5 mL) was stirred at room temperature overnight. The solvents were evaporated under reduced pressure and the residue diluted with water (5 mL). The mixture was adjusted to pH=3 with 1 N HCl aqueous solution. The precipitate was collected and purified with prep-HPLC to afford the title compound (23 mg, 30%) as a white solid. ¹H NMR (400 MHz, DMSO-d₆) δ 13.12 (s, 2H), 9.14 (d, J=1.6 Hz, 1H), 8.92 (d, J=1.6 Hz, 1H), 8.40 (d, J=8.8 Hz, 1H), 8.13 (d, J=9.2 Hz, 1H), 7.23 (d, J=13.2 Hz, 1H), 6.85 (d, J=8.8 Hz, 1H), 5.47 (s, 2H), 3.99 (s, 3H), 2.97-2.93 (m, 1H), 2.08 (d, J=6.0 Hz, 3H), 1.13-1.07 (m, 4H). LC-MS: 452.2 [M+H]⁺.

### Example 80

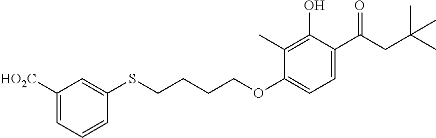 [SMILES: Cc1c(OCCCCSc2cccc(C(=O)O)c2)ccc(C(=O)CC(C)(C)C)c1O]

### 3-((4-(4-(3,3-Dimethylbutanoyl)-3-hydroxy-2-methylphenoxy)butyl)thio)benzoic acid

Methyl 3-mercaptobenzoate, 1,4-dibromobutane, 1-(dihydroxy-3-methylphenyl)-3,3-dimethyl butan-1-one, K₂CO₃ and LiOH were processed according to general method B, C and D. White solid (0.12 g, 55.7%). ¹H NMR (DMSO-d₆): δ 7.83 (d, J=9.2 Hz, 1H), 7.78 (d, J=1.8 Hz, 1H), 7.69 (d, J=7.8 Hz, 1H), 7.53-7.51 (m, 1H), 7.39 (d, J=7.8 Hz, 1H), 6.54 (d, J=8.7 Hz, 1H), 4.06 (t, J=6.4 Hz, 2H), 4.06 (t, J=6.8 Hz, 2H), 2.79 (s, 2H), 1.97 (s, 3H), 1.87-1.70 (m, 4H), 0.95 (s, 9H). ¹³C NMR (DMSO-d₆): δ 205.8, 166.8, 162.4, 161.5, 136.9, 132.1, 131.6, 131.2, 129.3, 128.3, 126.4, 114.4, 111.8, 103.1, 67.5, 48.5, 31.6, 31.5, 29.9, 29.6, 27.6, 24.9, 7.4. LC-MS (ESI) Calcd for C₂₄H₃₀O₅S [M+H]⁺: 431.18. Found: 431.00. HRMS (ESI) Calcd for C₂₄H₃₀O₅S [M−H]⁻: 429.1792. Found: 429.1741.

### Example 81

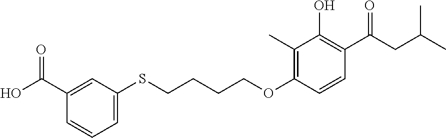 [SMILES: Cc1c(OCCCCSc2cccc(C(=O)O)c2)ccc(C(=O)CC(C)C)c1O]

### 3-((4-(3-Hydroxy-2-methyl-4-(3-methylbutanoyl)phenoxy)butyl)thio)benzoic acid

Methyl 3-mercaptobenzoate, 1,4-dibromobutane, 1-(2,4-dihydroxy-3-methylphenyl)-3-methyl butan-1-one, K₂CO₃ and LiOH were processed according to general method B, C and D. White solid (0.013 g, 6%). ¹H NMR (DMSO-d₆): δ 7.82-7.79 (m, 2H), 7.72 (d, J=7.8 Hz, 1H), 7.55 (d, J=8.2 Hz, 1H), 7.44-7.33 (m, 1H), 6.60 (d, J=9.2 Hz, 1H), 4.10 (t, J=6.0 Hz, 2H), 3.11 (t, J=6.8 Hz, 2H), 2.84 (d, J=6.9 Hz, 2H), 2.15-2.11 (m, 1H), 1.91 (s, 3H), 1.91-1.77 (m, 4H), 0.82 (d, J=6.9 Hz, 6H). ¹³C NMR (DMSO-d₆): δ 206.7, 166.8, 162.4, 161.3, 136.9, 132.1, 131.6, 130.3, 129.3, 128.3, 126.4, 113.4, 111.9, 103.3, 67.5, 45.9, 31.5, 27.6, 25.5, 24.9, 22.4, 7.4. LC-MS (ESI) Calcd for C₂₃H₂₈O₅S [M+H]⁺: 417.17. Found: 417.00. HRMS (ESI) Calcd for C₂₃H₂₈O₅S [M+H]⁺: 417.1647. Found: 417.1719.

### Example 82

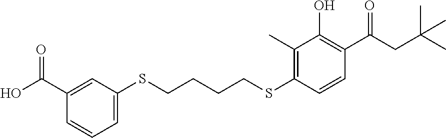 [SMILES: Cc1c(SCCCCSc2cccc(C(=O)O)c2)ccc(C(=O)CC(C)(C)C)c1O]

### 3-{4-[4-(3,3-Dimethylbutanoyl)-3-hydroxy-2-methylphenylthio]butylthio}benzoic acid

1-(4-Bromo-2-hydroxy-3-methylphenyl)-3,3-dimethylbutan-1-one (0.570 g, 2 mmol), CuSO₄·H₂O (0.025 g, 0.1 mmol), KOH (0.561 g, 10 mmol) were taken in DMSO:H₂O (4:0.4 mL). After flushing with argon, ethane-1,2-dithiol (0.377 g, 4 mmol) was added, and the mixture was heated at 110° C. for 20 h. After being cooled to rt, the reaction mixture was partitioned in aqueous HCl and EtOAc. The organic layer was separated was washed with water, brine, and dried over Na₂SO₄. Evaporation of the solvent followed by silica gel column chromatography yielded 1-(2-hydroxy-4-mercapto-3-methylphenyl)-3,3-dimethylbutan-1-one as a brown solid (0.340 g, 71.3%). Methyl 3-mercaptobenzoate, 1,4-dibromobutane, 1-(2-hydroxy-4-mercapto-3-methylphenyl)-3,3-dimethylbutan-1-one, K₂CO₃ and LiOH were processed according to general method B, C and D. White solid (0.117 g, 52.4%). ¹H NMR (DMSO-d₆): δ 7.80-7.77 (m, 2H), 7.77 (d, J=7.8 Hz, 1H), 7.52-7.51 (m, 1H), 7.40 (t, J=8.2 Hz, 1H), 6.81 (d, J=9.2 Hz, 1H), 3.30-3.01 (m, 4H), 2.84 (s, 2H), 2.06 (s, 3H), 1.74-1.70 (m, 4H), 0.96 (s, 9H). HRMS (ESI) Calcd for C₂₄H₃₀O₄S₂ [M+H]⁺:447.1608. Found: 447.1675.

### Example 83

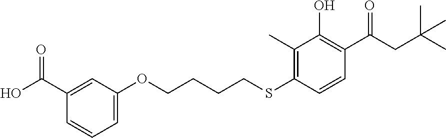 [SMILES: Cc1c(SCCCCOc2cccc(C(=O)O)c2)ccc(C(=O)CC(C)(C)C)c1O]

### 3-{4-[4-(3,3-Dimethylbutanoyl)-3-hydroxy-2-methylphenylthio]butoxy}-4-methoxybenzoic acid

Methyl 3-hydroxybenzoate, 1,4-dibromobutane, 1-(2-hydroxy-4-mercapto-3-methylphenyl)-3,3-dimethylbutan-1-one, K₂CO₃ and LiOH were processed according to general method B, C and D. White solid (0.145 g, 67.4%). ¹H NMR (DMSO-d₆): δ 7.77 (d, J=8.7 Hz, 1H), 7.47-7.35 (m, 3H), 7.15-7.14 (m, 1H), 6.83 (d, J=8.7 Hz, 1H), 4.05 (t, J=6.2 Hz, 2H), 3.13 (t, J=6.8 Hz, 2H), 2.83 (s, 2H), 2.13 (s, 3H), 1.87-1.75 (m, 4H), 0.94 (s, 9H). HRMS (ESI) Calcd for C₂₄H₃₀O₅S [M−H]⁺:429.1854. Found: 429.1781.

### Example 84

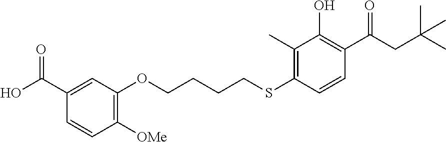 [SMILES: COc1ccc(C(=O)O)cc1OCCCCSc1ccc(C(=O)CC(C)(C)C)c(O)c1C]

### 3-{4-[4-(3,3-Dimethylbutanoyl)-3-hydroxy-2-methylphenylthio]butoxy}-4-methoxybenzoic acid

Methyl 3-hydroxy-4-methoxybenzoate, 1,4-dibromobutane, 1-(2-hydroxy-4-mercapto-3-methyl phenyl)-3,3-dimethylbutan-1-one, K₂CO₃ and LiOH were processed according to general method B, C and D. White solid (0.129 g, 56%). ¹H NMR (DMSO-d₆): δ 7.77 (d, J=8.7 Hz, 1H), 7.53-7.50 (m, 1H), 7.41 (s, 1H), 7.01 (d, J=8.7 Hz, 1H), 6.82 (d, J=8.7 Hz, 1H), 4.02 (t, J=6.1 Hz, 2H), 3.14 (t, J=6.8 Hz, 2H), 2.83 (s, 2H), 2.13 (s, 3H), 1.87-1.75 (m, 4H), 0.94 (s, 9H). HRMS (ESI) Calcd for C₂₅H₃₂O₆S [M+H]⁺:461.1928. Found: 461.1781.

### Example 85

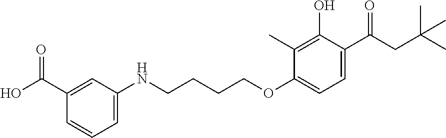 [SMILES: Cc1c(OCCCCNc2cccc(C(=O)O)c2)ccc(C(=O)CC(C)(C)C)c1O]

### 3-((4-(4-(3,3-Dimethylbutanoyl)-3-hydroxy-2-methylphenoxy)butyl)amino)benzoic acid

Methyl 3-aminobenzoate (0.076 g, 0.5 mmol), 1-(4-(4-bromobutoxy)-2-hydroxy-3-methyl phenyl)-3,3-dimethylbutan-1-one (0.179 g, 0.5 mmol), K₂CO₃ (0.138 g, 1 mmol) and 2M LiOH (1 mL) were processed according to general method C and D. White solid (0.013 g, 6.3%). ¹H NMR (DMSO-d₆): δ 7.84 (d, J=9.2 Hz, 1H), 7.13-7.05 (m, 3H), 6.72 (d, J=9.2 Hz, 1H), 6.58 (d, J=9.2 Hz, 1H), 5.84 (brs, 1H), 4.09 (t, J=6.4 Hz, 2H), 3.05 (t, J=6.8 Hz, 2H), 2.80 (s, 2H), 2.02 (s, 3H), 1.94-1.67 (m, 4H), 0.96 (s, 9H). ¹³C NMR (DMSO-d₆): δ 205.8, 168.0, 162.5, 161.6, 148.9, 131.9, 131.2, 128.8, 116.4, 115.8, 114.4, 112.5, 111.8, 103.1, 67.9, 48.5, 42.4, 31.6, 29.9, 26.4, 25.1, 7.5. LC-MS (ESI) Calcd for C₂₄H₃₁NO₅ [M+H]⁺: 414.22. Found: 414.00. HRMS (ESI) Calcd for C₂₄H₃₁NO₅ [M+H]⁺: 414.2207. Found: 414.2278.

### Example 86

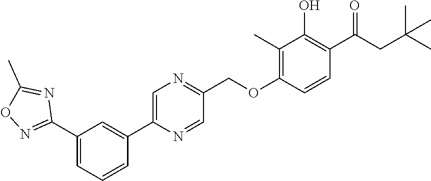 [SMILES: Cc1nc(-c2cccc(-c3cnc(COc4ccc(C(=O)CC(C)(C)C)c(O)c4C)cn3)c2)no1]

### 1-(2-Hydroxy-3-methyl-4-((5-(3-(5-methyl-1,2,4-oxadiazol-3-yl)phenyl)pyrazin-2-yl)methoxy)phenyl)-3,3-dimethylbutan-1-one

(Z)-3-(5-((4-(3,3-Dimethylbutanoyl)-3-hydroxy-2-methylphenoxy)methyl)pyrazin-2-yl)-N′-hydroxybenzimidamide (0.150 g, 0.334 mmol), EtOAc (90.06 g, 0.668 mmol) and NaOMe (0.018 g, 0.334 mmol) were taken in MeOH (5 mL) and heated under reflux for 8 h, the organic phase was evaporated in vacuo. The crude material was partitioned between water and EtOAc. The aqueous layer was extracted with EtOAc (3×10 mL). The organic phase was dried using Na₂SO₄ and evaporated to give the crude product, which was purified by reverse phase HPLC. White solid (0.087 g, 55%). ¹H NMR (DMSO-d₆): δ 9.36 (s, 1H), 8.89 (s, 1H), 8.75 (s, 1H), 8.33 (d, J=7.8 Hz, 1H), 8.08 (d, J=7.8 Hz, 1H), 7.90 (d, J=9.2 Hz, 1H), 7.69 (t, J=7.8 Hz, 1H), 6.76 (d, J=9.1 Hz, 1H), 5.42 (s, 2H), 3.31 (s, 3H), 2.85 (s, 2H), 2.05 (s, 3H), 0.95 (s, 9H). ¹³C NMR (DMSO-d₆): δ 206.6, 178.2, 167.9, 162.2, 151.3, 150.2, 143.4, 141.8, 131.8, 130.6, 130.0, 128.8, 127.7, 125.7, 115.4, 112.9, 104.1, 69.2, 49.1, 32.1, 29.8, 12.6, 8.3. LC-MS (ESI) m/z calcd for C₂₇H₂₈N₄O₄ [M+H]⁺: 473.21. Found: 473.15.

### Example 87

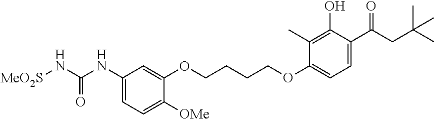 [SMILES: COc1ccc(NC(=O)NS(C)(=O)=O)cc1OCCCCOc1ccc(C(=O)CC(C)(C)C)c(O)c1C]

### N-((3-(4-(4-(3,3-dimethylbutanoyl)-3-hydroxy-2-methylphenoxy)butoxy)-4-methoxyphenyl)carbamoyl)methanesulfonamide

To a stirred mixture of -carboxylic acid (0.133 g, 0.3 mmol), sulfonamide (0.029 g, 0.3 mmol) and triethylamine (0.061 g, 0.6 mmol) in benzene (3 mL) was added diphenylphosphoryl azide (0.099 g, 0.36 mmol) under nitrogen. The mixture was then heated at 85° C. for 2 h. The cooled mixture was concentrated in vacuo, dissolved in a small volume of ACN and subjected to reverse phase HPLC to yield the product. White solid (0.150 g, 93%). ¹H NMR (DMSO-d₆): δ 8.59 (s, 1H), 7.86 (d, J=9.2 Hz, 1H), 7.14 (s, 1H), 6.84-6.81 (m, 2H), 6.59 (d, J=9.2 Hz, 1H), 4.13-3.95 (m, 4H), 3.66 (s, 3H), 3.20 (s, 3H), 2.81 (s, 2H), 1.99 (s, 3H), 1.94-1.87 (m, 4H), 0.96 (s, 9H). ¹³C NMR (DMSO-d₆): δ 205.9, 162.6, 161.6, 150.9, 147.9, 144. 9, 131.9, 131.3, 114.4, 112.4, 111.8, 111.2, 105.6, 103.1, 67.9, 55.7, 48.1, 39.9, 31.6, 29.9, 12.5, 25.4, 7.6. LC-MS (ESI) calcd for C₂₆H₃₆N₂O₈S [M+H]⁺: 537.22. Found: 537.00.

### Example 88

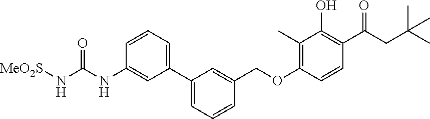 [SMILES: Cc1c(OCc2cccc(-c3cccc(NC(=O)NS(C)(=O)=O)c3)c2)ccc(C(=O)CC(C)(C)C)c1O]

### N-((3′-((4-(3,3-Dimethylbutanoyl)-3-hydroxy-2-methylphenoxy)methyl)-[1,1′-biphenyl]-3-yl)carbamoyl)methanesulfonamide

To a stirred mixture of 3′-((4-(3,3-dimethylbutanoyl)-3-hydroxy-2-methylphenoxy)methyl)-[1,1′-biphenyl]-3-carboxylic acid (0.043 g, 0.1 mmol), methanesulfonamide (0.009 g, 0.1 mmol) and Et₃N (0.020 g, 0.2 mmol) in benzene (1 mL) was added diphenylphosphoryl azide (0.029 g, 0.120 mmol) under nitrogen. The mixture was then heated at 85° C. for 2 h. The cooled mixture was concentrated in vacuo, dissolved in a small volume of ACN and subjected to reverse phase HPLC. White solid (0.038 g, 72.4%). ¹H NMR (DMSO-d₆): δ 8.87 (s, 2H), 7.86 (d, J=9.2 Hz, 11H), 7.82 (s, 11H), 7.70 (s, 11H), 7.67-7.33 (m, 5H), 6.70 (d, J=9.2 Hz, 2H), 5.29 (s, 2H), 3.17 (s, 3H), 2.87 (s, 2H), 2.03 (s, 3H), 0.96 (s, 9H). ¹³C NMR (DMSO-d₆): δ 206.5, 162.7, 162.1, 152.7, 138.0, 131.7, 129.9, 129.7, 126.8, 126.2, 121.5, 118.5, 117.5, 115.2, 104.2, 70.1, 49.2, 41.3, 32.1, 30.4, 8.3. LC-MS (ESI) m/z calcd for C₂₈H₃₂N₂O₆S [M+H]⁺: 525.20. Found: 525.00. HRMS (ESI) m/z calcd for C₂₈H₃₂N₂O₆S [M−H]⁻: 523. 1958. Found: 523.1901.

### Example 89

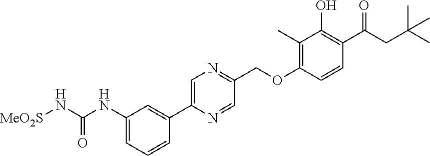 [SMILES: Cc1c(OCc2cnc(-c3cccc(NC(=O)NS(C)(=O)=O)c3)cn2)ccc(C(=O)CC(C)(C)C)c1O]

### N-((3-(5-((4-(3,3-Dimethylbutanoyl)-3-hydroxy-2-methylphenoxy)methyl)pyrazin-2-yl)phenyl)carbamoyl)methanesulfonamide

To a stirred mixture of 3-(5-((4-(3,3-dimethylbutanoyl)-3-hydroxy-2-methylphenoxy)methyl)pyrazin-2-yl)benzoic acid (0.043 g, 0.1 mmol), methanesulfonamide (0.009 g, 0.1 mmol) and Et₃N (0.020 g, 0.2 mmol) in benzene (1 mL) was added diphenylphosphoryl azide (0.029 g, 0.120 mmol) under nitrogen. The mixture was then heated at 85° C. for 2 h. The cooled mixture was concentrated in vacuo, dissolved in a small volume of ACN and subjected to reverse phase HPLC. White solid (0.040 g, 76%). ¹H NMR (DMSO-d₆): δ. 9.19 (s, 1H), 9.02 (s, 1H), 8.86 (s, 1H), 8.28 (s, 1H), 7.91 (d, J=9.2 Hz, 1H), 7.79 (d, J=7.8 Hz, 1H), 7.53 (d, J=8.7 Hz, 1H), 7.45 (t, J=7.8 Hz, 1H), 6.75 (d, J=9.2 Hz, 1H), 5.43 (s, 2H), 3.25 (s, 3H), 2.88 (s, 2H), 2.06 (s, 3H), 0.98 (s, 9H). ¹³C NMR (DMSO-d₆): δ 206.7, 162.3, 161.1, 151.8, 151.0, 143.1, 141.6, 140.0, 136.7, 131.8, 130.1, 121.7, 120.9, 117.6, 115.4, 112.9, 104.1, 69.2, 49.1, 41.8, 32.1, 30.4, 8.3. LC-MS (ESI) m/z calcd for C₂₆H₃₀N₄O₆S [M+H]f: 527.19. Found: 527.00. HRMS (ESI) m/z calcd for C₂₆H₃₀N₄O₆S [M+H]⁺: 527.1912. Found: 527.1978.

### Example 90

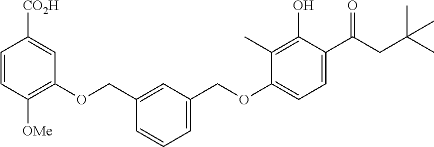 [SMILES: COc1ccc(C(=O)O)cc1OCc1cccc(COc2ccc(C(=O)CC(C)(C)C)c(O)c2C)c1]

### 3-((3-((4-(3,3-Dimethylbutanoyl)-3-hydroxy-2-methylphenoxy)methyl)benzyl)oxy)-4-methoxybenzoic acid

To a solution of 1-(2,4-dihydroxy-3-methylphenyl)-3,3-dimethylbutan-1-one (0.110 g, 0.5 mmol) and 1,3-bis(bromomethyl)benzene (0.132 g, 0-0.5 mmol) in ACN (5 mL) was added K₂CO₃ (0.138 g, 1 mmol) and heated at 80° C. for 2 h (LC-MS showed the complete consumption of starting materials). The reaction mixture was cooled to room temperature. Methyl 3-hydroxy-4-methoxybenzoate (0.091 g, 0.5 mmol) and K₂CO₃ (0.138 g, 1 mmol) were added to the same reaction vessel and heated again at 80 for 2 h. After that time, the solvent was removed under reduced pressure, and partitioned between DCM and water, the organic phase collected, aqueous phase extracted three times with DCM and combined organic phases were washed with water, brine and dried over Na₂SO₄, upon removal of the solvent yielded methyl 3-((3-((4-(3,3-dimethylbutanoyl)-3-hydroxy-2-methylphenoxy)methyl) benzyl)oxy)-4-methoxybenzoate. Crude ester was taken in dioxane (5 mL) and 2M LiOH (2.5 mL, 5 mmol) was added and heated at 80° C. for 30 min. The mixture was cooled to room temperature, diluted with water, acidified using dil. HCl and extracted with EtOAc. Removal of the solvent followed by reverse phase HPLC yielded the title compound as a white solid (0.154 g, 62.5%). ¹H NMR (DMSO-d₆): δ 7.87 (d, J=9.2 Hz, 1H), 7.55-7.49 (m, 3H), 7.40-7.39 (m, 3H), 7.03 (d, J=8.2 Hz, 1H), 6.66 (d, J=9.2 Hz, 1H), 5.23 (s, 2H), 5.12 (s, 2H), 3.79 (s, 3H), 2.81 (s, 2H), 2.03 (s, 3H), 0.97 (s, 9H). ¹³C NMR (DMSO-d₆): δ 203.9, 167.0, 162.1, 161.6, 152.9, 147.2, 137.2, 136.9, 131.1, 128.6, 127.3, 126.8, 126.4, 123.5, 122.9, 114.6, 113.6, 112.2, 111.2, 103.6, 69.6, 69.4, 55.7, 48.7, 31.6, 29.9, 7.7. LC-MS (ESI) Calcd for C₂₉H₃₂O₇ [M+H]⁺: 493.21. Found: 493.00. HRMS (ESI) Calcd for C₂₉H₃₂O₇ [M+H]⁺: 493.2172. Found: 493.2230.

### Example 91

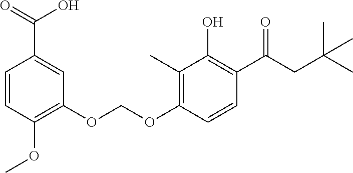 [SMILES: COc1ccc(C(=O)O)cc1OCOc1ccc(C(=O)CC(C)(C)C)c(O)c1C]

### 3-{[4-(3,3-Dimethylbutanoyl)-3-hydroxy-2-methylphenoxy]methoxy}-4-methoxybenzoic acid

Methyl 3-hydroxy-4-methoxybenzoate, diiodomethane, 1-(2,4-dihydroxy-3-methylphenyl)-3,3-dimethylbutan-1-one, K₂CO₃ and LiOH were processed according to general method B, C and D. White solid. ¹H NMR (DMSO-d₆): δ 7.94 (d, J=9.2 Hz, 1H), 7.68-7.63 (m, 2H), 7.08 (d, J=8.7 Hz, 1H), 6.87 (d, J=9.2 Hz, 1H), 5.93 (s, 2H), 3.80 (s, 3H), 2.86 (s, 2H), 1.95 (s, 3H), 0.99 (s, 9H). HRMS (ESI) Calcd for C₂₂H₂₆O₇ [M+H]⁺: 403.1684. Found: 403.1762.

### Example 92

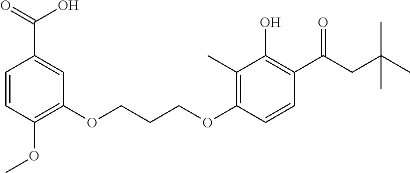 [SMILES: COc1ccc(C(=O)O)cc1OCCCOc1ccc(C(=O)CC(C)(C)C)c(O)c1C]

### 3-{3-[4-(3,3-Dimethylbutanoyl)-3-hydroxy-2-methylphenoxy]propoxy}-4-methoxybenzoic acid

Methyl 3-hydroxy-4-methoxybenzoate, 1,3-dibromopropane, 1-(2,4-dihydroxy-3-methyl phenyl)-3,3-dimethylbutan-1-one, K₂CO₃ and LiOH were processed according to general method B, C and D. White solid (0.120 g, 55.7%). ¹H NMR (DMSO-d₆): δ 7.86 (d, J=9.2 Hz, 1H), 7.53 (dd, J=1.8 Hz, 8.7 Hz, 1H), 7.43 (s, 1H)), 7.00 (d, J=8.7 Hz, 1H), 6.60 (d, J=9.2 Hz, 1H), 4.23 (t, J=6.0 Hz, 2H), 4.15 (t, J=6.0 Hz, 2H), 3.76 (s, 3H), 2.80 (s, 2H), 2.19-2.16 (m, 2H), 1.95 (s, 3H), 0.95 (s, 9H). LC-MS (ESI) Calcd for C₂₄H₃₀O₇ [M+H]⁺: 431.20. Found: 431.05.

### Example 93

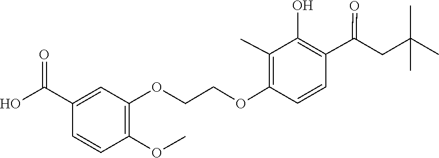 [SMILES: COc1ccc(C(=O)O)cc1OCCOc1ccc(C(=O)CC(C)(C)C)c(O)c1C]

### 3-{2-[4-(3,3-Dimethylbutanoyl)-3-hydroxy-2-methylphenoxy]ethoxy}-4-methoxybenzoic acid

Methyl 3-hydroxy-4-methoxybenzoate, 1,3-dibromoethane, 1-(2,4-dihydroxy-3-methylphenyl)-3,3-dimethylbutan-1-one, K₂CO₃ and LiOH were processed according to general method B, C and D. White solid (0.110 g, 53%). ¹H NMR (DMSO-d₆): δ 7.86 (d, J=9.2 Hz, 1H), 7.56-7.49 (m, 2H), 7.03 (d, J=8.2 Hz, 1H), 6.66 (d, J=9.2 Hz, 1H), 4.40-4.34 (m, 4H), 3.76 (s, 3H), 2.82 (s, 2H), 1.95 (s, 3H), 0.95 (s, 9H). LC-MS (ESI) Calcd for C₂₃H₂₈O₇ [M+H]⁺: 417.18. Found: 417.05.

### Example 94

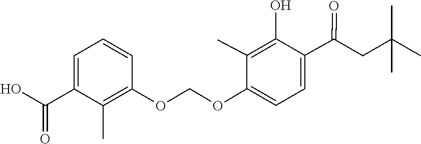 [SMILES: Cc1c(OCOc2ccc(C(=O)CC(C)(C)C)c(O)c2C)cccc1C(=O)O]

### 3-{[4-(3,3-Dimethylbutanoyl)-3-hydroxy-2-methylphenoxy]methoxy}-2-methylbenzoic acid

Methyl 3-hydroxy-2-methybenzoate, diiodomethane, 1-(2,4-dihydroxy-3-methylphenyl)-3,3-dimethylbutan-1-one, K₂CO₃ and LiOH were processed according to general method B, C and D. White solid. ¹H NMR (DMSO-d₆): δ 7.92 (d, J=9.2 Hz, 1H), 7.39-7.23 (m, 3H), 6.85 (d, J=9.2 Hz, 1H), 5.98 (s, 2H), 2.84 (s, 2H), 2.21 (s, 3H), 1.91 (s, 3H), 0.96 (s, 9H). LC-MS (ESI) Calcd for C₂₂H₂₆O₆ [M+H]⁺: 387.17. Found: 387.05.

### Example 95

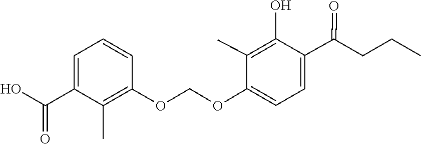 [SMILES: CCCC(=O)c1ccc(OCOc2cccc(C(=O)O)c2C)c(C)c1O]

### 3-[(4-Butanoyl-3-hydroxy-2-methylphenoxy)methoxy]-2-methylbenzoic acid

Methyl 3-hydroxy-2-methylbenzoate, diiodomethane, 1-(2,4-dihydroxy-3-methylphenyl)butan-1-one, K₂CO₃ and LiOH were processed according to general method B, C and D. White solid (0.125 g, 70%). ¹H NMR (DMSO-d₆): δ 7.85 (d, J=9.2 Hz, 1H), 7.36-7.20 (m, 3H), 6.87 (d, J=9.2 Hz, 1H), 6.00 (s, 2H), 2.95 (t, J=7.3 Hz, 2H), 2.27 (s, 3H), 2.91 (s, 3H), 1.65-1.55 (m, 2H), 0.89 (t, J=7.8 Hz, 3H). LC-MS (ESI) Calcd for C₂₀H₂₂O₆ [M+H]⁺: 359.14. Found: 359.05.

### Example 96

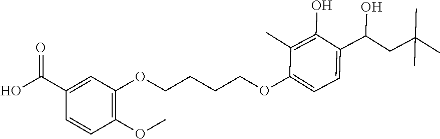 [SMILES: COc1ccc(C(=O)O)cc1OCCCCOc1ccc(C(O)CC(C)(C)C)c(O)c1C]

### 3-{4-[3-Hydroxy-4-(1-hydroxy-3,3-dimethylbutyl)-2-methylphenoxy]butoxy}-4-methoxybenzoic acid

To a solution of ethyl 3-(4-(4-(3,3-dimethylbutanoyl)-3-hydroxy-2-methylphenoxy)butoxy)-4-methoxybenzoate (0.142 g, 0.3 mmol) in MeOH (5 mL) at 0° C. was added NaBH₄ (0.113 g, 3 mmol). The resulting mixture was stirred until LC-MS showed consumption of starting material. The mixture was quenched with water and solvent removed under reduced pressure. The crude reaction mixture was partitioned between water and EtOAc. Organic layer collected, washed with brine, and dried over Na₂SO₄. Removal of the solvent yielded crude intermediate which was taken in dioxane (5 mL) and 2M LiOH (1.5 mL) was added to it. The resulting mixture was heated at 80° C. for 30 min. then cooled to room temperature and diluted with water. The pH was adjusted to 1 by the addition of 1N HCl and EtOAc (20 mL) was added. The organic layer was separated and dried over Na₂SO₄. The solvents were removed by rotary evaporation and the product was isolated by reverse phase HPLC. White solid (0.07 g, 52.3%). ¹H NMR (400 MHz, Chloroform-d): δ 7.68 (dd, J=1.8 Hz, 8.7 Hz, 1H), 7.53-7.52 (m, 1H), 6.88 (d, J=8.5 Hz, 1H), 6.71 (d, J=8.3 Hz, 1H), 6.34 (d, J=8.4 Hz, 1H), 4.94 (t, J=3.3 Hz, 1H), 4.15 (t, J=6.2 Hz, 2H), 4.01 (t, J=5.9 Hz, 2H), 3.91 (s, 3H), 2.09 (s, 3H), 2.08-1.91 (m, 6H), 1.58 (m, 1H), (s, 9H). ¹³C NMR (100 MHz, CDCl₃): δ 171.37, 154.59, 154.21, 148.16, 124.75, 123.81, 121.74, 114.52, 113.99, 110.72, 102.65, 74.32, 68.84, 67.74, 56.21, 50.38, 30.61, 30.13, 26.22, 26.08, 8.21.

### Example 97

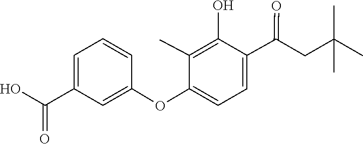 [SMILES: Cc1c(Oc2cccc(C(=O)O)c2)ccc(C(=O)CC(C)(C)C)c1O]

### 3-[4-(3,3-Dimethylbutanoyl)-3-hydroxy-2-methylphenoxy]benzoic acid

A mixture containing 3-bromobenzoic acid (0.101 g, 0.5 mmol), 1-(2,4-dihydroxy-3-methylphenyl)-3,3-dimethylbutan-1-one (0.133 g, 0.6 mmol), CuI (9.52 mg, 0.05 mmol), N,N-dimethylglycine (0.015 g, 0.15 mmol), and Cs₂CO₃ (0.326 g, 1 mmol) in 1,4-dioxane (4 mL) was vigorously stirred at 90° C. under nitrogen atmosphere for 24 h. The solvent was evaporated, and the residue was partitioned between EtOAc and water (10 mL). The combined organic layers were washed with brine (20 mL), dried over Na₂SO₄, and concentrated in vacuo. The residual oil was purified by reverse phase HPLC. White solid (0.073 g, 42.6%). ¹H NMR (400 MHz, DMSO-d₆) δ 7.93 (d, J=9.2 Hz, 1H), 7.76 (d, J=7.8 Hz, 1H), 7.56 (t, J=8.3 Hz, 1H), 7.43-7.42 (m, 1H), 7.33-7.30 (m, 1H), 6.34 (d, J=8.7 Hz, 1H), 2.86 (s, 2H), 2.06 (s, 3H), 0.99 (s, 9H). LC-MS (ESI) Calcd for C₂₀H₂₂O₅ [M+H]⁺: 343.12. Found: 342.95.

### Example 98

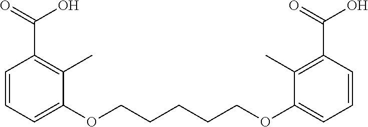 [SMILES: Cc1c(OCCCCCOc2cccc(C(=O)O)c2C)cccc1C(=O)O]

### 3-[5-(3-Carboxy-2-methylphenoxy)pentyloxy]-2-methylbenzoic acid

To a solution of 1,5-dibromopentane (0.230 g, 1 mmol) and methyl 3-hydroxy-2-methylbenzoate (0.332 g, 2 mmol) in ACN was added K₂CO₃ (0.553 g, 4 mmol). The mixture was heated at 80° C. for 2 h (monitored by LC-MS) then cooled to rt, filtered and upon concentration of the filtrate under reduced pressure yielded the intermediate ester. Crude ester was taken in dioxane (5 mL) and heated at 80° C. with 2M LiOH (5 mL). After completion of the reaction, the mixture was diluted with water and acidified. The precipitate was collected by filtration and dried. The product was further purified by silica gel column chromatography using 0 to 20% MeOH in DCM. White solid (0.270 g, 72.5%). White solid (0.2 g, 85%). ¹H NMR (DMSO-d₆): δ 7.26 (d, J=6.4 Hz, 2H), 7.17 (t, J=8.2 Hz, 2H), 7.00 (d, J=7.8 Hz, 2H), 3.99-3.93 (m, 4H), 2.29 (s, 6H), 1.82-1.75 (m, 4H), 1.64-1.58 (m, 2H). LC-MS (ESI) Calcd for C₂₁H₂₄O₆ [M+H]⁺: 373.16. Found: 373.05.

### Example 99

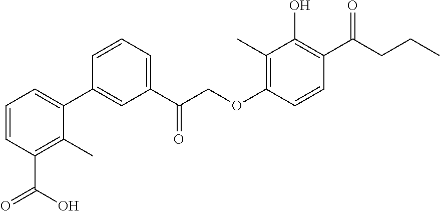 [SMILES: CCCC(=O)c1ccc(OCC(=O)c2cccc(-c3cccc(C(=O)O)c3C)c2)c(C)c1O]

### 3-{3-[2-(4-Butanoyl-3-hydroxy-2-methylphenoxy)acetyl]phenyl}-2-methylbenzoic acid

To a solution of 1-(4-(2-(3-bromophenyl)-2-oxoethoxy)-2-hydroxy-3-methylphenyl)butan-1-one (0.391 g, 1 mmol) in dioxane (10 mL) was added hexamethyldistannane (0.327 g, 1 mmol) and Pd(PPh₃)₄ (0.156 g, 0.01 mmol). The mixture was heated at 80° C. for 30 min (LC-MS showed the consumption of the starting material), then cooled to rt and methyl 3-bromo-2-methylbenzoate (0.344 g, 1.5 mmol) and Pd(PPh₃)₄ (0.115 g, 0.01 mmol) was added to the same mixture and heated at reflux for another 1 h. The mixture was cooled to rt and passed through a short pad of celite. Concentration of the filtrate under reduced pressure yielded the crude ester. This was taken in THF and heated at 80° C. in presence of 2M LiOH (2.5 mL) for 30 min. then diluted with water, acidified using dil. HCl and extracted with EtOAc (3×25 mL). The combined organic layer washed with water, brine and dried over Na₂SO₄. Removal of the solvent followed by reverse phase column chromatography yielded the title compound. White solid (0.261 g, 58.5%). ¹H NMR (400 MHz, DMSO-d₆): δ 8.05-7.99 (m, 1H), 7.97 (dd, J=1.9, 1.0 Hz, 1H), 7.81-7.74 (m, 2H), 7.70-7.65 (m, 2H), 7.43-7.35 (m, 2H), 6.62 (d, J=9.1 Hz, 1H), 5.80 (s, 2H), 2.97 (t, J=7.2 Hz, 2H), 2.36 (s, 3H), 2.08 (s, 3H), 1.64 (m, 2H), 0.92 (t, J=7.4 Hz, 3H). LC-MS (ESI) Calcd for C₂₇H₂₆O₆ [M+H]⁺: 447.17. Found: 447.15.

### Example 100

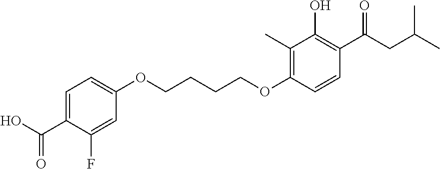 [SMILES: Cc1c(OCCCCOc2ccc(C(=O)O)c(F)c2)ccc(C(=O)CC(C)C)c1O]

### 2-Fluoro-4-{4-[3-hydroxy-2-methyl-4-(3-methylbutanoyl)phenoxy]butoxy}benzoic acid

Methyl 2-fluoro-4-hydroxybenzoate, 1,4-dibomobutane, 1-(2,4-dihydroxy-3-methylphenyl)-3-methylbutan-1-one, K₂CO₃ and LiOH were processed according to general method B, C and D. White solid (0.310 g, 74%). ¹H NMR (400 MHz, DMSO-d₆) δ 7.81-7.74 (m, 2H), 6.87-6.80 (m, 2H), 6.59 (d, J=9.1 Hz, 1H), 4.12-4.08 (m, 4H), 2.82 (d, J=6.9 Hz, 2H), 2.13-2.08 (m, 1H), 1.94 (s, 3H), 1.86-1.83 (m, 4H), 0.93 (d, J=6.4 Hz, 6H). LC-MS (ESI) Calcd for C₂₃H₂₇FO₆ [M+H]⁺: 419.18. Found: 419.15.

### Example 101

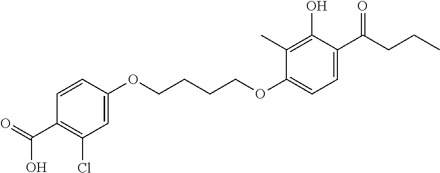 [SMILES: CCCC(=O)c1ccc(OCCCCOc2ccc(C(=O)O)c(Cl)c2)c(C)c1O]

### 4-[4-(4-Butanoyl-3-hydroxy-2-methylphenoxy)butoxy]-2-chlorobenzoic acid

Methyl 2-chloro-4-hydroxybenzoate, 1,4-dibomobutane, 1-(2,4-dihydroxy-3-methylphenyl) butan-1-one, K₂CO₃ and LiOH were processed according to general method B, C and D. White solid (0.343 g, 81.5%). ¹H NMR (400 MHz, DMSO-d₆) δ 7.80 (dd, J=2.6 Hz, 8.8 Hz, 2H), 7.08 (dd, J=1.2 Hz, 2.6, Hz, 1H), 6.94 (m, 1H), 6.64 (d, J=9.1 Hz, 1H), 4.11-4.06 (m, 4H), 2.98 (t, J=7.3 Hz, 2H), 1.98 (s, 3H), 1.86-1.83 (m, 4H), 1.63-1.61 (m, 2H), 0.94 (t, J=7.4 Hz, 3H). LC-MS (ESI) Calcd for C₂₂H₂₅ClO₆ [M+H]⁺: 421.13. Found: 421.00.

### Example 102

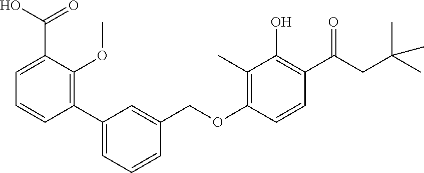 [SMILES: COc1c(C(=O)O)cccc1-c1cccc(COc2ccc(C(=O)CC(C)(C)C)c(O)c2C)c1]

### 3-(3-{[4-(3,3-Dimethylbutanoyl)-3-hydroxy-2-methylphenoxy]methyl}phenyl)-2-methoxybenzoic acid

1-(4-((3-Bromobenzyl)oxy)-2-hydroxy-3-methylphenyl)-3,3-dimethylbutan-1-one (0.2 g, 0.5 mmol), 3-borono-2-methoxybenzoic acid (0.146 g, 0.8 mmol), Pd(PPh₃)₄ (0.06 g, 0.05 mmol) and 2M Na₂CO₃ (1.25 mL) were processed according to general method F. White solid (0.123 g, 61%). ¹H NMR (400 MHz, DMSO-d₆): δ 7.88 (d, J=9.1 Hz, 1H), 7.61-7.59 (m, 2H), 7.47-7.41 (m, 4H), 7.23 (t, J=7.7 Hz, 1H), 6.69 (d, J=9.1 Hz, 1H), 5.32 (s, 2H), 3.36 (s, 3H), 2.85 (s, 2H), 2.05 (s, 3H), 0.97 (s, 9H). LC-MS (ESI) Calcd for C₂₈H₃₀O₆ [M+H]⁺: 463.20. Found: 463.25.

### Example 103

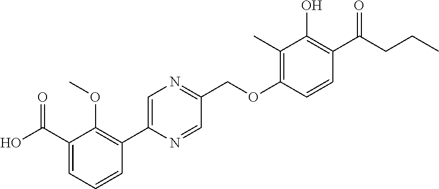 [SMILES: CCCC(=O)c1ccc(OCc2cnc(-c3cccc(C(=O)O)c3OC)cn2)c(C)c1O]

### 3-{5-[(4-Butanoyl-3-hydroxy-2-methylphenoxy)methyl]pyrazin-2-yl}-2-methoxybenzoic acid

1-(4-((5-Bromopyrazin-2-yl)methoxy)-2-hydroxy-3-methylphenyl)butan-1-one (0.100 g, 0.274 mmol), 3-borono-2-methoxybenzoic acid (0.08 g, 0.4 mmol), Pd(PPh₃)₄ (0.03 g, 0.03 mmol) and 2M Na₂CO₃ (0.7 mL) were processed according to general method F. White solid (0.087 g, 73%). ¹H NMR (400 MHz, DMSO-d₆): δ 9.03 (s, 1H), 8.90 (s, 1H), 7.86-7.77 (m, 3H), 7.33 (t, J=7.8 Hz, 1H), 6.79 (d, J=9.2 Hz, 1H), 5.43 (s, 2H), 3.58 (s, 3H), 2.98 (t, J=7.3 Hz, 2H), 2.06 (s, 3H), 1.65 (m, 2H), 0.93 (t, J=7.4 Hz, 3H). LC-MS (ESI) Calcd for C₂₄H₂₄N₂O₆ [M+H]⁺: 437.16. Found: 437.15.

### Example 104

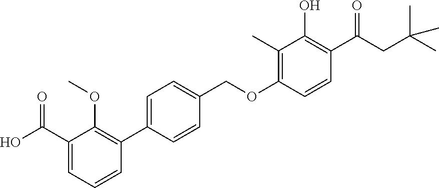 [SMILES: COc1c(C(=O)O)cccc1-c1ccc(COc2ccc(C(=O)CC(C)(C)C)c(O)c2C)cc1]

### 3-(4-{[4-(3,3-Dimethylbutanoyl)-3-hydroxy-2-methylphenoxy]methyl}phenyl)-2-methoxybenzoic acid

1-(4-((4-Bromobenzyl)oxy)-2-hydroxy-3-methylphenyl)-3,3-dimethylbutan-1-one (0.393 g, 1 mmol), 3-borono-2-methoxybenzoic acid (0.295 g, 1.5 mmol), Pd(PPh₃)₄ (0.115 g, 0.1 mmol) and 2M Na₂CO₃ (2.5 mL) were processed according to general method F. White solid (0.340 g, 73%). ¹H NMR (400 MHz, DMSO-d₆): δ 7.90 (d, J=9.1 Hz, 1H), 7.61 (dd, J=1.8 Hz, 7.7 Hz, 1H), 7.59-7.46 (m, 5H), 7.24 (t, J=7.8 Hz, 1H), 6.71 (d, J=9.1 Hz, 1H), 5.32 (s, 2H), 3.39 (s, 3H), 2.83 (s, 2H), 2.04 (s, 3H), 0.97 (s, 9H). LC-MS (ESI) Calcd for C₂₈H₃₀O₆ [M+H]⁺: 463.20. Found: 463.25.

### Example 105

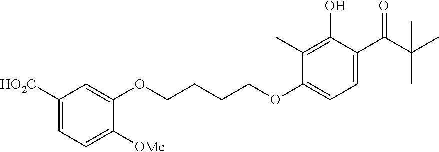 [SMILES: COc1ccc(C(=O)O)cc1OCCCCOc1ccc(C(=O)C(C)(C)C)c(O)c1C]

### 3-(4-(3-Hydroxy-2-methyl-4-pivaloylphenoxy)butoxy)-4-methoxybenzoic acid

Methyl 3-hydroxy-4-methoxybenzoate, 1,4-dibomobutane, 1-(2,4-dihydroxy-3-methylphenyl)-2,2-dimethylpropan-1-one, K₂CO₃ and LiOH were processed according to general method B, C and D. White solid (0.232 g, 54%). ¹H NMR (DMSO-d₆): δ 7.96 (d, J=9.2 Hz, 1H), 7.53 (d, J=10.0 Hz, 1H), 7.45 (s, 1H), 7.02 (d, J=8.7 Hz, 1H), 6.59 (d, J=9.2 Hz, 1H), 4.16-4.07 (m, 4H), 3.80 (s, 3H), 1.97 (s, 3H), 1.91-1.90 (m, 4H), 1.35 (s, 9H). ¹³C NMR (DMSO-d₆): δ 210.9, 165.9, 162.0, 161.4, 152.7, 147.5, 130.3, 123.2, 113.1, 112.5, 111.3, 111.1, 102.5, 67.9, 67.7, 55.6, 42.3, 28.4, 25.4, 7.7. LC-MS (ESI) Calcd for C₂₄H₃₀O₇ [M+H]⁺: 431.20. Found: 431.15. HRMS (ESI) Calcd for C₂₄H₃₀O₇ [M+H]⁺: 431.1985. Found: 431.2012.

### Example 106

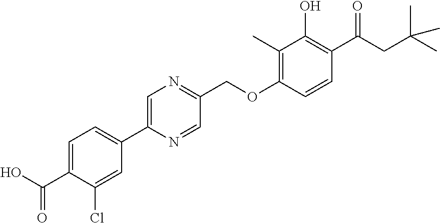 [SMILES: Cc1c(OCc2cnc(-c3ccc(C(=O)O)c(Cl)c3)cn2)ccc(C(=O)CC(C)(C)C)c1O]

### 4-(5-{[4-(3,3-Dimethylbutanoyl)-3-hydroxy-2-methylphenoxy]methyl}pyrazin-2-yl)-2-chlorobenzoic acid

1-(4-((5-Bromopyrazin-2-yl)methoxy)-2-hydroxy-3-methylphenyl)butan-1-one (0.393 g, 1 mmol), 4-borono-2-chlorobenzoic acid (0.300 g, 1.5 mmol), Pd(PPh₃)₄ (0.115 g, 0.1 mmol) and 2M Na₂CO₃ (2.5 mL) were processed according to general method F. White solid (0.348 g, 72%). ¹H NMR (400 MHz, DMSO-d₆): δ 8.99 (s, 1H), 8.94 (s, 1H), 8.04 (d, J=1.7 Hz, 1H), 8.01 (dd, J=1.8 Hz, 9.6 Hz, 1H), 7.93 (d, J=9.2 Hz, 1H), 7.79 (d, J=8.2 Hz, 1H), 6.76 (d, J=9.2 Hz, 1H), 5.47 (s, 2H), 2.84 (s, 2H), 2.06 (s, 3H), 0.98 (s, 9H). LC-MS (ESI) Calcd for C₂₅H₂₅N₂O₅ [M+H]⁺: 469.14. Found: 469.20.

### Example 107

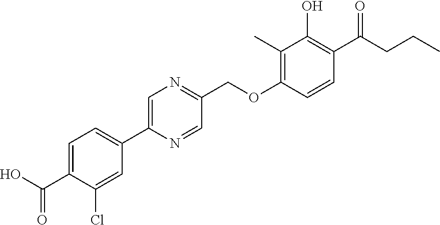 [SMILES: CCCC(=O)c1ccc(OCc2cnc(-c3ccc(C(=O)O)c(Cl)c3)cn2)c(C)c1O]

### 4-{5-[(4-Butanoyl-3-hydroxy-2-methylphenoxy)methyl]pyrazin-2-yl}-2-chlorobenzoic acid

1-(4-((5-Bromopyrazin-2-yl)methoxy)-2-hydroxy-3-methylphenyl)butan-1-one (0.182 g, 0.5 mmol), 4-borono-2-chlorobenzoic acid (0.15 g, 0.75 mmol), Pd(PPh₃)₄ (0.057 g, 0.0.05 mmol) and 2M Na₂CO₃ (1 mL) were processed according to general method F. White solid (0.164 g, 74%). ¹H NMR (400 MHz, DMSO-d₆): δ 8.99 (s, 1H), 8.96 (s, 1H), 8.08 (d, J=1.5 Hz, 1H), 8.02 (dd, J=1.6 Hz, 8.2, Hz, 1H), 7.84 (d, J=9.2 Hz, 1H), 7.77 (d, J=8.2 Hz, 1H), 6.77 (d, J=8.7 Hz, 1H), 5.46 (s, 2H), 2.99 (t, J=7.3 Hz, 2H), 2.06 (s, 3H), 1.64-1.58 (m 2H), 0.93 (t, J=7.4 Hz, 3H). LC-MS (ESI) Calcd for C₂₃H₂₁N₂O₅ [M+H]⁺: 441.11. Found: 441.10.

### Example 108

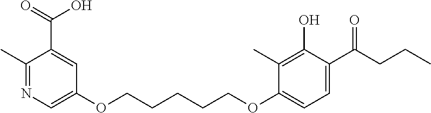 [SMILES: CCCC(=O)c1ccc(OCCCCCOc2cnc(C)c(C(=O)O)c2)c(C)c1O]

### 5-[5-(4-Butanoyl-3-hydroxy-2-methylphenoxy)pentyloxy]-2-methylpyridine-3-carboxylic acid

Ethyl 5-((5-bromopentyl)oxy)-2-methylnicotinate (0.330 g, 1 mmol) 1-(2,4-dihydroxy-3-methyl phenyl)butan-1-one (0.194 g, 1 mmol), K₂CO₃ (0.276, 2 mmol) and 2M LiOH were processed according to general methods C and D. White solid (0.328 g, 79%). ¹H NMR (400 MHz, DMSO-d₆): δ 8.28 (d, J=3.0 Hz, 1H), 7.79 (d, J=8.7 Hz, 1H), 7.62 (d, J=3.0 Hz, 1H), 6.59 (d, J=9.1 Hz, 1H), 4.09-4.03 (m, 4H), 2.94 (t, J=7.3 Hz, 2H), 2.58 (s, 3H), 1.95 (s, 3H), 1.81-1.72 (m, 4H), 1.64-1.54 (m, 4H), 0.93 (t, J=7.4 Hz, 3H). LC-MS (ESI) Calcd for C₂₃H₂₉NO₆ [M+H]⁺: 416.20. Found: 416.70.

### Example 109

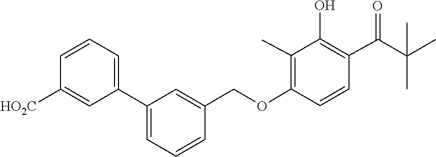 [SMILES: Cc1c(OCc2cccc(-c3cccc(C(=O)O)c3)c2)ccc(C(=O)C(C)(C)C)c1O]

### 3′-((4-Benzoyl-3-hydroxy-2-methylphenoxy)methyl)-[1,1′-biphenyl]-3-carboxylic acid

1-(2,4-Dihydroxy-3-methylphenyl)-2,2-dimethylpropan-1-one (0.104 g, 0.5 mmol), methyl 3′-(bromomethyl)-[1,1′-biphenyl]-3-carboxylate (0.153 g, 0.5 mmol), K₂CO₃ (0.138 g, 1 mmol) and 2M LiOH (2 mL) were processed according to general method E. White solid (0.143 g, 68.3%). ¹H NMR (DMSO-d₆): δ 8.20 (s, 1H), 7.99-7.91 (m, 3H), 7.80 (s, 1H), 7.67 (d, J=7.3 Hz, 1H), 7.62 (t, J=7.8 Hz, 1H), 7.54-7.48 (m, 2H), 6.71 (d, J=9.2 Hz, 1H), 5.33 (s, 2H), 2.06 (s, 3H), 1.35 (s, 9H). ¹³C NMR (DMSO-d₆): δ 211.1, 167.2, 161.9, 161.0, 140.1, 139.4, 137.8, 131.7, 131.1, 130.3, 129.4, 128.4, 127.3, 126.8, 126.4, 125.8, 112.9, 11.8, 103.1, 69.4, 43.9, 28.4, 8.0. LC-MS (ESI) m/z calcd for C₂₆H₂₆O₅ [M+H]⁺: 419.19. Found: 419.00. HRMS (ESI) m/z calcd for C₂₆H₂₆O₅ [M+H]⁺: 419. 1792. Found: 419.1865.

### Example 110

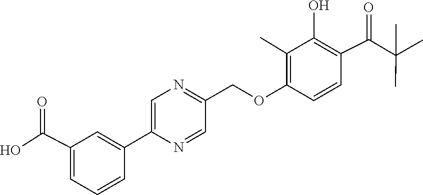 [SMILES: Cc1c(OCc2cnc(-c3cccc(C(=O)O)c3)cn2)ccc(C(=O)C(C)(C)C)c1O]

### 3-(5-((3-Hydroxy-2-methyl-4-pivaloylphenoxy)methyl)pyrazin-2-yl)benzoic acid

1-(2,4-Dihydroxy-3-methylphenyl)-2,2-dimethylpropan-1-one, 2-bromo-5-(bromomethyl) pyrazine, K₂CO₃, 3-borono-benzoic acid, Pd(PPh₃)₄ and 2M Na₂CO₃ according to general method F. White solid (0.071 g, 67.5%). ¹H NMR (DMSO-d₆): δ 9.23 (s, 1H), 8.88 (s, 1H), 8.69 (s, 1H), 8.38 (d, J=7.8 Hz, 1H), 8.03 (d, J=7.8 Hz, 1H), 7.96 (d, J=9.2 Hz, 1H), 7.65 (t, J=7.8 Hz, 1H), 6.72 (d, J=9.2 Hz, 1H), 5.42 (s, 2H), 2.06 (s, 3H), 1.33 (s, 9H). ¹³C NMR (DMSO-d₆): δ 211.7, 167.6, 162.4, 161.1, 151.2, 150.3, 143.3, 141.8, 136.5, 132.3, 131.4, 131.2, 130.8, 129.9, 128.0, 127.2, 120.2, 113.7, 112.7, 103.5, 69.2, 44.5, 28.9, 8.6. LC-MS (ESI) m/z calcd for C₂₄H₂₄N₂O₅ [M+H]⁺: 421.17. Found: 421.05. HRMS (ESI) m/z calcd for C₂₄H₂₄N₂O₅ [M+H]⁺: 421.1696. Found: 421.1758.

### Example 111

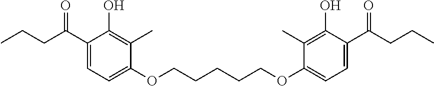 [SMILES: CCCC(=O)c1ccc(OCCCCCOc2ccc(C(=O)CCC)c(O)c2C)c(C)c1O]

### 1-{4-[5-(4-Butanoyl-3-hydroxy-2-methylphenoxy)pentyloxy]-2-hydroxy-3-methylphenyl}butan-1-one

1-(2,4-Dihydroxy-3-methylphenyl)butan-1-one (0.194 g, 1 mmol), 1,5-dibromopentane (0.115 g, 0.5 mmol) and K₂CO₃ (0.553 g, 4 mmol) were taken in ACN (5 mL) and the resulting mixture was heated at 80° C. for 2 h. The reaction mixture was cooled to rt and filtered. Concentration of the filtrate followed by reverse phase HPLC yielded the title compound. White solid (0.2 g, 85%). ¹H NMR (DMSO-d₆): δ 7.77 (d, J=8.7 Hz, 2H), 6.58 (d, J=8.7 Hz, 2H), 4.06-4.00 (m, 4H), 2.93 (t, J=6.8 Hz, 4H), 1.93 (s, 6H), 1.77-1.47 (m, 10H), 0.88 (t, J=7.3 Hz, 6H). LC-MS (ESI) Calcd for C₂₇H₃₆O₆ [M+H]⁺: 457.25. Found: 457.05.

### Example 112

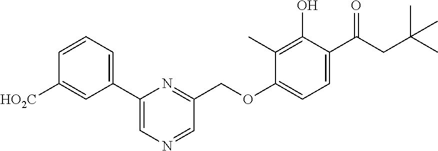 [SMILES: Cc1c(OCc2cncc(-c3cccc(C(=O)O)c3)n2)ccc(C(=O)CC(C)(C)C)c1O]

### 3-(6-((4-(3,3-Dimethylbutanoyl)-3-hydroxy-2-methylphenoxy)methyl)pyrazin-2-yl)benzoic acid

1-(4-((6-bromopyrazin-2-yl)methoxy)-2-hydroxy-3-methylphenyl)-3,3-dimethylbutan-1-one (0.295 g, 0.75 mmol), 3-boronobenzoic acid (0.187 g, 1.25 mmol), 0.450 mmol), Pd(PPh₃)₄ (0.087 g, 0.075 mmol) and 2M Na₂CO₃ solution (1.5 mL) according to general method F (Suzuki cross coupling reaction step). White solid (0.130 g, 39.9%). ¹H NMR (DMSO-d₆): δ 9.27 (s, 1H), 8.77 (s, 1H), 8.69 (s, 1H), 8.36 (d, J=7.8 Hz, 1H), 8.29 (d, J=20.1 Hz, 1H), 8.04 (d, J=7.8 Hz, 1H), 7.88 (t, J=9.6 Hz, 1H), 7.64 (t, J=7.8 Hz, 1H), 6.77 (d, J=9.2 Hz, 1H), 5.47 (s, 2H), 2.82 (s, 2H), 2.07 (s, 3H), 0.96 (s, 9H). LC-MS (ESI) calcd for C₂₅H₂₆N₂O₅ [M+H]⁺: 435.18. Found: 435.00. HRMS (ESI) calcd for C₂₅H₂₆N₂O₅ [M−H]⁻: 433.1823. Found: 433.1754. ¹³C NMR (DMSO-d₆): δ 206.6, 167.5, 162.2, 162.1, 159.5, 151.8, 150.1, 148.5, 142.4, 141.8, 136.4, 135.2, 135.1, 132.3, 131.7, 131.5, 131.3, 130.0, 128.2, 115.4, 115.3, 113.0, 113.0, 104.2, 71.8, 49.1. 32.1, 30.4, 8.3, 8.2.

### Example 113

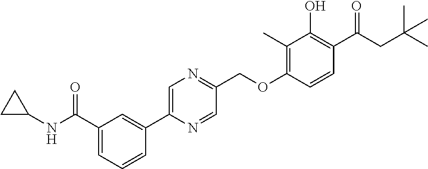 [SMILES: Cc1c(OCc2cnc(-c3cccc(C(=O)NC4CC4)c3)cn2)ccc(C(=O)CC(C)(C)C)c1O]

### N-Cyclopropyl-3-(5-((4-(3,3-dimethylbutanoyl)-3-hydroxy-2-methylphenoxy)methyl)pyrazin-2-yl)benzamide

The benzoic acid derivative (0.025 g, 0.058 mmol) was dissolved in DMF (2 mL) at room temperature. 1-Hydroxybenzotriazole (HOBt, 0 012 g, 0.086 mmol) was added in one portion followed by EDC (0.013 g, 0.086 mmol). The resulting mixture was stirred at room temperature for 30 min. Next, cyclopropylamine (0.004 g, 0.069 mmol) and triethylamine (0.01 mL, 0.069 mmol) were added and stirred for 2 h, after which time the organic phase was removed under reduced pressure and the crude material was partitioned between water and ethyl acetate. The aqueous layer was extracted with ethyl acetate (3×5 mL). The combined organic phase was dried using Na₂SO₄ and evaporated to give 3-(4-(4-(3,3-dimethylbutanoyl)-3-hydroxy-2-methylphenoxy)butoxy)-4-methoxy-N-methylbenzamide. The crude product was purified by HPLC using acetonitrile:water as the solvent system to afford amide as a colorless solid (0.015 g, 55%). LC-MS (ESI) Calcd for C₂₈H₃₁N₃O₄ [M+H]⁺: 474.23. Found: 474.00.

### Example 114

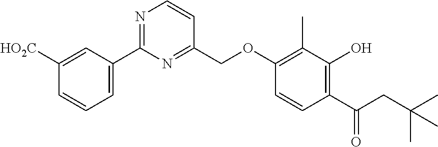 [SMILES: Cc1c(OCc2ccnc(-c3cccc(C(=O)O)c3)n2)ccc(C(=O)CC(C)(C)C)c1O]

### 3-(4-((4-(3,3-Dimethylbutanoyl)-3-hydroxy-2-methylphenoxy)methyl)pyrimidin-2-yl)benzoic acid

1-(4-((2-bromopyrimidin-4-yl)methoxy)-2-hydroxy-3-methylphenyl)-3,3-dimethylbutan-1-one (0.118 g, 0.3 mmol), 3-boronobenzoic acid (0.0.75 g, 0.450 mmol), Pd(PPh₃)₄ (0.035 g, 0.035 mmol) and 2M Na₂CO₃ solution according to general method F (Suzuki cross coupling reaction step). White solid (0.062 g, 48%). LC-MS (ESI) calcd for C₂₅H₂₆N₂O₅ [M+H]⁺: 435.18. Found: 435.00.

### Example 115

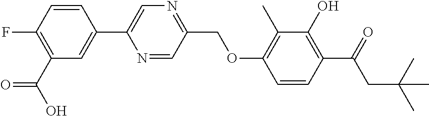 [SMILES: Cc1c(OCc2cnc(-c3ccc(F)c(C(=O)O)c3)cn2)ccc(C(=O)CC(C)(C)C)c1O]

### 5-(5-((4-(3,3-Dimethylbutanoyl)-3-hydroxy-2-methylphenoxy)methyl)pyrazin-2-yl)-2-fluorobenzoic acid

1-(4-((5-bromopyrazin-2-yl)methoxy)-2-hydroxy-3-methylphenyl)-3,3-dimethylbutan-1-one (0.197 g, 0.5 mmol), 5-borono-2-fluoro-benzoic acid (0.138 g, 0.75 mmol), Pd(PPh₃)₄ (0.057 g, 0.05 mmol) and 2M Na₂CO₃ solution according to general method F (Suzuki cross coupling reaction step). White solid (0.097 g, 43%). ¹H NMR (DMSO-d₆): δ 9.29 (s, 1H), 8.87 (s, 1H), 8.60 (d, J=9.2 Hz, 1H), 8.38-8.34 (m, 1H), 7.90 (d, J=9.2 Hz, 1H), 7.45 (t, J=9.2 Hz, 1H), 6.75 (d, J=9.2 Hz, 1H), 5.43 (s, 2H), 2.83 (s, 2H), 2.05 (s, 3H), 0.98 (s, 9H). ¹³C NMR (DMSO-d₆): δ 206.6, 165.4, 162.3, 162.1, 150.9, 149.5, 143.3, 141.6, 132.8, 132.3, 131.8, 130.8, 118.4, 118.2, 115.4, 112.9, 104.1, 69.2, 49.0, 32.1, 30.4, 8.3. LC-MS (ESI) m/z calcd for C₂₅H₂₅FN₂O₅ [M+H]⁺: 453.17. Found: 453.10. HRMS (ESI) m/z calcd for C₂₅H₂₅FN₂O₅ [M−H]⁻: 451.1704. Found: 451.1642.

### Example 116

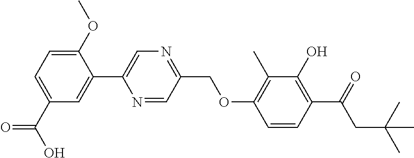 [SMILES: COc1ccc(C(=O)O)cc1-c1cnc(COc2ccc(C(=O)CC(C)(C)C)c(O)c2C)cn1]

### 3-(5-((4-(3,3-Dimethylbutanoyl)-3-hydroxy-2-methylphenoxy)methyl)pyrazin-2-yl)-4-methoxybenzoic acid

1-(4-((5-bromopyrazin-2-yl)methoxy)-2-hydroxy-3-methylphenyl)-3,3-dimethylbutan-1-one (0.280 g, 0.712 mmol), 3-borono-4-methoxybenzoic acid (0.209 g, 1.068 mmol), Pd(PPh₃)₄ (0.082 g, 0.071 mmol) and 2M Na₂CO₃ solution were processed according to general method F (Suzuki cross coupling reaction step). White solid (0.155 g, 47%). ¹H NMR (DMSO-d₆): δ 9.12 (s, 1H), 8.89 (s, 1H), 8.36 (s, 1H), 8.02 (d, J=8.7 Hz, 1H), 7.91 (d, J=9.2 Hz, 1H), 7.27 (d, J=9.2 Hz, 1H), 6.75 (d, J=9.2 Hz, 1H), 5.42 (s, 2H), 3.92 (s, 3H), 2.83 (s, 2H), 2.05 (s, 3H), 0.97 (s, 9H). ¹³C NMR (DMSO-d₆): δ 206.7, 163.5, 162.2, 149.9, 148.7, 144.4, 142.9, 132.2, 132.8, 131.7, 123.9, 115.5, 113.6, 104.1, 69.3, 56.2, 49.1, 31.6, 29.7, 8.3. LC-MS (ESI) m/z calcd for C₂₆H₂₈N₂O₆ [M+H]⁺: 465.19. Found: 465.15. HRMS (ESI) m/z calcd for C₂₆H₂₈N₂O₆ [M+H]⁺: 465.1920. Found: 465.2003.

### Example 117

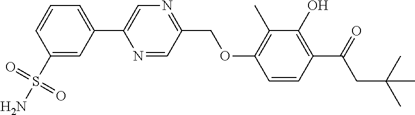 [SMILES: Cc1c(OCc2cnc(-c3cccc(S(N)(=O)=O)c3)cn2)ccc(C(=O)CC(C)(C)C)c1O]

### 3-(5-((4-(3,3-Dimethylbutanoyl)-3-hydroxy-2-methylphenoxy)methyl)pyrazin-2-yl)benzenesulfonamide

1-(4-((5-Bromopyrazin-2-yl)methoxy)-2-hydroxy-3-methylphenyl)-3,3-dimethylbutan-1-one (0.03 g, 0.1 mmol), (3-sulfamoylphenyl)boronic acid (0.03 g, 0.15 mmol), 2M Na₂CO₃ solution (0.2 mL) and Pd(PPh₃)₄ (1.56 mg) were processed according to the general method F (Suzuki cross coupling reaction step). White solid (0.028 g, 59.6%). ¹H NMR (DMSO-d₆): δ 9.31 (d, J=0.9 Hz, 1H), 8.91 (s, 1H), 8.60 (s, 1H), 8.35 (d, J=8.2 Hz, 1H), 7.92-7.89 (m, 2H), 7.72 (t, J=8.0 Hz, 1H), 7.46 (s, 2H), 6.75 (d, J=9.2 Hz, 1H), 5.44 (s, 2H), 2.83 (s, 2H), 2.05 (s, 3H), 0.97 (s, 9H). LC-MS (ESI) [M+H]⁺: 470.05. HRMS (ESI) m/z calcd for C₂₄H₂₇N₃O₅S [M+H]⁺: 470.197. Found: 470.1741.

### Example 118

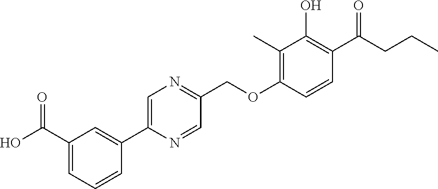 [SMILES: CCCC(=O)c1ccc(OCc2cnc(-c3cccc(C(=O)O)c3)cn2)c(C)c1O]

### 3-(5-((4-Butyryl-3-hydroxy-2-methylphenoxy)methyl)pyrazin-2-yl)benzoic acid

1-(4-((5-bromopyrazin-2-yl)methoxy)-2-hydroxy-3-methylphenyl)butan-1-one (0.365 g, 1 mmol), 3-boronobenzoic acid (0.249 g, 1.5 mmol), 2M Na₂CO₃ (2 mL) solution and Pd(PPh₃)₄ (12 mg) were processed according to the general method F (Suzuki cross coupling reaction step). White solid (0.249 g, 61.3%). ¹H NMR (DMSO-d₆): δ 9.30 (s, 1H), 8.86 (s, 1H), 8.67 (s, 1H), 8.36 (d, J=7.3 Hz, 1H), 8.02 (d, J=7.3 Hz, 1H), 7.82 (d, J=8.7 Hz, 1H), 7.64 (t, J=7.6 Hz, 1H), 6.75 (d, J=9.2 Hz, 1H), 5.41 (s, 2H), 2.94 (t, J=7.1 Hz, 2H), 2.04 (s, 3H), 1.62-1.55 (m, 2H), 0.89 (t, J=7.3 Hz, 3H). LC-MS (ESI) [M+H]⁺: 407.10. HRMS (ESI) m/z calcd for C₂₃H₂₂N₂O₅ [M−H]⁺: 405.1529. Found: 405.1452.

### Example 119

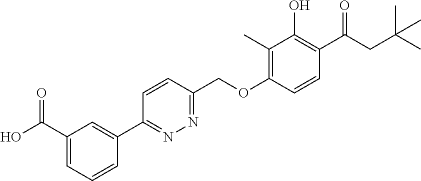 [SMILES: Cc1c(OCc2ccc(-c3cccc(C(=O)O)c3)nn2)ccc(C(=O)CC(C)(C)C)c1O]

### 3-(6-((4-(3,3-Dimethylbutanoyl)-3-hydroxy-2-methylphenoxy)methyl)pyridazin-3-yl)benzoic acid

1-(4-((6-bromopyridazin-3-yl)methoxy)-2-hydroxy-3-methylphenyl)-3,3-dimethylbutan-1-one (0.0.098 g, 0.25 mmol), 3-borono-4-methoxybenzoic acid (00.062 g, 0.375 mmol), Pd(PPh₃)₄ (3 mg) and 2M Na₂CO₃ solution (0.5 mL) were processed according to the general method F (Suzuki cross coupling reaction step). White solid (0.063 g, 58%). ¹H NMR (DMSO-d₆): δ 8.71 (s, 1H), 8.36 (d, J=8.7 Hz, 2H), 8.08 (d, J=7.8 Hz, 1H), 7.92 (dd, J=9.2, 1.8 Hz, 2H), 7.69 (t, J=7.8 Hz, 1H), 6.78 (d, J=9.2 Hz, 1H), 5.60 (s, 2H), 2.84 (s, 2H), 2.07 (s, 3H), 0.98 (s, 9H). LC-MS (ESI) [M+H]⁺: 435.15. HRMS (ESI) m/z calcd for C₂₅H₂₆N₂O₅ [M+H]⁺: 435.1842; Found: 435.1886.

### Example 120

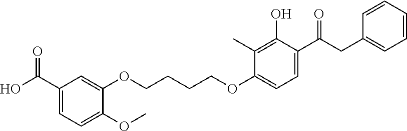 [SMILES: COc1ccc(C(=O)O)cc1OCCCCOc1ccc(C(=O)Cc2ccccc2)c(O)c1C]

### 3-(4-(3-Hydroxy-2-methyl-4-(2-phenylacetyl)phenoxy)butoxy)-4-methoxybenzoic acid

Methyl 3-hydroxy-4-methoxybenzoate, 1,4-dibromobutane, 2-phenylacetyl chloride, 2-methylbenzene-1,3-diol, K₂CO₃ and LiOH were processed according to general methods A, B, C and D. White solid (0.292 g, 63%). ¹H NMR (DMSO-d₆): δ 7.98 (d, J=9.1 Hz, 1H), 7.54 (d, J=8.2 Hz, 1H), 7.43 (s, 1H), 7.29-7.28 (m, 5H), 7.01 (d, J=8.2 Hz, 1H), 6.65 (d, J=9.1 Hz, 1H), 4.34 (s, 2H), 4.16-4.06 (m, 4H), 3.79 (s, 3H), 1.96 (s, 3H), 1.91-1.90 (m, 4H). ¹³C NMR (DMSO-d₆): δ 203.2, 165.3, 162.6, 161.4, 152.7, 147.5, 135.2, 130.8, 129.5, 128.4, 126.6, 123.1, 113.2, 112.8, 112.0, 111.1, 103.5, 67.9, 55.6, 42.1, 25.5, 7.5. LC-MS (ESI) Calcd for C₂₇H₂₈O₇ [M+H]⁺: 465.18. Found: 465.15.

### Example 121

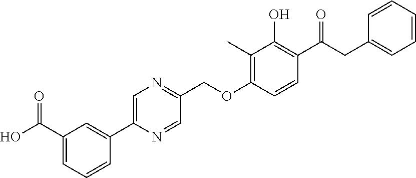 [SMILES: Cc1c(OCc2cnc(-c3cccc(C(=O)O)c3)cn2)ccc(C(=O)Cc2ccccc2)c1O]

### 3-(5-((3-Hydroxy-2-methyl-4-(2-phenylacetyl)phenoxy)methyl)pyrazin-2-yl)benzoic acid

To a solution of 1-(2,4-dihydroxy-3-methylphenyl)-2-phenylethan-1-one (0.061 g, 0.25 mmol), and 2-bromo-5-(bromomethyl)pyrazine (0.063 g, 0.25 mmol) in ACN (5 mL) was added potassium carbonate (0.069 g, 0.5 mmol). The resulting mixture was heated under reflux for 2 h, cooled, and filtered. Removal of the solvent in vacuo yielded the intermediate 1-(4-((5-bromopyrazin-2-yl)methoxy)-2-hydroxy-3-methylphenyl)-2-phenylethan-1-one, which was used without further purification in the next step. 1-(4-((5-Bromopyrazin-2-yl)methoxy)-2-hydroxy-3-methylphenyl)-2-phenylethan-1-one and 3-boronobenzoic acid (0.06 g, 0.375 mmol) were taken up in DME (5 mL) and heated at reflux for 5 h under nitrogen (g). The resulting mixture was heated overnight and cooled to room temperature, and acidified using 1N HCl. The aqueous phase was extracted with ethyl acetate (3×10 mL) and the combined organic layer was washed with brine, followed by drying over anhydrous Na₂SO₄. Filtration and removal of the solvent afforded crude product that was further purified by automated prep-HPLC to yield the title compound. White solid (0.075 g, 66%). ¹H NMR (DMSO-d₆): δ 9.29 (s, 1H), 8.87 (s, 1H), 8.68 (s, 1H), 8.36 (d, J=7.3 Hz, 1H), 8.01 (t, J=9.8 Hz, 1H), 7.64 (t, J=7.6 Hz, 2H), 7.30-7.19 (m, 5H), 6.79 (d, J=8.7 Hz, 1H), 5.44 (s, 2H), 4.33 (s, 2H), 2.04 (s, 3H). LC-MS (ESI) [M+H]⁺: 455.15. HRMS (ESI) m/z calcd for C₂₇H₂₈N₂O₄ [M−H]⁺: 453.1467. Found: 453.1390.

### Example 122

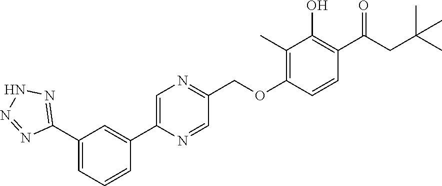 [SMILES: Cc1c(OCc2cnc(-c3cccc(-c4nn[nH]n4)c3)cn2)ccc(C(=O)CC(C)(C)C)c1O]

### 1-(4-((5-(3-(2H-Tetrazol-5-yl)phenyl)pyrazin-2-yl)methoxy)-2-hydroxy-3-methylphenyl)-3,3-dimethylbutan-1-one

3-(5-((4-(3,3-Dimethylbutanoyl)-3-hydroxy-2-methylphenoxy)methyl)pyrazin-2-yl)benzonitrile (0.12 g, 0.289 mmol), sodium azide (0.225 g, 3.47 mmol) and ammonium chloride (0.185 g, 3.47 mmol) were taken up in DMF (3 mL) and heated at 80° C. for 1 h. The reaction mixture was cooled, then quenched by the addition of NaHCO₃ solution, and washed with ethyl acetate. The aqueous phase was neutralized by the addition of dil. HCl and extracted with EtOAc. The combined organic phase was dried and evaporated to give the crude product, which was purified by reverse phase HPLC. White solid (0.086 g, 65%). ¹H NMR (DMSO-d₆): δ 9.34 (d, J=0.9 Hz, 1H), 8.91 (s, 1H), 8.81 (s, 1H), 8.33 (d, J=7.8 Hz, 1H), 8.13 (d, J=7.8 Hz, 1H), 7.90 (d, J=9.2 Hz, 1H), 7.75 (t, J=7.8 Hz, 1H), 6.75 (d, J=9.2 Hz, 1H), 5.44 (s, 2H), 2.82 (s, 2H), 2.05 (s, 3H), 0.97 (s, 9H). LCMS (ESI) [M+H]⁺: 459.00.

### Example 123

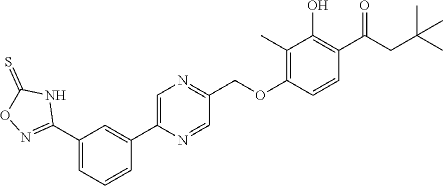 [SMILES: Cc1c(OCc2cnc(-c3cccc(-c4noc(=S)[nH]4)c3)cn2)ccc(C(=O)CC(C)(C)C)c1O]

### 1-(2-Hydroxy-3-methyl-4-((5-(3-(5-thioxo-4,5-dihydro-1,2,4-oxadiazol-3-yl)phenyl)pyrazin-2-yl)methoxy)phenyl)-3,3-dimethylbutan-1-one

A mixture of (Z)-3-(5-((4-(3,3-dimethylbutanoyl)-3-hydroxy-2-methylphenoxy)methyl)pyrazin-2-yl)-N′-hydroxybenzimidamide (0.140 g, 0.312 mmol), thiocarbonyl diimidazole (0.083 g, 0.468 mmol) and DBU (0.190 g, 1.249 mmol) in dioxane (5 mL) was heated at reflux for 3 h. After cooling the reaction mixture the solvent was removed under vacuum. The residue was diluted with water, the pH adjusted to 4-5 using dil. HCl, and then extracted with ethyl acetate. The combined organic extracts were concentrated in vacuo, the residue dissolved in 1N NaOH, and then washed with diethyl ether. The aqueous layer was adjusted to pH 4 with 1N HCl and extracted again with ethyl acetate. Removal of the solvent followed by reverse phase HPLC afforded the title compound as a white solid (0.106 g, 69%). ¹H-NMR (DMSO-d₆): δ 9.30 (s, 1H), 8.89 (s, 1H), 8.64 (s, 1H), 8.36 (d, J=7.8 Hz, 1H), 7.97 (d, J=8.2 Hz, 1H), 7.90 (d, J=9.2 Hz, 1H), 7.71 (t, J=7.8 Hz, 1H), 6.75 (d, J=9.2 Hz, 1H), 5.43 (s, 2H), 2.83 (s, 2H), 2.05 (s, 3H), 0.93 (s, 9H). LCMS (ESI) [M+H]⁺: 491.15.

### Example 124

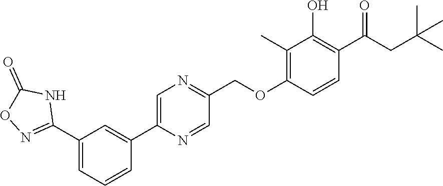 [SMILES: Cc1c(OCc2cnc(-c3cccc(-c4noc(=O)[nH]4)c3)cn2)ccc(C(=O)CC(C)(C)C)c1O]

### 3-(3-(5-((4-(3,3-Dimethylbutanoyl)-3-hydroxy-2-methylphenoxy)methyl)pyrazin-2-yl)phenyl)-1,2,4-oxadiazol-5(4H)-one

A mixture of (Z)-3-(5-((4-(3,3-dimethylbutanoyl)-3-hydroxy-2-methylphenoxy)methyl)pyrazin-2-yl)-N′-hydroxybenzimidamide (0.150 g, 0.334 mmol), carbonyl diimidazole (0.081 g, 0.502 mmol) and DBU (0.204 g, 1.338 mmol) in dioxane (5 mL) was heated at reflux for 3 h. After cooling the reaction mixture the solvent was removed under vacuum. The residue was diluted with water, the pH adjusted to 4-5 using dil. HCl, and then extracted with ethyl acetate. Removal of the solvent in vacuo followed by reverse phase HPLC afforded the title compound. White solid (0.106 g, 67%). ¹H NMR (DMSO-d₆): δ 9.31 (s, 1H), 8.91 (s, 1H), 8.58 (s, 1H), 8.36 (d, J=7.8 Hz, 1H), 7.91 (d, J=8.7 Hz, 2H), 7.72 (t, J=7.8 Hz, 1H), 6.76 (d, J=9.2 Hz, 1H), 5.43 (s, 2H), 2.84 (s, 2H), 2.06 (s, 3H), 0.94 (m, 9H). HRMS (ESI) m/z calcd for C₂₆H₂₆N₄O₅ [M−H]⁺: 475.19. Found: 475.15. LC-MS (ESI) m/z calcd for C₂₆H₂₆N₄O₅ [M+H]⁺: 473.1947. Found: 473.1875.

### Example 125

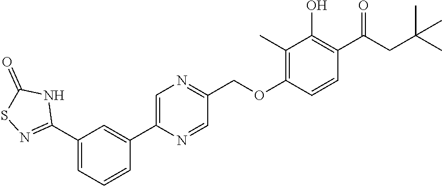 [SMILES: Cc1c(OCc2cnc(-c3cccc(-c4nsc(=O)[nH]4)c3)cn2)ccc(C(=O)CC(C)(C)C)c1O]

### 3-(3-(5-((4-(3,3-Dimethylbutanoyl)-3-hydroxy-2-methylphenoxy)methyl)pyrazin-2-yl)phenyl)-1,2,4-thiadiazol-5(4H)-one

A mixture of (Z)-3-(5-((4-(3,3-dimethylbutanoyl)-3-hydroxy-2-methylphenoxy)methyl)pyrazin-2-yl)-N′-hydroxybenzimidamide (0.150 g, 0.334 mmol), thiocarbonyl diimidazole (0.072 g, 0.401 mmol) THF (5 mL) was stirred at rt for 30 min. The mixture was diluted with water and extracted with EtOAc. The organic extract was washed with water and concentrated. The residue was dissolved in THF, BF₃etherate (0.237 g, 1.672 mmol) was added, and the reaction mixture stirred at rt for 1 h. The reaction mixture was diluted with water and extracted with EtOAc. The combined organic extracts were washed with 1N HCl and dried over Na₂SO₄. Removal of the solvent followed by reverse phase HPLC yielded the title compound. Yellow solid (0.102 g, 62%). ¹H NMR (DMSO-d₆): δ 9.30 (d, J=0.9 Hz, 1H), 8.88 (s, 1H), 8.69 (s, 1H), 8.29 (d, J=7.8 Hz, 1H), 8.03 (d, J=7.8 Hz, 1H), 7.89 (d, J=9.2 Hz, 1H), 7.66 (t, J=7.8 Hz, 1H), 6.76 (d, J=9.2 Hz, 1H), 5.42 (s, 2H), 2.82 (s, 2H), 2.04 (s, 3H), 1.00 (m, 9H).

### Example 126

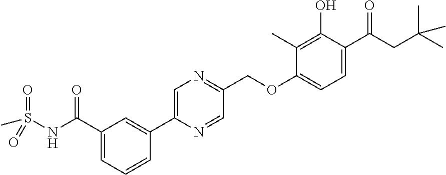 [SMILES: Cc1c(OCc2cnc(-c3cccc(C(=O)NS(C)(=O)=O)c3)cn2)ccc(C(=O)CC(C)(C)C)c1O]

### 3-(5-((4-(3,3-Dimethylbutanoyl)-3-hydroxy-2-methylphenoxy)methyl)pyrazin-2-yl)-N-(methylsulfonyl)benzamide

To a stirred solution of 3-(5-((4-(3,3-dimethylbutanoyl)-3-hydroxy-2-methylphenoxy)methyl)pyrazin-2-yl)benzoic acid (0.140 g, 0.322 mmol) and Et₃N (0.033 g, 0.322 mmol) in anhydrous ACN (5 mL) was added trichlorotriazine (0.018 g, 0.3 mmol) and alumina (9.86 mg, 0.097 mmol). The resulting mixture was stirred at rt for 5 min. A solution of methanesulfonamide (0.031 g, 0.322 mmol) and Et₃N (10.033 g, 0.322 mmol) in ACN (2 mL) was added and the resulting mixture was stirred at rt for 2 h. The reaction mixture was filtered and the filtrate was concentrated under vacuum. The resulting mixture was purified by reverse phase HPLC to yield the title compound. White solid (0.089 g, 54%).

### Example 127

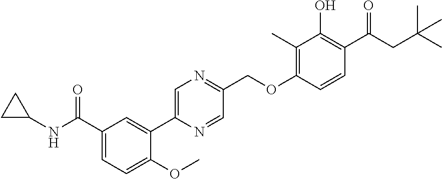 [SMILES: COc1ccc(C(=O)NC2CC2)cc1-c1cnc(COc2ccc(C(=O)CC(C)(C)C)c(O)c2C)cn1]

### N-Cyclopropyl-3-(5-((4-(3,3-dimethylbutanoyl)-3-hydroxy-2-methylphenoxy)methyl)pyrazin-2-yl)-4-methoxybenzamide

3-(5-((4-(3,3-Dimethylbutanoyl)-3-hydroxy-2-methylphenoxy)methyl)pyrazin-2-yl)-4-methoxybenzoic acid (0.1 g, 0.215 mmol) was dissolved in DMF (5 mL) at room temperature. HOBt (0.044 g, 0.323 mmol) was added in one portion followed by EDC (0.05 g, 0.323 mmol). The resulting mixture was stirred at room temperature for 30 min. Cyclopropylamine (0.015 g, 0.258 mmol) and triethylamine (0.028 g, 0.238 mmol) were added and the reaction mixture was stirred for 2 h. The organic phase was removed under reduced pressure and the crude material was partitioned between water and ethyl acetate. The aqueous layer was extracted with ethyl acetate (3×5 mL). The combined organic phase was dried using Na₂SO₄ and evaporated to give N-cyclopropyl-3-(5-((4-(3,3-dimethylbutanoyl)-3-hydroxy-2-methylphenoxy)methyl)pyrazin-2-yl)-4-methoxybenzamide. The crude product was purified by HPLC using acetonitrile:water as the solvent system to afford the amide as a colorless solid (0.087 g, 80%). LCMS (ESI) Calcd for C₂₉H₃₃N₃O₅ [M+H]⁺: 504.24. Found: 504.20. HRMS (ESI) Calcd for C₂₉H₃₃N₃O₅ [M−H]⁺: 502.2421. Found: 502.2367.

### Example 128

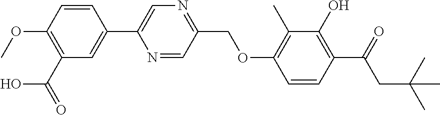 [SMILES: COc1ccc(-c2cnc(COc3ccc(C(=O)CC(C)(C)C)c(O)c3C)cn2)cc1C(=O)O]

### 5-(5-((4-(3,3-Dimethylbutanoyl)-3-hydroxy-2-methylphenoxy)methyl)pyrazin-2-yl)-2-methoxybenzoic acid

Prepared according to general method F using 1-(4-((5-Bromopyrazin-2-yl)methoxy)-2-hydroxy-3-methylphenyl)-3,3-dimethylbutan-1-one (0.150 g, 0.381 mmol), 5-borono-2-methoxybenzoic acid (0.112 g, 0.572 mmol), Pd(PPh₃)₄ (4.4 mg, 0.0038 mmol) and Na₂CO₃ (0.162 g, 1.526 mmol) were processed according to the general method F (Suzuki cross coupling reaction step). The reagents were taken up in DME (5 mL) and heated at reflux for 4 h under N₂ (g). The reaction mixture was cooled, diluted with water and acidified using dil. HCl. Following the standard work up with ethyl acetate the crude material was purified by reverse phase HPLC. White solid (0.079 g, 44.6). ¹H NMR (400 MHz, DMSO-D₆) δ 9.26 (d, J=1.4 Hz, 1H), 8.84 (d, J=1.8 Hz, 1H), 8.44 (d, J=2.3 Hz, 1H), 8.31 (dd, J=8.7, 2.3 Hz, 1H), 7.93 (d, J=9.2 Hz, 1H), 7.28 (d, J=8.7 Hz, 1H), 6.77 (d, J=9.2 Hz, 1H), 5.42 (s, 2H), 3.89 (s, 3H), 3.56 (s, 1H), 2.86 (s, 2H), 2.07 (s, 3H), 1.00 (s, 9H). ¹³C NMR (101 MHz, DMSO-D₆) δ 206.65, 167.61, 162.32, 162.14, 159.93, 150.31, 150.01, 143.23, 141.11, 131.77, 131.60, 129.53, 127.88, 122.77, 115.39, 113.53, 112.98, 104.13, 69.29, 66.88, 56.57, 49.10, 32.14, 30.39, 8.26. HRMS calcd for C₂₆H₂₉N₂O₆ [M+H]⁺: 465.20, found 465.20.

### Example 129

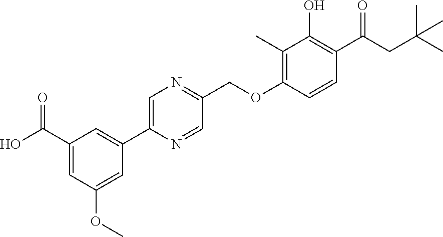 [SMILES: COc1cc(C(=O)O)cc(-c2cnc(COc3ccc(C(=O)CC(C)(C)C)c(O)c3C)cn2)c1]

### 3-(5-((4-(3,3-Dimethylbutanoyl)-3-hydroxy-2-methylphenoxy)methyl)pyrazin-2-yl)-5-methoxybenzoic acid

1-(4-((5-bromopyrazin-2-yl)methoxy)-2-hydroxy-3-methylphenyl)-3,3-dimethylbutan-1-one (0.150 g, 0.38 mmol), 3-borono-5-methoxybenzoic acid (0.112 g, 0.572 mmol), Pd(PPh₃)₄) and 2M Na₂CO₃ solution were processed according to the general method F (Suzuki cross coupling reaction step). White solid. (0.092 g, 51.9%). ¹H NMR (DMSO-d₆): δ 9.32 (d, J=1.4 Hz, 1H), 8.87 (d, J=1.4 Hz, 1H), 8.27 (t, J=1.4 Hz, 1H), 7.91-7.88 (m, 2H), 7.54-7.51 (m, 1H), 6.74 (d, J=9.2 Hz, 1H), 5.42 (s, 2H), 3.87 (s, 3H), 2.82 (s, 2H), 2.05 (s, 3H), 0.95 (s, 9H). LC-MS (ESI) [M+H]⁺: 465.00. HRMS (ESI) m/z calcd for C₂₉H₂₄O₆ [M+H]⁺: 465.1947. Found: 465.1979.

### Example 130

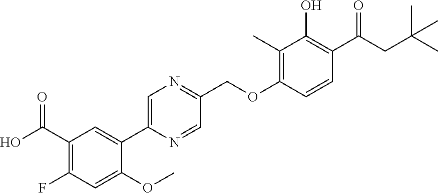 [SMILES: COc1cc(F)c(C(=O)O)cc1-c1cnc(COc2ccc(C(=O)CC(C)(C)C)c(O)c2C)cn1]

### 5-(5-((4-(3,3-Dimethylbutanoyl)-3-hydroxy-2-methylphenoxy)methyl)pyrazin-2-yl)-2-fluoro-4-methoxybenzoic acid

1-(2-Hydroxy-3-methyl-4-((5-(trimethylstannyl)pyrazin-2-yl)methoxy)phenyl)-3,3-dimethylbutan-1-one (0.095 g, 0.2 mmol), 5-bromo-2-fluoro-4-methoxybenzoic acid (0.060 g, 0.240 mmol) and Pd(PPh₃)₄ (0.023 g, 0.0220 mmol) were taken up in DME (5 mL) and heated at reflux for 12 h under an atmosphere of nitrogen. The reaction mixture was cooled to rt, the solvent removed under reduced pressure, and purified by reverse phase HPLC. Light yellow solid (0.017 g, 17.62%). ¹H NMR (DMSO-d₆): δ 9.09 (d, J=1.4 Hz, 1H), 8.88 (d, J=1.4 Hz, 1H), 8.64 (d, J=0.9 Hz, 1H), 8.53 (d, J=0.9 Hz, 1H), 7.17 (d, J=12.8 Hz, 1H), 6.73 (dd, J=15.1, 9.2 Hz, 1H), 5.31 (s, 2H), 3.93 (s, 3H), 2.83 (s, 2H), 1.97 (s, 3H), 0.95 (m, 9H). LC-MS (ESI) [M+H]⁺: 483.00. HRMS (ESI) m/z calcd for C₂₆H₂₇FN₂O₆ [M+H]⁺: 483.1863. Found: 483.1922.

### Example 131

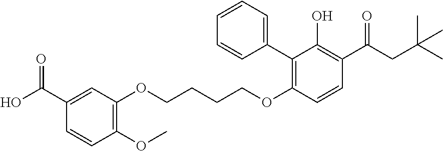 [SMILES: COc1ccc(C(=O)O)cc1OCCCCOc1ccc(C(=O)CC(C)(C)C)c(O)c1-c1ccccc1]

### 3-(4-((5-(3,3-Dimethylbutanoyl)-6-hydroxy-[1,1′-biphenyl]-2-yl)oxy)butoxy)-4-methoxybenzoic acid

1-(2,6-Dihydroxy-[1,1′-biphenyl]-3-yl)-3,3-dimethylbutan-1-one, methyl 3-(4-bromobutoxy)-4-methoxybenzoate, potassium carbonate and lithium hydroxide were processed according to general method B, C and D. White solid (0.178 g, 70.3%). ¹H NMR (DMSO-d₆): δ 8.03 (d, J=9.2 Hz, 1H), 7.52-7.50 (m, 1H), 7.36-7.20 (m, 7H), 6.98 (d, J=8.2 Hz, 1H), 6.72 (d, J=8.7 Hz, 1H), 4.31-3.90 (m, 4H), 3.75 (s, 3H), 2.86 (s, 2H), 1.69 (s, 3H), 0.98 (s, 9H). ¹³C-NMR (DMSO-d₆) δ 206.6, 167.7, 162.4, 161.7, 153.2, 148.0, 133.8, 133.2, 131.3, 128.0, 127.3, 123.8, 123.7, 118.0, 115.3, 113.7, 111.6, 104.4, 68.5, 68.3, 56.2, 49.2, 32.2, 30.4, 25.8, 25.6. LC-MS (ESI) [M+H]⁺: 507.10.

### Example 132

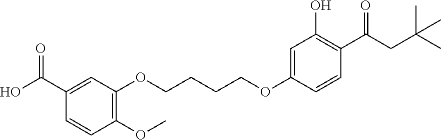 [SMILES: COc1ccc(C(=O)O)cc1OCCCCOc1ccc(C(=O)CC(C)(C)C)c(O)c1]

### 3-(4-(4-(3,3-Dimethylbutanoyl)-3-hydroxyphenoxy)butoxy)-4-methoxybenzoic acid

1-(2,4-Dihydroxyphenyl)-3,3-dimethylbutan-1-one, methyl 3-(4-bromobutoxy)-4-methoxybenzoate, potassium carbonate and lithium hydroxide were processed according to the general methods B, C and D. White solid (0.17 g, 79%). ¹H NMR (DMSO-d₆): δ 7.88 (d, J=8.7 Hz, 1H), 7.52 (dd, J=8.2, 1.8 Hz, 1H), 7.41 (d, J=1.8 Hz, 1H), 7.00 (d, J=8.2 Hz, 1H), 6.48-6.42 (m, 2H), 4.10-4.02 (m, 4H), 3.78 (s, 3H), 2.80 (s, 2H), 1.92-1.85 (overlapping singlet and multiplets, 5H), 0.97 (s, 9H). ¹³C-NMR (DMSO-d₆): δ 205.9, 167.6, 165.7, 165.3, 153.3, 148.1, 134.1, 123.7, 123.4, 115.0, 113.6, 111.6, 108.0, 101.7, 68.4, 68.4, 56.2, 49.2, 32.1, 30.4, 25.9, 25.8. LC-MS (ESI) [M+H]⁺: 431.20.

### Example 133

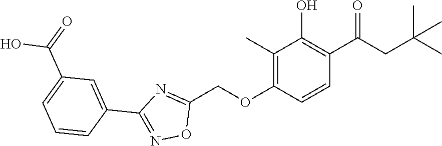 [SMILES: Cc1c(OCc2nc(-c3cccc(C(=O)O)c3)no2)ccc(C(=O)CC(C)(C)C)c1O]

### 3-(5-((4-(3,3-Dimethylbutanoyl)-3-hydroxy-2-methylphenoxy)methyl)-1,2,4-oxadiazol-3-yl)benzoic acid

To a solution of 1-(2,4-dihydroxy-3-methylphenyl)-3,3-dimethylbutan-1-one (0.111 g, 0.5 mmol) and methyl 3-(5-(chloromethyl)-1,2,4-oxadiazol-3-yl)benzoate (0.126 g, 0.5 mmol) in ACN (10 mL) was added potassium carbonate (0.138 g, 1 mmol). The reaction mixture was heated at reflux for 2 h. Following cooling to rt, filtration and evaporation of the solvents yielded the crude product which was used without further purification for the next step. The crude ester was taken in dioxane (5 mL) and 2M LiOH solution (1.25 mL) was added. The resulting mixture was heated at reflux for 30 min. The reaction was quenched with HCl (5% aq.) and ethyl acetate (10 mL) was added. The organic layer was separated and dried over Na₂SO₄. The solvents were removed in vacuo, the product was isolated by reverse phase HPLC and lyophilized to provide the title compound. White solid (0.149 g, 70.2%). ¹H NMR (DMSO-d₆): δ 8.58 (t, J=1.6 Hz, 1H), 8.27 (dt, J=7.9, 1.4 Hz, 1H), 8.19 (dt, J=7.8, 1.4 Hz, 1H), 7.98 (d, J=9.2 Hz, 1H), 7.75 (t, J=7.8 Hz, 1H), 6.80 (d, J=9.2 Hz, 1H), 5.81 (s, 2H), 2.90 (s, 2H), 2.12 (s, 3H), 1.01 (d, J=14.7 Hz, 9H). LC-MS: 425.0 [M+H]⁺.

### Example 134

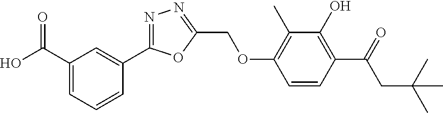 [SMILES: Cc1c(OCc2nnc(-c3cccc(C(=O)O)c3)o2)ccc(C(=O)CC(C)(C)C)c1O]

### 3-(5-((4-(3,3-Dimethylbutanoyl)-3-hydroxy-2-methylphenoxy)methyl)-1,3,4-oxadiazol-2-yl)benzoic acid

A mixture of 2-(4-(3,3-dimethylbutanoyl)-3-hydroxy-2-methylphenoxy)acetic acid (0.28 g, 1 mmol), methyl 3-(hydrazinecarbonyl)benzoate (0.194 g, 1 mmol) and POCl₃ (2 mL) was heated under reflux for 2 h. The excess POCl₃ was removed under reduced pressure and the remainder was poured onto crushed ice and extracted with ethyl acetate, washed with brine and dried over anhydrous sodium sulfate. Removal of the solvent yielded the crude methyl ester, which was taken up in dioxane (5 mL) and 2M LiOH solution (2.5 mL) was added. The resulting mixture was heated at reflux for 30 min, the reaction was quenched with HCl (5% aq.) and ethyl acetate (10 mL) was added. The organic layer was separated and dried over Na₂SO₄. The solvents were removed in vacuo, the product was isolated by reverse phase HPLC and lyophilized to provide the final compound. White solid (0.149 g, 70.2%). LC-MS: 425.0 [M+H]⁺.

### Example 135

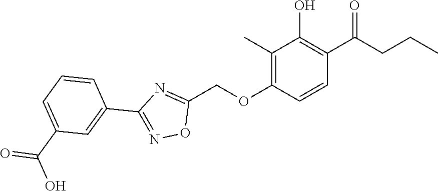 [SMILES: CCCC(=O)c1ccc(OCc2nc(-c3cccc(C(=O)O)c3)no2)c(C)c1O]

### 3-(5-((4-Butyryl-3-hydroxy-2-methylphenoxy)methyl)-1,2,4-oxadiazol-3-yl)benzoic acid

To a solution of 1-(2,4-dihydroxy-3-methylphenyl)butan-1-one (0.0.097 g, 0.5 mmol) and methyl 3-(5-(chloromethyl)-1,2,4-oxadiazol-3-yl)benzoate (0.126 g, 0.5 mmol) in ACN (10 mL) was added potassium carbonate (0.138 g, 1 mmol). The reaction mixture was heated at reflux for 2 h. Following cooling of the reaction mixture, filtration and evaporation of solvents yielded the crude product which was used without further purification for the next step. The crude ester was taken up in dioxane (5 mL), 2M LiOH solution (1.25 mL) was added, and the resulting mixture was heated at reflux for 30 min. The reaction was quenched with HCl (5% aq.) and ethyl acetate (10 mL) was added. The organic layer was separated and dried over Na₂SO₄. The solvents were removed in vacuo and the products were isolated by reverse phase HPLC and lyophilized to provide the final compound. White solid (0.149 g, 70.2%). ¹H NMR (DMSO-d₆): δ 8.49 (t, J=1.6 Hz, 1H), 8.18 (dt, J=7.9, 1.6 Hz, 1H), 8.09 (dt, J=7.8, 1.4 Hz, 1H), 7.82 (d, J=9.2 Hz, 1H), 7.66 (t, J=7.8 Hz, 1H), 6.73 (d, J=9.2 Hz, 1H), 5.71 (s, 2H), 2.93 (t, J=7.3 Hz, 2H), 2.00 (s, 3H), 1.58 (q, J=7.3 Hz, 2H), 0.87 (t, J=7.3 Hz, 3H). ¹³C NMR (DMSO-d₆): δ 206.3, 175.9, 167.3, 166.6, 161.3, 160.8, 132.4, 132.0, 131.1, 130.2, 130.0, 127.8, 126.2, 114.1, 112.9, 103.6, 61.5, 17.7, 13.6, 7.7. LC-MS: 397.0 [M+H]⁺.

### Example 136

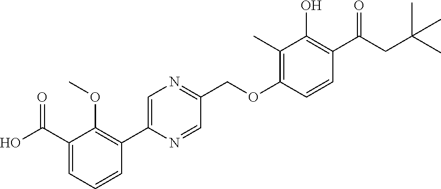

### 3-(5-((4-(3,3-Dimethylbutanoyl)-3-hydroxy-2-methylphenoxy)methyl)pyrazin-2-yl)-2-methoxybenzoic acid

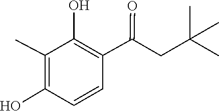 [SMILES: Cc1c(O)ccc(C(=O)CC(C)(C)C)c1O]

### Step 1: 1-(2,4-Dihydroxy-3-methylphenyl)-3,3-dimethylbutan-1-one

A solution of 3,3-dimethylbutanoyl chloride (6.7 g, 50 mmol, 1 equiv) in CH₂Cl₂ (50 mL) was added dropwise to a stirred solution of AlCl₃ (6.7 g, 50 mmol, 1 equiv) in CH₂Cl₂ (500 mL) at 0° C. under nitrogen. 2-Methylresorcinol (6.2 g, 50 mmol, 1 equiv) was added to the reaction mixture, and the reaction was gradually warmed to room temperature and stirred at room temperature for 12 h. The pre-cooled reaction mixture was quenched by the dropwise addition of HCl (5% aq.) and diluted with water. The organic layer was separated, and the aqueous layer extracted with CH₂Cl₂ (3×100 mL) and the combined organic extracts were washed with water, brine and dried over anhydrous sodium sulfate, filtered and concentrated under reduced pressure to obtain the crude product, which was filtered with hexane and dried under vacuum to yield a tan solid (9.5 g, 85%). Purification by silica gel column chromatography provided the title compound as a white solid, LC-MS ESI m/z: 223 [M+H]⁺.

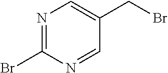

### Step 2: 2-Bromo-5-(bromomethyl)pyrazine

To a stirred solution of 2-bromo-5-methylpyrazine (2.5 g, 14.45 mmol) in carbon tetrachloride (100 mL) under nitrogen atmosphere were added NBS (2.83 g, 15.89 mmol) and AIBN (0.118 g, 0.722 mmol) and the resulting mixture was heated at 75° C. for 16 h. After cooling, the precipitated succinimide was removed by filtration. The filtrate was concentrated in vacuo to afford the crude product. The crude material was purified by silica gel column chromatography (Hexanes-10% ethyl acetate in hexanes) to provide the title compound as a clear liquid (2.46 g, 68%) which solidified at lower temperature. The 2-bromo-5-(bromomethyl)pyrazine produced with this method was used immediately for the next step.

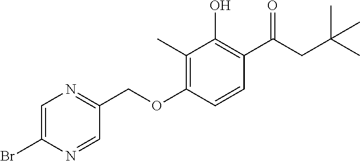 [SMILES: Cc1c(OCc2cnc(Br)cn2)ccc(C(=O)CC(C)(C)C)c1O]

### Step 3: 1-(4-((5-Bromopyrazin-2-yl)methoxy)-2-hydroxy-3-methylphenyl)-3,3-dimethylbutan-1-one

To a stirred solution of 1-(2,4-dihydroxy-3-methylphenyl)-3,3-dimethylbutan-1-one (1.8 g, 8 mmol) in acetonitrile (100 mL) under an inert atmosphere were added 2-bromo-5-(bromomethyl)pyrazine (2.42 g, 9.6 mmol) and potassium carbonate (2.2 g, 16 mmol). The reaction mixture was heated to 80° C. and stirred for 2-4 h. The progress of the reaction was monitored by LC-MS. After completion of the reaction, the volatiles were removed under reduced pressure, the reaction mixture was diluted with water (100 mL) and extracted with CH₂Cl₂ (3×100 mL). The combined organic extracts were washed with water, then brine, dried over sodium sulfate, filtered and concentrated under reduced pressure to obtain the crude product. White solid (2.94 g, 93%). The crude product was used in the next step without further purification.

### Step 4: 3-(5-((4-(3,3-Dimethylbutanoyl)-3-hydroxy-2-methylphenoxy)methyl)pyrazin-2-yl)-2-methoxybenzoic acid

3-Borono-2-methoxybenzoic acid (1.5 g, 7.6 mmol) was added to a stirred solution of 1-(4-((5-bromopyrazin-2-yl)methoxy)-2-hydroxy-3-methylphenyl)-3,3-dimethylbutan-1-one (2 g, 5.1 mmol) in dimethoxyethane (50 mL) under a nitrogen atmosphere. The reaction mixture was purged with nitrogen for 15 min. and then Pd(dppf)₂Cl₂ (0.093 g, 0.127 mmol) and 2M sodium carbonate solution (10 mL) were added. The mixture was heated to 80° C. and stirred under inert atmosphere overnight. The hot suspension was filtered and concentrated to half of the original volume and washed with dichloromethane. The aqueous phase was adjusted to pH=1 with hydrochloric acid (1 M) and the precipitated product was collected by filtration and washed with water and dried. The crude product was purified by silica gel column chromatography [Eluent: CH₂Cl₂ to 20% CH₃OH in CH₂Cl₂] to afford the title compound as a white solid (2 g, 85%). ¹H NMR (DMSO-d₆): δ 9.03 (S, 1H), 8.91 (S, 1H), 7.91 (d, J=9.2 Hz, 1H), 7.85 (dd, J=7.8, 1.8 Hz, 1H), 7.78 (dd, J=7.8, 1.8 Hz, 1H), 7.78 (dd, J=7.8, 1.8 Hz, 1H), 7.32 (t, J=7.8 Hz, 1H), 6.76 (d, J=9.2 Hz, 1H), 5.43 (s, 2H), 3.58 (s, 3H), 2.83 (s, 2H), 2.06 (s, 3H), 0.94 (s, 9H). LC-MS (ESI) [M+H]⁺: 465.10. HRMS (ESI) m/z calcd for C₂₆H₂₈N₂O₆ [M+H]⁺: 465.19439. Found: 465.1999.

### Example 137

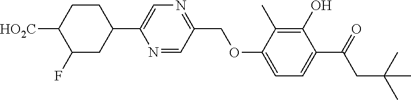 [SMILES: Cc1c(OCc2cnc(C3CCC(C(=O)O)C(F)C3)cn2)ccc(C(=O)CC(C)(C)C)c1O]

### 4-(5-((4-(3,3-Dimethylbutanoyl)-3-hydroxy-2-methylphenoxy)methyl)pyrazin-2-yl)-2-fluorobenzoic acid

A 2M Na₂CO₃ solution (1 mL) was added to a mixture of 1-(4-((5-bromopyrazin-2-yl)methoxy)-2-hydroxy-3-methylphenyl)-3,3-dimethylbutan-1-one (0.197 g, 0.5 mmol), 4-borono-2-fluorobenzoic acid (0.138 g, 0.75 mmol) and tetrakistriphenylphosphinepalladium(0) (0.057 g, 0.05 mmol) in DME (6 mL). The resulting solution was heated at reflux under an atmosphere of N₂ (g) for 2 h. The reaction mixture was cooled to room temperature, diluted with water and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude product which was further purified by automated prep-HPLC to yield the title compound as a white solid (0.175 g, 74.6%). ¹H NMR (400 MHz, DMSO-d₆) δ 9.35 (d, J=1.5 Hz, 2H), 8.90 (d, J=1.4 Hz, 2H), 8.12-8.03 (m, 3H), 6.74 (d, J=9.1 Hz, 1H), 5.44 (s, 2H), 2.83 (s, 2H), 2.06 (s, 2H), 0.98 (s, 9H). LC-MS (ESI) Calcd for C₂₅H₂₅FN₂O₅ [M+H]⁺: 453.17. Found: 453.20.

### Example 138

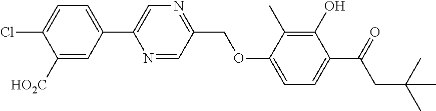 [SMILES: Cc1c(OCc2cnc(-c3ccc(Cl)c(C(=O)O)c3)cn2)ccc(C(=O)CC(C)(C)C)c1O]

### 2-Chloro-5-(5-((4-(3,3-Dimethylbutanoyl)-3-hydroxy-2-methylphenoxy)methyl)pyrazin-2-yl)benzoic acid

A 2M Na₂CO₃ solution (1 mL) was added to a mixture of 1-(4-((5-bromopyrazin-2-yl)methoxy)-2-hydroxy-3-methylphenyl)-3,3-dimethylbutan-1-one (0.197 g, 0.5 mmol), 5-borono-2-chlorobenzoic acid (0.150 g, 0.75 mmol) and tetrakistriphenylphosphinepalladium(0) (0.057 g, 0.05 mmol) in DME (6 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude product that was further purified by automated prep-HPLC to yield the title compound. White solid (0.175 g, 74.6%). ¹H NMR (400 MHz, DMSO-d₆) δ 9.33 (d, J=2.6 Hz, 1H), 8.88 (d, J=3.0 Hz, 1H), 8.54 (t, J=2.8 Hz, 1H), 8.29-7.71 (m, 3H), 6.76 (dt, J=9.1, 2.6 Hz, 1H), 5.45 (s, 2H), 2.84 (s, 2H), 2.06 (s, 3H), 0.99 (s, 9H). LC-MS (ESI) Calcd for C₂₅H₂₅ClN₂O₅ [M+H]⁺: 469.15. Found: 469.00.

### Example 139

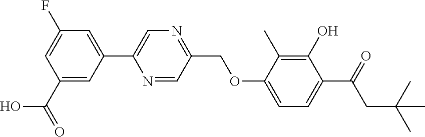 [SMILES: Cc1c(OCc2cnc(-c3cc(F)cc(C(=O)O)c3)cn2)ccc(C(=O)CC(C)(C)C)c1O]

### 3-(5-((4-(3,3-Dimethylbutanoyl)-3-hydroxy-2-methylphenoxy)methyl)pyrazin-2-yl)-5-fluorobenzoic acid

A 2M Na₂CO₃ solution (1 mL) was added to a mixture of 1-(4-((5-bromopyrazin-2-yl)methoxy)-2-hydroxy-3-methylphenyl)-3,3-dimethylbutan-1-one (0.197 g, 0.5 mmol), 3-borono-5-fluoro benzoic acid (0.137 g, 0.75 mmol) and tetrakistriphenylphosphinepalladium(0) (0.057 g, 0.05 mmol) in DME (6 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude product that was further purified by automated prep-HPLC to yield the title compound. White solid (0.135 g, 59.7%). ¹H NMR (400 MHz, DMSO-d₆) δ 9.34 (s, 1H), 8.89 (s, 1H), 8.23 (d, J=9.6 Hz, 1H), 7.88 (d, J=9.2 Hz, 1H), 7.74 (d, J=8.2 Hz, 1H), 6.73 (d, J=9.2 Hz, 1H), 5.41 (s, 2H), 2.81 (s, 2H), 2.06 (s, 3H), 0.99 (s, 9H). LC-MS (ESI) Calcd for C₂₅H₂₅FN₂O₅ [M+H]⁺: 453.17. Found: 453.00.

### Example 140

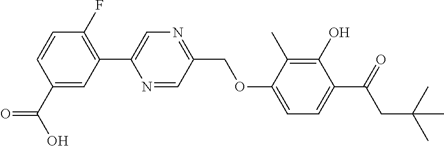 [SMILES: Cc1c(OCc2cnc(-c3cc(C(=O)O)ccc3F)cn2)ccc(C(=O)CC(C)(C)C)c1O]

### 3-(5-{[4-(3,3-Dimethylbutanoyl)-3-hydroxy-2-methylphenoxy]methyl}pyrazin-2-yl)-4-fluorobenzoic acid

A 2M Na₂CO₃ solution (1 mL) was added to a mixture of 1-(4-((5-bromopyrazin-2-yl)methoxy)-2-hydroxy-3-methylphenyl)-3,3-dimethylbutan-1-one (0.097 g, 0.25 mmol), 3-borono-4-fluoro benzoic acid (0.069 g, 0.375 mmol) and tetrakistriphenylphosphinepalladium(0) (0.029 g, 0.025 mmol) in DME (6 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude product that was further purified by automated prep-HPLC to yield the title compound. White solid (0.058 g, 51%). ¹H NMR (400 MHz, DMSO-d₆) δ 9.35 (d, J=1.6 Hz, 1H), 8.89 (d, J=1.4 Hz, 1H), 8.54 (t, J=1.5 Hz, 1H), 8.24 (d, J=9.8 Hz, 1H), 7.89 (d, J=9.1 Hz, 1H), 7.75 (d, J=7.7 Hz, 1H), 6.74 (d, J=9.1 Hz, 1H), 5.43 (s, 2H), 2.82 (s, 2H), 2.05 (s, 3H), 0.98 (s, 9H). LC-MS (ESI) Calcd for C₂₅H₂₅FN₂O₅[M+H]⁺: 453.17. Found: 453.20.

### Example 141

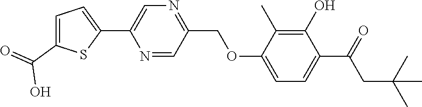 [SMILES: Cc1c(OCc2cnc(-c3ccc(C(=O)O)s3)cn2)ccc(C(=O)CC(C)(C)C)c1O]

### 5-(5-((4-(3,3-Dimethylbutanoyl)-3-hydroxy-2-methylphenoxy)methyl)pyrazin-2-yl)thiophene-2-carboxylic acid

A 2M Na₂CO₃ solution (0.5 mL) was added to a mixture of 1-(4-((5-bromopyrazin-2-yl)methoxy)-2-hydroxy-3-methylphenyl)-3,3-dimethylbutan-1-one (0.100 g, 0.254 mmol), 5-boronothiophene-2-carboxylic acid (0.066 g, 0.381 mmol) and tetrakistriphenylphosphinepalladium(0) (0.029 g, 0.025 mmol) in DME (4 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude product that was further purified by automated prep-HPLC to yield the desired compound. Light yellow solid (0.049 g, 44%). ¹H NMR (400 MHz, DMSO-d₆) δ 9.32 (d, J=1.5 Hz, 1H), 8.79 (d, J=1.4 Hz, 1H), 8.02 (d, J=4.0 Hz, 1H), 7.91 (d, J=9.1 Hz, 1H), 7.78 (d, J=3.9 Hz, 1H), 6.75 (d, J=9.1 Hz, 1H), 5.41 (s, 2H), 2.84 (s, 2H), 2.06 (s, 3H), 0.99 (s, 9H). LC-MS (ESI) Calcd for C₂₃H₂₄N₂O₅S [M+H]⁺: 441.14. Found: 441.15.

### Example 142

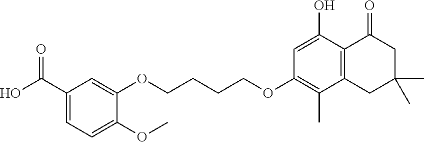 [SMILES: COc1ccc(C(=O)O)cc1OCCCCOc1cc(O)c2c(c1C)CC(C)(C)CC2=O]

### 4-Methoxy-3-(4-((2,2,8-trimethyl-4-oxochroman-7-yl)oxy)butoxy)benzoic acid

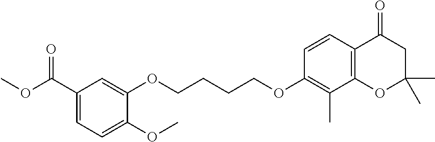

### Step 1: Methyl 4-methoxy-3-(4-((2,2,8-trimethyl-4-oxochroman-7-yl)oxy)butoxy)benzoate

To a stirred solution of 1-(2,4-dihydroxy-3-methylphenyl)-3-methylbut-2-en-1-one (0.103 g, 0.5 mmol) and methyl 3-(4-bromobutoxy)-4-methoxybenzoate (0.159 g, 0.5 mmol) in CAN (10 mL) was added potassium carbonate (0.138 g, 1 mmol). The resulting mixture was heated at 80° C. under an inert atmosphere for 2 h. The precipitated solids were filtered off and the solvent removed from the filtrate under reduced pressure. The crude product was partitioned between water and ethyl acetate. The organic layer was collected, and the aqueous layer extracted with ethyl acetate twice. The combined organic layers were washed with water, then brine, dried over anhydrous sodium sulfate, filtered and concentrated under reduced pressure to obtain the title compound which was used in the next step without further purification.

### Step 2: 4-Methoxy-3-(4-((2,2,8-trimethyl-4-oxochroman-7-yl)oxy)butoxy)benzoic acid

To a solution of methyl 4-methoxy-3-(4-((2,2,8-trimethyl-4-oxochroman-7-yl)oxy)butoxy)benzoate (0.5 mmol) in dioxane (10 mL) was added 2M LiOH aq. solution (1.25 mL, 2.5 mmol), and the resulting mixture was heated at 50° C. until the starting material was consumed (˜1 h). After completion of the reaction, the reaction mixture was cooled to rt and diluted with water. The pH was adjusted to 1 by the addition of 1N HCl, and ethyl acetate (50 mL) was added. The organic layer was separated and dried over Na₂SO₄. The solvents were removed by rotary evaporation and the product was purified by reverse phase HPLC and lyophilized to provide the title compound. White solid (0.176 g, 82.2%). ¹H NMR (400 MHz, CHLOROFORM-D) δ 7.68 (dd, J=8.5, 2.1 Hz, 1H), 7.62 (d, J=9.2 Hz, 1H), 7.55 (d, J=2.3 Hz, 1H), 6.88 (d, J=8.2 Hz, 1H), 6.72-6.65 (m, 1H), 6.41 (d, J=9.2 Hz, 1H), 4.15 (dt, J=11.9, 6.0 Hz, 4H), 3.90 (d, J=6.9 Hz, 6H), 2.17 (d, J=1.4 Hz, 3H), 2.10 (s, 3H), 2.09-2.03 (m, 3H), 2.01-2.00 (d, J=1.8 Hz, 3H). HRMS calcd for C₂₄H₂₈O₇ [M+H]⁺: 429.19, found 429.60.

### Example 143

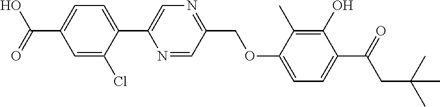 [SMILES: Cc1c(OCc2cnc(-c3ccc(C(=O)O)cc3Cl)cn2)ccc(C(=O)CC(C)(C)C)c1O]

### 3-Chloro-4-(5-((4-(3,3-Dimethylbutanoyl)-3-hydroxy-2-methylphenoxy)methyl)pyrazin-2-yl)benzoic acid

A 2M Na₂CO₃ solution (0.5 mL) was added to a mixture of 1-(4-((5-bromopyrazin-2-yl)methoxy)-2-hydroxy-3-methylphenyl)-3,3-dimethylbutan-1-one (0.100 g, 0.254 mmol), 4-borono-3-chlorobenzoic acid (0.076 g, 0.381 mmol) and tetrakistriphenylphosphinepalladium(0) (0.029 g, 0.025 mmol) in DME (4 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded crude product that was further purified by automated prep-HPLC to yield the desired compound. White solid (0.078 g, 65%). ¹H NMR (400 MHz, DMSO-d₆) δ 8.99 (d, J=8.9 Hz, 2H), 8.07 (s, 1H), 8.02 (d, J=7.9 Hz, 1H), 7.94 (s, 1H), 7.80 (d, J=10.4 Hz, 1H), 6.79 (d, J=8.7 Hz, 1H), 5.48 (s, 2H), 2.86 (s, 2H), 2.08 (s, 3H), 1.00 (s, 9H). LC-MS (ESI) Calcd for C₂₅H₂₅FN₂O₅ [M+H]⁺: 469.15. Found: 469.15.

### Example 144

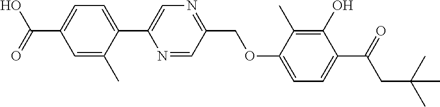 [SMILES: Cc1cc(C(=O)O)ccc1-c1cnc(COc2ccc(C(=O)CC(C)(C)C)c(O)c2C)cn1]

### 4-(5-{[4-(3,3-Dimethylbutanoyl)-3-hydroxy-2-methylphenoxy]methyl}pyrazin-2-yl)-3-methylbenzoic acid

A 2M Na₂CO₃ solution (0.5 mL) was added to a mixture of 1-(4-((5-bromopyrazin-2-yl)methoxy)-2-hydroxy-3-methylphenyl)-3,3-dimethylbutan-1-one (0.100 g, 0.254 mmol), 4-borono-3-methylbenzoic acid (0.068 g, 0.381 mmol) and tetrakistriphenylphosphinepalladium(0) (0.029 g, 0.025 mmol) in DME (4 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded crude product that was further purified by automated prep-HPLC to yield the title compound. White solid (0.081 g, 71%). ¹H NMR (400 MHz, DMSO-d₆) δ 8.92 (d, J=5.7 Hz, 2H), 8.03 (d, J=2.0 Hz, 1H), 7.98-7.90 (m, 2H), 7.50 (d, J=7.9 Hz, 1H), 6.80 (d, J=9.0 Hz, 1H), 5.47 (s, 2H), 2.86 (s, 2H), 2.43 (s, 3H), 2.09 (s, 3H), 1.00 (s, 9H). LC-MS (ESI) Calcd for C₂₆H₂₈N₂O₅ [M+H]⁺: 449.20. Found: 449.00.

### Example 145

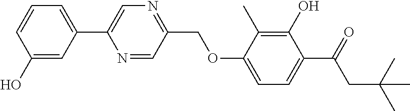 [SMILES: Cc1c(OCc2cnc(-c3cccc(O)c3)cn2)ccc(C(=O)CC(C)(C)C)c1O]

### 1-(2-Hydroxy-4-{[5-(3-hydroxyphenyl)pyrazin-2-yl]methoxy}-3-methylphenyl)-3,3-dimethylbutan-1-one

A 2M Na₂CO₃ solution (0.5 mL) was added to a mixture of 1-(4-((5-bromopyrazin-2-yl)methoxy)-2-hydroxy-3-methylphenyl)-3,3-dimethylbutan-1-one (0.100 g, 0.254 mmol), (3-hydroxyphenyl)boronic acid (0.052 g, 0.381 mmol) and tetrakistriphenylphosphinepalladium(0) (0.029 g, 0.025 mmol) in DME (4 mL). The resulting solution was heated under reflux in an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded crude product that was further purified by automated prep-HPLC to yield the title compound. White solid (0.069 g, 67%). ¹H NMR (400 MHz, DMSO-d₆) δ 9.77 (s, 1H), 8.85 (d, J=1.5 Hz, 1H), 7.92 (d, J=9.1 Hz, 1H), 7.58-7.52 (m, 2H), 7.33 (t, J=7.9 Hz, 1H), 6.90 (dd, J=7.9, 2.5 Hz, 1H), 6.78 (s, 1H), 5.42 (s, 2H), 2.85 (s, 2H), 2.07 (s, 3H), 1.00 (s, 9H). LC-MS (ESI) Calcd for C₂₄H₂₆N₂O₄ [M+H]⁺: 407.19. Found: 407.00.

### Example 146

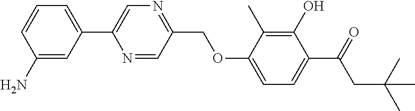 [SMILES: Cc1c(OCc2cnc(-c3cccc(N)c3)cn2)ccc(C(=O)CC(C)(C)C)c1O]

### 1-(4-{[5-(3-Aminophenyl)pyrazin-2-yl]methoxy}-2-hydroxy-3-methylphenyl)-3,3-dimethylbutan-1-one

A 2M Na₂CO₃ solution (0.5 mL) was added to a mixture of 1-(4-((5-bromopyrazin-2-yl)methoxy)-2-hydroxy-3-methylphenyl)-3,3-dimethylbutan-1-one (0.100 g, 0.254 mmol), (3-aminophenyl)boronic acid (0.052 g, 0.381 mmol) and tetrakistriphenylphosphinepalladium(0) (0.029 g, 0.025 mmol) in DME (4 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded crude product that was further purified by automated prep-HPLC to yield the title compound. White solid (0.049 g, 48%). ¹H NMR (400 MHz, DMSO-d₆) δ 9.06 (d, J=1.5 Hz, 1H), 8.78 (d, J=1.4 Hz, 1H), 7.88 (d, J=9.1 Hz, 1H), 7.32 (t, J=2.0 Hz, 1H), 7.25-7.20 (m, 1H), 7.13 (t, J=7.8 Hz, 1H), 6.73 (d, J=9.1 Hz, 1H), 6.68-6.66 (m, 1H), 5.37 (s, 2H), 5.25 (s, 2H), 2.81 (s, 2H), 2.07 (s, 3H), 0.96 (s, 9H. LC-MS (ESI) Calcd for C₂₄H₂₇N₃O₃ [M+H]⁺: 406.20. Found: 406.00.

### Example 147

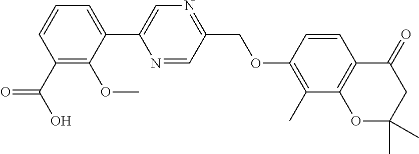 [SMILES: COc1c(C(=O)O)cccc1-c1cnc(COc2ccc3c(c2C)OC(C)(C)CC3=O)cn1]

### 2-Methoxy-3-(5-(((2,2,8-trimethyl-4-oxochroman-7-yl)oxy)methyl)pyrazin-2-yl)benzoic acid

A mixture of 1-(4-((5-bromopyrazin-2-yl)methoxy)-2-hydroxy-3-methylphenyl)-3-methylbut-2-en-1-one (0.189 g, 0.5 mmol), 3-borono-2-methoxybenzoic acid (0.098 g, 0.50 mmol) and tetrakistriphenylphosphinepalladium(0) (0.058 mg, 0.050 mmol) was dissolved in DME (10 mL) and then treated with 2M Na₂CO₃ (1 mL). The resulting mixture was heated at reflux under an atmosphere of N₂ (g) for 12 h. The reaction mixture was cooled to room temperature, and the solvents were removed in vacuo. The residue was dissolved in water and neutralized using 1M HCl. The aqueous layer was extracted with ethyl acetate and the organic layer was dried over anhydrous Na₂SO₄. The solvent was evaporated in vacuo to obtain the crude acid as a yellow solid. The crude residue was purified using preparative HPLC to afford the title compound 2 (0.139 g, 62%) as a white solid. (0.139 g, 62%). ¹H NMR (400 MHz, DMSO-D₆) δ 9.06-8.94 (d, J=7.6 Hz, 1H), 7.79 (d, J=7.7 Hz, 1H), 7.88-7.86 (d, J=8.7 Hz, 1H), 7.80-7.78 (d, J=9.8 Hz, 1H), 7.65-7.49 (m, J=7.49-7.62, 4H), 7.37-7.33 (t, J=7.7 Hz, 1H), 6.90-6.88 (d, J=8.9 Hz, 1H), 5.42 (s, 2H), 3.62 (s, 3H), 2.72 (s, 2H), 2.10 (s, 3H), 1.39 (s, 6H). ¹³C NMR (101 MHz, DMSO-D₆) δ 206.82, 198.14, 191.14, 181.32, 161.73, 158.29, 144.31, 142.90, 124.72, 125.10, 124.29, 115.41, 105.44, 105.40, 79.16, 47.72, 26.30, 8.34. LCMS calcd for C₂₆H₂₆N₂O₅ [M+H]⁺: 449.17, found 450.27.

### Example 148

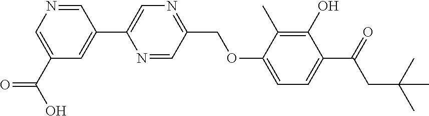 [SMILES: Cc1c(OCc2cnc(-c3cncc(C(=O)O)c3)cn2)ccc(C(=O)CC(C)(C)C)c1O]

### 5-(5-((4-(3,3-Dimethylbutanoyl)-3-hydroxy-2-methylphenoxy)methyl)pyrazin-2-yl)nicotinic acid

A 2M Na₂CO₃ solution (0.5 mL) was added to a mixture of 1-(4-((5-bromopyrazin-2-yl)methoxy)-2-hydroxy-3-methylphenyl)-3,3-dimethylbutan-1-one (0.118 g, 0.3 mmol), (5-cyanopyridin-3-yl)boronic acid (0.067 g, 045 mmol) and tetrakistriphenylphosphinepalladium(0) (0.035 g, 0.03 mmol) in DME (4 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded 5-(4-((4-(3,3-dimethylbutanoyl)-3-hydroxy-2-methylphenoxy)methyl)phenyl)nicotinonitrile. The crude product was re-dissolved in THF:H₂O (10 mL, 1:1) and NaOH (0.120 g, 3 mmol) was added to the solution. The resulting mixture was heated overnight and cooled to rt, acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude product that was further purified by automated prep-HPLC to yield the title compound. White solid (0.056 g, 43%). ¹H NMR (400 MHz, DMSO-d₆) δ 9.44 (d, J=1.9 Hz, 1H), 9.40 (d, J=1.4 Hz, 1H), 9.12 (d, J=2.1 Hz, 1H), 8.95 (d, J=1.5 Hz, 1H), 8.90 (t, J=2.2 Hz, 1H), 7.92 (d, J=9.1 Hz, 1H), 6.78 (d, J=9.1 Hz, 1H), 5.47 (s, 2H), 2.85 (s, 2H), 2.07 (s, 3H), 0.99 (s, 9H). LC-MS (ESI) Calcd for C₂₄H₂₅N₃O₅ [M+H]⁺: 436.18. Found: 436.15.

### Example 149

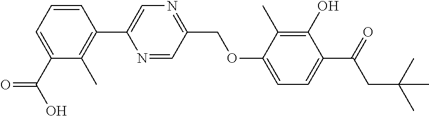 [SMILES: Cc1c(C(=O)O)cccc1-c1cnc(COc2ccc(C(=O)CC(C)(C)C)c(O)c2C)cn1]

### 3-(5-((4-(3,3-Dimethylbutanoyl)-3-hydroxy-2-methylphenoxy)methyl)pyrazin-2-yl)-2-methylbenzoic acid

A 2M Na₂CO₃ solution (1 mL) was added to a mixture of 1-(4-((5-bromopyrazin-2-yl)methoxy)-2-hydroxy-3-methylphenyl)-3,3-dimethylbutan-1-one (0.118 g, 0.3 mmol), (3-cyano-2-methylphenyl)boronic acid (0.072 g, 045 mmol) and tetrakistriphenylphosphinepalladium(0) (0.035 g, 0.03 mmol) in DME (4 mL). The resulting solution was heated at reflux under an atmosphere of N₂ (g) for 2 h. The reaction mixture was cooled to room temperature, diluted with water, and then acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude intermediate, which was re-dissolved in THF:H₂O (10 mL, 1:1), and NaOH (0.120 g, 3 mmol) was added to the solution. The resulting mixture was heated overnight and cooled to rt before being acidified using 1N HCl. The aqueous phase was extracted with ethyl acetate (3×10 mL) and the combined organic layer was washed with brine, followed by drying over anhydrous Na₂SO₄. Filtration and removal of the solvent afforded the crude product which was purified by automated prep-HPLC to yield the title compound. White solid (0.065 g, 48%). ¹H NMR (400 MHz, DMSO-d₆) δ 8.91 (s, 1H), 8.81 (s, 1H), 7.93 (s, 1H), 7.86 (s, 1H), 7.42 (m, 2H), 6.80 (d, J=8.9 Hz, 1H), 5.46 (s, 2H), 2.86 (s, 2H), 2.31 (s, 3H), 2.09 (s, 3H), 1.00 (s, 9H). LC-MS (ESI) Calcd for C₂₆H₂₈N₂O₅ [M+H]⁺: 449.20. Found: 449.00.

### Example 152

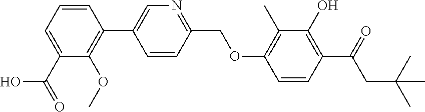 [SMILES: COc1c(C(=O)O)cccc1-c1ccc(COc2ccc(C(=O)CC(C)(C)C)c(O)c2C)nc1]

### 3-(6-((4-(3,3-Dimethylbutanoyl)-3-hydroxy-2-methylphenoxy)methyl)pyridin-3-yl)-2-methoxybenzoic acid

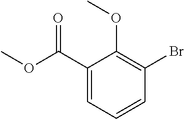

### Step 1: Methyl 3-bromo-2-methoxybenzoate

To a solution of 3-bromo-2-methoxybenzoic acid (1 g, 4.3 mmol) in MeOH (20 mL) was added SOCl₂ (2 mL) at 0° C. and the mixture was then stirred at 60° C. for 2 h. After cooling to room temperature the reaction mixture was concentrated and then extracted with ethyl acetate (20 mL×3). The combined organic layers were washed with water (60 mL) and brine (60 mL), then dried over Na₂SO₄ and filtered. The filtrate was concentrated to afford methyl 3-bromo-2-methoxybenzoate (849 mg, 80.1%) as a colorless oil. LC-MS (ESI): 246.0 (M+H)⁺.

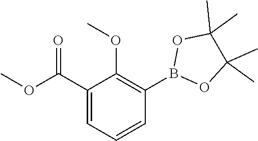

### Step 2: Methyl 2-methoxy-3-(4,4,5,5-tetramethyl-1,3,2-dioxaborolan-2-yl)benzoate

A mixture of methyl 3-bromo-2-methoxybenzoate (849 mg, 3.46 mmol), 4,4,4′,4′,5,5,5′,5′-octamethyl-2,2′-bi(1,3,2-dioxaborolane) (1.32 g, 5.19 mmol), Pd(dppf)Cl₂ (404 mg, 0.35 mmol), and KOAc (679 mg, 6.92 mmol) in dioxane (50 mL) was stirred at 105° C. for 4 h under N₂ (g). The reaction mixture was concentrated and purified by flash column chromatography (silica gel, eluting with 20% EA/PE) to afford methyl 2-methoxy-3-(4,4,5,5-tetramethyl-1,3,2-dioxaborolan-2-yl)benzoate (566 mg, 56.0%) as a colorless oil. LC-MS (ESI): 293.2 (M+H)⁺.

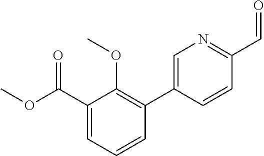 [SMILES: COC(=O)c1cccc(-c2ccc(C=O)nc2)c1OC]

### Step 3: Methyl 3-(6-formylpyridin-3-yl)-2-methoxybenzoate

Methyl 2-methoxy-3-(4,4,5,5-tetramethyl-1,3,2-dioxaborolan-2-yl)benzoate (460 mg, 1.57 mmol), 5-bromopicolinaldehyde (186 mg, 1.0 mmol), Pd(PPh₃)₄ (115 mg, 0.1 mmol) and K₂CO₃ (276 mg, 2.0 mmol) in a mixture of dioxane (25 mL) and H₂O (2.5 mL) was stirred at 100° C. for 16 h under N₂ (g). The reaction mixture was concentrated in vacuo and purified by flash column chromatography (silica gel, eluting with 16% EA/PE) to afford methyl 3-(6-formylpyridin-3-yl)-2-methoxybenzoate (200 mg, 73.8%) as a colorless oil. LC-MS (ESI): 272.2 (M+H)⁺.

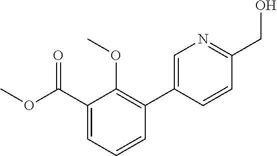 [SMILES: COC(=O)c1cccc(-c2ccc(CO)nc2)c1OC]

### Step 4: Methyl 3-(6-(hydroxymethyl)pyridin-3-yl)-2-methoxybenzoate

To a solution of methyl 3-(6-formylpyridin-3-yl)-2-methoxybenzoate (150 mg, 0.55 mmol) in MeOH (10 mL) was added NaBH₄ (25 mg, 0.66 mmol) portionwise. The mixture was stirred at room temperature for 2 h. The reaction mixture was quenched with water (50 mL), and then extracted with ethyl acetate (10 mL×3). The combined organic layers were washed with water (50 mL) and brine (50 mL), dried over Na₂SO₄ and filtered. The filtrate was concentrated and purified by flash column chromatography (silica gel, eluting with 50% EA/PE) to afford methyl 3-(6-(hydroxymethyl)pyridin-3-yl)-2-methoxybenzoate (130 mg, 86.1%) as a yellow oil. LC-MS (ESI): 274.2 (M+H)⁺.

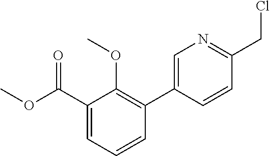 [SMILES: COC(=O)c1cccc(-c2ccc(CCl)nc2)c1OC]

### Step 5: Methyl 3-(6-(chloromethyl)pyridin-3-yl)-2-methoxybenzoate

To a solution of methyl 3-(6-(hydroxymethyl)pyridin-3-yl)-2-methoxybenzoate (100 mg, 0.37 mmol) in CH₂Cl₂ (6 mL) was added SOCl₂ (132 mg, 1.11 mmol) at 0° C. The mixture was stirred at room temperature for 2 h. The reaction mixture was concentrated and then extracted with ethyl acetate (10 mL×3). The combined organic layers were washed with water (30 mL) and brine (30 mL), dried over Na₂SO₄ and filtered. The filtrate was concentrated to afford methyl 3-(6-(chloromethyl)pyridin-3-yl)-2-methoxybenzoate (110 mg, 100%) as a colorless oil. LC-MS (ESI): 292.0 (M+H)⁺.

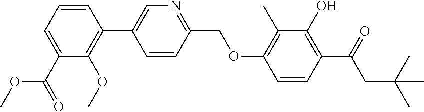 [SMILES: COC(=O)c1cccc(-c2ccc(COc3ccc(C(=O)CC(C)(C)C)c(O)c3C)nc2)c1OC]

### Step 6: Methyl 3-(6-((4-(3,3-dimethylbutanoyl)-3-hydroxy-2-methylphenoxy)methyl)pyridin-3-yl)-2-methoxybenzoate

To a solution of 1-(2,4-dihydroxy-3-methylphenyl)-3,3-dimethylbutan-1-one (109 mg, 0.49 mmol) in DMF (3 mL) were added potassium carbonate (105 mg, 0.76 mmol) and methyl 3-(6-(chloromethyl)pyridin-3-yl)-2-methoxybenzoate (110 mg, 0.38 mmol) successively. The mixture was stirred at 60° C. for 18 h. The reaction mixture was quenched with water (50 mL), and then extracted with ethyl acetate (10 mL×3). The combined organic layers were washed with water (30 mL×3) and brine (30 mL), dried over Na₂SO₄ and filtered. The filtrate was concentrated and purified by flash column chromatography (silica gel, eluting with 25% EA/PE) to afford methyl 3-(6-((4-(3,3-dimethylbutanoyl)-3-hydroxy-2-methylphenoxy)methyl)pyridin-3-yl)-2-methoxybenzoate (160 mg, 88.9%) as a colorless oil. LC-MS (ESI): 478.2 (M+H)⁺.

### Step 7: 3-(6-((4-(3,3-Dimethylbutanoyl)-3-hydroxy-2-methylphenoxy)methyl)pyridin-3-yl)-2-methoxybenzoic acid

To a solution of methyl 3-(6-((4-(3,3-dimethylbutanoyl)-3-hydroxy-2-methylphenoxy)methyl)pyridin-3-yl)-2-methoxybenzoate (160 mg, 0.34 mmol) in THF (5 mL) and H₂O (5 mL) was added LiOH·H₂O (57 mg, 1.36 mmol). The mixture was stirred at room temperature for 16 h. The reaction mixture was concentrated and diluted with water (5 mL). The mixture was acidified to pH=4 with HCl aqueous (1N). The resulting precipitate was filtered and dried in vacuo to afford the title compound (122 mg, 78.6%) as a white solid. ¹H NMR (400 MHz, DMSO-d₆) δ 13.26 (s, 1H), 13.08 (s, 1H), 8.73 (d, J=2.0 Hz, 1H), 8.03 (dd, J=8.4, 2.4 Hz, 1H), 7.93 (d, J=9.2 Hz, 1H), 7.73 (dd, J=7.6, 1.6 Hz, 1H), 7.65-7.60 (m, 2H), 7.32 (t, J=7.6 Hz, 1H), 6.75 (d, J=8.8 Hz, 1H), 5.40 (s, 2H), 3.48 (s, 3H), 2.87 (s, 2H), 2.12 (s, 3H), 1.01 (s, 9H). LC-MS (ESI): 464.2 (M+H)⁺.

### Example 153

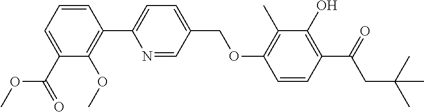 [SMILES: COC(=O)c1cccc(-c2ccc(COc3ccc(C(=O)CC(C)(C)C)c(O)c3C)cn2)c1OC]

### 3-(5-((4-(3,3-Dimethylbutanoyl)-3-hydroxy-2-methylphenoxy)methyl)pyridin-2-yl)-2-methoxybenzoic acid

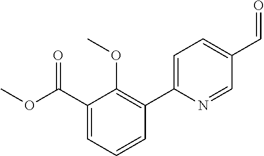 [SMILES: COC(=O)c1cccc(-c2ccc(C=O)cn2)c1OC]

### Step 1: Methyl 3-(5-formylpyridin-2-yl)-2-methoxybenzoate

A mixture of methyl 2-methoxy-3-(4,4,5,5-tetramethyl-1,3,2-dioxaborolan-2-yl)benzoate (950 mg, 3.24 mmol), 6-chloronicotinaldehyde (913 mg, 6.48 mmol), Pd(PPh₃)₄ (374 mg, 0.32 mmol) and Na₂CO₃ (894 mg, 6.48 mmol) in dioxane (10 mL) and H₂O (1 mL) was stirred at 100° C. for 16 h. The reaction mixture was concentrated and then extracted with ethyl acetate (10 mL×3). The combined organic layers were washed with water (30 mL) and brine (30 mL), dried over Na₂SO₄ and filtered. The filtrate was concentrated and purified by flash column chromatography (silica gel, eluting with 20% EA/PE) to afford methyl 3-(5-formylpyridin-2-yl)-2-methoxybenzoate (475 mg, 54.1%) as a white solid. LC-MS (ESI): 272.2 (M+H)⁺.

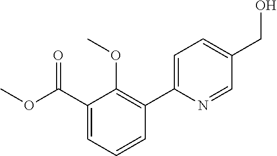 [SMILES: COC(=O)c1cccc(-c2ccc(CO)cn2)c1OC]

### Step 2: Methyl 3-(5-(hydroxymethyl)pyridin-2-yl)-2-methoxybenzoate

To a solution of methyl 3-(5-formylpyridin-2-yl)-2-methoxybenzoate (475 mg, 1.75 mmol) in CH₃OH (5 mL) was added NaBH₄ (140 mg, 3.50 mmol) portionwise. The mixture was stirred at room temperature for 3 h before being quenched with water (15 mL) and extracted with ethyl acetate (5 mL×3). The combined organic layers were washed with water (20 mL) and brine (20 mL), dried over Na₂SO₄ and filtered. The filtrate was concentrated and purified by PLC (CH₂Cl₂/MeOH=10/1) to afford methyl 3-(5-(hydroxymethyl)pyridin-2-yl)-2-methoxybenzoate (320 mg, 67.2%) as a white solid. HPLC/UV purity: 95%; LC-MS (ESI): 274.2 (M+H)⁺.

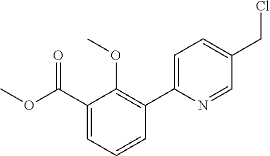 [SMILES: COC(=O)c1cccc(-c2ccc(CCl)cn2)c1OC]

### Step 3: Methyl 3-(5-(chloromethyl)pyridin-2-yl)-2-methoxybenzoate

To a solution of methyl 3-(5-(hydroxymethyl)pyridin-2-yl)-2-methoxybenzoate (320 mg, 1.16 mmol) in CH₂Cl₂ (10 mL) was added SOCl₂ (2 mL) at 0° C. The mixture was stirred at room temperature for 2 h before being concentrated and then extracted with ethyl acetate (20 mL×3). The combined organic layers were washed with water (60 mL) and brine (60 mL), dried over Na₂SO₄ and filtered. The filtrate was concentrated and purified by flash column chromatography (silica gel, eluting with 15% EA/PE) to afford methyl 3-(5-(chloromethyl)pyridin-2-yl)-2-methoxybenzoate (250 mg, 74.1%) as a colorless oil. LC-MS (ESI): 292.0 (M+H)⁺.

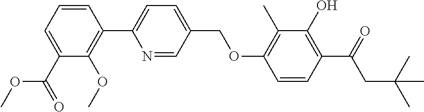 [SMILES: COC(=O)c1cccc(-c2ccc(COc3ccc(C(=O)CC(C)(C)C)c(O)c3C)cn2)c1OC]

### Step 4: Methyl 3-(5-((4-(3,3-dimethylbutanoyl)-3-hydroxy-2-methylphenoxy)methyl)pyridin-2-yl)-2-methoxybenzoate

To a solution of 1-(2,4-dihydroxy-3-methylphenyl)-3,3-dimethylbutan-1-one (60 mg, 0.27 mmol) in DMF (2 mL) were added K₂CO₃ (37 mg, 0.27 mmol) and methyl 3-(5-(chloromethyl)pyridin-2-yl)-2-methoxybenzoate (40 mg, 0.14 mmol) successively. The mixture was stirred at 60° C. for 12 h before being quenched with water (35 mL), and then extracted with ethyl acetate (5 mL×3). The combined organic layers were washed with water (10 mL×3) and brine (30 mL), dried over Na₂SO₄ and filtered. The filtrate was concentrated and purified by flash column chromatography (silica gel, eluting with 5% MeOH/CH₂Cl₂) to afford methyl 3-(5-((4-(3,3-dimethylbutanoyl)-3-hydroxy-2-methylphenoxy)methyl)pyridin-2-yl)-2-methoxybenzoate (60 mg, 93.8%) as a colorless oil. LC-MS (ESI): 478.5 (M+H)⁺.

### Step 5: 3-(5-((4-(3,3-Dimethylbutanoyl)-3-hydroxy-2-methylphenoxy)methyl)pyridin-2-yl)-2-methoxybenzoic acid

To a solution of methyl 3-(5-((4-(3,3-dimethylbutanoyl)-3-hydroxy-2-methylphenoxy)methyl)pyridin-2-yl)-2-methoxybenzoate (60 mg, 0.12 mmol) in THF (3 mL) and H₂O (3 mL) was added LiOH·H₂O (9 mg, 0.36 mmol). The reaction mixture was stirred at room temperature for 16 h before being concentrated and acidified to pH=4 with aqueous HCl (1N). The mixture was then extracted with ethyl acetate (5 mL×3). The combined organic layers were washed with water (10 mL) and brine (10 mL), dried over Na₂SO₄ and filtered. The filtrate was concentrated and purified by prep HPLC to afford the title compound (24 mg, 42.2%) as a white solid. ¹H NMR (400 MHz, DMSO-d₆) δ 13.25 (s, 1H), 13.05 (s, 1H), 8.81 (s, 1H), 8.06-7.62 (m, 4H), 7.30 (t, J=7.6 Hz, 1H), 6.79 (d, J=9.2 Hz, 1H), 5.37 (s, 2H), 3.56 (s, 3H), 2.88 (s, 2H), 2.06 (s, 3H), 1.02 (s, 9H). LC-MS (ESI): 464.2 (M+H)⁺.

### Example 154

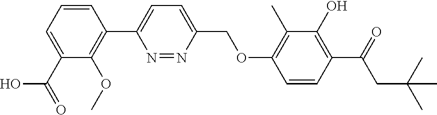 [SMILES: COc1c(C(=O)O)cccc1-c1ccc(COc2ccc(C(=O)CC(C)(C)C)c(O)c2C)nn1]

### 3-(6-((4-(3,3-Dimethylbutanoyl)-3-hydroxy-2-methylphenoxy)methyl)pyridazin-3-yl)-2-methoxybenzoic acid

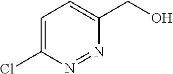

### Step 1: 6-Chloropyridazin-3-yl)methanol

To a solution of methyl 6-chloropyridazine-3-carboxylate (500 mg, 2.9 mmol) in THF (10 mL) was added NaBH₄ (348 mg, 8.7 mmol) portionwise at 0° C. The reaction mixture was stirred at 0° C. for 12 h, concentrated and then extracted with ethyl acetate (10 mL×3). The combined organic layers were washed with water (10 mL) and brine (10 mL), dried over Na₂SO₄ and filtered. The filtrate was concentrated and purified by flash column chromatography (silica gel, eluting with 30% EA/PE) to afford (6-chloropyridazin-3-yl)methanol (240 mg, 57.4%) as a white solid. LC-MS (ESI): 145.5 (M+H)⁺.

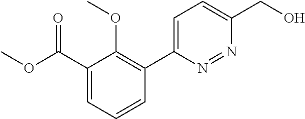 [SMILES: COC(=O)c1cccc(-c2ccc(CO)nn2)c1OC]

### Step 2: Methyl 3-(6-(hydroxymethyl)pyridazin-3-yl)-2-methoxybenzoate

A mixture of (6-chloropyridazin-3-yl)methanol (190 mg, 1.32 mmol), methyl 2-methoxy-3-(4,4,5,5-tetramethyl-1,3,2-dioxaborolan-2-yl)benzoate (462 mg, 1.58 mmol), Pd(PPh₃)₄ (160 mg, 0.132 mmol) and Na₂CO₃ (279 mg, 2.64 mmol) in dioxane (10 mL) and H₂O (1 mL) was stirred at 90° C. for 15 h. After cooling to room temperature the reaction mixture was concentrated and then extracted with ethyl acetate (10 mL×3). The combined organic layers were washed with water (30 mL) and brine (30 mL), dried over Na₂SO₄ and filtered. The filtrate was concentrated and purified by flash column chromatography (silica gel, eluting with 15% EA/PE) to afford methyl 3-(6-(hydroxymethyl)pyridazin-3-yl)-2-methoxybenzoate (140 mg, 16.9%) as a yellow oil. LC-MS (ESI): 275.2 (M+H)⁺.

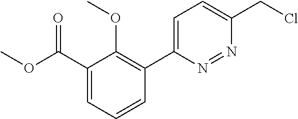 [SMILES: COC(=O)c1cccc(-c2ccc(CCl)nn2)c1OC]

### Step 3: Methyl 3-(6-(chloromethyl)pyridazin-3-yl)-2-methoxybenzoate

To a solution of methyl 3-(6-(hydroxymethyl)pyridazin-3-yl)-2-methoxybenzoate (140 mg, 0.51 mmol) in CH₂Cl₂ (5 mL) was added SOCl₂ (182 mg, 1.53 mmol) at 0° C. The mixture was stirred at room temperature for 2 h. The reaction mixture was then quenched with water (10 mL) and extracted with ethyl acetate (10 mL×3). The combined organic layers were washed with 10% aqueous NaHCO₃ (15 mL), water (10 mL) and brine (10 mL), dried over Na₂SO₄ and filtered. The filtrate was concentrated under reduced pressure to afford methyl 3-(6-(chloromethyl)pyridazin-3-yl)-2-methoxybenzoate (134 mg, 89.9%) as a brown oil which was used in the next step without further purification. LC-MS (ESI): 293.0 (M+H)⁺.

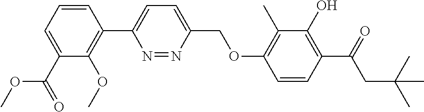 [SMILES: COC(=O)c1cccc(-c2ccc(COc3ccc(C(=O)CC(C)(C)C)c(O)c3C)nn2)c1OC]

### Step 4: Methyl 3-(6-((4-(3,3-dimethylbutanoyl)-3-hydroxy-2-methylphenoxy)methyl)pyridazin-3-yl)-2-methoxybenzoate

To a solution of 1-(2,4-dihydroxy-3-methylphenyl)-3,3-dimethylbutan-1-one (111 mg, 0.5 mmol) in DMF (3 mL) were added potassium carbonate (124 mg, 0.9 mmol) and methyl 3-(6-(chloromethyl)pyridazin-3-yl)-2-methoxybenzoate (134 mg, 0.45 mmol) sequentially. The resulting mixture was stirred at 60° C. for 18 h and after cooling to rt was quenched with water (50 mL), and extracted with ethyl acetate (10 mL×3). The combined organic layers were washed with water (30 mL×3) and brine (30 mL), dried over Na₂SO₄ and filtered. The filtrate was concentrated and purified by flash column chromatography (silica gel, eluting with 25% EA/PE) to afford methyl 3-(6-((4-(3,3-dimethylbutanoyl)-3-hydroxy-2-methylphenoxy)methyl)pyridazin-3-yl)-2-methoxybenzoate (120 mg, 54.8%) as a colorless oil. LC-MS (ESI): 479.2 (M+H)⁺.

### Step 5: 3-(6-((4-(3,3-Dimethylbutanoyl)-3-hydroxy-2-methylphenoxy)methyl)pyridazin-3-yl)-2-methoxybenzoic acid

To a solution of methyl 3-(6-((4-(3,3-dimethylbutanoyl)-3-hydroxy-2-methylphenoxy)methyl)pyridazin-3-yl)-2-methoxybenzoate (120 mg, 0.25 mmol) in THF (5 mL) and H₂O (5 mL) was added LiOH·H₂O (42 mg, 1.0 mmol). The mixture was stirred at room temperature for 16 h. After this time the solvents were removed under reduced pressure and diluted with water (5 mL). The mixture was acidified to pH=4 with HCl aqueous (1N). The resulting precipitate was filtered and dried in vacuo to afford the title compound (70 mg, 60.1%) as a white solid. ¹H NMR (400 MHz, DMSO-d₆) δ 13.26 (s, 1H), 13.16 (s, 1H), 8.11 (d, J=8.8 Hz, 1H), 7.97-7.90 (m, 3H), 7.85 (dd, J=8.0, 2.0 Hz, 1H), 7.38 (t, J=7.6 Hz, 1H), 6.82 (d, J=9.2 Hz, 1H), 5.63 (s, 2H), 3.57 (s, 3H), 2.88 (s, 2H), 2.10 (s, 3H), 1.02 (s, 9H). LC-MS (ESI): 465.2 (M+H)⁺.

### Example 155

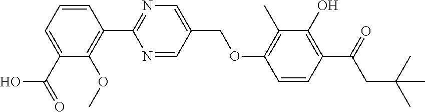 [SMILES: COc1c(C(=O)O)cccc1-c1ncc(COc2ccc(C(=O)CC(C)(C)C)c(O)c2C)cn1]

### 3-(5-((4-(3,3-Dimethylbutanoyl)-3-hydroxy-2-methylphenoxy)methyl)pyrimidin-2-yl)-2-methoxybenzoic acid

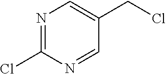

### Step 1: 2-Chloro-5-(chloromethyl)pyrimidine

To a solution of (2-chloropyrimidin-5-yl)methanol (100 mg, 1.16 mmol) in CH₂Cl₂ (5 mL) was added SOCl₂ (1.5 mL) at 0° C. The reaction mixture was stirred at rt for 2 h and then concentrated and then extracted with ethyl acetate (20 mL×3). The combined organic layers were washed with water (60 mL) and brine (60 mL), dried over Na₂SO₄ and filtered. The filtrate was concentrated and purified by flash column chromatography (silica gel, eluting with 25% EA/PE) to afford 2-chloro-5-(chloromethyl)pyrimidine (98 mg, 86.7%) as a colorless oil. LC-MS (ESI): 164.0 (M+H)⁺.

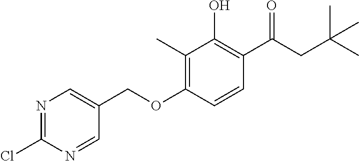 [SMILES: Cc1c(OCc2cnc(Cl)nc2)ccc(C(=O)CC(C)(C)C)c1O]

### Step 2: 1-(4-((2-chloropyrimidin-5-yl)methoxy)-2-hydroxy-3-methylphenyl)-3,3-dimethylbutan-1-one

To a solution of 1-(2,4-dihydroxy-3-methylphenyl)-3,3-dimethylbutan-1-one (268 mg, 1.2 mmol) in DMF (4 mL) were added K₂CO₃ (166 mg, 1.2 mmol) and 2-chloro-5-(chloromethyl)pyrimidine (98 mg, 0.6 mmol) successively. The resulting mixture was stirred at 60° C. for 12 h. The reaction mixture was quenched with water (30 mL), and then extracted with ethyl acetate (10 mL×3). The combined organic layers were washed with water (10 mL×3) and brine (10 mL), dried over Na₂SO₄ and filtered. The filtrate was concentrated and purified by flash column chromatography (silica gel, eluting with 5% MeOH/CH₂Cl₂) to afford 1-(4-((2-chloropyrimidin-5-yl)methoxy)-2-hydroxy-3-methylphenyl)-3,3-dimethylbutan-1-one (60 mg, 28.6%) as a white solid. LC-MS (ESI): 349.2 (M+H)⁺.

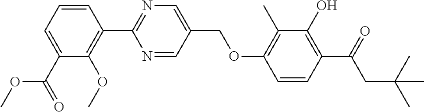 [SMILES: COC(=O)c1cccc(-c2ncc(COc3ccc(C(=O)CC(C)(C)C)c(O)c3C)cn2)c1OC]

### Step 3: Methyl 3-(5-((4-(3,3-dimethylbutanoyl)-3-hydroxy-2-methylphenoxy)methyl)pyrimidin-2-yl)-2-methoxybenzoate

A mixture of 2-chloro-5-(chloromethyl)pyrimidine (60 mg, 0.13 mmol), methyl 2-methoxy-3-(4,4,5,5-tetramethyl-1,3,2-dioxaborolan-2-yl)benzoate (100 mg, 0.26 mmol), Pd(PPh₃)₄ (15 mg, 0.06 mmol) and Na₂CO₃ (14 mg, 1.2 mmol) in dioxane (10 mL) and H₂O (1 mL) was stirred at 90° C. for 12 h. The reaction mixture was concentrated and then extracted with ethyl acetate (10 mL×3). The combined organic layers were washed with water (10 mL×3) and brine (10 mL), dried over Na₂SO₄ and filtered. The filtrate was concentrated and purified by flash column chromatography (silica gel, eluting with 9% MeOH/CH₂Cl₂) to afford methyl 3-(5-((4-(3,3-dimethylbutanoyl)-3-hydroxy-2-methylphenoxy)methyl)pyrimidin-2-yl)-2-methoxybenzoate (45 mg, 75.1%) as a white solid. LC-MS (ESI): 479.2 (M+H)⁺.

### Step 4: 3-(5-((4-(3,3-Dimethylbutanoyl)-3-hydroxy-2-methylphenoxy)methyl)pyrimidin-2-yl)-2-methoxybenzoic acid

To a solution of methyl 3-(5-((4-(3,3-dimethylbutanoyl)-3-hydroxy-2-methylphenoxy)methyl)pyrimidin-2-yl)-2-methoxybenzoate (20 mg, 0.04 mmol) in THF (3 mL) and H₂O (3 mL) was added LiOH·H₂O (7 mg, 0.16 mmol). The mixture was stirred at room temperature for 16 h. The reaction mixture was concentrated and then diluted with water (5 mL). The mixture was acidified to pH=4 with HCl aqueous (1N). Then the mixture was extracted with ethyl acetate (5 mL×3). The combined organic layers were washed with water (20 mL) and brine (20 mL), dried over Na₂SO₄ and filtered. The filtrate was concentrated and purified by PLC (CH₂Cl₂/MeOH=10/1) to afford the title compound (8 mg, 41.2%) as a white solid. ¹H NMR (400 MHz, DMSO-d₆) δ 13.25 (s, 1H), 9.05 (s, 2H), 7.98 (d, J=8.8 Hz, 1H), 7.73 (t, J=7.6 Hz, 2H), 7.26 (t, J=7.6 Hz, 1H), 6.81 (d, J=8.8 Hz, 1H), 5.39 (s, 2H), 3.72 (s, 3H), 2.89 (s, 2H), 2.06 (s, 3H), 1.02 (s, 9H). LC-MS (ESI): 465.2 (M+H)⁺.

### Example 156

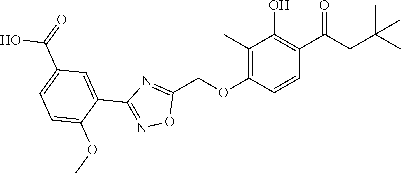 [SMILES: COc1ccc(C(=O)O)cc1-c1noc(COc2ccc(C(=O)CC(C)(C)C)c(O)c2C)n1]

### 3-(5-((4-(3,3-Dimethylbutanoyl)-3-hydroxy-2-methylphenoxy)methyl)-1,2,4-oxadiazol-3-yl)-4-methoxybenzoic acid

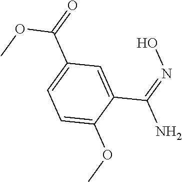 [SMILES: COC(=O)c1ccc(OC)c(/C(N)=N\O)c1]

### Step 1: Methyl (E)-3-(N′-hydroxycarbamimidoyl)-4-methoxybenzoate

To a solution of methyl 3-cyano-4-methoxybenzoate (1.0 g, 5.24 mmol, 1.0 equiv) in ethanol (10 mL) was added hydroxylamine (50% wt in water; 863.8 mg, 26.18 mmol, 5.0 equiv). The mixture was stirred at 100° C. for 1 h and then concentrated to afford methyl (E)-3-(N-hydroxycarbamimidoyl)-4-methoxybenzoate (1.05 g, 89.6%) as a yellow solid. LC-MS (ESI): 224.1 [M+H]⁺.

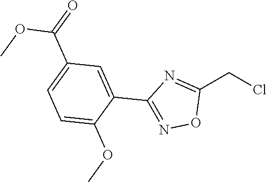 [SMILES: COC(=O)c1ccc(OC)c(-c2noc(CCl)n2)c1]

### Step 2: Methyl 3-(5-(chloromethyl)-1,2,4-oxadiazol-3-yl)-2-methoxybenzoatemethyl 3-(5-(chloromethyl)-1,2,4-oxadiazol-3-yl)-4-methoxybenzoate

A mixture of methyl (E)-3-(N-hydroxycarbamimidoyl)-4-methoxybenzoate (1.05 g, 4.69 mmol, 1.0 equiv) and 2-chloroacetyl chloride (629.4 mg, 5.62 mmol, 1.2 equiv) in toluene (10 mL) was stirred at 110° C. overnight. Then the mixture was concentrated in vacuo to provide a residue which was purified by flash column chromatography (silica gel, eluting with 25% EA in PE) to afford methyl 3-(5-(chloromethyl)-1,2,4-oxadiazol-3-yl)-4-methoxybenzoate (850 mg, 67.5%) as a white solid. LC-MS (ESI): 282.0 [M+H]⁺.

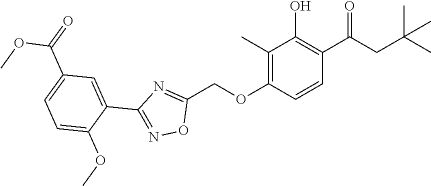 [SMILES: COC(=O)c1ccc(OC)c(-c2noc(COc3ccc(C(=O)CC(C)(C)C)c(O)c3C)n2)c1]

### Step 3: Methyl 3-(5-((4-(3,3-dimethylbutanoyl)-3-hydroxy-2-methylphenoxy)methyl)-1,2,4-oxadiazol-3-yl)-2-methoxybenzoate

A mixture of methyl 3-(5-(chloromethyl)-1,2,4-oxadiazol-3-yl)-4-methoxybenzoate (300 mg, 1.06 mmol, 1.0 equiv), 1-(2,4-dihydroxy-3-methylphenyl)-3,3-dimethylbutan-1-one (245.1 mg, 1.28 mmol, 1.2 equiv.) and K₂CO₃ (293 mg, 2.12 mmol, 2.0 equiv.) in DMF (9 mL) was stirred at 60° C. under N₂ (g) for 3 h. The reaction mixture was poured into water (30 mL) and extracted with ethyl acetate (15 mL×3). The combined organic layers were washed with water (30 mL×3) and brine (10 mL), dried over Na₂SO₄ and filtered. The filtrate was concentrated and purified by flash column chromatography (silica gel, eluting with 30% EA in PE) to afford methyl 3-(5-((4-(3,3-dimethylbutanoyl)-3-hydroxy-2-methylphenoxy)methyl)-1,2,4-oxadiazol-3-yl)-4-methoxybenzoate (94 mg, 18.8%) as a white solid. LC-MS (ESI): 468.2 [M+H]⁺.

### Step 4: 3-(5-((4-(3,3-Dimethylbutanoyl)-3-hydroxy-2-methylphenoxy)methyl)-1,2,4-oxadiazol-3-yl)-4-methoxybenzoic acid

Methyl 3-(5-((4-(3,3-dimethylbutanoyl)-3-hydroxy-2-methylphenoxy)methyl)-1,2,4-oxadiazol-3-yl)-2-methoxybenzoate (94 mg, 0.20 mmol, 1.0 equiv) and lithium hydroxide (14.5 mg, 0.60 mmol, 3.0 equiv) in water (1 mL) was added to a mixture of THF (4 mL) and MeOH (2 mL) and the reaction mixture was stirred at room temperature overnight. The solvents were evaporated under reduced pressure and then diluted with water (5 mL). The mixture was adjusted to pH=3 with 1 N HCl aqueous solution. The precipitate was collected and purified with prep-HPLC to afford the title compound (24.5 mg, 26.9%) as a white solid. ¹H NMR (400 MHz, DMSO-D₆) δ 13.23 (s, 1H), 8.47 (d, J=2.0 Hz, 1H), 8.13 (dd, J=8.8, 8.8 Hz, 1H), 7.96 (d, J=9.2 Hz, 1H), 7.36 (d J=8.8 Hz, 1H), 6.76 (d, J=9.2 Hz, 1H), 5.76 (s, 2H), 3.97 (s, 4H), 2.88 (s, 2H), 2.09 (s, 3H), 1.01 (s, 9H). LC-MS: 454.2 [M+H]⁺.

### Example 157

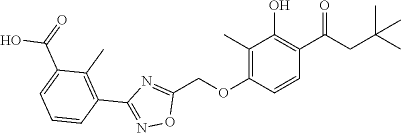 [SMILES: Cc1c(C(=O)O)cccc1-c1noc(COc2ccc(C(=O)CC(C)(C)C)c(O)c2C)n1]

### 3-(5-((4-(3,3-Dimethylbutanoyl)-3-hydroxy-2-methylphenoxy)methyl)-1,2,4-oxadiazol-3-yl)-2-methylbenzoic acid

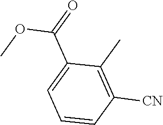

### Step 1: Methyl 3-cyano-2-methylbenzoate

To a solution of methyl 3-bromo-2-methylbenzoate (2.0 g, 8.77 mmol, 1.0 equiv) in DMA (10 mL) were added potassium hexacyanoferrate(III) (631.6 mg, 2.19 mmol, 0.25 equiv), sodium carbonate (926.3 mg, 8.77 mmol, 1.0 equiv.) and palladium acetate (98.5 mg, 0.44 mmol, 0.05 equiv.) successively. The mixture was stirred at 120° C. for 16 h. The reaction mixture was poured into water (30 mL) and extracted with ethyl acetate (20 mL×20). The combined organic layers were washed with water (30 mL×3) and brine (10 mL), dried over Na₂SO₄ and filtered. The filtrate was concentrated in vacuo and purified by flash column chromatography (silica gel, eluting with 15% EA in PE) to afford methyl 3-cyano-2-methylbenzoate (1.1 g, 71.6%) as a white solid. LC-MS (ESI): 296.2 (M+H)⁺.

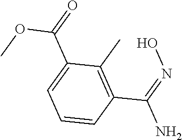

### Step 2: Methyl (E)-3-(N′-hydroxycarbamimidoyl)-2-methylbenzoate

To a solution of methyl 3-cyano-2-methylbenzoate (1.1 g, 6.21 mmol, 1.0 equiv) in ethanol (10 mL) was added hydroxylamine (50% wt in water; 1024.8 mg, 31.06 mmol, 5.0 equiv.). The mixture was stirred at 100° C. for 1 h and then concentrated in vacuo to obtain the crude methyl (E)-3-(N-hydroxycarbamimidoyl)-2-methylbenzoate (0.95 g, 73.1%) as a yellow solid which was used in the next step without further purification. LC-MS (ESI): 208.1 [M+H]⁺.

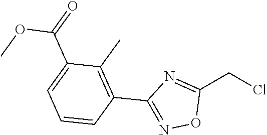 [SMILES: COC(=O)c1cccc(-c2noc(CCl)n2)c1C]

### Step 3: Methyl 3-(5-(chloromethyl)-1,2,4-oxadiazol-3-yl)-2-methylbenzoate

To a solution of methyl (E)-3-(N′-hydroxycarbamimidoyl)-2-methylbenzoate (0.95 g, 4.57 mmol, 1.0 equiv.) in toluene (10 mL) was added 2-chloroacetyl chloride (613.8 mg, 5.48 mmol, 1.2 equiv.). The reaction mixture was stirred at 110° C. overnight and then, after cooling to room temperature, concentrated under reduced pressure to provide a residue which was purified by flash column chromatography (silica gel, eluting with 25% EA in PE) to afford methyl 3-(5-(chloromethyl)-1,2,4-oxadiazol-3-yl)-2-methylbenzoate (560 mg, 46.1%) as a white solid. LC-MS (ESI): 266.1 [M+H]⁺.

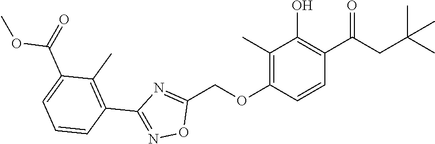 [SMILES: COC(=O)c1cccc(-c2noc(COc3ccc(C(=O)CC(C)(C)C)c(O)c3C)n2)c1C]

### Step 4: Methyl 3-(5-((4-(3,3-dimethylbutanoyl)-3-hydroxy-2-methylphenoxy)methyl)-1,2,4-oxadiazol-3-yl)-2-methylbenzoate

To a solution of methyl 3-(5-(chloromethyl)-1,2,4-oxadiazol-3-yl)-2-methylbenzoate (300 mg, 1.13 mmol, 1.0 equiv) in DMF (8 mL) were added 1-(2,4-dihydroxy-3-methylphenyl)-3,3-dimethylbutan-1-one (300.5 mg, 1.35 mmol, 1.2 equiv) and K₂CO₃ (311.9 mg, 2.26 mmol, 2.0 equiv) successively. The resulting mixture was stirred at 60° C. for 3 h under N₂. The reaction mixture was poured into water (30 mL) and extracted with ethyl acetate (15 mL×3). The combined organic layers were washed with water (30 mL×3) and brine (10 mL), dried over Na₂SO₄ and filtered. The filtrate was concentrated in vacuo and purified by flash column chromatography (silica gel, eluting with 30% EA in PE) to afford methyl 3-(5-((4-(3,3-dimethylbutanoyl)-3-hydroxy-2-methylphenoxy)methyl)-1,2,4-oxadiazol-3-yl)-2-methylbenzoate (360 mg, 70.6%) as a white solid. LC-MS (ESI): 452.2 [M+H]⁺.

### Step 5: 3-(5-((4-(3,3-Dimethylbutanoyl)-3-hydroxy-2-methylphenoxy)methyl)-1,2,4-oxadiazol-3-yl)-2-methylbenzoic acid

A mixture of methyl 3-(5-((4-(3,3-dimethylbutanoyl)-3-hydroxy-2-methylphenoxy)methyl)-1,2,4-oxadiazol-3-yl)-2-methylbenzoate (360 mg, 0.79 mmol, 1.0 equiv) and lithium hydroxide (57.3 mg, 2.39 mmol, 3.0 equiv) in water (2 mL) was added to a mixture of THF (8 mL) and CH₃OH (4 mL) and the reaction mixture was stirred at room temperature overnight. The solvents were evaporated under reduced pressure and then diluted with water (5 mL). The resulting mixture was adjusted pH=3 with 1 N HCl aqueous solution. The precipitate was collected and purified with prep-HPLC to afford the title compound (267.4 mg, 76.7%) as a white solid. ¹H NMR (400 MHz, DMSO-d₆) δ 13.24 (s, 1H), 7.96 (d, J=9.2 Hz, 1H), 7.90 (t, J=6.8 Hz, 2H), 7.47 (t, J=8.0 Hz, 1H), 6.79 (d J=9.2 Hz, 1H), 5.79 (s, 2H), 2.88 (s, 2H), 2.59 (s, 3H), 2.08 (d, J=5.2 Hz, 3H), 0.01 (s, 9H). LC-MS: 438.2 [M+H]⁺.

### Example 158

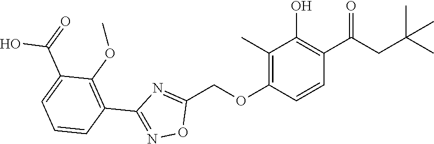 [SMILES: COc1c(C(=O)O)cccc1-c1noc(COc2ccc(C(=O)CC(C)(C)C)c(O)c2C)n1]

### 3-(5-((4-(3,3-Dimethylbutanoyl)-3-hydroxy-2-methylphenoxy)methyl)-1,2,4-oxadiazol-3-yl)-2-methoxybenzoic acid

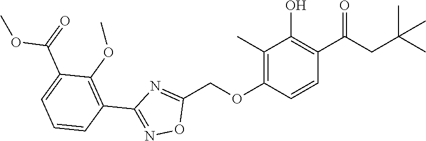 [SMILES: COC(=O)c1cccc(-c2noc(COc3ccc(C(=O)CC(C)(C)C)c(O)c3C)n2)c1OC]

### Step 1: Methyl 3-(5-((4-(3,3-dimethylbutanoyl)-3-hydroxy-2-methylphenoxy)methyl)-1,2,4-oxadiazol-3-yl)-2-methoxybenzoate

To a solution of 1-(2,4-dihydroxy-3-methylphenyl)-3,3-dimethylbutan-1-one (245 mg, 1.28 mmol, 1.2 equiv.) in DMF (6 mL) were added potassium carbonate (294 mg, 2.13 mmol, 2.0 equiv.) and methyl 3-(5-(chloromethyl)-1,2,4-oxadiazol-3-yl)benzoate (300 mg, 1.06 mmol, 1.0 equiv.) successively. The mixture was stirred at 60° C. under N₂ (g) for 3 h. The reaction mixture was poured into water (30 mL) and extracted with ethyl acetate (15 mL×3). The combined organic layers were washed with water (30 mL×3) and brine (10 mL), dried over Na₂SO₄ and filtered. The filtrate was concentrated and purified by flash column chromatography (silica gel, eluting with 30% EA in PE) to afford methyl 3-(5-((4-(3,3-dimethylbutanoyl)-3-hydroxy-2-methylphenoxy)methyl)-1,2,4-oxadiazol-3-yl)-2-methoxybenzoate (320 mg, 64.3%) as a white solid. LC-MS (ESI): 468.2 [M+H]⁺.

### Step 2: 3-(5-((4-(3,3-Dimethylbutanoyl)-3-hydroxy-2-methylphenoxy)methyl)-1,2,4-oxadiazol-3-yl)-2-methoxybenzoic acid

A mixture of methyl 3-(5-((4-(3,3-dimethylbutanoyl)-3-hydroxy-2-methylphenoxy)methyl)-1,2,4-oxadiazol-3-yl)-2-methoxybenzoate (320 mg, 0.68 mmol, 1.0 equiv.) and lithium hydroxide (49.2 mg, 2.05 mmol, 3.0 equiv.) in THF:CH₃OH:H₂O (8:4:2 mL) was stirred at room temperature overnight. The solvents were then removed under reduced pressure, and the remainder was combined with water (5 mL). The resulting mixture was adjusted to pH=3 with 1 N HCl aqueous solution and a precipitate formed. The precipitate was collected and purified by prep-HPLC to afford the title compound (227.5 mg, 14.9%) as a white solid. ¹H NMR (400 MHz, CDCl₃) δ 13.24 (s, 2H), 8.04 (dd, J=8.0, 7.6 Hz, 1H), 7.96 (d, J=9.2 Hz, 1H), 7.92 (dd, J=8.0, 8.0 Hz, 1H), 7.40 (t, J=7.8 Hz, 1H), 6.79 (d, J=9.2 Hz, 2H), 5.79 (s, 2H), 3.79 (s, 3H), 2.88 (s, 2H), 2.09 (s, 3H), 1.01 (s, 9H). LC-MS: 454.2 [M+H]⁺.

### Example 159

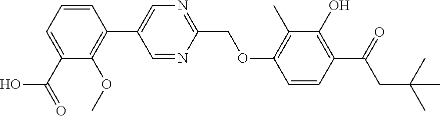 [SMILES: COc1c(C(=O)O)cccc1-c1cnc(COc2ccc(C(=O)CC(C)(C)C)c(O)c2C)nc1]

### 3-(2-((4-(3,3-Dimethylbutanoyl)-3-hydroxy-2-methylphenoxy)methyl)pyrimidin-5-yl)-2-methoxybenzoic acid

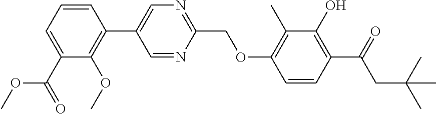 [SMILES: COC(=O)c1cccc(-c2cnc(COc3ccc(C(=O)CC(C)(C)C)c(O)c3C)nc2)c1OC]

### Step 1: Methyl 3-(2-((4-(3,3-dimethylbutanoyl)-3-hydroxy-2-methylphenoxy)methyl)pyrimidin-5-yl)-2-methoxybenzoate

A mixture of 1-(4-((5-bromopyrimidin-2-yl)methoxy)-2-hydroxy-3-methylphenyl)-3,3-dimethylbutan-1-one (350 mg, 0.89 mmol, 1.0 equiv), sodium carbonate (189 mg, 1.79 mmol, 2.0 equiv.), methyl 2-methoxy-3-(4,4,5,5-tetramethyl-1,3,2-dioxaborolan-2-yl)benzoate (238 mg, 1.07 mmol, 1.2 equiv.) and tetrakistriphenylphosphine palladium (103 mg, 0.09 mmol, 0.1 equiv.) in a mixture of dioxane (3 mL) and water (0.5 mL) was stirred at 100° C. overnight. After cooling to room temperature the mixture was concentrated in vacuo to give a residue which was purified with silica gel column chromatography (silica gel, 0˜25% EtOAc:PE) to afford methyl 3-(2-((4-(3,3-dimethylbutanoyl)-3-hydroxy-2-methylphenoxy)methyl)pyrimidin-5-yl)-2-methoxybenzoate (180 mg, 42.2%) as a white solid. LC-MS (ESI): 478.2 [M+H]⁺.

### Step 2: 3-(2-((4-(3,3-Dimethylbutanoyl)-3-hydroxy-2-methylphenoxy)methyl)pyrimidin-5-yl)-2-methoxybenzoic acid

A mixture of methyl 3-(2-((4-(3,3-dimethylbutanoyl)-3-hydroxy-2-methylphenoxy)methyl)pyrimidin-5-yl)-2-methoxybenzoate (180 mg, 0.37 mmol, 1 equiv.) and lithium hydroxide (27.1 mg, 1.13 mmol, 3 equiv) in water (H₂O) was added to THF:H₂O (4:2 mL) and stirred overnight at room temperature. The resulting mixture was concentrated in vacuo and then diluted with water (4 mL). The mixture was adjusted to pH=3 with 1 N HCl aqueous solution. The precipitate was collected and purified with prep-HPLC to afford the title compound (34.2 mg, 35.3%) as a white solid. ¹H NMR (400 MHz, CDCl₃) δ 13.24 (s, 1H), 9.05 (s, 1H), 8.20 (d, J=7.6 Hz, 1H), 7.62-7.59 (m, 2H), 7.43 (t, J=8.0 Hz, 1H), 6.49 (d, J=8.8 Hz, 2H), 5.48 (s, 2H), 3.63 (s, 3H), 2.77 (s, 2H), 2.34 (s, 3H), 1.06 (s, 9H). LC-MS: 464.2 [M+H]⁺.

### Example 160

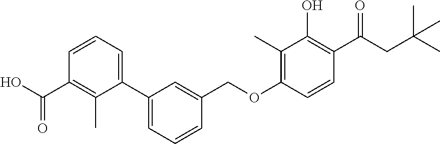 [SMILES: Cc1c(C(=O)O)cccc1-c1cccc(COc2ccc(C(=O)CC(C)(C)C)c(O)c2C)c1]

### 3′-((4-(3,3-Dimethylbutanoyl)-3-hydroxy-2-methylphenoxy)methyl)-2-methyl-[1,1′-biphenyl]-3-carboxylic acid

Prepared according to general method F. Colorless solid (0.115 g, 45%). ¹H NMR (400 MHz, DMSO-d₆): δ 7.90 (d, J=9.2 Hz, 1H), 7.75-7.71 (m, 1H), 7.49-7.27 (m, 7H), 6.72 (d, J=9.2 Hz, 1H), 5.31 (s, 2H), 2.84 (s, 2H), 2.30 (s, 3H), 2.04 (s, 3H), 0.99 (s, 9H). LC-MS m z calcd for C₂₈H₃₁O₅ [M+H]⁺: 447.21. Found: 447.00.

### Example 161

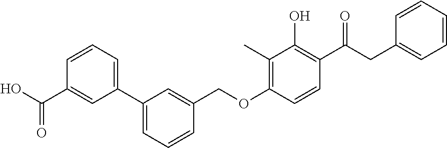 [SMILES: Cc1c(OCc2cccc(-c3cccc(C(=O)O)c3)c2)ccc(C(=O)Cc2ccccc2)c1O]

### 3′-((3-Hydroxy-2-methyl-4-(2-phenylacetyl)phenoxy)methyl)-[1,1′-biphenyl]-3-carboxylic acid

To a solution of 1-(2,4-dihydroxy-3-methylphenyl)-2-phenylethan-1-one (0.061 g, 0.25 mmol), methyl 3′-(bromomethyl)-[1,1′-biphenyl]-3-carboxylate (0.076 g, 0.250 mmol) in ACN (5 mL) was added potassium carbonate (0.069 g, 0.5 mmol) and a catalytic amount of potassium iodide. The reaction mixture was heated at reflux for 4 h. After cooling to room temperature, filtration and evaporation of the solvents yielded the crude product which was used without further purification for the next step. The crude ester was taken up in dioxane (5 mL), 2M LiOH solution (0.625 mL) was added, and the resulting mixture was heated at reflux for 30 min. The reaction was quenched with HCl (5% aq.), and ethyl acetate (10 mL) was added. The organic layer was separated and dried over Na₂SO₄. The solvents were removed by rotary evaporation and the products was isolated by reverse phase HPLC and lyophilized to provide the title compound. White solid (0.085 g, 75%). ¹H NMR (DMSO-d₆): δ 8.17 (d, J=4.6 Hz, 1H), 7.99-7.75 (m, 4H), 7.63-7.44 (m, 4H), 7.27-7.19 (m, 5H), 6.74 (t, J=8.2 Hz, 1H), 5.31 (s 2H), 4.30 (s 2H), 2.01 (s, 3H). LC-MS (ESI) [M+H]⁺: 453.00. HRMS (ESI) m/z calcd for C₂₉H₂₄O₅ [M+H]⁺: 453.1624. found: 453.1713.

### Example 162

### N-Cyano-3′-((4-(3,3-dimethylbutanoyl)-3-hydroxy-2-methylphenoxy)methyl)-[1,1′-biphenyl]-3-carboxamide

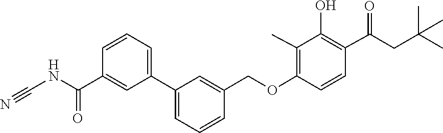 [SMILES: Cc1c(OCc2cccc(-c3cccc(C(=O)NC#N)c3)c2)ccc(C(=O)CC(C)(C)C)c1O]

To a solution of 3′-((4-(3,3-dimethylbutanoyl)-3-hydroxy-2-methylphenoxy)methyl)-[1,1′-biphenyl]-3-carboxylic acid in THF (5 mL) was added N(CH₂CH₃)₃ (0.028 g, 0.277 mmol) and methyl chloroformate (0.026 g, 0.277 mmol). The resulting mixture was stirred at rt for 30 min. The reaction mixture was diluted with water and extracted with EtOAc. The organic layer washed with water, brine and dried over sodium sulfate. Removal of the solvent under reduced pressure yielded the crude anhydride which was used in the next step without further purification. To a stirred solution of the mixed anhydride in anhydrous THF (5 mL) was added a stoichiometric amount of cyanamide (9.72 mg, 0.231 mmol) dissolved in 2 M NaOH solution (0.14 mL). The resulting reaction mixture was stirred at 25° C. for 3-4 h before being acidified with 0.1 N HCl to pH ˜3. The reaction mixture was extracted with EtOAc and the organic phase was washed with brine, dried over MgSO₄ and the solvent evaporated in vacuo to afford the crude product, which was purified by reverse phase HPLC to afford the title compound. White solid (0.061 g, 57.8%). ¹H-NMR (400 MHz, DMSO-D6) δ 8.16 (s, 1H), 7.93-7.86 (m, 3H), 7.80 (s, 1H), 7.68 (d, J=7.3 Hz, 1H), 7.60 (t, J=7.8 Hz, 1H), 7.53-7.47 (m, 3H), 6.72 (d, J=9.2 Hz, 1H), 5.31 (s, 2H), 2.82 (s, 2H), 2.01 (s, 3H), 0.98 (s, 9H). HRMS (ESI) m/z calcd for C₂₈H₂₈N₂O₄ [M+H]⁺: 457.2049. Found: 457.2132.

### Example 163

### 3′-((4-(3,3-Dimethylbutanoyl)-3-hydroxy-2-methylphenoxy)methyl)-[1,1′-biphenyl]-3-sulfonamide

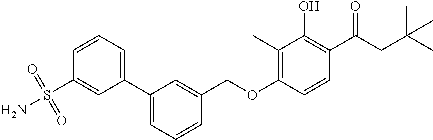 [SMILES: Cc1c(OCc2cccc(-c3cccc(S(N)(=O)=O)c3)c2)ccc(C(=O)CC(C)(C)C)c1O]

(3-((4-(3,3-Dimethylbutanoyl)-3-hydroxy-2-methylphenoxy)methyl)phenyl)boronic acid (0.356 g, 1 mmol), 3-bromobenzenesulfonamide (0.354 g, 1.5 mmol) and Pd(PPh₃)₄ (0.012 g, 0.01 mmol) were dissolved in DME (10 mL) and to this was added 2M Na₂CO₃ solution (2 mL). The resulting solution was heated at reflux under an atmosphere of N₂ for 2 h. The reaction mixture was cooled to room temperature, diluted with water, and then acidified using 1N HCl. The aqueous phase was extracted with ethyl acetate (3×10 mL) and the combined organic layers were washed with brine, followed by drying over anhydrous Na₂SO₄. Filtration and removal of the solvent afforded the crude product which was further purified by automated prep-HPLC to yield the title compound. White solid (0.359 g, 77%). ¹H NMR (DMSO-d₆): δ 8.09 (s, 1H), 7.89 (t, J=4.6 Hz, 2H), 7.80 (d, J=8.7 Hz, 2H), 7.65 (t, J=7.6 Hz, 2H), 7.53-7.49 (m, 2H), 7.40 (s, 2H), 6.72 (d, J=8.7 Hz, 1H), 5.31 (s, 2H), 2.81 (s, 2H), 2.02 (s, 3H), 0.96 (s, 9H). LCMS (ESI): 468.0. [M+H]⁺. HRMS (ESI) m/z calcd for C₂₆H₂₉NO₅S [M−H]⁺: 466.1756. Found: 466.1708.

### Example 164

### 3′-((4-(3,3-dimethylbutanoyl)-3-hydroxy-2-methylphenoxy)methyl)-4-fluoro-6-methoxy-[1,1′-biphenyl]-3-carboxylic acid

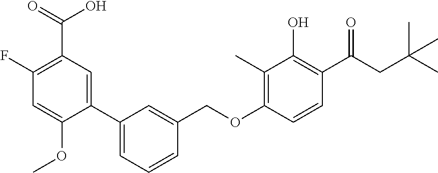 [SMILES: COc1cc(F)c(C(=O)O)cc1-c1cccc(COc2ccc(C(=O)CC(C)(C)C)c(O)c2C)c1]

5-Bromo-2-fluoro-4-methoxybenzoic acid (0.125 g, 0.5 mmol), (3-((4-(3,3-dimethylbutanoyl)-3-hydroxy-2-methylphenoxy)methyl)phenyl)boronic acid (0.267 g, 0.75 mmol), Pd(PPh₃)₄ and 2M Na₂CO₃ solution were processed according to the general method B. White solid. (0.067 g, 28%). ¹H NMR (DMSO-d₆): δ 7.88 (d, J=9.2 Hz, 1H), 7.74 (d, J=8.7 Hz, 1H), 7.53 (s, 1H), 7.41 (s, 3H), 7.08 (d, J=13.3 Hz, 1H), 6.70 (d, J=9.2 Hz, 1H), 5.27 (s, 2H), 3.80 (s, 3H), 2.82 (s, 2H), 2.01 (s, 3H), 0.96 (s, 9H). HRMS (ESI) m/z calcd for C₂₈H₂₉FO₆ [M+H]⁺: 481.1919. Found: 481.1973.

### Example 165

### 3′-(((4-(3,3-Dimethylbutanoyl)-3-hydroxy-2-methylphenyl)thio)methyl)-[1,1′-biphenyl]-3-carboxylic acid

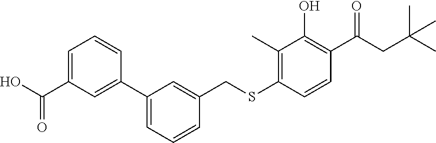 [SMILES: Cc1c(SCc2cccc(-c3cccc(C(=O)O)c3)c2)ccc(C(=O)CC(C)(C)C)c1O]

To a solution of 1-(2-hydroxy-4-mercapto-3-methylphenyl)-3,3-dimethylbutan-1-one (0.119 g, 0.5 mmol) and methyl 3′-(bromomethyl)-[1,1′-biphenyl]-3-carboxylate (0.153 g, 0.5 mmol) in ACN (10 mL) was added potassium carbonate (0.138 g, 1 mmol). The reaction mixture was heated at reflux for 2 h. Following cooling to room temperature, filtration and evaporation of the solvents yielded the crude product which was used without further purification for the next step. The crude ester was taken up in dioxane (5 mL), 2M LiOH solution (1.25 mL) was added, and the resulting mixture was heated at reflux for 30 min. The reaction was quenched with HCl (5% aq.) and ethyl acetate (10 mL) was added. The organic layer was separated and dried over Na₂SO₄. The solvents were removed by rotary evaporation and the products isolated by reverse phase HPLC and lyophilized to afford the final compound. White solid (0.16 g, 71.3%). ¹H NMR (DMSO-d₆): δ 8.14 (t, J=1.8 Hz, 1H), 7.92-7.76 (m, 4H), 7.59-7.54 (m, 2H), 7.46-7.40 (m, 2H), 6.94 (d, J=8.7 Hz, 1H), 4.43 (s, 2H), 2.84 (s, 2H), 2.07 (s, 3H), 0.97-0.93 (m, 9H). LC-MS (ESI) [M+H]⁺: 449.10.

### PHARMACEUTICAL COMPOSITION EXAMPLES

### Example A1: Parenteral Composition

To prepare a parenteral pharmaceutical composition suitable for administration by injection, 100 mg of a water-soluble salt of a compound of Formula (I), (Ia), (II), or (III), or a pharmaceutically acceptable salt or solvate thereof, is dissolved in 2% HPMC, 1% Tween 80 in DI water, pH 2.2 with MSA, q.s. to at least 20 mg/mL. The mixture is incorporated into a dosage unit form suitable for administration by injection.

### Example A2: Oral Composition

To prepare a pharmaceutical composition for oral delivery, 100 mg of a compound of Formula (I), (Ia), (II), or (III), or a pharmaceutically acceptable salt or solvate thereof, is mixed with 750 mg of starch. The mixture is incorporated into an oral dosage unit for, such as a hard gelatin capsule, which is suitable for oral administration.

### BIOLOGY EXAMPLES

### Example B1: mGlu Receptor In Vitro Assays

Human Embryonic Kidney (HEK-293) cell lines co-expressing rat mGlu receptors 2, 3, 4, 6, 7 or 8 and G protein-coupled inwardly-rectifying potassium (GIRK) channels were grown in Growth Media containing 45% DMEM, 45% F-12, 10% FBS, 20 mM HEPES, 2 mM L-glutamine, antibiotic/antimycotic, non-essential amino acids, 700 μg/mL G418, and 0.6 μg/ml puromycin at 37° C. in the presence of 5% CO₂. Cells expressing rat mGlu₁ and mGlu₅ receptor were cultured as described in Hemstapat et al (Mol. Pharmacol. 2006, 70, 616-626). All cell culture reagents were purchased from Invitrogen Corp. (Carlsbad, CA) unless otherwise noted. Calcium assays were used to assess activity of compounds at mGlu₁ and mGlu₅, as previously described in Engers et al (J. Med. Chem. 2009, 52, 4115-4118). Calcium assays at mGlu3 were performed as described for mGlu₅ with the exception that TREx293 mGlu3 G_{α15} cells were treated with tetracycline at 20 ng/mL for 20 h prior to assay.

Compound activity at the group II (mGlu2 and mGlu3) and group III (mGlu₄, mGlu₆, mGlu₇, and mGlu₈) was assessed using thallium flux through GIRK channels, a method that has been described in detail. Briefly, cells were plated into 384-well, black-walled, clear-bottomed poly-D-lysine-coated plates at a density of 15,000 cells/20 μL/well in DMEM containing 10% dialyzed FBS, 20 mM HEPES, and 100 units/mL penicillin/streptomycin (assay media). Plated cells were incubated overnight at 37° C. in the presence of 5% CO₂. The following day, the medium was exchanged from the cells to assay buffer [Hanks' balanced salt solution (Invitrogen) containing 20 mM HEPES, pH 7.3] using an ELX405 microplate washer (BioTek), leaving 20 μL/well, followed by the addition of 20 μL/well FluoZin2-AM (330 nM final concentration) indicator dye (Invitrogen; prepared as a stock in DMSO and mixed in a 1:1 ratio with Pluronic acid F-127) in assay buffer. Cells were incubated for 1 h at room temperature, and the dye exchanged to assay buffer using an ELX405, leaving 20 μL/well. Test compounds were diluted to 2 times their final desired concentration in assay buffer (0.3% DMSO final concentration). Agonists were diluted in thallium buffer [125 mM sodium bicarbonate (added fresh the morning of the experiment), 1 mM magnesium sulfate, 1.8 mM calcium sulfate, 5 mM glucose, 12 mM thallium sulfate, and 10 mM HEPES, pH 7.3] at 5 times the final concentration to be assayed. Cell plates and compound plates were loaded onto a kinetic imaging plate reader (FDSS 6000 or 7000; Hamamatsu Corporation, Bridgewater, NJ). Appropriate baseline readings were taken (10 images at 1 Hz; excitation, 470±20 nm; emission, 540±30 nm) and test compounds were added in a 20 μL volume and incubated for approximately 2.5 min before the addition of 10 μL of thallium buffer with or without agonist. After the addition of agonist, data were collected for approximately an additional 2.5 min. Data were analyzed using Excel (Microsoft Corp, Redmond, WA). The slope of the fluorescence increase beginning 5 s after thallium/agonist addition and ending 15 s after thallium/agonist addition was calculated, corrected to vehicle and maximal agonist control slope values, and plotted in using either XLfit (ID Business Solutions Ltd) or Prism software (GraphPad Software, San Diego, CA) to generate concentration-response curves. Potencies were calculated from fits using a four-point parameter logistic equation. For concentration-response curve experiments, compounds were serially diluted 1:3 into 10-point concentration response curves and were transferred to daughter plates using an Echo acoustic plate reformatter (Labcyte, Sunnyvale, CA). Test compounds were applied and followed by EC₂₀ concentrations of glutamate. For selectivity experiments, full concentration-response curves of glutamate or L-AP4 (for mGlu₇) were performed in the presence of a 10 μM concentration of compound and compounds that affected the concentration-response by less than 2-fold in terms of potency or efficacy were designated as inactive.

### Example B2

Representative in vitro biochemical data for mGlu2 is presented in Table 5.

TABLE 5
In vitro potency data.

| | Rat mGlu2 | Rat mGlu3 | Human mGlu2 | Human mGlu3 |
|---|---|---|---|---|
| Ex. | AVG EC₅₀ (μM) | AVG EC₅₀ (μM) | AVG EC₅₀ (μM) | AVG EC₅₀ (μM) |
| 1 | 0.018 | 0.052 | 0.011 | 0.049 |
| 2 | 0.456 | 0.091 | 0.144 | 0.079 |
| 3 | NT | NT | 1.346 | 0.750 |
| 4 | 0.067 | 0.047 | 0.056 | 0.061 |
| 5 | 0.027 | >10 | 0.026 | >10 |
| 6 | 0.004 | 2.587 | 0.006 | 1.265 |
| 7 | 0.158 | >30 | 0.116 | >10 |
| 8 | 0.019 | 0.489 | 0.010 | 0.534 |
| 9 | 0.073 | >10 | 0.050 | >10 |
| 10 | 0.092 | 0.039 | 0.015 | 0.016 |
| 11 | 0.035 | 0.315 | 0.007 | 0.126 |
| 12 | 0.023 | 0.070 | 0.005 | 0.015 |
| 13 | 0.114 | >10 | 0.052 | >10 |
| 14 | 0.016 | 2.303 | 0.009 | >30 |
| 15 | 0.018 | >30 | 0.008 | >30 |
| 16 | 0.056 | >10 | 0.028 | >30 |
| 17 | 0.355 | 1.523 | 0.153 | 0.768 |
| 18 | NT | NT | 0.882 | >10 |
| 19 | 0.288 | 0.818 | 0.165 | 0.472 |
| 20 | NT | NT | 0.334 | 1.057 |
| 21 | 0.038 | 0.073 | 0.019 | 0.080 |
| 22 | 0.016 | 0.020 | 0.004 | 0.039 |
| 23 | 0.091 | 0.491 | 0.045 | 0.327 |
| 24 | 0.176 | 0.649 | 0.082 | 0.462 |
| 25 | 0.137 | 0.578 | 0.060 | 0.311 |
| 26 | 2.162 | 4.086 | 0.156 | 1.619 |
| 27 | NT | NT | 0.710 | 1.118 |
| 28 | NT | NT | 0.415 | 0.995 |
| 29 | 0.215 | 1.033 | 0.147 | 0.634 |
| 30 | 0.265 | >10 | 0.055 | >10 |
| 31 | NT | NT | 0.373 | >30 |
| 32 | 0.127 | 0.200 | 0.047 | 0.084 |
| 33 | 0.217 | 0.197 | 0.132 | 0.195 |
| 34 | 0.031 | 1.902 | 0.010 | 0.428 |
| 35 | 0.174 | >10 | 0.066 | >10 |
| 36 | 0.089 | >30 | 0.014 | >30 |
| 37 | >10 | >30 | NT | NT |
| 38 | >10 | >30 | NT | NT |
| 39 | >10 | >30 | NT | NT |
| 40 | 0.591 | >30 | NT | NT |
| 41 | 0.015 | 1.002 | 0.008 | 0.886 |
| 42 | 0.027 | 0.552 | 0.028 | 0.371 |
| 43 | 0.033 | >10 | 0.008 | 0.479 |
| 44 | 0.044 | 1.028 | 0.020 | 1.131 |
| 45 | 0.066 | 1.086 | 0.013 | 0.943 |
| 46 | 0.039 | 0.427 | 0.012 | 0.414 |
| 47 | 0.113 | 2.040 | NT | NT |
| 48 | 0.039 | >10 | 0.012 | >10 |
| 49 | 0.426 | >10 | NT | NT |
| 50 | 0.043 | >10 | 0.021 | >10 |
| 51 | 0.022 | 2.376 | 0.009 | >30 |
| 52 | 0.036 | >30 | 0.025 | >30 |
| 53 | 0.019 | 0.920 | 0.006 | 0.708 |
| 54 | 0.008 | 0.577 | 0.006 | 0.300 |
| 55 | 0.182 | 1.172 | NT | NT |
| 56 | NT | NT | 0.001 | 0.031 |
| 57 | NT | NT | 0.148 | 2.631 |
| 58 | NT | NT | 0.008 | 0.540 |
| 59 | 0.182 | 0.222 | 0.325 | 0.408 |
| 60 | 0.117 | >10 | 0.060 | 0.598 |
| 61 | NT | NT | 1.137 | 0.903 |
| 62 | NT | NT | 1.173 | 1.599 |
| 63 | NT | NT | 0.507 | 0.557 |
| 64 | NT | NT | 0.621 | 0.994 |
| 65 | 1.151 | 3.026 | 0.143 | 0.342 |
| 66 | 0.769 | 2.275 | NT | NT |
| 67 | 1.796 | 0.625 | NT | NT |
| 68 | 0.186 | 3.731 | 0.056 | 0.698 |
| 69 | 0.049 | 0.028 | 0.033 | 0.023 |
| 70 | 0.824 | 2.551 | NT | NT |
| 71 | NT | NT | 0.609 | 0.743 |
| 72 | NT | NT | 0.887 | 1.004 |
| 73 | NT | NT | 2.029 | 0.685 |
| 74 | NT | NT | 0.924 | 1.651 |
| 75 | NT | NT | 2.712 | 1.339 |
| 76 | NT | NT | >10 | >10 |
| 77 | NT | NT | 0.119 | >10 |
| 78 | NT | NT | 0.041 | 1.932 |
| 79 | NT | NT | 0.016 | 0.013 |
| 80 | NT | NT | 0.228 | 0.461 |
| 81 | 0.326 | 1.493 | 0.163 | 0.966 |
| 82 | NT | NT | 0.423 | 2.996 |
| 83 | 0.549 | 3.291 | 0.187 | 1.689 |
| 84 | 0.382 | 0.953 | 0.126 | 0.340 |
| 85 | 0.128 | 0.810 | 0.086 | 0.408 |
| 86 | >30 | >10 | NT | NT |
| 87 | 1.317 | 2.394 | NT | NT |
| 88 | 1.531 | >10 | NT | NT |
| 89 | 2.385 | 4.674 | NT | NT |
| 90 | 0.322 | 0.923 | 0.164 | 0.624 |
| 91 | 0.348 | >30 | 0.179 | >30 |
| 92 | 0.057 | 0.234 | 0.016 | 0.151 |
| 93 | 0.207 | 1.217 | 0.039 | 0.218 |
| 94 | 0.441 | >30 | 0.100 | >10 |
| 95 | 0.663 | >30 | 0.131 | >30 |
| 96 | 1.638 | >10 | NT | NT |
| 97 | 1.385 | >10 | NT | NT |
| 98 | >10 | >30 | NT | NT |
| 99 | 0.167 | >30 | NT | NT |
| 100 | 0.079 | 0.732 | 0.015 | 0.773 |
| 101 | 0.033 | >30 | 0.016 | >30 |
| 102 | 0.093 | 0.493 | 0.054 | 0.159 |
| 103 | 0.198 | 0.481 | 0.054 | 0.375 |
| 104 | 0.303 | 0.069 | 0.190 | 0.222 |
| 105 | 0.288 | 0.743 | NT | NT |
| 106 | 0.535 | 0.523 | NT | NT |
| 107 | 0.254 | 2.142 | NT | NT |
| 108 | 0.110 | >30 | 0.066 | >30 |
| 109 | 0.430 | 1.550 | NT | NT |
| 110 | 1.036 | 0.327 | NT | NT |
| 111 | >10 | >30 | NT | NT |
| 112 | 0.217 | 0.281 | NT | NT |
| 113 | 1.271 | 0.333 | NT | NT |
| 114 | 0.391 | 0.258 | NT | NT |
| 115 | 0.356 | 0.043 | 0.355 | 0.156 |
| 116 | 0.117 | 0.028 | 0.234 | 0.213 |
| 117 | >30 | >30 | NT | NT |
| 118 | 0.482 | 1.019 | NT | NT |
| 119 | 0.916 | 0.616 | NT | NT |
| 120 | 0.220 | 0.642 | NT | NT |
| 121 | 0.782 | 0.593 | NT | NT |
| 122 | 0.539 | 0.282 | NT | NT |
| 123 | 2.521 | 2.638 | NT | NT |
| 124 | 2.477 | 1.689 | NT | NT |
| 125 | >10 | >30 | NT | NT |
| 126 | >10 | 2.139 | NT | NT |
| 127 | 0.373 | 0.134 | 0.189 | 0.140 |
| 128 | 0.430 | 0.104 | 0.297 | 0.148 |
| 129 | NT | NT | 0.729 | 0.364 |
| 130 | 0.254 | 0.053 | 0.076 | 0.031 |
| 131 | NT | NT | 0.231 | 1.048 |
| 132 | 0.403 | 0.309 | 0.129 | 0.114 |
| 133 | 0.268 | 0.247 | 0.074 | 0.079 |
| 134 | NT | NT | 0.243 | 0.327 |
| 135 | 0.106 | 0.660 | 0.133 | 0.748 |
| 136 | 0.129 | 0.053 | 0.075 | 0.046 |
| 137 | NT | NT | 0.913 | 3.953 |
| 138 | NT | NT | 0.445 | 0.104 |
| 139 | NT | NT | 1.745 | 1.084 |
| 140 | NT | NT | 1.410 | 1.160 |
| 141 | NT | NT | 0.547 | 0.603 |
| 142 | NT | NT | >10 | >30 |
| 143 | NT | NT | 0.291 | 0.580 |
| 144 | NT | NT | 0.112 | 0.089 |
| 145 | NT | NT | >10 | >10 |
| 146 | NT | NT | >10 | >10 |
| 147 | NT | NT | >30 | >30 |
| 148 | NT | NT | >10 | >30 |
| 149 | NT | NT | 1.483 | 3.263 |
| 150 | NT | NT | >10 | >30 |
| 151 | NT | NT | >30 | >30 |
| 152 | NT | NT | 0.130 | 0.074 |
| 153 | NT | NT | 0.069 | 0.133 |
| 154 | NT | NT | 0.281 | 0.184 |
| 155 | NT | NT | 0.391 | 0.574 |
| 156 | NT | NT | 0.078 | 0.117 |
| 157 | NT | NT | 0.048 | 0.148 |
| 158 | NT | NT | 0.053 | 0.104 |
| 159 | NT | NT | 0.044 | 0.085 |
| 160 | 0.092 | 0.855 | 0.203 | 1.476 |
| 161 | 0.200 | 1.034 | 0.291 | 1.679 |
| 162 | 0.166 | 0.145 | 0.234 | 0.593 |
| 163 | 1.355 | >30 | NT | NT |
| 164 | 0.110 | 0.559 | 0.039 | 0.242 |
| 165 | 0.337 | 2.526 | 0.160 | 1.600 |

NT = not tested.

While preferred embodiments have been shown and described herein, it will be understood by those skilled in the art that such embodiments are provided by way of example only. It is not intended that the embodiments be limited by the specific examples provided within the specification. Numerous variations, changes, and substitutions will now occur to those skilled in the art without departing from the embodiments. It should be understood that various alternatives to the embodiments described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A compound, or a pharmaceutically acceptable salt thereof, having the structure of formula (I): 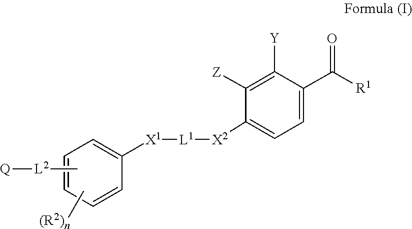
wherein:
R¹ is substituted or unsubstituted C₂-C₆ alkyl, substituted or unsubstituted C₁-C₆ fluoroalkyl, substituted or unsubstituted aryl, substituted or unsubstituted heteroaryl, substituted or unsubstituted cycloalkyl, or substituted or unsubstituted heterocycloalkyl;
X¹ is absent, —O—, —NR⁵—, —S—, —S(═O)—, or —S(═O)₂—;
X² is —O—, —NR⁶—, —S—, —S(═O)—, or —S(═O)₂—;
R⁵ and R⁶ are each independently hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
L¹ is substituted C₄ alkylene, substituted or unsubstituted C₅-C₁₀ alkylene, substituted or unsubstituted C₂-C₁₀ alkenylene, substituted or unsubstituted C₂-C₁₀ alkynylene, -L³-X³-L⁴-, or -L⁵-X⁴-L⁶-;
L³ and L⁴ are each independently substituted or unsubstituted C₁-C₆ alkylene, substituted or unsubstituted C₂-C₆ alkenylene, or optionally substituted or unsubstituted C₂-C₆ alkynylene;
X³ is —C(R′)₂—, —C(R⁹)═C(R⁹)—, —C≡C—, —O—, —NR⁷—, —S—, —S(═O)—, or —S(═O)₂—;
L⁵ and L⁶ are each —C(D)₂- and X⁴ is substituted or unsubstituted C₁-C₄ alkylene;
R⁷ is hydrogen, substituted or unsubstituted C₁-C₆alkyl, substituted or unsubstituted C₁-C₆fluoroalkyl, substituted or unsubstituted cycloalkyl, substituted or unsubstituted heterocycloalkyl, substituted or unsubstituted aryl, substituted or unsubstituted heteroaryl, —C(═O)R⁴, —S(═O)₂R⁴, or —C(═O)OR³;
each R⁸ is independently D, halogen, —OH, substituted or unsubstituted C₁-C₆alkyl, or substituted or unsubstituted C₁-C₆fluoroalkyl;
or two R⁸ are taken together with the carbon atom to which they are attached to form a substituted or unsubstituted cycloalkyl or a substituted or unsubstituted heterocycloalkyl;
each R⁹ is independently hydrogen, D, halogen, —OH, substituted or unsubstituted C₁-C₆alkyl, or substituted or unsubstituted C₁-C₆fluoroalkyl;
L² is absent or C₁-C₆ alkylene;
Q is —C(═O)OH, —C(═O)OR⁴, —C(═O)NHOR³, —C(═O)NHCN, —C(═O)NR³R³, —C(═O)NHS(═O)₂R⁴, —C(═O)R⁴, —S(═O)₂NR³R³, —S(═O)₂NHC(═O)R⁴, —NHC(═O)NHS(═O)₂R⁴, 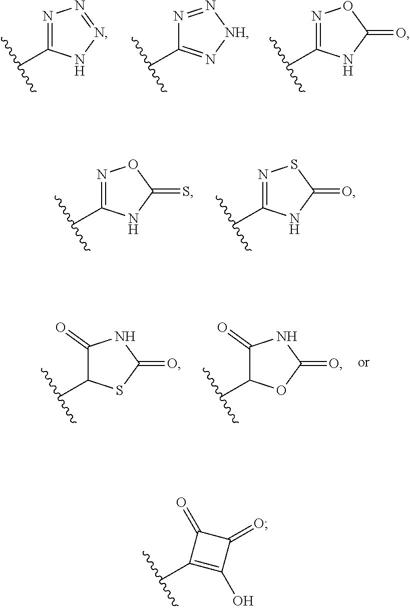
each R² is independently halogen, nitro, —CN, —OH, —OR⁴, substituted or unsubstituted C₁-C₆alkyl, substituted or unsubstituted C₁-C₆fluoroalkyl, or substituted or unsubstituted cycloalkyl;
n is 0, 1, 2, 3, or 4;
Y is —OH, —OR⁴, halogen, substituted or unsubstituted C₁-C₆alkyl, substituted or unsubstituted C₁-C₆fluoroalkyl, or substituted or unsubstituted cycloalkyl;
Z is —OH, —OR⁴, halogen, substituted or unsubstituted C₁-C₆alkyl, substituted or unsubstituted C₁-C₆fluoroalkyl, or substituted or unsubstituted cycloalkyl;
each R³ is independently hydrogen, substituted or unsubstituted C₁-C₆alkyl, substituted or unsubstituted C₁-C₆fluoroalkyl, substituted or unsubstituted cycloalkyl, or substituted or unsubstituted aryl;
or two R³ taken together with the nitrogen to which they are attached to form a substituted or unsubstituted C₂-C₈heterocycloalkyl;
each R⁴ is independently substituted or unsubstituted C₁-C₆alkyl, substituted or unsubstituted cycloalkyl substituted or unsubstituted aryl or substituted or unsubstituted heteroaryl.

2. The compound of claim 1, or a pharmaceutically acceptable salt or solvate thereof, wherein: Y is —OH.

3. (canceled)

4. (canceled)

5. The compound of claim 1, or a pharmaceutically acceptable salt or solvate thereof, wherein: Z is substituted or unsubstituted C₁-C₆alkyl.

6. The compound of claim 5, or a pharmaceutically acceptable salt or solvate, wherein: Z is methyl.

7. The compound of claim 1, or a pharmaceutically acceptable salt or solvate thereof, wherein: L² is C₁-C₆ alkylene.

8. The compound of claim 1, or a pharmaceutically acceptable salt or solvate thereof, wherein: L² is absent.

9. The compound of claim 1, or a pharmaceutically acceptable salt or solvate thereof, wherein: Q is —C(═O)OH, —C(═O)OR⁴, —C(═O)NHOR³, —C(═O)NR³R³, —C(═O)NHS(═O)₂R⁴, —C(═O)R⁴, —S(═O)₂NR³R³, —S(═O)₂NHC(═O)R⁴, 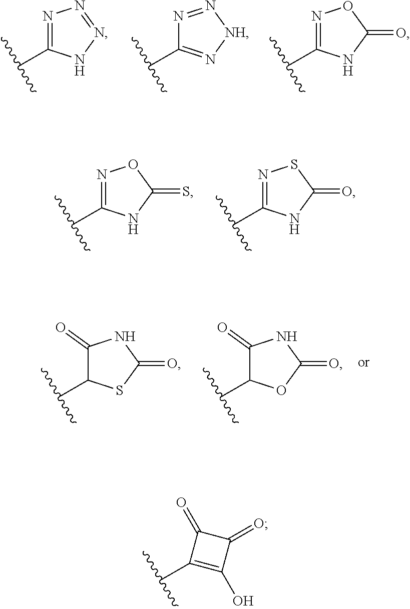
and
wherein each R³ and R⁴ is independently hydrogen or substituted or unsubstituted C₁-C₆alkyl.

10. The compound of claim 1, or a pharmaceutically acceptable salt or solvate thereof, wherein: Q is —C(═O)OH or —C(═O)OR⁴; and wherein R⁴ is methyl or ethyl.

11. (canceled)

12. (canceled)

13. The compound of claim 1, or a pharmaceutically acceptable salt or solvate thereof, wherein: R¹ is unsubstituted C₂-C₆ alkyl.

14. (canceled)

15. (canceled)

16. The compound of claim 1, or a pharmaceutically acceptable salt or solvate thereof, wherein: each R² is independently halogen, nitro, —CN, —OH, —OR⁴, substituted or unsubstituted C₁-C₆alkyl, or substituted or unsubstituted C₁-C₆fluoroalkyl; and wherein R⁴ is substituted or unsubstituted C₁-C₆alkyl.

17.-25. (canceled)

26. The compound of claim 1, or a pharmaceutically acceptable salt or solvate thereof, wherein: X¹ is —O—.

27.-29. (canceled)

30. The compound of claim 1, or a pharmaceutically acceptable salt or solvate thereof, wherein: X² is —O—.

31. (canceled)

32. (canceled)

33. The compound of claim 1, or a pharmaceutically acceptable salt or solvate thereof, wherein: L¹ is substituted C₄ alkylene, substituted or unsubstituted C₅-C₁₀ alkylene, substituted or unsubstituted C₂-C₁₀ alkenylene, or substituted or unsubstituted C₂-C₁₀ alkynylene.

34.-51. (canceled)

52. The compound of any claim 1, or a pharmaceutically acceptable salt or solvate thereof, wherein the compound of Formula (I) has the structure of Formula (Ia), or a pharmaceutically acceptable salt, or solvate thereof: 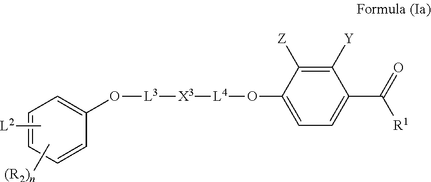
wherein:
L³ and L⁴ are each independently C₁-C₄alkylene;
X³ is —C(R⁸)₂, —C(R⁹)═C(R⁹)—, —C≡C—, —O—, —NR⁷—, —S—, —S(═O)—, or —S(═O)₂—;
each R⁸ is independently D, halogen, —OH, substituted or unsubstituted C₁-C₆alkyl, or substituted or unsubstituted C₁-C₆fluoroalkyl;
or two R⁸ are taken together with the carbon atom to which they are attached to form a substituted or unsubstituted cycloalkyl or a substituted or unsubstituted heterocycloalkyl;
each R⁹ is independently hydrogen, D, halogen, —OH, substituted or unsubstituted C₁-C₆alkyl, or substituted or unsubstituted C₁-C₆fluoroalkyl.

53-126. (canceled)

127. A compound selected from the group consisting of: 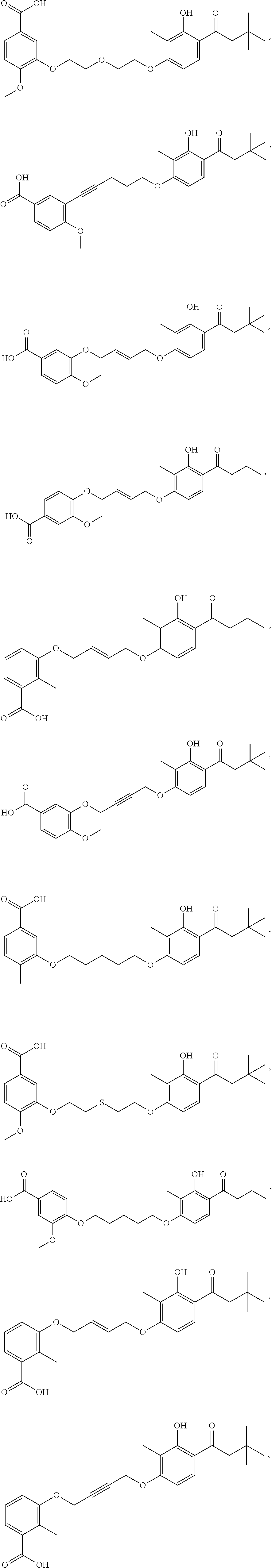 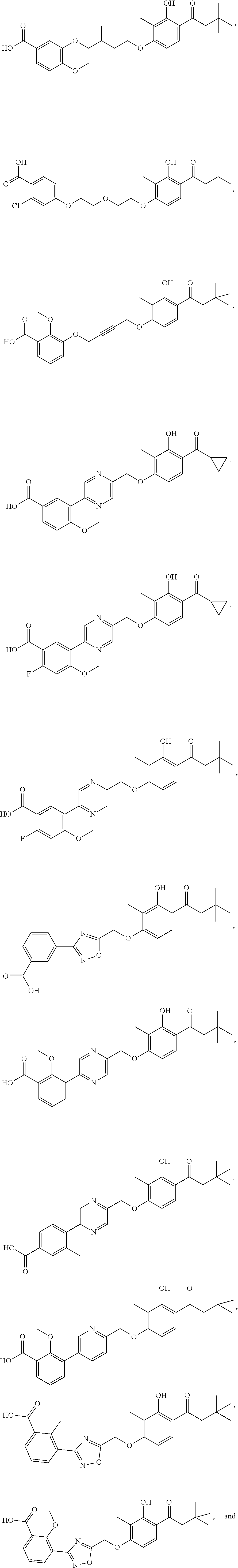 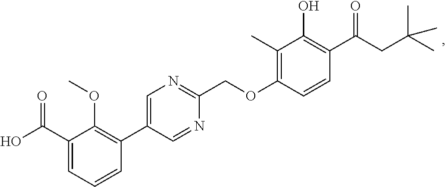 [SMILES: COc1c(C(=O)O)cccc1-c1cnc(COc2ccc(C(=O)CC(C)(C)C)c(O)c2C)nc1]
or a pharmaceutically acceptable salt or solvate thereof.

128. A pharmaceutical composition comprising a compound of claim 1, or a pharmaceutically acceptable salt, or solvate thereof, and at least one pharmaceutically acceptable excipient.

129. (canceled)

130. (canceled)

131. A method of treating a central nervous disorder (CNS) disorder, the method comprising the step of administering to a subject in need thereof, an effective amount of the compound of claim 1, thereby treating the disorder.

132.-138. (canceled)

139. A method of treating substance abuse, the method comprising the step of administering to a subject in need thereof, an effective amount of the compound of claim 1, wherein the effective amount is sufficient to diminish, inhibit or eliminate desire for and/or consumption of the substance in the subject.

140. The method of claim 139, wherein the substance is nicotine, alcohol, opiates, amphetamines, methamphetamines, or cocaine.

141. A method for treating an addictive disorder, the method comprising the steps of: a) administering to a subject in need thereof, an effective amount of the compound of claim 1, during a first time period, wherein the first time period is a time period wherein the subject expects to be in an environment wherein, or exposed to stimuli in the presence of which, the subject habitually uses an addictive substance; and b) administering an effective amount of the compound of claim 1 during a second time period, wherein the second time period is a time period wherein the subject is suffering from withdrawal.
